(19) United States
(12) Patent Application Publication
(10) Pub. No.: US 2025/0361273 A1
(43) Pub. Date: Nov. 27, 2025

(54) METHODS AND COMPOSITIONS

(71) Applicant: Flagship Pioneering Innovations VII, LLC, Cambridge, MA (US)

(72) Inventors: Soumitra Shanker GHOSH, San Diego, CA (US); Krystian Andrzej Kozek, Boston, MA (US); Tracy Anne Stone, Toronto, CA (US); Glenn Larry Butterfoss, Glen Mills, PA (US); Alexander Kitaygorodsky, Newmarket, CA (US); Ashlin Kanawaty, Toronto (CA); Mark Oliver Fingerhuth, Toronto (CA); Christopher Edward Ing, Toronto (CA)

(21) Appl. No.: 19/217,717

(22) Filed: May 23, 2025

Related U.S. Application Data
(60) Provisional application No. 63/651,487, filed on May 24, 2024.
(60) Provisional application No. 63/651,488, filed on May 24, 2024.
(60) Provisional application No. 63/651,491, filed on May 24, 2024.
(60) Provisional application No. 63/651,492, filed on May 24, 2024.
(60) Provisional application No. 63/651,493, filed on May 24, 2024.
(60) Provisional application No. 63/651,498, filed on May 24, 2024.
(60) Provisional application No. 63/726,993, filed on Dec. 2, 2024.
(60) Provisional application No. 63/726,988, filed on Dec. 2, 2024.
(60) Provisional application No. 63/726,999, filed on Dec. 2, 2024.

(51) Int. Cl. C07K 7/06; A61K 38/00; A61P 3/04

(52) U.S. Cl. CPC C07K 7/06; A61P 3/04; A61K 38/00

(57) ABSTRACT

The present disclosure relates to, inter alia, MC4R agonists and enhanced selectivity of MC4R over MC1R. Furthermore, the present disclosure relates to methods of treating metabolic disorders or diseases.

### CROSS-REFERENCE TO RELATED APPLICATIONS

This disclosure claims priority to and benefit from U.S. Provisional Patent Application Nos. 63/651,487, filed May 24, 2024, 63/651,488, filed May 24, 2024, 63/651,491, filed May 24, 2024, 63/651,492, filed May 24, 2024, 63/651,493, filed May 24, 2024, 63/651,498, filed May 24, 2024, 63/726,988, filed Dec. 2, 2024, 63/726,993, filed Dec. 2, 2024, and 63/726,999, filed Dec. 2, 2024, all of which are incorporated by reference herein in their entireties.

### FIELD

This disclosure provides compositions and methods for treating diseases and disorders related to melanocortin 4 receptor (MC4R).

### BACKGROUND

The melanocortin-4 receptor (MC4R) regulates adipose tissue formation and energy homeostasis and thus may be targeted for anti-obesity intervention. Treatments of MC4R-related diseases and disorders due to malfunctions or absence of signaling cascades entailing MC4R signaling, such as genetic and/or hypothalamic obesity, may be treated by administration of pan-melanocortin receptor activating peptides. Additionally, it is possible that overactivation of the MC4R signaling cascades in patients suffering from general obesity may provide anti-obesity benefits to those patients. While MC4R agonists have been characterized in vitro, undesirable side effects frequently appeared during clinical trials and in real world use, including, but not limited to hyperpigmentation due, it is believed, to cross-activation of other melanocortin receptors, such as MC1R.

Accordingly, there is a need for compositions and methods for treating diseases and disorders that could benefit from pharmacologically-induced melanocortin 4 receptor (MC4R) activity, preferably with reduced undesirable side effects.

### SUMMARY

In aspects and embodiments, there is provided a method of treating diseases and disorders related to melanocortin 4 receptor (MC4R), including, but not limited to, reducing undesirable side effects of cross-activation of other melanocortin receptors, such as hyperpigmentation, by administering a peptide of the present disclosure alone or in combination with a therapeutical agent.

In aspects and embodiments, there is provided a peptide comprising the amino acid sequence of formula (I):

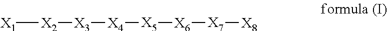

wherein in formula (I):
X₃ is 3-aminooxetane-3-carboxylic acid (Aib(O-cyclic));
X₄ is glutamine (Gln), homocitrulline (hCit), citrulline (Cit), 3-(3-pyridyl)-L-alanine (3-Pal), L-homoglutamine (hGln), histidine (His), or L-ornithine (Orn); and
X₁, X₂, X₅, X₆, X₇, and X₈ are each independently a canonical or non-canonical amino acid.

In embodiments, the peptide of formula (I) is a peptide of any one of formula (Ia): X₋₄-X₋₃-X₋₂-X₋₁-X₁-X₂-X₃-X₄-X₅-X₆-X₇-X₈, formula (Ib): X₋₃-X₋₂-X₋₁-X₁-X₂-X₃-X₄-X₅-X₆-X₇-X₈, formula (Ic): X₋₂-X₋₁-X₁-X₂-X₃-X₄-X₅-X₆-X₇-X₈, formula (Id): X₋₁-X₁-X₂-X₃-X₄-X₅-X₆-X₇-X₈, formula (Ie): X₋₂-X₋₁-X₁-X₂-X₃-X₄-X₅-X₆-X₇-X₈-X₉-X₁₀, or formula (If): X₋₁-X₁-X₂-X₃-X₄-X₅-X₆-X₇-X₈-X₉-X₁₀, wherein X₋₁, X₋₂, X₁, X₂, X₃, X₄, X₅, X₆, X₇, X₈, X₉, and X₁₀ are each independently an amino acid selected from Table 1, Table 2, Table 3, Table A1, Table AA, Table A2, and Table A2A or a linker.

In embodiments, X₄ is Gln.

In embodiments, X₄ is Cit.

In embodiments, X₄ is hCit.

In embodiments, X₄ is 3-Pal.

In embodiments, X₄ is hGln.

In embodiments, X₄ is His.

In embodiments, X₄ is Orn.

In embodiments, X₅ is selected from 4-fluoro-D-phenylalanine (D-Phe(4-F)), D-phenylalanine (D-Phe), and 4-methyl-D-phenylalanine (D-Phe(4-Me)).

In embodiments, X₅ is D-Phe(4-F).

In embodiments, X₅ is D-Phe.

In embodiments, X₅ is D-Phe(4-Me).

In embodiments, X₆ is arginine (Arg).

In embodiments, X₇ is 6-fluoro-L-tryptophan (Trp(6-F))

In embodiments, X₈ is cysteine (Cys).

In embodiments, X₈ is penicillamine (Pen).

In embodiments, X₁ is selected from D-norarginine (D-Nar) and beta-homo-L-arginine (Beta-homoArg).

In embodiments, X₁ is D-Nar.

In embodiments, X₂ is Cys.

In embodiments, the peptide of formula (I) is selected from Table 1, Table 2, Table A1, Table A1A, Table A2, and Table A2A.

In embodiments, the peptide is a cyclic peptide.

In embodiments, the cyclic peptide comprises a disulfide bridge or a lactam bridge.

In embodiments, the cyclic peptide is a peptide of formula (II):

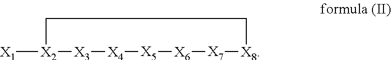

In embodiments, the cyclic peptide of formula (II) is a cyclic peptide of any one of formula (IIa)-(IIf):

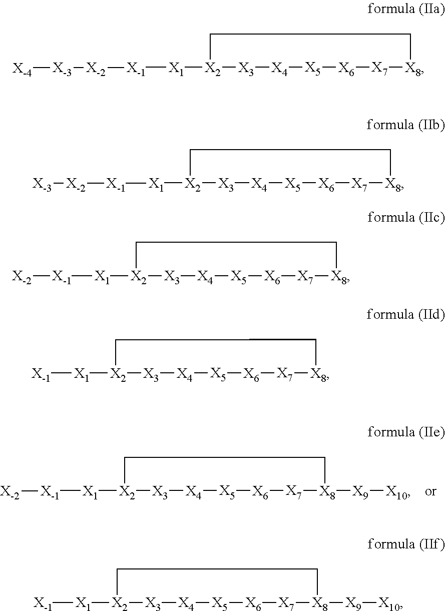

wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker.

In embodiments, the peptide further comprises one or more amino acids conjugated to X₁ and/or X₈, optionally wherein the one or more amino acids are selected from D-arginine (D-Arg), glycine (Gly), gamma-Glu peptides (e.g., gamma-glu dipeptides such as γ-Glu-Gly, γ-Glu-Ala, γ-Glu-Ser, γ-Glu-Val, γ-Glu-Thr, γ-Glu-taurine, γ-Glu-Leu, γ-Glu-Gln, γ-Glu-Lys, γ-Glu-Glu, γ-Glu-Met, γ-Glu-His, γ-Glu-Phe, γ-Glu-Arg, γ-Glu-citrulline, γ-Glu-Tyr, γ-Glu-Trp) and L-Lys(AEEAc-AEEAc-L-γ-Glu-17-carboxyheptadecanoyl) (Lys*).

In embodiments, the peptide further comprises one or more lipids conjugated to X₁ and/or X₈.

In embodiments, the peptide further comprises one or more PEG linkers conjugated to X₁ and/or X₈.

In embodiments, the peptide is capped with N-terminal acetyl and/or C-terminal amide groups.

In embodiments, the peptide is selected from Table 1, Table 2, Table A1, Table A1A, Table A2, and Table A2A.

In aspects and embodiments, there is provided a peptide of formula (III):

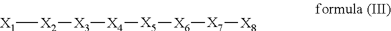

wherein in formula (III):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen).

In embodiments, the peptide of formula (III) is a cyclic peptide.

In embodiments, the cyclic peptide comprises a disulfide bridge or a lactam bridge.

In embodiments, the cyclic peptide consists of the amino acid sequence as set forth in formula (IV):

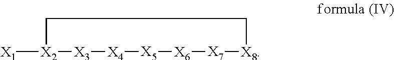

wherein in formula (IV):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen), wherein

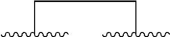 [SMILES: *CC*]

represents a disulfide bridge, and the peptide is capped with N-terminal acetyl.

In embodiments, the peptide is capped with N-terminal acetyl and/or C-terminal amide groups. In embodiments, the peptide is capped with N-terminal acetyl.

In embodiments, the disclosed peptide (e.g., peptide of formula I and/or peptide of formula III) demonstrates increased selectivity for MC4R over MC1R when administered to a subject compared to a control.

In embodiments, the peptide demonstrates increased selectivity for MC4R over MC1R as measured by an in vitro, ex vivo, or in vivo assay when compared to a control.

In embodiments, the peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

In embodiments, the peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

In embodiments, the peptide demonstrates an increased ratio of MC4R intracellular signaling to MC1R intracellular signaling when administered to a subject compared to a control.

In embodiments, the peptide demonstrates increased selectivity for MC4R intracellular signaling to MC1R intracellular signaling as measured by an in vitro, ex vivo, or in vivo assay when compared to a control.

In embodiments, the peptide demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when administered to a subject compared to a control.

In embodiments, the peptide demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when administered to a subject compared to a control.

In embodiments, the peptide demonstrates enhanced melanocortin 4 receptor (MC4R) function in a subject when compared to before the peptide is administered or to a pre-treatment or non-treatment state, or a subject treated with control.

In embodiments, the peptide demonstrates decreased melanocortin 1 receptor (MC1R) function in a subject when compared to before the peptide is administered or to a pre-treatment or non-treatment state, or a subject treated with control.

In embodiments, the peptide demonstrates enhanced melanocortin 4 receptor (MC4R) function as measured by an in vitro, ex vivo, or in vivo assay when compared to a control.

In embodiments, the peptide demonstrates decreased melanocortin 1 receptor (MC1R) function as measured by an in vitro, ex vivo, or in vivo assay when compared to a control.

In embodiments, the peptide demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

In embodiments, the peptide demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

In embodiments, the cyclic peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

In embodiments, the cyclic peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

In embodiments, the peptide comprises a half-life extending moiety, optionally wherein the half-life extending moiety comprises polyethylene glycol (PEG), recombinant PEG mimetics, glycosylation of carbohydrates, Fc-fusion proteins or conjugates, albumin fusion proteins or conjugates, albumin-binding fusion proteins or conjugates, polypropylene glycol (PPG), XTEN fusion protein or conjugates, or a combination thereof.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

In embodiments, the peptide further comprises a therapeutic, diagnostic, and/or imaging moiety, optionally wherein the therapeutic, diagnostic, and/or imaging moiety comprises a small molecule, a biological (e.g., a biopolymer, a protein, a nucleic acid, a polysaccharide), or a radionuclide.

In embodiments, the peptide of formula (I) or formula (III) is conjugated to or co-formulated with an additional therapeutic agent or therapeutic moiety.

In embodiments, the peptide of formula (II) is conjugated to or co-formulated with an additional therapeutic agent or therapeutic moiety.

In embodiments, the additional therapeutic agent or therapeutic moiety comprises an incretin, an incretin analogue, or a modulator of an incretin receptor.

In embodiments, the modulator is an agonist. In embodiments, the agonist is an agonist of GLP-1, GIP, and/or glucagon receptor. In embodiments, the agonist is a GLP-1 analogue. In embodiments, the GLP-1 analogue comprises a non-canonical amino acid. In embodiments, the GLP-1 analogue comprises tirzepatide, tirzepatide analogue, liraglutide, retatrutide, exenatide, lixisenatide, semaglutide or a semaglutide derivative.

In embodiments, the agonist is an agonist of GIP receptor. In embodiments, the agonist is a dual GLP-1-GIP receptor co-agonist. In embodiments, the agonist is a triple hormone receptor agonist. In embodiments, the agonist comprises retatrutide.

In embodiments, there is provided a protein comprising the peptide of the present disclosure.

In embodiments, the protein has a size of at least about 10 amino acid residues, or at least about 15 residues, or at least about 20 residues, or at least about 25 residues, or at least about 30 residues, or at least about 35 residues, or at least about 40 residues, or at least about 45 residues, or at least about 50 residues, or at least about 60 residues, or at least about 70 residues, or at least about 80 residues, or at least about 90 residues, or at least about 100 residues, or at least about 250 residues, or at least about 500 residues, at least about 750 residues, at least about 1,000 residues, at least about 1,250 residues, at least about 1,500 residues, at least about 1,750 residues, at least about 2,000 residues, at least about 3,000 residues, at least about 4,000 residues, or at least about 5,000 residues.

In embodiments, there is provided a nucleic acid encoding the peptide of the present disclosure, or the protein of the present disclosure.

In embodiments, there is provided a solid synthesis device conjugated to the peptide of the present disclosure, the protein of the present disclosure, or the nucleic acid of the present disclosure.

In embodiments, there is provided a pharmaceutical composition comprising a peptide of the present disclosure, or the proteins of the present disclosure, or the nucleic acid of the present disclosure and a pharmaceutically acceptable excipient or carrier.

In embodiments, the pharmaceutical composition further comprises an additional therapeutic, diagnostic, and/or imaging moiety.

In embodiments, the pharmaceutical composition is conjugated to or co-formulated with an additional therapeutic agent or therapeutic moiety.

In embodiments, the pharmaceutical composition is administered orally, parenterally, by inhalation spray, topically, rectally, nasally, buccally, sub-lingually, vaginally or via an implanted reservoir.

In embodiments, the pharmaceutical composition is administered via intravenous injection, intramuscular injection, subcutaneous injection, or depot injection.

In embodiments, there is provided a method of selectively increasing the activity of MC4R over MC1R comprising administering a peptide of the present disclosure, or the pharmaceutical composition of the present disclosure to a subject in need thereof.

In embodiments, the method prevents, reduces, or eliminates hyperpigmentation in the subject when compared to an untreated or pre-treatment subject, or subject treated with a control.

In embodiments, the hyperpigmentation is reduced by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% when compared to an untreated or pre-treatment subject, or subject treated with a control.

In embodiments, the hyperpigmentation is reduced by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold when compared to an untreated or pre-treatment subject, or subject treated with a control.

In embodiments, there is provided a method of treating a disease or disorder associated with MC4R function comprising administering a peptide of the present disclosure, or the pharmaceutical composition of the present disclosure to a subject in need thereof.

In embodiments, the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency, and Prader-Willi syndrome.

In embodiments, the method further comprises co-administration of an additional therapeutic agent.

In embodiments, the additional therapeutic agent comprises incretin, an incretin analogue, or a modulator of an incretin receptor.

In embodiments, the modulator is an agonist.

In embodiments, the agonist is an agonist of GLP-1, GIP, and/or glucagon receptor.

In embodiments, the agonist is a GLP-1 analogue.

In embodiments, the GLP-1 analogue comprises a non-canonical amino acid.

In embodiments, the GLP-1 analogue comprises tirzepatide, tirzepatide analogue, liraglutide, retatrutide, exenatide, lixisenatide, semaglutide or semaglutide derivative.

In embodiments, the subject is receiving and/or previously received treatment comprising incretin, an incretin analogue, or a modulator of an incretin receptor.

In embodiments, the subject has not or is not receiving treatment comprising incretin, an incretin analogue, or a modulator of an incretin receptor.

In embodiments, there is provided a method of treating obesity or abnormal or excessive fat accumulation comprising administering a peptide of the present disclosure, or the pharmaceutical composition of the present disclosure to a subject in need thereof.

In embodiments, there is provided a method of treating a disease or disorder associated with MC4R function comprising administering a peptide of the present disclosure, or the pharmaceutical composition of the present disclosure to a subject receiving and/or previously received incretin.

In embodiments, there is provided a method of treating a disease or disorder associated with MC4R function comprising administering a peptide of the present disclosure, or the pharmaceutical composition of the present disclosure to a subject not receiving incretin.

In embodiments, the peptide or pharmaceutical composition is administered from a prefilled dosage form device, such as syringe or autoinjector.

### BRIEF DESCRIPTION OF THE DRAWINGS

FIG. 1 shows the results of a weight loss study in mice administered with various nonlipidated peptides. The graph represents a comparison of the percentage of weight loss over three days in diet induced obese (DIO) mice with treatment of exemplary peptides.

FIG. 2 is a bar graph of the amount of food intake in DIO mice after treatment with exemplary peptides.

FIG. 3 is a graph of the amount of weight loss in DIO mice after treatment with 10 mg/kg of a MC4R agonist peptide (molecule 1158) of the disclosure dosed once daily.

FIG. 4 is a table listing various exemplary peptides and their related results when assessing the selectivity of MC4R versus MC1R as well as the bias of MC4R B-arrestin versus MC4R cAMP. When comparing the selectivity of MC4R v MC1R, larger values indicate selectivity towards MC4R. When comparing the bias of MC4R B-arrestin v MC4R cAMP, larger values indicate bias towards B-arrestin. * denotes MC4R vs MC1R selectivity having a range of 0.01 to <1.00, ** denotes MC4R vs MC1R selectivity having a range of ≥1.00 to ≤7.40, *** denotes MC4R vs MC1R selectivity of about >7.40. + denotes MC4R B-arrestin v MC4R cAMP bias having a range of >0.00 to ≤2.00, ++ denotes MC4R B-arrestin v MC4R cAMP bias having a range of 2.00 to 10.00, +++ denotes MC4R B-arrestin v MC4R cAMP bias having a value of >10.00. Grey color denotes peptides with a lipidated state. N/A denotes peptides with no data collected. IA denotes an inactive state.

### DETAILED DESCRIPTION

In aspects, the disclosure provides novel peptides useful for modulating the melanocortin 4 receptor (MC4R) useful for treating MC4R related diseases and disorders, such as, but not limited to, genetic obesity and hypothalamic obesity. In embodiments, the peptides of the disclosure have enhanced selectivity for MC4R over MCR1, and therefore may reduce or eliminate skin hyperpigmentation observed in subjects treated with non-selective MC4R agonists (e.g. setmelanotide).

The G protein-coupled receptor (GPCR) superfamily is collectively the largest family of transmembrane receptors in humans, consisting of nearly 800 distinct genes and their corresponding gene products. The general structure of GPCRs has been largely conserved through evolution and manifests as glycoproteins possessing seven transmembrane spanning segments that organize themselves in the cell's plasma membrane. As they possess both extracellular and intracellular elements, they serve as ideal transducers of stimuli across the cellular membrane. GPCRs can recognize a wide variety of extracellular stimuli, including small molecules, ions, photons, peptides, and proteins, and communicate the stimuli across an impermeable membrane barrier to the intracellular domain to effect changes in cell function. Signaling is transmitted through a cascade of protein interactions and activation events, thereby evoking changes in the levels of intracellular biochemical mediators of cell function (e.g., second messengers), resulting in the regulation of several physiological functions.

Of the GPCR superfamily, the melanocortin receptor (MCR) subfamily plays significant physiological roles. MCRs are derived from five distinct genes encoding receptors (MC1R-MC5R). MC1R governs the mammalian skin and hair color by regulating the production of melanin. MC2R is mainly located in the adrenal cortex and controls the glucocorticoids that are responsible for modulating the immune system, regulating blood sugar level, and helping trigger nerve cell signaling in the brain. MC3R has been shown to regulate accumulation of adipose tissue. MC4R is known to regulate adipose tissue formation and energy homeostasis and is believed to be a monogenic target for anti-obesity therapeutics. MC5R is involved in exocrine gland dysfunction, sebum regulation, and waxy skin fatty protection.

Treatment of genetic obesity or hypothalamic obesity due to malfunctions of signaling cascades terminating in, or including, MC4R signaling typically involves administration of pan-melanocortin receptor activating peptides and small molecule agonists. While viable agonists have been characterized in vitro, undesirable side effects frequently appeared during clinical trials.

For example, setmelanotide has demonstrated the undesired ability to generate skin hyperpigmentation in human trials and also in real world use, which is due to the molecule's activity on the MC1R receptor.

In embodiments, these undesirable hyperpigmentation side effects can be reduced or eliminated by identifying a MC4R agonist (e.g., without limitation, a peptide agonist) that is selective for MC4R over MC1R, and which also demonstrates potent binding to, and in vivo effects on MC4R. In embodiments, the peptides of the present disclosure have demonstrated increased ratios of MC4R intracellular signaling to MC1R intracellular signaling as determined by cAMP production. In embodiments, the peptides of the present disclosure, which are shown to be selective for MC4R over MC1R, demonstrate that MC4R agonism is a viable route to weight loss for a genetic, hypothalamic, and/or general obese population while reducing or eliminating a major unwanted side-effect of known MC4R agonists (i.e., hyperpigmentation) that could otherwise cause patients to reject or avoid treatment with a melanocortin agonist.

In embodiments, MC4R variants can be weight-reducing as opposed to weight-inducing. While insufficiency of MC4R activity leads to weight gain, MC4R gene sequence variants that promote signaling preferentially through the B-arrestin pathway during MC4R activation have been demonstrated to lead to a reduction in weight in humans. In embodiments, the peptide or pharmaceutical composition of the present disclosure preferentially signals through the B-arrestin pathway, as compared to the canonical cAMP pathway, and can induce weight loss in a general-obesity population.

In aspects and embodiments, the disclosure provides macrocyclic peptides containing non-canonical amino acid residues, where such macrocyclic peptides are potent and selective agonists of the genetically-validated, obesity-driving, G protein-coupled MC4R receptor. In embodiments, the peptides of the present disclosure are functionally selective for MC4R as compared to the MC1R. Without being bound to a particular theory, selections of two adjacent amino acids in the sequence within the ring of the macrocycle in positions X₃ and X₄, were found to be important to a significant loss of MC1R functional activity. In embodiments, the peptides described herein comprise multiple pairs of X₃ and X₄ that result in increased MC4R selectivity. In embodiments, while at times selectivity within a molecular class is achieved at the expense of some efficacy on the main target, the peptides of the present disclosure retained robust MC4R functional activity (cAMP production, and B-arrestin signaling). In embodiments, the peptides of the present disclosure are efficacious as weight-reducing agents.

### Peptides

In embodiments, the disclosure provides selective melanocortin 4 receptor (MC4R) agonist peptides, and corresponding methods of making and using the same, e.g., without limitation, for the treating and/or prevention of diseases or disorders associated with upregulation of MC4R. In embodiments, the peptides described herein, demonstrate enhanced MC4R function. In embodiments, the peptides described herein are MC4R agonistic peptides that display superior selectivity towards MC4R as compared with the other melanocortin receptors (such as MC1R). In embodiments, the peptides described herein display varying activity on G-protein coupled pathways stemming from the MC4R, namely one or more of Gs-coupled (e.g, cAMP), Gq-coupled, and B-arrestin dependent signaling pathways. In embodiments, the peptides of the present disclosure have increased in vitro selectivity and potency, in vivo effectiveness, pharmacokinetic attributes, and/or stability when compared to other melanocortin receptor binding peptides.

In aspects and embodiments, there is provided a peptide comprising the amino acid sequence of formula (I):

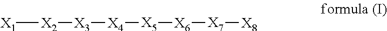

wherein in formula (I):
X₃ is 3-aminooxetane-3-carboxylic acid (Aib(O-cyclic));
X₄ is glutamine (Gln), homocitrulline (hCit), citrulline (Cit), 3-(3-pyridyl)-L-alanine (3-Pal), L-homoglutamine (hGln), histidine (His), or L-ornithine (Orn); and
X₁, X₂, X₅, X₆, X₇, and X₈ are each independently a canonical or non-canonical amino acid.

In aspects and embodiments, there is provided a peptide comprising the amino acid sequence of formula (I):

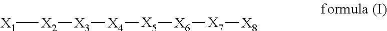

wherein in formula (I): X₁, X₂, X₃, X₄, X₅, X₆, X₇, and X₈ are each independently an amino acid selected from Table 1.

In aspects and embodiments, there is provided a peptide comprising the amino acid sequence of formula (I):

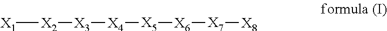

wherein in formula (I): X₁, X₂, X₃, X₄, X₅, X₆, X₇, and X₈ are each independently an amino acid selected from Table 1, Table 2, Table 3, Table A1, Table A1A, Table A2, and Table A2A.

In embodiments, the peptide consists of the amino acid sequence as set forth in formula (I).

In embodiments, the peptides of Table 1, Table 2, Table 3, Table A1, Table A1A, Table A2, and Table A2A are not limited to N-terminal functional group, C-terminal functional group, and/or status or type of cyclic function.

In embodiments, the peptide is a cyclic peptide.

In embodiments, the cyclic peptide comprises a disulfide bridge or a lactam bridge.

In embodiments, the cyclic peptide has the formula (II):

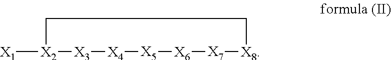

In embodiments, the cyclic peptide of formula (II) is a cyclic peptide of any one of:

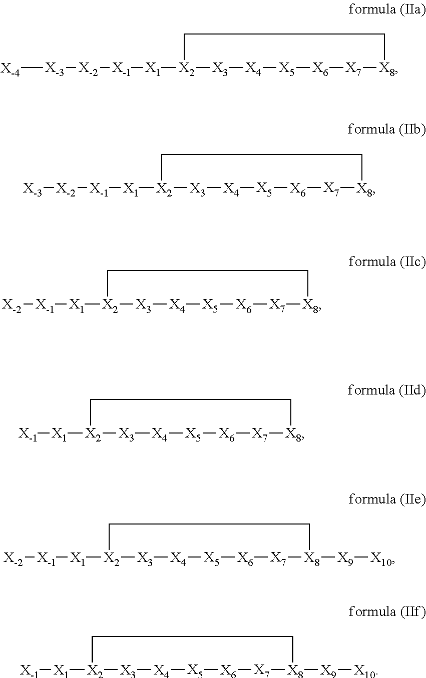

wherein X₋₁, X₋₂, X₋₃, X₋₄, X₁, X₂, X₃, X₄, X₅, X₆, X₇, X₈, X₉, and X₁₀ are each independently an amino acid selected from Table 1 and Table 2.

In embodiments, the cyclic peptide of formula (II) is a cyclic peptide of any one of formula (IIa), formula (IIb), formula (IIc), formula (IId), formula (IIe), or formula (IIf), wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3.

In embodiments, the cyclic peptide of formula (II) is a cyclic peptide of any one of formula (IIa), formula (IIb), formula (IIc), formula (IId), formula (IIe), or formula (IIf), wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide consists of the amino acid sequence as set forth in formula (II). In embodiments, the peptide consists of the amino acid sequence as set forth in formula (IIa). In embodiments, the peptide consists of the amino acid sequence as set forth in formula (IIb). In embodiments, the peptide consists of the amino acid sequence as set forth in formula (IIc). In embodiments, the peptide consists of the amino acid sequence as set forth in formula (IId). In embodiments, the peptide consists of the amino acid sequence as set forth in formula (IIe). In embodiments, the peptide consists of the amino acid sequence as set forth in formula (IIf).

In embodiments, the cyclic peptide has a formula selected from formula (BII), formula (CII), formula (DII), formula (EII), and formula (FII).

In embodiments, the cyclic peptide of formula (II) is a cyclic peptide of any one of formula (IIa), formula (IIb), formula (IIc), formula (IId), formula (IIe), or formula (IIf).

In embodiments, the cyclic peptide of formula (BII) is a cyclic peptide of any one of formula (BIIa), formula (BIIb), formula (BIIc), formula (BIId), formula (BIIe), or formula (BIIf).

In embodiments, the cyclic peptide of formula (CII) is a cyclic peptide of any one of formula (CIIa), formula (CIIb), formula (CIIc), formula (CIId), formula (CIIe), or formula (CIIf).

In embodiments, the cyclic peptide of formula (DII) is a cyclic peptide of any one of formula (DIIa), formula (DIIb), formula (DIIc), formula (DIId), formula (IIe), or formula (DIIf).

In embodiments, the cyclic peptide of formula (EII) is a cyclic peptide of any one of formula (EIIa), formula (EIIb), formula (EIIc), formula (EIId), formula (EIIe), or formula (EIIf).

In embodiments, the cyclic peptide of formula (FII) is a cyclic peptide of any one of formula (FIIa), formula (FIIb), formula (FIIc), formula (FIId), formula (FIIe), or formula (IFIf).

In embodiments, the peptide consists of the amino acid sequence as set forth in formula (BII). In embodiments, the peptide consists of the amino acid sequence as set forth in formula (BIIa). In embodiments, the peptide consists of the amino acid sequence as set forth in formula (BIIb). In embodiments, the peptide consists of the amino acid sequence as set forth in formula (BIIc). In embodiments, the peptide consists of the amino acid sequence as set forth in formula (BIId). In embodiments, the peptide consists of the amino acid sequence as set forth in formula (BIIe). In embodiments, the peptide consists of the amino acid sequence as set forth in formula (BIIf).

In embodiments, the peptide consists of the amino acid sequence as set forth in formula (CII). In embodiments, the peptide consists of the amino acid sequence as set forth in formula (CIIa). In embodiments, the peptide consists of the amino acid sequence as set forth in formula (CIIb). In embodiments, the peptide consists of the amino acid sequence as set forth in formula (CIIc). In embodiments, the peptide consists of the amino acid sequence as set forth in formula (CIId). In embodiments, the peptide consists of the amino acid sequence as set forth in formula (CIIe). In embodiments, the peptide consists of the amino acid sequence as set forth in formula (CIIf).

In embodiments, the peptide consists of the amino acid sequence as set forth in formula (DII). In embodiments, the peptide consists of the amino acid sequence as set forth in formula (DIIa). In embodiments, the peptide consists of the amino acid sequence as set forth in formula (DIIb). In embodiments, the peptide consists of the amino acid sequence as set forth in formula (DIIc). In embodiments, the peptide consists of the amino acid sequence as set forth in formula (DIId). In embodiments, the peptide consists of the amino acid sequence as set forth in formula (DIIe). In embodiments, the peptide consists of the amino acid sequence as set forth in formula (DIIf).

In embodiments, the peptide consists of the amino acid sequence as set forth in formula (EII). In embodiments, the peptide consists of the amino acid sequence as set forth in formula (EIIa). In embodiments, the peptide consists of the amino acid sequence as set forth in formula (EIIb). In embodiments, the peptide consists of the amino acid sequence as set forth in formula (EIIc). In embodiments, the peptide consists of the amino acid sequence as set forth in formula (EIId). In embodiments, the peptide consists of the amino acid sequence as set forth in formula (EIIe). In embodiments, the peptide consists of the amino acid sequence as set forth in formula (EIIf).

In embodiments, the peptide consists of the amino acid sequence as set forth in formula (FII). In embodiments, the peptide consists of the amino acid sequence as set forth in formula (FIIa). In embodiments, the peptide consists of the amino acid sequence as set forth in formula (FIIb). In embodiments, the peptide consists of the amino acid sequence as set forth in formula (FIIc). In embodiments, the peptide consists of the amino acid sequence as set forth in formula (FIId). In embodiments, the peptide consists of the amino acid sequence as set forth in formula (FIIe). In embodiments, the peptide consists of the amino acid sequence as set forth in formula (FIIf).

In embodiments, in formula (BIIa), formula (BIIb), formula (BIIc), formula (BIId), formula (BIIe), or formula (BIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, in formula (CIIa), formula (CIIb), formula (CIIc), formula (CIId), formula (CIIe), or formula (CIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, in formula (DIIa), formula (DIIb), formula (DIIc), formula (DIId), formula (DIIe), or formula (DIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, in formula (EIIa), formula (EIIb), formula (EIIc), formula (EIId), formula (EIIe), or formula (EIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, in formula (FIIa), formula (FIIb), formula (FIIc), formula (FIId), formula (FIIe), or formula (FIIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (II) is selected from Table 2, Table A2 and Table A2A.

In embodiments, the peptide of formula (I) or formula (II) is selected from Table 1, Table 2, Table A1, Table A1A, Table A2, and Table A2A.

In embodiments, the peptides of formula (I) or formula (II) comprise a N-terminal functional group, a C-terminal functional group, and/or a cyclic function.

In embodiments, the peptide of formula (I) or formula (II) is selected from Table 1, Table 2, Table A1, Table A1A, Table A2, and Table A2A, wherein the N-terminal, C-terminal and/or cyclic structure are optional features.

In embodiments, the peptide is a cyclic peptide (e.g., Table 1). In embodiments, the cyclic peptide comprises a disulfide bridge or a lactam bridge.

In embodiments, the peptide of formula (I) or formula (II) is lipidated (e.g., Table 2).

In embodiments, the peptide of formula (I) is selected from Table A1, Table A1A, Table A2 and Table A2A.

In embodiments, the peptides of Table 1, Table 2, Table A1, Table A1A, Table A2 and Table A2A are not limited to N-terminal functional group, C-terminal functional group, and/or status or type of cyclic function.

TABLE A1
Exemplary peptides.

| Molecule | | | | | | | | |
|---|---|---|---|---|---|---|---|---|
| Name | X₁ | X₂ | X₃ | X₄ | X₅ | X₆ | X₇ | X₈ |
| 1093-1 | D-Nar | Cys | Aib(O- | Gln | D-Phe(4-F) | Arg | Trp(6-F) | Cys |
| | | | cyclic) | | | | | |
| 1092-1 | D-Nar | Cys | Aib(O- | Gln | D-Phe | Arg | Trp(6-F) | Cys |
| | | | cyclic) | | | | | |
| 1107-1 | D-Nar | Cys | Aib(O- | hCit | D-Phe(4-F) | Arg | Trp(6-F) | Cys |
| | | | cyclic) | | | | | |
| 1106-1 | D-Nar | Cys | Aib(O- | Cit | D-Phe(4-F) | Arg | Trp(6-F) | Cys |
| | | | cyclic) | | | | | |
| 1103-1 | D-Nar | Cys | Aib(O- | Cit | D-Phe | Arg | Trp(6-F) | Cys |
| | | | cyclic) | | | | | |
| 1105-1 | D-Nar | Cys | Aib(O- | Cit | D-Phe(4-Me) | Arg | Trp(6-F) | Cys |
| | | | cyclic) | | | | | |
| 1095-1 | D-Nar | Cys | Aib(O- | Gln | D-Phe(4-Me) | Arg | Trp(6-F) | Cys |
| | | | cyclic) | | | | | |
| 1122 -1 | D-Nar | Cys | Aib(O- | 3-Pal | D-Phe(4-F) | Arg | Trp(6-F) | Cys |
| | | | cyclic) | | | | | |
| 1102-1 | D-Nar | Cys | Aib(O- | hGln | D-Phe | Arg | Trp(6-F) | Cys |
| | | | cyclic) | | | | | |
| 1058-1 | D-Nar | Cys | Aib(O- | His | D-Phe | Arg | Trp(6-F) | Cys |
| | | | cyclic) | | | | | |
| 1123-1 | D-Nar | Cys | Aib(O- | Orn | D-Phe(4-F) | Arg | Trp(6-F) | Cys |
| | | | cyclic) | | | | | |
| 1158-1 | D-Nar | Cys | Aib(O- | Gln | D-Phe(4-F) | Arg | Trp(6-F) | Pen |
| | | | cyclic) | | | | | |

In embodiments, the peptide of formula (I) is selected from Table B1, Table B1A, Table B2 and Table B2A.

In embodiments, the peptides of Table B1, Table B1A, Table B2 and Table B32A are not limited to N-terminal functional group, C-terminal functional group, and/or status or type of cyclic function.

TABLE B1
Exemplary peptide.

| Molecule | | | | | | | | |
|---|---|---|---|---|---|---|---|---|
| Name | X₁ | X₂ | X₃ | X₄ | X₅ | X₆ | X₇ | X₈ |
| 1119-1 | D-Nar | Cys | Phg | 3-Pal | D-Phe(4-F) | Arg | Trp(6-F) | Cys |

In embodiments, the peptide of formula (I) is selected from Table B1A.

In embodiments, the peptide of formula (I) is selected from Table B1A, wherein the N-terminal, C-terminal and/or cyclic structure are optional feature.

TABLE B1A
Exemplary peptide with N-terminal, C-terminal and/or cyclic structure as optional feature.

| Molecule | | | | | | | | | | | |
|---|---|---|---|---|---|---|---|---|---|---|---|
| Name | X₁ | X₂ | X₃ | X₄ | X₅ | X₆ | X₇ | X₈ | N-term | C-term | Cyclic |
| 1119 | D-Nar | Cys | Phg | 3-Pal | D-Phe(4-F) | Arg | Trp(6-F) | Cys | Ac | NH₂ | Disulfide |

In embodiments, the peptide of formula (I) is selected from Table B2.

TABLE B2
Exemplary lipidated peptides.

| Molecule | | | | | | | | | | | | |
|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ |
| 1146-2 | Lys* | D-Arg | Gly | D-Arg | D-Nar | Cys | Phg | 3-Pal | D-Phe(4-F) | Arg | Trp(6-F) | Cys |
| 1139-2 | | Lys* | Gly | D-Arg | D-Nar | Cys | Phg | 3-Pal | D-Phe(4-F) | Arg | Trp(6-F) | Cys |
| 1145-2 | | Lys* | PEG1 | PEG1 | D-Nar | Cys | Phg | 3-Pal | D-Phe(4-F) | Arg | Trp(6-F) | Cys |
| 1147-2 | Lys* | D-Arg | PEG1 | D-Arg | Beta- | Cys | Phg | 3-Pal | D-Phe(4-F) | Arg | Trp(6-F) | Cys |
| | | | | | homoArg | | | | | | | |

In embodiments, the peptide of formula (II) is selected from Table B2A.

In embodiments, the peptide of formula (II) is selected from Table B2A, wherein the N-terminal, C-terminal and/or cyclic structure are optional feature.

TABLE B2A
Exemplary lipidated peptides.

| Molecule | | | | | | | | | | | | | | | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | N-term | C-term | Cyclic |
| 1146 | Lys* | D-Arg | Gly | D-Arg | D-Nar | Cys | Phg | 3-Pal | D-Phe(4-F) | Arg | Trp(6-F) | Cys | Ac | NH₂ | Disulfide |
| 1139 | | Lys* | Gly | D-Arg | D-Nar | Cys | Phg | 3-Pal | D-Phe(4-F) | Arg | Trp(6-F) | Cys | Ac | NH₂ | Disulfide |
| 1145 | | Lys* | PEG1 | PEG1 | D-Nar | Cys | Phg | 3-Pal | D-Phe(4-F) | Arg | Trp(6-F) | Cys | Ac | NH₂ | Disulfide |
| 1147 | Lys* | D-Arg | PEG1 | D-Arg | Beta- | Cys | Phg | 3-Pal | D-Phe(4-F) | Arg | Trp(6-F) | Cys | Ac | NH₂ | Disulfide |
| | | | | | homoArg | | | | | | | | | | |

In embodiments, the peptide of formula (I) is selected from Table C1, Table C1A, Table C2 and Table C2A.

In embodiments, the peptides of Table C1, Table C1A, Table C2 and Table C2A are not limited to N-terminal functional group, C-terminal functional group, and/or status or type of cyclic function.

TABLE C1
Exemplary peptide.

| Molecule | | | | | | | | |
|---|---|---|---|---|---|---|---|---|
| Name | X₁ | X₂ | X₃ | X₄ | X₅ | X₆ | X₇ | X₈ |
| 1094-1 | D-Nar | Cys | D- | Gln | D-Phe | Arg | Trp(6-F) | Cys |
| | | | aMeOrn | | | | | |

In embodiments, the peptide of formula (I) is selected from Table C1A.

In embodiments, the peptide of formula (I) is selected from Table C1A, wherein the N-terminal, C-terminal and/or cyclic structure are optional feature.

TABLE C1A
Exemplary peptides.

| Molecule | | | | | | | | | | | |
|---|---|---|---|---|---|---|---|---|---|---|---|
| Name | X₁ | X₂ | X₃ | X₄ | X₅ | X₆ | X₇ | X₈ | N-term | C-term | Cyclic |
| 1094 | D-Nar | Cys | D- | Gln | D-Phe | Arg | Trp(6-F) | Cys | Ac | NH₂ | Disulfide |
| | | | aMeOrn | | | | | | | | |

In embodiments, the peptide of formula (II) is selected from Table C2.

TABLE C2
Exemplary lipidated peptides

| Molecule | | | | | | | | | | | | |
|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ |
| 1148-2 | Lys* | D-Arg | Gly | D-Arg | D-Nar | Cys | D- | Gln | D-Phe | Arg | Trp(6-F) | Cys |
| | | | | | | | aMeOrn | | | | | |
| 1149-2 | | Lys* | Gly | D-Arg | D-Nar | Cys | D- | Gln | D-Phe | Arg | Trp(6-F) | Cys |
| | | | | | | | aMeOrn | | | | | |
| 1137-2 | | Lys* | PEG1 | PEG1 | D-Nar | Cys | D- | Gln | D-Phe | Arg | Trp(6-F) | Cys |
| | | | | | | | aMeOrn | | | | | |
| 1136-2 | Lys* | D-Arg | PEG1 | D-Arg | Beta- | Cys | D- | Gln | D-Phe | Arg | Trp(6-F) | Cys |
| | | | | | homoArg | | aMeOrn | | | | | |

In embodiments, the peptide of formula (II) is selected from Table C2A.

In embodiments, the peptide of formula (II) is selected from Table C2A, wherein the N-terminal, C-terminal and/or cyclic structure are optional feature.

TABLE C2A
Exemplary lipidated peptides

| Molecule | | | | | | | | | | | | | | | |
|---|---|---|---|---|---|---|---|---|---|---|---|---|---|---|---|
| Name | X-4 | X-3 | X-2 | X-1 | X1 | X2 | X3 | X4 | X5 | X6 | X7 | X8 | N-term | C-term | Cyclic |
| 1148 | Lys* | D-Arg | Gly | D-Arg | D-Nar | Cys | D- | Gln | D-Phe | Arg | Trp(6-F) | Cys | Ac | NH₂ | Disulfide |
| | | | | | | | aMeOrn | | | | | | | | |
| 1149 | | Lys* | Gly | D-Arg | D-Nar | Cys | D- | Gln | D-Phe | Arg | Trp(6-F) | Cys | Ac | NH₂ | Disulfide |
| | | | | | | | aMeOrn | | | | | | | | |
| 1137 | | Lys* | PEG1 | PEG1 | D-Nar | Cys | D- | Gln | D-Phe | Arg | Trp(6-F) | Cys | Ac | NH₂ | Disulfide |
| | | | | | | | aMeOrn | | | | | | | | |
| 1136 | Lys* | D-Arg | PEG1 | D-Arg | Beta- | Cys | D- | Gln | D-Phe | Arg | Trp(6-F) | Cys | Ac | NH₂ | Disulfide |
| | | | | | homoArg | | aMeOrn | | | | | | | | |

In embodiments, the peptides of Table D1, Table D1A, Table D2 and Table D2A are not limited to N-terminal functional group, C-terminal functional group, and/or status or type of cyclic function.

In embodiments, the peptide of formula (I) is selected from Table D1.

TABLE D1
Exemplary peptides.

| Molecule | | | | | | | | |
|---|---|---|---|---|---|---|---|---|
| Name | X₁ | X₂ | X₃ | X₄ | X₅ | X₆ | X₇ | X₈ |
| 1119-1 | D-Nar | Cys | Phg | 3-Pal | D-Phe(4-F) | Arg | Trp(6-F) | Cys |
| 1094-1 | D-Nar | Cys | D-aMeOrn | Gln | D-Phe | Arg | Trp(6-F) | Cys |
| 1093-1 | D-Nar | Cys | Aib(O-cyclic) | Gln | D-Phe(4-F) | Arg | Trp(6-F) | Cys |
| 1092-1 | D-Nar | Cys | Aib(O-cyclic) | Gln | D-Phe | Arg | Trp(6-F) | Cys |
| 1107-1 | D-Nar | Cys | Aib(O-cyclic) | hCit | D-Phe(4-F) | Arg | Trp(6-F) | Cys |
| 1106-1 | D-Nar | Cys | Aib(O-cyclic) | Cit | D-Phe(4-F) | Arg | Trp(6-F) | Cys |
| 1015-1 | D-Nar | Cys | L-aMeGlu | His | D-Phe | Arg | Trp(6-Me) | Cys |
| 1035-1 | D-Nar | Cys | L-aMeGlu | His | D-Phe | Arg | Trp(6-F) | Cys |
| 1091-1 | D-Nar | Cys | hGlu | Gln | D-Phe | Arg | Trp(6-F) | Cys |
| 1096-1 | D-Nar | Cys | D-aMeSer | Gln | D-Phe | Arg | Trp(6-F) | Cys |
| 1043-1 | D-Nar | Cys | L-aMeGlu | His | D-Phe | Arg | Trp(5-Me) | Cys |
| 1012-1 | Arg | Cys | Cyclo-Leu | Gln | D-Phe | Arg | Trp | Cys |
| 1049-1 | D-Nar | Cys | hGlu | His | D-Phe | Arg | Trp(6-Me) | Cys |
| 1041-1 | D-Nar | Cys | L-aMeGlu | His | D-Phe(4-F) | Arg | Trp(6-Me) | Cys |
| 1099-1 | D-Nar | Cys | Ala(2-Me) | Gln | D-Phe(4-F) | Arg | Trp(6-F) | Cys |
| 1030-1 | Beta- | Cys | L-aMeGlu | His | D-Phe(3-CF3) | Arg | Trp | Cys |
| | homoArg | | | | | | | |
| 1121-1 | D-Nar | Glu | L-aMeAsp | His | D-Phe(4-F) | Arg | Trp(6-F) | Dap |
| 1042-1 | D-Nar | Cys | L-aMeAsp | His | D-Phe | Arg | Trp(6-F) | Cys |
| 1024-1 | Beta- | Cys | L-aMeGlu | His | D-Phe | Arg | Trp(6-Me) | Cys |
| | homoArg | | | | | | | |
| 1064-1 | D-Nar | Cys | L-aMeAsp | His | D-Phe | Arg | Trp(6-Me) | Cys |
| 1037-1 | D-Nar | Cys | Ala(2-Me) | His | D-Phe | Arg | Trp(6-F) | Cys |
| 1019-1 | D-Nar | Cys | L-aMeGlu | His | D-Phe | Arg | Trp | Cys |
| 1085-1 | D-Nar | Cys | L-aMeGlu | His | D-Phe(3-F) | Arg | Trp(6-Me) | Cys |
| 1016-1 | D-Arg | Cys | L-aMeGlu | His | D-Phe | Arg | Trp(6-Me) | Cys |
| 1111-1 | D-Nar | Cys | Phg | His | D-Phe(4-F) | Arg | Trp(6-F) | Cys |
| 1108-1 | D-Nar | Cys | Cyclo-Leu | 3-Pal | D-Phe | Arg | Trp(6-F) | Cys |
| 1050-1 | D-Nar | Cys | L-aMeGlu | His | D-Phe(3-CF3) | Arg | Trp(6-Me) | Cys |
| 1044-1 | D-Nar | Cys | D-bhGlu | His | D-Phe | Arg | Trp(6-Me) | Cys |
| 1040-1 | D-Nar | Cys | L-aMeGlu | His | D-Phe(3-F) | Arg | Trp(6-F) | Cys |
| 1039-1 | D-Nar | Cys | D-aMeSer | His | D-Phe | Arg | Trp(6-Me) | Cys |
| 1033-1 | Beta- | Cys | bhGlu | His | D-Phe | Arg | Trp | Cys |
| | homoArg | | | | | | | |
| 1013-1 | Arg | Cys | Ala(2-Me) | Gln | D-Phe | Arg | Trp | Cys |
| 1124-1 | D-Nar | Cys | D-aMeOrn | 3-Pal | D-Phe(4-F) | Arg | Trp(6-F) | Cys |
| 1122-1 | D-Nar | Cys | Aib(O-cyclic) | 3-Pal | D-Phe(4-F) | Arg | Trp(6-F) | Cys |
| 1126-1 | D-Nar | Cys | D-aMeOrn | 3-Pal | D-Phe | Arg | Trp(6-F) | Cys |
| 1158-1 | D-Nar | Cys | Aib(O-cyclic) | Gln | D-Phe(4-F) | Arg | Trp(6-F) | Pen |

In embodiments, the peptide of formula (I) is selected from Table D1A.

In embodiments, the peptide of formula (I) is selected from Table D1A, wherein the N-terminal, C-terminal and/or cyclic structure are optional feature.

TABLE D1A
Exemplary peptides

| Molecule | | | | | | | | | | | |
|---|---|---|---|---|---|---|---|---|---|---|---|
| Name | X₁ | X₂ | X₃ | X₄ | X₅ | X₆ | X₇ | X₈ | N-term | C-term | Cyclic |
| 1119 | D-Nar | Cys | Phg | 3-Pal | D-Phe(4-F) | Arg | Trp(6-F) | Cys | Ac | NH₂ | Disulfide |
| 1094 | D-Nar | Cys | D- | Gln | D-Phe | Arg | Trp(6-F) | Cys | Ac | NH₂ | Disulfide |
| | | | aMeOrn | | | | | | | | |
| 1093 | D-Nar | Cys | Aib(O- | Gln | D-Phe(4-F) | Arg | Trp(6-F) | Cys | Ac | NH₂ | Disulfide |
| | | | cyclic) | | | | | | | | |
| 1092 | D-Nar | Cys | Aib(O- | Gln | D-Phe | Arg | Trp(6-F) | Cys | Ac | NH₂ | Disulfide |
| | | | cyclic) | | | | | | | | |
| 1107 | D-Nar | Cys | Aib(O- | hCit | D-Phe(4-F) | Arg | Trp(6-F) | Cys | Ac | NH₂ | Disulfide |
| | | | cyclic) | | | | | | | | |
| 1106 | D-Nar | Cys | Aib(O- | Cit | D-Phe(4-F) | Arg | Trp(6-F) | Cys | Ac | NH₂ | Disulfide |
| | | | cyclic) | | | | | | | | |
| 1015 | D-Nar | Cys | L- | His | D-Phe | Arg | Trp(6-Me) | Cys | Ac | NH₂ | Disulfide |
| | | | aMeGlu | | | | | | | | |
| 1035 | D-Nar | Cys | L- | His | D-Phe | Arg | Trp(6-F) | Cys | Ac | NH | Disulfide |
| | | | aMeGlu | | | | | | | | |
| 1091 | D-Nar | Cys | hGlu | Gln | D-Phe | Arg | Trp(6-F) | Cys | Ac | NH₂ | Disulfide |
| 1096 | D-Nar | Cys | D- | Gln | D-Phe | Arg | Trp(6-F) | Cys | Ac | NH₂ | Disulfide |
| | | | aMeSer | | | | | | | | |
| 1043 | D-Nar | Cys | L- | His | D-Phe | Arg | Trp(5-Me) | Cys | Ac | NH₂ | Disulfide |
| | | | aMeGlu | | | | | | | | |
| 1012 | Arg | Cys | Cyclo- | Gln | D-Phe | Arg | Trp | Cys | Ac | NH₂ | Disulfide |
| | | | Leu | | | | | | | | |
| 1049 | D-Nar | Cys | hGlu | His | D-Phe | Arg | Trp(6-Me) | Cys | Ac | NH₂ | Disulfide |
| 1041 | D-Nar | Cys | L- | His | D-Phe(4-F) | Arg | Trp(6-Me) | Cys | Ac | NH₂ | Disulfide |
| | | | aMeGlu | | | | | | | | |
| 1099 | D-Nar | Cys | Ala(2-Me) | Gln | D-Phe(4-F) | Arg | Trp(6-F) | Cys | Ac | NH₂ | Disulfide |
| 1030 | Beta- | Cys | L- | His | D-Phe(3-CF3) | Arg | Trp | Cys | Ac | NH₂ | Disulfide |
| | homoArg | | aMeGlu | | | | | | | | |
| 1121 | D-Nar | Glu | L- | His | D-Phe(4-F) | Arg | Trp(6-F) | Dap | Ac | NH₂ | Lactam |
| | | | aMeAsp | | | | | | | | |
| 1042 | D-Nar | Cys | L- | His | D-Phe | Arg | Trp(6-F) | Cys | Ac | NH₂ | Disulfide |
| | | | aMeAsp | | | | | | | | |
| 1024 | Beta- | Cys | L- | His | D-Phe | Arg | Trp(6-Me) | Cys | Ac | NH₂ | Disulfide |
| | homoArg | | aMeGlu | | | | | | | | |
| 1064 | D-Nar | Cys | L- | His | D-Phe | Arg | Trp(6-Me) | Cys | Ac | NH₂ | Disulfide |
| | | | aMeAsp | | | | | | | | |
| 1037 | D-Nar | Cys | Ala(2-Me) | His | D-Phe | Arg | Trp(6-F) | Cys | Ac | NH₂ | Disulfide |
| 1019 | D-Nar | Cys | L- | His | D-Phe | Arg | Trp | Cys | Ac | NH₂ | Disulfide |
| | | | aMeGlu | | | | | | | | |
| 1085 | D-Nar | Cys | L- | His | D-Phe(3-F) | Arg | Trp(6-Me) | Cys | Ac | NH₂ | Disulfide |
| | | | aMeGlu | | | | | | | | |
| 1016 | D-Arg | Cys | L- | His | D-Phe | Arg | Trp(6-Me) | Cys | Ac | NH₂ | Disulfide |
| | | | aMeGlu | | | | | | | | |
| 1111 | D-Nar | Cys | Phg | His | D-Phe(4-F) | Arg | Trp(6-F) | Cys | Ac | NH₂ | Disulfide |
| 1108 | D-Nar | Cys | Cyclo- | 3-Pal | D-Phe | Arg | Trp(6-F) | Cys | Ac | NH₂ | Disulfide |
| | | | Leu | | | | | | | | |
| 1050 | D-Nar | Cys | L- | His | D-Phe(3-CF3) | Arg | Trp(6-Me) | Cys | Ac | NH₂ | Disulfide |
| | | | aMeGlu | | | | | | | | |
| 1044 | D-Nar | Cys | D-bhGlu | His | D-Phe | Arg | Trp(6-Me) | Cys | Ac | NH₂ | Disulfide |
| 1040 | D-Nar | Cys | L- | His | D-Phe(3-F) | Arg | Trp(6-F) | Cys | Ac | NH₂ | Disulfide |
| | | | aMeGlu | | | | | | | | |
| 1039 | D-Nar | Cys | D- | His | D-Phe | Arg | Trp(6-Me) | Cys | Ac | NH₂ | Disulfide |
| | | | aMeSer | | | | | | | | |
| 1033 | Beta- | Cys | bhGlu | His | D-Phe | Arg | Trp | Cys | Ac | NH₂ | Disulfide |
| | homoArg | | | | | | | | | | |
| 1013 | Arg | Cys | Ala(2-Me) | Gln | D-Phe | Arg | Trp | Cys | Ac | NH₂ | Disulfide |
| 1124 | D-Nar | Cys | D- | 3-Pal | D-Phe(4-F) | Arg | Trp(6-F) | Cys | Ac | NH₂ | Disulfide |
| | | | aMeOrn | | | | | | | | |
| 1122 | D-Nar | Cys | Aib(O- | 3-Pal | D-Phe(4-F) | Arg | Trp(6-F) | Cys | Ac | NH₂ | Disulfide |
| | | | cyclic) | | | | | | | | |
| 1126 | D-Nar | Cys | D- | 3-Pal | D-Phe | Arg | Trp(6-F) | Cys | Ac | NH₂ | Disulfide |
| | | | aMeOrn | | | | | | | | |
| 1158 | D-Nar | Cys | Aib(O- | Gln | D-Phe(4-F) | Arg | Trp(6-F) | Pen | Ac | NH₂ | Disulfide |
| | | | cyclic) | | | | | | | | |

In embodiments, the peptide of formula (II) is selected from Table D2.

TABLE D2
Exemplary lapidated peptides.

| Molecule | | | | | | | | | | | | | | | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₉ | X₉ | X₁₀ | X₁₁ |
| 1129-2 | | Lys* | Gly | D-Arg | D-Nar | Cys | L- | His | D-Phe | Arg | Trp(6-Me) | Cys | | | |
| | | | | | | | aMeGlu | | | | | | | | |
| 1128-2 | | Lys* | Glu | PRO | D-Nar | Cys | L- | His | D-Phe | Arg | Trp(6-Me) | Cys | | | |
| | | | | | | | aMeGlu | | | | | | | | |
| 1131-2 | | Lys* | PEG1 | PEG1 | D-Nar | Cys | L- | His | D-Phe | Arg | Trp(6-Me) | Cys | | | |
| | | | | | | | aMeAsp | | | | | | | | |
| 1130-2 | | Lys* | Gly | Gly | beta- | Cys | L- | His | D-Phe | Arg | Trp(6-Me) | Cys | | | |
| | | | | | homoArg | | aMeAsp | | | | | | | | |
| 1146-2 | Lys* | D-Arg | Gly | D-Arg | D-Nar | Cys | Phg | 3-Pal | D-Phe(4-F) | Arg | Trp(6-F) | Cys | | | |
| 1139-2 | | Lys* | Gly | D-Arg | D-Nar | Cys | Phg | 3-Pal | D-Phe(4-F) | Arg | Trp(6-F) | Cys | | | |
| 1145-2 | | Lys* | PEG1 | PEG1 | D-Nar | Cys | Phg | 3-Pal | D-Phe(4-F) | Arg | Trp(6-F) | Cys | | | |
| 1147-2 | Lys* | D-Arg | PEG1 | D-Arg | Beta- | Cys | Phg | 3-Pal | D-Phe(4-F) | Arg | Trp(6-F) | Cys | | | |
| | | | | | homoArg | | | | | | | | | | |
| 1148-2 | Lys* | D-Arg | Gly | D-Arg | D-Nar | Cys | D- | Gln | D-Phe | Arg | Trp(6-F) | Cys | | | |
| | | | | | | | aMeOrn | | | | | | | | |
| 1149-2 | | Lys* | Gly | D-Arg | D-Nar | Cys | D- | Gln | D-Phe | Arg | Trp(6-F) | Cys | | | |
| | | | | | | | aMeOrn | | | | | | | | |
| 1137-2 | | Lys* | PEG1 | PEG1 | D-Nar | Cys | D- | Gln | D-Phe | Arg | Trp(6-F) | Cys | | | |
| | | | | | | | aMeOrn | | | | | | | | |
| 1136-2 | Lys* | D-Arg | PEG1 | D-Arg | Beta- | Cys | D- | Gln | D-Phe | Arg | Trp(6-F) | Cys | | | |
| | | | | | homoArg | | aMeOrn | | | | | | | | |
| 1150-2 | Lys* | D-Arg | Gly | D-Arg | D-Nar | Cys | Aib(O- | Gln | D-Phe(4-F) | Arg | Trp(6-F) | Cys | | | |
| | | | | | | | cyclic) | | | | | | | | |
| 1142-2 | | Lys* | Gly | D-Arg | D-Nar | Cys | Aib(O- | Gln | D-Phe(4-F) | Arg | Trp(6-F) | Cys | | | |
| | | | | | | | cyclic) | | | | | | | | |
| 1144-2 | | Lys* | PEG1 | PEG1 | D-Nar | Cys | Aib(O- | Gln | D-Phe(4-F) | Arg | Trp(6-F) | Cys | | | |
| | | | | | | | cyclic) | | | | | | | | |
| 1151-2 | Lys* | D-Arg | PEG1 | D-Arg | Beta- | Cys | Aib(O- | Gln | D-Phe(4-F) | Arc | Trp(6-F) | Cys | | | |
| | | | | | homcArg | | cyclic) | | | | | | | | |
| 1152-2 | Lys* | D-Arg | Gly | D-Arg | D-Nar | Cys | Aib(O- | 3-Pal | D-Phe(4-F) | Arg | Trp(6-F) | Cys | | | |
| | | | | | | | cyclic) | | | | | | | | |
| 1154-2 | Lys* | D-Arg | Gly | D-Arg | D-Nar | Cys | D- | 3-Pal | D-Phe(4-F) | Arg | Trp(6-F) | Cys | | | |
| | | | | | | | aMeOrn | | | | | | | | |
| 1156-2 | Lys* | D-Arg | Gly | D-Arg | D-Nar | Cys | D- | 3-Pal | D-Phe | Arg | Trp(6-F) | Cys | | | |
| | | | | | | | aMeOrn | | | | | | | | |
| 1200-2 | | | | | D-Arg | Cys | L- | His | D-Phe | Arg | Trp(6-Me) | Cys | Gly | Gly | Lys* |
| | | | | | | | aMeAsp | | | | | | | | |

In embodiments, the peptide of formula (II) is selected from Table D2A.

In embodiments, the peptide of formula (II) is selected from Table D2A, wherein the N-terminal, C-terminal and/or cyclic structure are an optional feature.

TABLE D2A
Exemplary lipidated peptides.

| Molecule | | | | | | | | | | | | | | | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | N-term | C-term | Cyclic |
| 1129 | | Lys* | Gly | D-Arg | D-Nar | Cys | L- | His | D-Phe | Arg | Trp(6-Me) | Cys | Ac | NH₂ | Disulfide |
| | | | | | | | aMeGlu | | | | | | | | |
| 1128 | | Lys* | Glu | PRO | D-Nar | Cys | L- | His | D-Phe | Arg | Trp(6-Me) | Cys | Ac | NH₂ | Disulfide |
| | | | | | | | aMeGlu | | | | | | | | |
| 1131 | | Lys* | PEG1 | PEG1 | D-Nar | Cys | L- | His | D-Phe | Arg | Trp(6-Me) | Cys | Ac | NH₂ | Disulfide |
| | | | | | | | aMeAsp | | | | | | | | |
| 1130 | | Lys* | Gly | Gly | beta- | Cys | L- | His | D-Phe | Arg | Trp(6-Me) | Cys | Ac | NH₂ | Disulfide |
| | | | | | homoArg | | aMeAsp | | | | | | | | |
| 1146 | Lys* | D-Arg | Gly | D-Arg | D-Nar | Cys | Phg | 3-Pal | D- | Arg | Trp(6-F) | Cys | Ac | NH₂ | Disulfide |
| | | | | | | | | | Phe(4-F) | | | | | | |
| 1139 | | Lys* | Gly | D-Arg | D-Nar | Cys | Phg | 3-Pal | D- | Arg | Trp(6-F) | Cys | Ac | NH₂ | Disulfide |
| | | | | | | | | | Phe(4-F) | | | | | | |
| 1145 | | Lys* | PEG1 | PEG1 | D-Nar | Cys | Phg | 3-Pal | D- | Arg | Trp(6-F) | Cys | Ac | NH₂ | Disulfide |
| | | | | | | | | | Phe(4-F) | | | | | | |
| 1147 | Lys* | D-Arg | PEG1 | D-Arg | Beta- | Cys | Phg | 3-Pal | D- | Arg | Trp(6-F) | Cys | Ac | NH₂ | Disulfide |
| | | | | | homoArg | | | | Phe(4-F) | | | | | | |
| 1148 | Lys* | D-Arg | Gly | D-Arg | D-Nar | Cys | D- | Gln | D-Phe | Arg | Trp(6-F) | Cys | Ac | NH₂ | Disulfide |
| | | | | | | | aMeOrn | | | | | | | | |
| 1149 | | Lys* | Gly | D-Arg | D-Nar | Cys | D- | Gln | D-Phe | Arg | Trp(6-F) | Cys | Ac | NH₂ | Disulfide |
| | | | | | | | aMeOrn | | | | | | | | |
| 1137 | | Lys* | PEG1 | PEG1 | D-Nar | Cys | D- | Gln | D-Phe | Arg | Trp(6-F) | Cys | Ac | NH₂ | Disulfide |
| | | | | | | | aMeOrn | | | | | | | | |
| 1136 | Lys* | D-Arg | PEG1 | D-Arg | Beta- | Cys | D- | Gln | D-Phe | Arg | Trp(6-F) | Cys | Ac | NH₂ | Disulfide |
| | | | | | homoArg | | aMeOrn | | | | | | | | |
| 1150 | Lys* | D-Arg | Gly | D-Arg | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1142 | | Lys* | Gly | D-Arg | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1144 | | Lys* | PEG1 | PEG1 | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1151 | Lys* | D-Arg | PEG1 | D-Arg | Beta- | Cys | Aib(O- | Gln | D- | Arg | Trp(6-F) | Cys | Ac | NH₂ | Disulfide |
| | | | | | homoArg | | cyclic) | | Phe(4-F) | | | | | | |
| 1152 | Lys* | D-Arg | Gly | D-Arg | D-Nar | Cys | Aib(O- | 3-Pal | D- | Arg | Trp(6-F) | Cys | Ac | NH₂ | Disulfide |
| | | | | | | | cyclic) | | Phe(4-F) | | | | | | |
| 1154v | Lys* | D-Arg | Gly | D-Arg | D-Nar | Cys | Aib(O- | 3-Pal | D- | Arg | Trp(6-F) | Cys | Ac | NH₂ | Disulfide |
| | | | | | | | cyclic) | | Phe(4-F) | | | | | | |
| 1156 | Lys* | D-Arg | Gly | D-Arg | D-Nar | Cys | Aib(O- | 3-Pal | D- | Arg | Trp(6-F) | Cys | Ac | NH₂ | Disulfide |
| | | | | | | | cyclic) | | Phe(4-F) | | | | | | |

| Molecule | | | | | | | | | | | | | | |
|---|---|---|---|---|---|---|---|---|---|---|---|---|---|---|
| Name | X₁ | X₂ | X₃ | X₄ | X₅ | X₆ | X₇ | X₈ | X₉ | X₁₀ | X₁₁ | N-term | C-term | Cyclic |
| 1200 | D-Arg | Cys | L- | His | D-Phe | Arg | Trp(6-Me) | Cys | Gly | Gly | Lys* | Ac | NH₂ | Disulfide |
| | | | aMeAsp | | | | | | | | | | | |

In embodiments, the peptides of Table E1, Table E1A, Table E2 and Table E2A are not limited to N-terminal functional group, C-terminal functional group, and/or status or type of cyclic function.

In embodiments, the peptide of formula (I) is selected from Table E1.

TABLE E1
Exemplary peptides.

| Molecule | | | | | | | | |
|---|---|---|---|---|---|---|---|---|
| Name | X₁ | X₂ | X₃ | X₄ | X₅ | X₆ | X₇ | X₈ |
| 1001-1 | Arg | Cys | Cyclo-Leu | His | D-Phe | Arg | Trp | Cys |
| 1002-1 | Arg | Cys | D-Ala | His | D-Phe(3,4-diMe) | Arg | Trp | Cys |
| 1003-1 | Arg | Cys | L-aMeGlu | His | D-Phe | Arg | Trp | Cys |
| 1004-1 | Arg | Cys | L-aMeAsp | His | D-Phe | Arg | Trp | Cys |
| 1005-1 | D-Arg | Cys | Cyclo-Leu | His | D-Phe | Arg | Trp | Cys |
| 1006-1 | Beta- | Cys | Cyclo-Leu | His | D-Phe | Arg | Trp | Cys |
| | homoArg | | | | | | | |
| 1007-1 | D-Arg | Cys | L-aMeGlu | His | D-Phe | Arg | Trp | Cys |
| 1008-1 | Beta- | Cys | Cyclo-Leu | His | D-Phe | Arg | Trp(6-Me) | Cys |
| | homoArg | | | | | | | |
| 1009-1 | D-Arg | Cys | Cyclo-Leu | His | D-Phe | Arg | Trp(6-Me) | Cys |
| 1010-1 | Arg | Cys | D-aMeOrn | His | D-Phe | Arg | Trp | Cys |
| 1011-1 | Arg | Cys | D-Ala | Gln | D-Phe | Arg | Trp | Cys |
| 1012-1 | Arg | Cys | Cyclo-Leu | Gln | D-Phe | Arg | Trp | Cys |
| 1013-1 | Arg | Cys | Ala(2-Me) | Gln | D-Phe | Arg | Trp | Cys |
| 1014-1 | Beta- | Cys | D-Dab | Gln | D-Phe | Arg | Trp | Cys |
| | homoArg | | | | | | | |
| 1015-1 | D-Arg | Cys | Cyclo-Leu | His | D-Phe(4-Me) | Arg | Trp(6-Me) | Cys |
| 1016-1 | D-Arg | Cys | L-aMeGlu | His | D-Phe | Arg | Trp(6-Me) | Cys |
| 1017-1 | D-Arg | Cys | L-aMeGlu | His | D-Phe(4-Me) | Arg | Trp | Cys |
| 1018-1 | D-Arg | Cys | L-aMeGlu | His | D-Phe | Arg | Trp | Cys |
| 1019-1 | D-Nar | Cys | L-aMeGlu | His | D-Phe | Arg | Trp | Cys |
| 1020-1 | D-Nar | Cys | L-aMeGlu | His | D-Phe | Arg | Trp(6-Me) | Cys |
| 1021-1 | D-Nar | Cys | L-aMeGlu | His | D-Phe(4-Me) | Arg | Trp | Cys |
| 1022-1 | Beta- | Cys | L-aMeGlu | His | D-Phe | Arg | Trp | Cys |
| | homoArg | | | | | | | |
| 1023-1 | Beta- | Cys | L-aMeGlu | His | D-Phe(4-Me) | Arg | Trp | Cys |
| | homoArg | | | | | | | |
| 1024-1 | Beta- | Cys | L-aMeGlu | His | D-Phe | Arg | Trp(6-Me) | Cys |
| | homoArg | | | | | | | |
| 1025-1 | Beta- | Cys | L-aMeGlu | His | D-Phe | Arg | Trp | Cys |
| | homoArg | | | | | | | |
| 1026-1 | Beta- | Cys | L-aMeGlu | His | D-Phe(4-Cl) | Arg | Trp(6-Me) | Cys |
| | homoArg | | | | | | | |
| 1027-1 | Beta- | Cys | Cyclo-Leu | His | D-Phe(4-Cl) | Arg | TRP | Cys |
| | homoArg | | | | | | | |
| 1028-1 | Beta- | Cys | L-aMeGlu | His | D-Phe | Arg | Trp(6-F) | Cys |
| | homoArg | | | | | | | |
| 1029-1 | L-hArg | Cys | L-aMeGlu | His | D-Phe | Arg | Trp(6-Me) | Cys |
| 1030-1 | Beta- | Cys | L-aMeGlu | His | D-Phe(3-CF3) | Arg | Trp | Cys |
| | homoArg | | | | | | | |
| 1031-1 | Beta- | Cys | L-aMeGlu | His | D-Phe(3-Cl) | Arg | TRP | Cys |
| | homoArg | | | | | | | |
| 1032-1 | Beta- | Cys | L-aMeGlu | His | D-Phe | Arg | Trp(6-Cl) | Cys |
| | homoArg | | | | | | | |
| 1033-1 | Beta- | Cys | bhGlu | His | D-Phe | Arg | Trp | Cys |
| | homoArg | | | | | | | |
| 1034-1 | Beta- | Cys | Phg | His | D-Phe | Arg | Trp | Cys |
| | homoArg | | | | | | | |
| 1035-1 | D-Nar | Cys | L-aMeGlu | His | D-Phe | Arg | Trp(6-F) | Cys |
| 1036-1 | D-Nar | Cys | D-aMeOrn | His | D-Phe | Arg | Trp(6-F) | Cys |
| 1037-1 | D-Nar | Cys | Ala(2-Me) | His | D-Phe | Arg | Trp(6-F) | Cys |
| 1038-1 | D-Nar | Cys | L-aMeGlu | His | D-Phe(4-F) | Arg | Trp(6-F) | Cys |
| 1039-1 | D-Nar | Cys | D-aMeSer | His | D-Phe | Arg | Trp(6-Me) | Cys |
| 1040-1 | D-Nar | Cys | L-aMeGlu | His | D-Phe(3-F) | Arg | Trp(6-F) | Cys |
| 1041-1 | D-Nar | Cys | L-aMeGlu | His | D-Phe(4-F) | Arg | Trp(6-Me) | Cys |
| 1042-1 | D-Nar | Cys | L-aMeAsp | His | D-Phe | Arg | Trp(6-F) | Cys |
| 1043-1 | D-Nar | Cys | L-aMeGlu | His | D-Phe | Arg | Trp(5-Me) | Cys |
| 1044-1 | D-Nar | Cys | D-bhGlu | His | D-Phe | Arg | Trp(6-Me) | Cys |
| 1045-1 | D-Nar | Cys | L-aMeGlu | His | D-Phe(4-F) | Arg | Trp(6-Cl) | Cys |
| 1046-1 | D-Nar | Cys | L-aMeGlu | His | D-Phe(3,4,5-triF) | Arg | Trp(6-Me) | Cys |
| 1047-1 | D-Nar | Cys | L-aMeGlu | His | D-Phe(3,4,5-triF) | Arg | Trp(6-F) | Cys |
| 1048-1 | D-Nar | Cys | L-aMeGlu | His | D-Phe(3-Cl) | Arg | Trp(6-Me) | Cys |
| 1049-1 | D-Nar | Cys | hGlu | His | D-Phe | Arg | Trp(6-Me) | Cys |
| 1050-1 | D-Nar | Cys | L-aMeGlu | His | D-Phe(3-CF3) | Arg | Trp(6-Me) | Cys |
| 1051-1 | D-Nar | Cys | L-aMeGlu | His | D-Phe(4-Cl) | Arg | Trp(6-F) | Cys |
| 1052-1 | Beta- | Cys | Cyclo-Leu | His | D-Phe | Arg | Trp(6-F) | Cys |
| | homoArg | | | | | | | |
| 1053-1 | Arg | Cys | L-aMeGlu | His | D-Phe(4-F) | Arg | Trp(6-F) | Cys |
| 1054-1 | L-hArg | Cys | L-aMeGlu | His | D-Phe(4-F) | Arg | Trp(6-F) | Cys |
| 1055-1 | D-Arg | Cys | D-aMeOrn | His | D-Phe | Arg | Trp(6-Me) | Cys |
| 1056-1 | Arg | Cys | L-aMeGlu | His | D-Phe(4-F) | Arg | Trp(6-Me) | Cys |
| 1057-1 | Arg | Cys | L-aMeGlu | His | D-Phe(3-F) | Arg | Trp(6-F) | Cys |
| 1058-1 | D-Nar | Cys | Aib(O- | His | D-Phe | Arg | Trp(6-F) | Cys |
| | | | cyclic) | | | | | |
| 1059-1 | Beta- | Cys | L-aMeGlu | His | D-Phe(4-F) | Arg | Trp(6-Me) | Cys |
| | homoArg | | | | | | | |
| 1060-1 | D-Arg | Cys | L-aMeGlu | His | D-Phe(4-F) | Arg | Trp(6-Me) | Cys |
| 1061-1 | L-hArg | Cys | L-aMeGlu | His | D-Phe(4-F) | Arg | Trp(6-Me) | Cys |
| 1062-1 | D-Nar | Cys | L-aMeAsp | His | D-Phe(4-F) | Arg | Trp(6-Me) | Cys |
| 1063-1 | D-Arg | Cys | L-aMeAsp | His | D-Phe(4-F) | Arg | Trp(6-Me) | Cys |
| 1064-1 | D-Nar | Cys | L-aMeAsp | His | D-Phe | Arg | Trp(6-Me) | Cys |
| 1065-1 | Beta- | Cys | L-aMeAsp | His | D-Phe | Arg | Trp(6-Me) | Cys |
| | homoArg | | | | | | | |
| 1066-1 | D-Nar | Glu | L-aMeGlu | His | D-Phe | Arg | Trp(6-Me) | Dap |
| 1067-1 | D-Nar | Asp | L-aMeGlu | His | D-Phe | Arg | Trp(6-Me) | Dap |
| 1068-1 | D-Nar | Glu | L-aMeAsp | His | D-Phe(4-F) | Arg | Trp(6-F) | Dap |
| 1069-1 | D-Nar | Glu | L-aMeGlu | His | D-Phe | Arg | Trp(6-Me) | Dap |
| 1070-1 | Arg | Cys | D-Ala | Gln | D-Phe | Arg | Trp | Cys |
| 1071-1 | D-Nar | Cys | L-aMeGlu | His | D-Phe(3-F,4-Me) | Arg | Trp(6-F) | Cys |
| 1072-1 | D-Nar | Cys | L-aMeGlu | His | D-Phe(4-CF3) | Arg | Trp(6-F) | Cys |
| 1073-1 | D-Nar | Cys | L-aMeGlu | His | D-Phe(2-F,4-Cl) | Arg | Trp(6-F) | Cys |
| 1074-1 | D-Nar | Cys | L-aMeGlu | His | D-Phe(3,4-diF) | Arg | Trp(6-F) | Cys |
| 1075-1 | D-Nar | Cys | L-aMeGlu | His | D-Phe | Arg | Trp(6-CF3) | Cys |
| 1076-1 | D-Nar | Cys | L-aMeGlu | His | D-Phe | Arg | Trp(4-F) | Cys |
| 1077-1 | D-Nar | Cys | L-aMeGlu | His | D-Phe | Arg | Trp(5-F) | Cys |
| 1078-1 | D-Nar | Cys | L-aMeGlu | His | D-Phe | Arg | Trp(7-F) | Cys |
| 1079-1 | D-Nar | Cys | L-aMeGlu | His | D-Phe | Arg | Trp(5-Cl) | Cys |
| 1080-1 | D-Nar | Cys | L-aMeGlu | His | D-Phe | Arg | Trp(6-Br) | Cys |
| 1081-1 | D-Nar | Cys | L-aMeGlu | His | D-Phe(3-F) | Arg | Trp(5-F) | Cys |
| 1082-1 | D-Nar | Cys | L-aMeGlu | His | D-Phe(2,4-diCl) | Arg | Trp(6-F) | Cys |
| 1083-1 | D-Nar | Cys | L-aMeGlu | His | D-Phe(2,3-diF) | Arg | Trp(6-F) | Cys |
| 1084-1 | D-Nar | Cys | L-aMeGlu | His | D-Phe(3-Cl) | Arg | Trp(6-F) | Cys |
| 1085-1 | D-Nar | Cys | L-aMeGlu | His | D-Phe(3-F) | Arg | Trp(6-Me) | Cys |
| 1086-1 | D-Nar | Cys | L-aMeGlu | His | D-Phe(3-Me) | Arg | Trp(6-F) | Cys |
| 1087-1 | D-Nar | Cys | L-aMeGlu | His | D-Phe(2,4-diF) | Arg | Trp(6-F) | Cys |
| 1088-1 | D-Nar | Cys | L-aMeGlu | His | D-Phe(2,4,5-triF) | Arg | Trp(6-F) | Cys |
| 1089-1 | D-Nar | Cys | L-aMeAsp | His | D-Phe(3-CF3) | Arg | Trp(6-F) | Cys |
| 1090-1 | D-Nar | Cys | L-aMeGlu | Gln | D-Phe | Arg | Trp(6-F) | Cys |
| 1091-1 | D-Nar | Cys | hGlu | Gln | D-Phe | Arg | Trp(6-F) | Cys |
| 1092-1 | D-Nar | Cys | Aib(O- | Gln | D-Phe | Arg | Trp(6-F) | Cys |
| | | | cyclic) | | | | | |
| 1093-1 | D-Nar | Cys | Aib(O- | Gln | D-Phe(4-F) | Arg | Trp(6-F) | Cys |
| | | | cyclic) | | | | | |
| 1094-1 | D-Nar | Cys | D-aMeOrn | Gln | D-Phe | Arg | Trp(6-F) | Cys |
| 1095-1 | D-Nar | Cys | Aib(O- | Gln | D-Phe(4-Me) | Arg | Trp(6-F) | Cys |
| | | | cyclic) | | | | | |
| 1096-1 | D-Nar | Cys | D-aMeSer | Gln | D-Phe | Arg | Trp(6-F) | Cys |
| 1097-1 | D-Nar | Cys | D-aMeSer | Gln | D-Phe(4-F) | Arg | Trp(6-F) | Cys |
| 1098-1 | D-Nar | Cys | bhGlu | Gln | D-Phe | Arg | Trp(6-F) | Cys |
| 1099-1 | D-Nar | Cys | Ala(2-Me) | Gln | D-Phe(4-F) | Arg | Trp(6-F) | Cys |
| 1100-1 | D-Nar | Cys | D-aMeOrn | hGln | D-Phe | Arg | Trp(6-F) | Cys |
| 1101-1 | D-Nar | Cys | D-aMeOrn | hGln | D-Phe(4-F) | Arg | Trp(6-F) | Cys |
| 1102-1 | D-Nar | Cys | Aib(O- | hGln | D-Phe | Arg | Trp(6-F) | Cys |
| | | | cyclic) | | | | | |
| 1103-1 | D-Nar | Cys | Aib(O- | Cit | D-Phe | Arg | Trp(6-F) | Cys |
| | | | cyclic) | | | | | |
| 1104-1 | D-Nar | Cys | L-aMeGlu | Cit | D-Phe | Arg | Trp(6-F) | Cys |
| 1105-1 | D-Nar | Cys | Aib(O- | Cit | D-Phe(4-Me) | Arg | Trp(6-F) | Cys |
| | | | cyclic) | | | | | |
| 1106-1 | D-Nar | Cys | Aib(O- | Cit | D-Phe(4-F) | Arg | Trp(6-F) | Cys |
| | | | cyclic) | | | | | |
| 1107-1 | D-Nar | Cys | Aib(O- | hCit | D-Phe(4-F) | Arg | Trp(6-F) | Cys |
| | | | cyclic) | | | | | |
| 1108-1 | D-Nar | Cys | Cyclo-Leu | 3-Pal | D-Phe | Arg | Trp(6-F) | Cys |
| 1109-1 | D-Nar | Cys | D-aMeOrn | 4-Pal | D-Phe | Arg | Trp(6-F) | Cys |
| 1110-1 | D-Nar | Cys | Phg | His | D-Phe | Arg | Trp(6-F) | Cys |
| 1111-1 | D-Nar | Cys | Phg | His | D-Phe(4-F) | Arg | Trp(6-F) | Cys |
| 1112-1 | D-Nar | Cys | Phg | His | D-Phe(4-F) | Arg | Trp(5-Me) | Cys |
| 1113-1 | D-Nar | Cys | Phg | His | D-Phe(4-F) | Arg | Trp(6-Me) | Cys |
| 1114-1 | Nar | Cys | L-aMeGlu | His | D-Phe | Arg | Trp(6-F) | Cys |
| 1115-1 | Nar | Cys | L-aMeGlu | His | D-Phe(4-F) | Arg | Trp(6-F) | Cys |
| 1116-1 | D-Nar | Cys | L-aMeGlu | 3-Pal | D-Phe | Arg | Trp(6-Me) | Cys |
| 1117-1 | D-Nar | Cys | L-aMeGlu | 3-Pal | D-Phe | Arg | Trp(6-Me) | Cys |
| 1118-1 | D-Nar | Cys | L-aMeGlu | 4-Pal | D-Phe | Arg | Trp(6-Me) | Cys |
| 1119-1 | D-Nar | Cys | Phg | 3-Pal | D-Phe(4-F) | Arg | Trp(6-F) | Cys |
| 1120-1 | D-Nar | Glu | L-aMeAsp | His | D-Phe | Arg | Trp(6-F) | Dap |
| 1121-1 | D-Nar | Glu | L-aMeAsp | His | D-Phe(4-F) | Arg | Trp(6-F) | Dap |
| 1122-1 | D-Nar | Cys | Aib(O- | 3-Pal | D-Phe(4-F) | Arg | Trp(6-F) | Cys |
| | | | cyclic) | | | | | |
| 1123-1 | D-Nar | Cys | Aib(O- | Orn | D-Phe(4-F) | Arg | Trp(6-F) | Cys |
| | | | cyclic) | | | | | |
| 1124-1 | D-Nar | Cys | D-aMeOrn | 3-Pal | D-Phe(4-F) | Arg | Trp(6-F) | Cys |
| 1125-1 | D-Nar | Cys | D-aMeOrn | Orn | D-Phe(4-F) | Arg | Trp(6-F) | Cys |
| 1126-1 | D-Nar | Cys | D-aMeOrn | 3-Pal | D-Phe | Arg | Trp(6-F) | Cys |
| 1127-1 | D-Nar | Cys | D-aMeOrn | Orn | D-Phe | Arg | Trp(6-F) | Cys |
| 1158-1 | D-Nar | Cys | Aib(O- | Gln | D-Phe(4-F) | Arg | Trp(6-F) | Pen |
| | | | cyclic) | | | | | |

In embodiments, the peptide of formula (I) is selected from Table E1A.

In embodiments, the peptide of formula (I) is selected from Table E1A, wherein the N-terminal, C-terminal and/or cyclic structure are an optional feature.

TABLE E1A
Exemplary peptides.

| Molecule | | | | | | | | | | | |
|---|---|---|---|---|---|---|---|---|---|---|---|
| Name | X₁ | X₂ | X₃ | X₄ | X₅ | X₆ | X₇ | X₈ | N-term | C-term | Cyclic |
| 1001 | Arg | Cys | Cyclo- | His | D-Phe | Arg | Trp | Cys | Ac | NH₂ | Disulfide |
| | | | Leu | | | | | | | | |
| 1002 | Arg | Cys | D-Ala | His | D- | Arg | Trp | Cys | Ac | NH₂ | Disulfide |
| | | | | | Phe(3,4-diMe) | | | | | | |
| 1003 | Arg | Cys | L- | His | D-Phe | Arg | Trp | Cys | Ac | NH₂ | Disulfide |
| | | | aMeGlu | | | | | | | | |
| 1004 | Arg | Cys | L- | His | D-Phe | Arg | Trp | Cys | Ac | NH₂ | Disulfide |
| | | | aMeAsp | | | | | | | | |
| 1005 | D-Arg | Cys | Cyclo- | His | D-Phe | Arg | Trp | Cys | Ac | NH₂ | Disulfide |
| | | | Leu | | | | | | | | |
| 1006 | Beta- | Cys | Cyclo- | His | D-Phe | Arg | Trp | Cys | Ac | NH₂ | Disulfide |
| | homoArg | | Leu | | | | | | | | |
| 1007 | D-Arg | Cys | L- | His | D-Phe | Arg | Trp | Cys | Ac | NH₂ | Disulfide |
| | | | aMeGlu | | | | | | | | |
| 1008 | Beta- | Cys | Cyclo- | His | D-Phe | Arg | Trp(6-Me) | Cys | Ac | NH₂ | Disulfide |
| | homoArg | | Leu | | | | | | | | |
| 1009 | D-Arg | Cys | Cyclo- | His | D-Phe | Arg | Trp(6-Me) | Cys | Ac | NH₂ | Disulfide |
| | | | Leu | | | | | | | | |
| 1010 | Arg | Cys | D- | His | D-Phe | Arg | Trp | Cys | Ac | NH₂ | Disulfide |
| | | | aMeOrn | | | | | | | | |
| 1011 | Arg | Cys | D-Ala | Gln | D-Phe | Arg | Trp | Cys | Ac | NH₂ | Disulfide |
| 1012 | Arg | Cys | Cyclo- | Gln | D-Phe | Arg | Trp | Cys | Ac | NH₂ | Disulfide |
| | | | Leu | | | | | | | | |
| 1013 | Arg | Cys | Ala(2- | Gln | D-Phe | Arg | Trp | Cys | Ac | NH₂ | Disulfide |
| | | | Me) | | | | | | | | |
| 1014 | Beta- | Cys | D-Dab | Gln | D-Phe | Arg | Trp | Cys | Ac | NH₂ | Disulfide |
| | homoArg | | | | | | | | | | |
| 1015 | D-Arg | Cys | Cyclo- | His | D- | Arg | Trp(6-Me) | Cys | Ac | NH₂ | Disulfide |
| | | | Leu | | Phe(4-Me) | | | | | | |
| 1016 | D-Arg | Cys | L- | His | D-Phe | Arg | Trp(6-Me) | Cys | Ac | NH₂ | Disulfide |
| | | | aMeGlu | | | | | | | | |
| 1017 | D-Arg | Cys | L- | His | D- | Arg | Trp | Cys | Ac | NH₂ | Disulfide |
| | | | aMeGlu | | Phe(4-Me) | | | | | | |
| 1018 | D-Arg | Cys | L- | His | D-Phe | Arg | Trp | Cys | Ac | NH₂ | Disulfide |
| | | | aMeGlu | | | | | | | | |
| 1019 | D-Nar | Cys | L- | His | D-Phe | Arg | Trp | Cys | Ac | NH₂ | Disulfide |
| | | | aMeGlu | | | | | | | | |
| 1020 | D-Nar | Cys | L- | His | D-Phe | Arg | Trp(6-Me) | Cys | Ac | NH₂ | Disulfide |
| | | | aMeGlu | | | | | | | | |
| 1021 | D-Nar | Cys | L- | His | D- | Arg | Trp | Cys | Ac | NH₂ | Disulfide |
| | | | aMeGlu | | Phe(4-Me) | | | | | | |
| 1022 | Beta- | Cys | L- | His | D-Phe | Arg | Trp | Cys | Ac | NH₂ | Disulfide |
| | homoArg | | aMeGlu | | | | | | | | |
| 1023 | Beta- | Cys | L- | His | D- | Arg | Trp | Cys | Ac | NH₂ | Disulfide |
| | homoArg | | aMeGlu | | Phe(4-Me) | | | | | | |
| 1024 | Beta- | Cys | L- | His | D-Phe | Arg | Trp(6-Me) | Cys | Ac | NH₂ | Disulfide |
| | homoArg | | aMeGlu | | | | | | | | |
| 1025 | Beta- | Cys | L- | His | D-Phe | Arg | Trp | Cys | Ac | NH₂ | Disulfide |
| | homoArg | | aMeGlu | | | | | | | | |
| 1026 | Beta- | Cys | L- | His | D- | Arg | Trp(6-Me) | Cys | Ac | NH₂ | Disulfide |
| | homoArg | | aMeGlu | | Phe(4-Cl) | | | | | | |
| 1027 | Beta- | Cys | Cyclo- | His | D- | Arg | TRP | Cys | Ac | NH₂ | Disulfide |
| | homoArg | | Leu | | Phe(4-Cl) | | | | | | |
| 1028 | Beta- | Cys | L- | His | D-Phe | Arg | Trp(6-F) | Cys | Ac | NH₂ | Disulfide |
| | homoArg | | aMeGlu | | | | | | | | |
| 1029 | L-hArg | Cys | L- | His | D-Phe | Arg | Trp(6-Me) | Cys | Ac | NH₂ | Disulfide |
| | | | aMeGlu | | | | | | | | |
| 1030 | Beta- | Cys | L- | His | D- | Arg | Trp | Cys | Ac | NH₂ | Disulfide |
| | homoArg | | aMeGlu | | Phe(3-CF3) | | | | | | |
| 1031 | Beta- | Cys | L- | His | D- | Arg | TRP | Cys | Ac | NH₂ | Disulfide |
| | homoArg | | aMeGlu | | Phe(3-Cl) | | | | | | |
| 1032 | Beta- | Cys | L- | His | D-Phe | Arg | Trp(6-Cl) | Cys | Ac | NH₂ | Disulfide |
| | homoArg | | aMeGlu | | | | | | | | |
| 1033 | Beta- | Cys | bhGlu | His | D-Phe | Arg | Trp | Cys | Ac | NH₂ | Disulfide |
| | homoArg | | | | | | | | | | |
| 1034 | Beta- | Cys | Phg | His | D-Phe | Arg | Trp | Cys | Ac | NH₂ | Disulfide |
| | homoArg | | | | | | | | | | |
| 1035 | D-Nar | Cys | L- | His | D-Phe | Arg | Trp(6-F) | Cys | Ac | NH₂ | Disulfide |
| | | | aMeGlu | | | | | | | | |
| 1036 | D-Nar | Cys | D- | His | D-Phe | Arg | Trp(6-F) | Cys | Ac | NH₂ | Disulfide |
| | | | aMeOrn | | | | | | | | |
| 1037 | D-Nar | Cys | Ala(2-Me) | His | D-Phe | Arg | Trp(6-F) | Cys | Ac | NH₂ | Disulfide |
| 1038 | D-Nar | Cys | L- | His | D- | Arg | Trp(6-F) | Cys | Ac | NH₂ | Disulfide |
| | | | aMeGlu | | Phe(4-F) | | | | | | |
| 1039 | D-Nar | Cys | D- | His | D-Phe | Arg | Trp(6-Me) | Cys | Ac | NH₂ | Disulfide |
| | | | aMeSer | | | | | | | | |
| 1040 | D-Nar | Cys | L- | His | D- | Arg | Trp(6-F) | Cys | Ac | NH₂ | Disulfide |
| | | | aMeGlu | | Phe(3-F) | | | | | | |
| 1041 | D-Nar | Cys | L- | His | D- | Arg | Trp(6-Me) | Cys | Ac | NH₂ | Disulfide |
| | | | aMeGlu | | Phe(4-F) | | | | | | |
| 1042 | D-Nar | Cys | L- | His | D-Phe | Arg | Trp(6-F) | Cys | Ac | NH₂ | Disulfide |
| | | | aMeAsp | | | | | | | | |
| 1043 | D-Nar | Cys | L- | His | D-Phe | Arg | Trp(5-Me) | Cys | Ac | NH₂ | Disulfide |
| | | | aMeGlu | | | | | | | | |
| 1044 | D-Nar | Cys | D- | His | D-Phe | Arg | Trp(6-Me) | Cys | Ac | NH₂ | Disulfide |
| | | | bhGlu | | | | | | | | |
| 1045 | D-Nar | Cys | L- | His | D- | Arg | Trp(6-Cl) | Cys | Ac | NH₂ | Disulfide |
| | | | aMeGlu | | Phe(4-F) | | | | | | |
| 1046 | D-Nar | Cys | L- | His | D- | Arg | Trp(6-Me) | Cys | Ac | NH₂ | Disulfide |
| | | | aMeGlu | | Phe(3,4,5-triF) | | | | | | |
| 1047 | D-Nar | Cys | L- | His | D- | Arg | Trp(6-F) | Cys | Ac | NH₂ | Disulfide |
| | | | aMeGlu | | Phe(3,4,5-triF) | | | | | | |
| 1048 | D-Nar | Cys | L- | His | D- | Arg | Trp(6-Me) | Cys | Ac | NH₂ | Disulfide |
| | | | aMeGlu | | Phe(3-Cl) | | | | | | |
| 1049 | D-Nar | Cys | hGlu | His | D-Phe | Arg | Trp(6-Me) | Cys | Ac | NH₂ | Disulfide |
| 1050 | D-Nar | Cys | L- | His | D- | Arg | Trp(6-Me) | Cys | Ac | NH₂ | Disulfide |
| | | | aMeGlu | | Phe(3-CF3) | | | | | | |
| 1051 | D-Nar | Cys | L- | His | D- | Arg | Trp(6-F) | Cys | Ac | NH₂ | Disulfide |
| | | | aMeGlu | | Phe(4-Cl) | | | | | | |
| 1052 | Beta- | Cys | Cyclo- | His | D-Phe | Arg | Trp(6-F) | Cys | Ac | NH₂ | Disulfide |
| | homoArg | | Leu | | | | | | | | |
| 1053 | Arg | Cys | L- | His | D- | Arg | Trp(6-F) | Cys | Ac | NH₂ | Disulfide |
| | | | aMeGlu | | Phe(4-F) | | | | | | |
| 1054 | L-hArg | Cys | L- | His | D- | Arg | Trp(6-F) | Cys | Ac | NH₂ | Disulfide |
| | | | aMeGlu | | Phe(4-F) | | | | | | |
| 1055 | D-Arg | Cys | D- | His | D-Phe | Arg | Trp(6-Me) | Cys | Ac | NH₂ | Disulfide |
| | | | aMeOrn | | | | | | | | |
| 1056 | Arg | Cys | L- | His | D- | Arg | Trp(6-Me) | Cys | Ac | NH₂ | Disulfide |
| | | | aMeGlu | | Phe(4-F) | | | | | | |
| 1057 | Arg | Cys | L- | His | D- | Arg | Trp(6-F) | Cys | Ac | NH₂ | Disulfide |
| | | | aMeGlu | | Phe(3-F) | | | | | | |
| 1058 | D-Nar | Cys | Aib(O- | His | D-Phe | Arg | Trp(6-F) | Cys | Ac | NH₂ | Disulfide |
| | | | cyclic) | | | | | | | | |
| 1059 | Beta- | Cys | L- | His | D- | Arg | Trp(6-Me) | Cys | Ac | NH₂ | Disulfide |
| | homoArg | | aMeGlu | | Phe(4-F) | | | | | | |
| 1060 | D-Arg | Cys | L- | His | D- | Arg | Trp(6-Me) | Cys | Ac | NH₂ | Disulfide |
| | | | aMeGlu | | Phe(4-F) | | | | | | |
| 1061 | L-hArg | Cys | L- | His | D- | Arg | Trp(6-Me) | Cys | Ac | NH₂ | Disulfide |
| | | | aMeGlu | | Phe(4-F) | | | | | | |
| 1062 | D-Nar | Cys | L- | His | D- | Arg | Trp(6-Me) | Cys | Ac | NH₂ | Disulfide |
| | | | aMeAsp | | Phe(4-F) | | | | | | |
| 1063 | D-Arg | Cys | L- | His | D- | Arg | Trp(6-Me) | Cys | Ac | NH₂ | Disulfide |
| | | | aMeAsp | | Phe(4-F) | | | | | | |
| 1064 | D-Nar | Cys | L- | His | D-Phe | Arg | Trp(6-Me) | Cys | Ac | NH₂ | Disulfide |
| | | | aMeAsp | | | | | | | | |
| 1065 | Beta- | Cys | L- | His | D-Phe | Arg | Trp(6-Me) | Cys | Ac | NH₂ | Disulfide |
| | homoArg | | aMeAsp | | | | | | | | |
| 1066 | D-Nar | Glu | L- | His | D-Phe | Arg | Trp(6-Me) | Dap | Ac | NH₂ | Lactam |
| | | | aMeGlu | | | | | | | | |
| 1067 | D-Nar | Asp | L- | His | D-Phe | Arg | Trp(6-Me) | Dap | Ac | NH₂ | Lactam |
| | | | aMeGlu | | | | | | | | |
| 1068 | D-Nar | Glu | L- | His | D- | Arg | Trp(6-F) | Dap | Ac | NH₂ | Lactam |
| | | | aMeAsp | | Phe(4-F) | | | | | | |
| 1069 | D-Nar | Glu | L- | His | D-Phe | Arg | Trp(6-Me) | Dap | Ac | NH₂ | Lactam |
| | | | aMeGlu | | | | | | | | |
| 1070 | Arg | Cys | D-Ala | Gln | D-Phe | Arg | Trp | Cys | Ac | NH₂ | Disulfide |
| 1071 | D-Nar | Cys | L- | His | D-Phe(3- | Arg | Trp(6-F) | Cys | Ac | NH₂ | Disulfide |
| | | | aMeGlu | | F, 4-Me) | | | | | | |
| 1072 | D-Nar | Cys | L- | His | D- | Arg | Trp(6-F) | Cys | Ac | NH₂ | Disulfide |
| | | | aMeGlu | | Phe(4-CF3) | | | | | | |
| 1073 | D-Nar | Cys | L- | His | D-Phe(2- | Arg | Trp(6-F) | Cys | Ac | NH₂ | Disulfide |
| | | | aMeGlu | | F, 4-Cl) | | | | | | |
| 1074 | D-Nar | Cys | L- | His | D- | Arg | Trp(6-F) | Cys | Ac | NH₂ | Disulfide |
| | | | aMeGlu | | Phe(3,4-diF) | | | | | | |
| 1075 | D-Nar | Cys | L- | His | D-Phe | Arg | Trp(6-CF3) | Cys | Ac | NH₂ | Disulfide |
| | | | aMeGlu | | | | | | | | |
| 1076 | D-Nar | Cys | L- | His | D-Phe | Arg | Trp(4-F) | Cys | Ac | NH₂ | Disulfide |
| | | | aMeGlu | | | | | | | | |
| 1077 | D-Nar | Cys | L- | His | D-Phe | Arg | Trp(5-F) | Cys | Ac | NH₂ | Disulfide |
| | | | aMeGlu | | | | | | | | |
| 1078 | D-Nar | Cys | L- | His | D-Phe | Arg | Trp(7-F) | Cys | Ac | NH₂ | Disulfide |
| | | | aMeGlu | | | | | | | | |
| 1079 | D-Nar | Cys | L- | His | D-Phe | Arg | Trp(5-Cl) | Cys | Ac | NH₂ | Disulfide |
| | | | aMeGlu | | | | | | | | |
| 1080 | D-Nar | Cys | L- | His | D-Phe | Arg | Trp(6-Br) | Cys | Ac | NH₂ | Disulfide |
| | | | aMeGlu | | | | | | | | |
| 1081 | D-Nar | Cys | L- | His | D- | Arg | Trp(5-F) | Cys | Ac | NH₂ | Disulfide |
| | | | aMeGlu | | Phe(3-F) | | | | | | |
| 1082 | D-Nar | Cys | L- | His | D- | Arg | Trp(6-F) | Cys | Ac | NH₂ | Disulfide |
| | | | aMeGlu | | Phe(2,4-diCl) | | | | | | |
| 1083 | D-Nar | Cys | L- | His | D- | Arg | Trp(6-F) | Cys | Ac | NH₂ | Disulfide |
| | | | aMeGlu | | Phe(2,3-diF) | | | | | | |
| 1084 | D-Nar | Cys | L- | His | D- | Arg | Trp(6-F) | Cys | Ac | NH₂ | Disulfide |
| | | | aMeGlu | | Phe(3-Cl) | | | | | | |
| 1085 | D-Nar | Cys | L- | His | D- | Arg | Trp(6-Me) | Cys | Ac | NH₂ | Disulfide |
| | | | aMeGlu | | Phe(3-F) | | | | | | |
| 1086 | D-Nar | Cys | L- | His | D- | Arg | Trp(6-F) | Cys | Ac | NH₂ | Disulfide |
| | | | aMeGlu | | Phe(3-Me) | | | | | | |
| 1087 | D-Nar | Cys | L- | His | D- | Arg | Trp(6-F) | Cys | Ac | NH₂ | Disulfide |
| | | | aMeGlu | | Phe(2,4-diF) | | | | | | |
| 1088 | D-Nar | Cys | L- | His | D- | Arg | Trp(6-F) | Cys | Ac | NH₂ | Disulfide |
| | | | aMeGlu | | Phe(2,4,5-triF) | | | | | | |
| 1089 | D-Nar | Cys | L- | His | D- | Arg | Trp(6-F) | Cys | Ac | NH₂ | Disulfide |
| | | | aMeAsp | | Phe(3-CF3) | | | | | | |
| 1090 | D-Nar | Cys | L- | Gln | D-Phe | Arg | Trp(6-F) | Cys | Ac | NH₂ | Disulfide |
| | | | aMeGlu | | | | | | | | |
| 1091 | D-Nar | Cys | hGlu | Gln | D-Phe | Arg | Trp(6-F) | Cys | Ac | NH₂ | Disulfide |
| 1092 | D-Nar | Cys | Aib(O- | Gln | D-Phe | Arg | Trp(6-F) | Cys | Ac | NH₂ | Disulfide |
| | | | cyclic) | | | | | | | | |
| 1093 | D-Nar | Cys | Aib(O- | Gln | D- | Arg | Trp(6-F) | Cys | Ac | NH₂ | Disulfide |
| | | | cyclic) | | Phe(4-F) | | | | | | |
| 1094 | D-Nar | Cys | D- | Gln | D-Phe | Arg | Trp(6-F) | Cys | Ac | NH₂ | Disulfide |
| | | | aMeOrn | | | | | | | | |
| 1095 | D-Nar | Cys | Aib(O- | Gln | D- | Arg | Trp(6-F) | Cys | Ac | NH₂ | Disulfide |
| | | | cyclic) | | Phe(4-Me) | | | | | | |
| 1096 | D-Nar | Cys | D- | Gln | D-Phe | Arg | Trp(6-F) | Cys | Ac | NH₂ | Disulfide |
| | | | aMeSer | | | | | | | | |
| 1097 | D-Nar | Cys | D- | Gln | D- | Arg | Trp(6-F) | Cys | Ac | NH₂ | Disulfide |
| | | | aMeSer | | Phe(4-F) | | | | | | |
| 1098 | D-Nar | Cys | bhGlu | Gln | D-Phe | Arg | Trp(6-F) | Cys | Ac | NH₂ | Disulfide |
| 1099 | D-Nar | Cys | Ala(2-Me) | Gln | D- | Arg | Trp(6-F) | Cys | Ac | NH₂ | Disulfide |
| | | | | | Phe(4-F) | | | | | | |
| 1100 | D-Nar | Cys | D- | hGln | D-Phe | Arg | Trp(6-F) | Cys | Ac | NH₂ | Disulfide |
| | | | aMeOrn | | | | | | | | |
| 1101 | D-Nar | Cys | D- | hGln | D- | Arg | Trp(6-F) | Cys | Ac | NH₂ | Disulfide |
| | | | aMeOrn | | Phe(4-F) | | | | | | |
| 1102 | D-Nar | Cys | Aib(O- | hGIn | D-Phe | Arg | Trp(6-F) | Cys | Ac | NH₂ | Disulfide |
| | | | cyclic) | | | | | | | | |
| 1103 | D-Nar | Cys | Aib(O- | Cit | D-Phe | Arg | Trp(6-F) | Cys | Ac | NH₂ | Disulfide |
| | | | cyclic) | | | | | | | | |
| 1104 | D-Nar | Cys | L- | Cit | D-Phe | Arg | Trp(6-F) | Cys | Ac | NH₂ | Disulfide |
| | | | aMeGlu | | | | | | | | |
| 1105 | D-Nar | Cys | Aib(O- | Cit | D- | Arg | Trp(6-F) | Cys | Ac | NH₂ | Disulfide |
| | | | cyclic) | | Phe(4-Me) | | | | | | |
| 1106 | D-Nar | Cys | Aib(O- | Cit | D- | Arg | Trp(6-F) | Cys | Ac | NH₂ | Disulfide |
| | | | cyclic) | | Phe(4-F) | | | | | | |
| 1107 | D-Nar | Cys | Aib(O- | hCit | D- | Arg | Trp(6-F) | Cys | Ac | NH₂ | Disulfide |
| | | | cyclic) | | Phe(4-F) | | | | | | |
| 1108 | D-Nar | Cys | Cyclo- | 3-Pal | D-Phe | Arg | Trp(6-F) | Cys | Ac | NH₂ | Disulfide |
| | | | Leu | | | | | | | | |
| 1109 | D-Nar | Cys | D- | 4-Pal | D-Phe | Arg | Trp(6-F) | Cys | Ac | NH₂ | Disulfide |
| | | | aMeOrn | | | | | | | | |
| 1110 | D-Nar | Cys | Phg | His | D-Phe | Arg | Trp(6-F) | Cys | Ac | NH₂ | Disulfide |
| 1111 | D-Nar | Cys | Phg | His | D- | Arg | Trp(6-F) | Cys | Ac | NH₂ | Disulfide |
| | | | | | Phe(4-F) | | | | | | |
| 1112 | D-Nar | Cys | Phg | His | D- | Arg | Trp(5-Me) | Cys | Ac | NH₂ | Disulfide |
| | | | | | Phe(4-F) | | | | | | |
| 1113 | D-Nar | Cys | Phg | His | D- | Arg | Trp(6-Me) | Cys | Ac | NH₂ | Disulfide |
| | | | | | Phe(4-F) | | | | | | |
| 1114 | Nar | Cys | L- | His | D-Phe | Arg | Trp(6-F) | Cys | Ac | NH₂ | Disulfide |
| | | | aMeGlu | | | | | | | | |
| 1115 | Nar | Cys | L- | His | D- | Arg | Trp(6-F) | Cys | Ac | NH₂ | Disulfide |
| | | | aMeGlu | | Phe(4-F) | | | | | | |
| 1116 | D-Nar | Cys | L- | 3-Pal | D-Phe | Arg | Trp(6-Me) | Cys | Ac | NH₂ | Disulfide |
| | | | aMeGlu | | | | | | | | |
| 1117 | D-Nar | Cys | L- | 3-Pal | D-Phe | Arg | Trp(6-Me) | Cys | Ac | NH₂ | Disulfide |
| | | | aMeGlu | | | | | | | | |
| 1118 | D-Nar | Cys | L- | 4-Pal | D-Phe | Arg | Trp(6-Me) | Cys | Ac | NH₂ | Disulfide |
| | | | aMeGlu | | | | | | | | |
| 1119 | D-Nar | Cys | Phg | 3-Pal | D- | Arg | Trp(6-F) | Cys | Ac | NH₂ | Disulfide |
| | | | | | Phe(4-F) | | | | | | |
| 1120 | D-Nar | Glu | L- | His | D-Phe | Arg | Trp(6-F) | Dap | Ac | NH₂ | Lactam |
| | | | aMeAsp | | | | | | | | |
| 1121 | D-Nar | Glu | L- | His | D- | Arg | Trp(6-F) | Dap | Ac | NH₂ | Lactam |
| | | | aMeAsp | | Phe(4-F) | | | | | | |
| 1122 | D-Nar | Cys | Aib(O- | 3-Pal | D- | Arg | Trp(6-F) | Cys | Ac | NH₂ | Disulfide |
| | | | cyclic) | | Phe(4-F) | | | | | | |
| 1123 | D-Nar | Cys | Aib(O- | Orn | D- | Arg | Trp(6-F) | Cys | Ac | NH₂ | Disulfide |
| | | | cyclic) | | Phe(4-F) | | | | | | |
| 1124 | D-Nar | Cys | D- | 3-Pal | D- | Arg | Trp(6-F) | Cys | Ac | NH₂ | Disulfide |
| | | | aMeOrn | | Phe(4-F) | | | | | | |
| 1125 | D-Nar | Cys | D- | Orn | D- | Arg | Trp(6-F) | Cys | Ac | NH₂ | Disulfide |
| | | | aMeOrn | | Phe(4-F) | | | | | | |
| 1126 | D-Nar | Cys | D- | 3-Pal | D-Phe | Arg | Trp(6-F) | Cys | Ac | NH₂ | Disulfide |
| | | | aMeOrn | | | | | | | | |
| 1127 | D-Nar | Cys | D- | Orn | D-Phe | Arg | Trp(6-F) | Cys | Ac | NH₂ | Disulfide |
| | | | aMeOrn | | | | | | | | |
| 1158 | D-Nar | Cys | Aib(O- | Gln | D- | Arg | Trp(6-F) | Pen | Ac | NH₂ | Disulfide |
| | | | cyclic) | | Phe(4-F) | | | | | | |

In embodiments, the peptide of formula (II) is selected from Table E2.

TABLE E2
Exemplary peptides.

| Molecule | | | | | | | | | | | | | | | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | X₉ | X₁₀ | X₁₁ |
| 1128-2 | | Lys* | Glu | Pro | D-Nar | Cys | L- | His | D-Phe | Arg | Trp(6-Me) | Cys | | | |
| | | | | | | | aMeGlu | | | | | | | | |
| 1129-2 | | Lys* | Glu | Pro | D-Nar | Cys | L- | His | D-Phe | Arg | Trp(6-Me) | Cys | | | |
| | | | | | | | aMeGlu | | | | | | | | |
| 1130-2 | | Lys* | Glu | Gly | beta- | Cys | L- | His | D-Phe | Arg | Trp(6-Me) | Cys | | | |
| | | | | | homoArg | | aMeAsp | | | | | | | | |
| 1131-2 | | Lys* | PEG1 | PEG1 | D-Nar | Cys | L- | His | D-Phe | Arg | Trp(6-Me) | Cys | | | |
| | | | | | | | aMeAsp | | | | | | | | |
| 1132-2 | Lys* | Glu | PEG1 | PEG1 | D-Nar | Cys | L- | His | D- | Arg | Trp(6-Me) | Cys | | | |
| | | | | | | | aMeGlu | | Phe(3,4,5-triF) | | | | | | |
| 1133-2 | | Lys* | Gly | gGlu | D-Nar | Cys | L- | His | D-Phe | Arg | Trp(6-Me) | Cys | | | |
| | | | | | | | aMeGlu | | | | | | | | |
| 1134-2 | Lys* | Glu | Glu | Pro | D-Nar | Cys | L- | His | D- | Arg | Trp(6-Me) | Cys | | | |
| | | | | | | | aMeGlu | | Phe(3,4,5-triF) | | | | | | |
| 1135-2 | | Lys* | Gly | Gly | D-Arg | Cys | L- | His | D-Phe | Arg | Trp(6-Me) | Cys | | | |
| | | | | | | | aMeAsp | | | | | | | | |
| 1136-2 | Lys* | D-Arg | PEG1 | D-Arg | Beta- | Cys | D- | Gln | D-Phe | Arg | Trp(6-F) | Cys | | | |
| | | | | | homoArg | | aMeOrn | | | | | | | | |
| 1137-2 | | Lys* | PEG1 | PEG1 | D-Nar | Cys | D- | Gln | D-Phe | Arg | Trp(6-F) | Cys | | | |
| | | | | | | | aMeOrn | | | | | | | | |
| 1138-2 | | Lys* | Gly | D-Arg | D-Nar | Cys | L- | His | D-Phe | Arg | Trp(6-F) | Cys | | | |
| | | | | | | | aMeGlu | | | | | | | | |
| 1139-2 | | Lys* | Gly | D-Arg | D-Nar | Cys | Phg | 3-Pal | D- | Arg | Trp(6-F) | Cys | | | |
| | | | | | | | | | Phe(4-F) | | | | | | |
| 1140-2 | | Lys* | D-Arg | γ-Glu | D-Nar | Cys | L- | His | D- | Arg | Trp(6-F) | Cys | | | |
| | | | | | | | aMeGlu | | Phe | | | | | | |
| 1141-2 | Lys* | Ser | Glu | Pro | D-Nar | Cys | L- | His | D- | Arg | Trp(6-F) | Cys | | | |
| | | | | | | | aMeGlu | | Phe | | | | | | |
| 1142-2 | | Lys* | Gly | D-Arg | D-Nar | Cys | Aib(O- | Gln | D- | Arg | Trp(6-F) | Cys | | | |
| | | | | | | | cyclic) | | Phe(4-F) | | | | | | |
| 1143-2 | Lys* | Gly | Gly | γ-Glu | D-Nar | Cys | L- | His | D- | Arg | Trp(6-F) | Cys | | | |
| | | | | | | | aMeGlu | | Phe | | | | | | |
| 1144-2 | | Lys* | PEG1 | PEG1 | D-Nar | Cys | Aib(O- | Gln | D- | Arg | Trp(6-F) | Cys | | | |
| | | | | | | | cyclic) | | Phe(4-F) | | | | | | |
| 1145-2 | | Lys* | PEG1 | PEG1 | D-Nar | Cys | Phg | 3-Pal | D- | Arg | Trp(6-F) | Cys | | | |
| | | | | | | | | | Phe(4-F) | | | | | | |
| 1146-2 | Lys* | D-Arg | Gly | D-Arg | D-Nar | Cys | Phg | 3-Pal | D- | Arg | Trp(6-F) | Cys | | | |
| | | | | | | | | | Phe(4-F) | | | | | | |
| 1147-2 | Lys* | D-Arg | PEG1 | D-Arg | Beta- | Cys | Phg | 3-Pal | D- | Arg | Trp(6-F) | Cys | | | |
| | | | | | homoArg | | | | Phe(4-F) | | | | | | |
| 1148-2 | Lys* | D-Arg | Gly | D-Arg | D-Nar | Cys | D- | Gln | D-Phe | Arg | Trp(6-F) | Cys | | | |
| | | | | | | | aMeOrn | | | | | | | | |
| 1149-2 | | Lys* | Gly | D-Arg | D-Nar | Cys | D- | Gln | D-Phe | Arg | Trp(6-F) | Cys | | | |
| | | | | | | | aMeOrn | | | | | | | | |
| 1150-2 | Lys* | D-Arg | Gly | D-Arg | D-Nar | Cys | Aib(O- | Gln | D- | Arg | Trp(6-F) | Cys | | | |
| | | | | | | | cyclic) | | Phe(4-F) | | | | | | |
| 1151-2 | Lys* | D-Arg | PEG1 | D-Arg | Beta- | Cys | Aib(O- | Gln | D- | Arg | Trp(6-F) | Cys | | | |
| | | | | | homoArg | | cyclic) | | Phe(4-F) | | | | | | |
| 1152-2 | Lys* | D-Arg | Gly | D-Arg | D-Nar | Cys | Aib(O- | 3-Pal | D- | Arg | Trp(6-F) | Cys | | | |
| | | | | | | | cyclic) | | Phe(4-F) | | | | | | |
| | | | | | | | | | D- | | | | | | |
| 1153-2 | Lys* | D-Arg | Gly | D-Arg | D-Nar | Cys | Aib(O- | Orn | Phe(4-F) | Arg | Trp(6-F) | Cys | | | |
| | | | | | | | cyclic) | | D- | | | | | | |
| | | | | | | | | | Phe(4-F) | | | | | | |
| 1154-2 | Lys* | D-Arg | Gly | D-Arg | D-Nar | Cys | D- | 3-Pal | D- | Arg | Trp(6-F) | Cys | | | |
| | | | | | | | aMeOrn | | Phe(4-F) | | | | | | |
| 1155-2 | Lys* | D-Arg | Gly | D-Arg | D-Nar | Cys | D- | Orn | D- | Arg | Trp(6-F) | Cys | | | |
| | | | | | | | aMeOrn | | Phe(4-F) | | | | | | |
| 1156-2 | Lys* | D-Arg | Gly | D-Arg | D-Nar | Cys | D- | 3-Pal | D-Phe | Arg | Trp(6-F) | Cys | | | |
| | | | | | | | aMeOrn | | | | | | | | |
| 1157-2 | Lys* | D-Arg | Gly | D-Arg | D-Nar | Cys | D- | Orn | D-Phe | Arg | Trp(6-F) | Cys | | | |
| | | | | | | | aMeOrn | | | | | | | | |
| 1200-2 | | | | | D-Arg | Cys | L- | His | D-Phe | Arg | Trp(6-Me) | Cys | Gly | Gly | Lys* |
| | | | | | | | aMeAsp | | | | | | | | |
| 1201-2 | | | | | D-Nar | Cys | L- | His | D-Phe | Arg | Trp(6-Me) | Cys | Gly | Gly | Lys* |
| | | | | | | | aMeAsp | | | | | | | | |

In embodiments, the peptide of formula (II) is selected from Table E2A.

In embodiments, the peptide of formula (II) is selected from Table E2A, wherein the N-terminal, C-terminal and/or cyclic structure are optional feature.

TABLE E2A
Exemplary lipidated molecules.

| Molecule | | | | | | | | | | | | | N- | C- | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | term | term | Cyclic |
| 1128 | | Lys* | Glu | Pro | D-Nar | Cys | L- | His | D-Phe | Arg | Trp(6-Me) | Cys | Ac | NH₂ | Disulfide |
| | | | | | | | aMeGlu | | | | | | | | |
| 1129 | | Lys* | Gly | D-Arg | D-Nar | Cys | L- | His | D-Phe | Arg | Trp(6-Me) | Cys | Ac | NH₂ | Disulfide |
| | | | | | | | aMeGlu | | | | | | | | |
| 1130 | | Lys* | Gly | Gly | beta- | Cys | L- | His | D-Phe | Arg | Trp(6-Me) | Cys | Ac | NH₂ | Disulfide |
| | | | | | homoArg | | aMeAsp | | | | | | | | |
| 1131 | | Lys* | PEG1 | PEG1 | D-Nar | Cys | L- | His | D-Phe | Arg | Trp(6-Me) | Cys | Ac | NH₂ | Disulfide |
| | | | | | | | aMeAsp | | | | | | | | |
| 1132 | Lys* | Glu | PEG1 | PEG1 | D-Nar | Cys | L- | His | D- | Arg | Trp(6-Me) | Cys | Ac | NH₂ | Disulfide |
| | | | | | | | aMeGlu | | Phe(3,4,5- | | | | | | |
| | | | | | | | | | triF) | | | | | | |
| 1133 | | Lys* | Gly | gGlu | D-Nar | Cys | L- | His | D-Phe | Arg | Trp(6-Me) | Cys | Ac | NH₂ | Disulfide |
| | | | | | | | aMeGlu | | | | | | | | |
| 1134 | Lys* | Glu | Glu | Pro | D-Nar | Cys | L- | His | D- | Arg | Trp(6-Me) | Cys | Ac | NH₂ | Disulfide |
| | | | | | | | aMeGlu | | Phe(3,4,5- | | | | | | |
| | | | | | | | | | triF) | | | | | | |
| 1135 | | Lys* | Gly | Gly | D-Arg | Cys | L- | His | D-Phe | Arg | Trp(6-Me) | Cys | Ac | NH₂ | Disulfide |
| | | | | | | | aMeAsp | | | | | | | | |
| 1136 | Lys* | D-Arg | PEG1 | D-Arg | Beta- | Cys | D- | Gln | D-Phe | Arg | Trp(6-F) | Cys | Ac | NH₂ | Disulfide |
| | | | | | homoArg | | aMeOrn | | | | | | | | |
| 1137 | | Lys* | PEG1 | PEG1 | D-Nar | Cys | D- | Gln | D-Phe | Arg | Trp(6-F) | Cys | Ac | NH₂ | Disulfide |
| | | | | | | | aMeOrn | | | | | | | | |
| 1138 | | Lys* | Gly | D-Arg | D-Nar | Cys | L- | His | D-Phe | Arg | Trp(6-F) | Cys | Ac | NH₂ | Disulfide |
| | | | | | | | aMeGlu | | | | | | | | |
| 1139 | | Lys* | Gly | D-Arg | D-Nar | Cys | Phg | 3-Pal | D- | Arg | Trp(6-F) | Cys | Ac | NH₂ | Disulfide |
| | | | | | | | | | Phe(4-F) | | | | | | |
| 1140 | | Lys* | D-Arg | γ-Glu | D-Nar | Cys | L- | His | D-Phe | Arg | Trp(6-F) | Cys | Ac | NH₂ | Disulfide |
| | | | | | | | aMeGlu | | | | | | | | |
| 1141 | Lys* | Ser | Glu | Pro | D-Nar | Cys | L- | His | D-Phe | Arg | Trp(6-F) | Cys | Ac | NH₂ | Disulfide |
| | | | | | | | aMeGlu | | | | | | | | |
| 1142 | | Lys* | Gly | D-Arg | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1143 | Lys* | Gly | Gly | γ-Glu | D-Nar | Cys | L- | His | D-Phe | Arg | Trp(6-F) | Cys | Ac | NH₂ | Disulfide |
| | | | | | | | aMeGlu | | | | | | | | |
| 1144 | | Lys* | PEG1 | PEG1 | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1145 | | Lys* | PEG1 | PEG1 | D-Nar | Cys | Phg | 3-Pal | D- | Arg | Trp(6-F) | Cys | Ac | NH₂ | Disulfide |
| | | | | | | | | | Phe(4-F) | | | | | | |
| 1146 | Lys* | D-Arg | Gly | D-Arg | D-Nar | Cys | Phg | 3-Pal | D- | Arg | Trp(6-F) | Cys | Ac | NH₂ | Disulfide |
| | | | | | | | | | Phe(4-F) | | | | | | |
| 1147 | Lys* | D-Arg | PEG1 | D-Arg | Beta- | Cys | Phg | 3-Pal | D- | Arg | Trp(6-F) | Cys | Ac | NH₂ | Disulfide |
| | | | | | homoArg | | | | Phe(4-F) | | | | | | |
| 1148 | Lys* | D-Arg | Gly | D-Arg | D-Nar | Cys | D- | Gln | D-Phe | Arg | Trp(6-F) | Cys | Ac | NH₂ | Disulfide |
| | | | | | | | aMeOrn | | | | | | | | |
| 1149 | | Lys* | Gly | D-Arg | D-Nar | Cys | D- | Gln | D-Phe | Arg | Trp(6-F) | Cys | Ac | NH₂ | Disulfide |
| | | | | | | | aMeOrn | | | | | | | | |
| 1150 | Lys* | D-Arg | Gly | D-Arg | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1151 | Lys* | D-Arg | PEG1 | D-Arg | Beta- | Cys | Aib(O- | Gln | D- | Arg | Trp(6-F) | Cys | Ac | NH₂ | Disulfide |
| | | | | | homoArg | | cyclic) | | Phe(4-F) | | | | | | |
| 1152 | Lys* | D-Arg | Gly | D-Arg | D-Nar | Cys | Aib(O- | 3-Pal | D- | Arg | Trp(6-F) | Cys | Ac | NH₂ | Disulfide |
| | | | | | | | cyclic) | | Phe(4-F) | | | | | | |
| 1153 | Lys* | D-Arg | Gly | D-Arg | D-Nar | Cys | Aib(O- | Orn | D- | Arg | Trp(6-F) | Cys | Ac | NH₂ | Disulfide |
| | | | | | | | cyclic) | | Phe(4-F) | | | | | | |
| 1154 | Lys* | D-Arg | Gly | D-Arg | D-Nar | Cys | D- | 3-Pal | D- | Arg | Trp(6-F) | Cys | Ac | NH₂ | Disulfide |
| | | | | | | | aMeOrn | | Phe(4-F) | | | | | | |
| 1155 | Lys* | D-Arg | Gly | D-Arg | D-Nar | Cys | D- | Orn | D- | Arg | Trp(6-F) | Cys | Ac | NH₂ | Disulfide |
| | | | | | | | aMeOrn | | Phe(4-F) | | | | | | |
| 1156 | Lys* | D-Arg | Gly | D-Arg | D-Nar | Cys | D- | 3-Pal | D-Phe | Arg | Trp(6-F) | Cys | Ac | NH₂ | Disulfide |
| | | | | | | | aMeOrn | | | | | | | | |
| 1157 | Lys* | D-Arg | Gly | D-Arg | D-Nar | Cys | D- | Orn | D-Phe | Arg | Trp(6-F) | Cys | Ac | NH₂ | Disulfide |
| | | | | | | | aMeOrn | | | | | | | | |

| Molecule | | | | | | | | | | | | | | |
|---|---|---|---|---|---|---|---|---|---|---|---|---|---|---|
| Name | X₁ | X₂ | X₃ | X₄ | X₅ | X₆ | X₇ | X₈ | X₉ | X₁₀ | X₁₁ | N-term | C-term | Cyclic |
| 1200 | D-Arg | Cys | L- | His | D-Phe | Arg | Trp(6-Me) | Cys | Gly | Gly | Lys* | Ac | NH₂ | Disulfide |
| | | | aMeAsp | | | | | | | | | | | |
| 1201 | D-Nar | Cys | L- | His | D-Phe | Arg | Trp(6-Me) | Cys | Gly | Gly | Lys* | Ac | NH₂ | Disulfide |
| | | | aMeAsp | | | | | | | | | | | |

In embodiments, the peptide of formula (I) is selected from Table F1 or Table F2.

In embodiments, the peptides of Table F1 and Table F2 are not limited to N-terminal functional group, C-terminal functional group, and/or status or type of cyclic function.

In embodiments, the peptide of formula (I) is selected from Table F1.

TABLE F1
Exemplary peptide.

| Molecule | | | | | | | | |
|---|---|---|---|---|---|---|---|---|
| Name | X₁ | X₂ | X₃ | X₄ | X₅ | X₆ | X₇ | X₈ |
| 1108-1 | D-Nar | Cys | Cyclo- | 3Pal | D-Phe | Arg | Trp(6-F) | Cys |
| | | | Leu | | | | | |

In embodiments, the peptide of formula (I) is selected from Table F2.

In embodiments, the peptide of formula (I) is selected from Table F2, wherein the N-terminal, C-terminal and/or cyclic structure are optional feature.

TABLE F2
Exemplary peptide.

| Molecule | | | | | | | | | | | |
|---|---|---|---|---|---|---|---|---|---|---|---|
| Name | X₁ | X₂ | X₃ | X₄ | X₅ | X₆ | X₇ | X₈ | N-term | C-term | Cyclic |
| 1108 | D-Nar | Cys | Cyclo- | 3Pal | D-Phe | Arg | Trp(6-F) | Cys | Ac | NH2 | Disulfide |
| | | | Leu | | | | | | | | |

TABLE 1
Exemplary peptides. Cyclic peptides include bridge (e.g. disulfide) between X₂ and X

| | | | | | | | | | | | | | N- | C- | |
|---|---|---|---|---|---|---|---|---|---|---|---|---|---|---|---|
| [symbol] | [symbol] | X [symbol] | X [symbol] | X [symbol] | X [symbol] | X [symbol] | X [symbol] | X [symbol] | X [symbol] | X [symbol] | X [symbol] | X [symbol] | term | term | Cyclic |
| [symbol] | [symbol] | D-Arg | Gly | D-Arg | D-Nar | Cys | Phg | 3P [symbol] l | D-Phe(4-F) | Arg | Trp(6-F) | Cys | Ac | NH₂ | Disulfide |
| [symbol] | [symbol] | Lys [symbol] | Gly | D-Arg | D-Nar | Cys | Phg | 3P [symbol] l | D-Phe(4-F) | Arg | Trp(6-F) | Cys | Ac | NH₂ | Disulfide |
| 1145 | | Lys [symbol] | PEG1 | PEG1 | D-Nar | Cys | Phg | 3P [symbol] l | D-Phe(4-F) | Arg | Trp(6-F) | Cys | Ac | NH₂ | Disulfide |
| 1147 | Lys [symbol] | D-Arg | PEG1 | D-Arg | Beta-homoArg | Cys | Phg | 3P [symbol] l | D-Phe(4-F) | Arg | Trp(6-F) | Cys | Ac | NH₂ | Disulfide |
| 1148 | Lys [symbol] | D-Arg | Gly | D-Arg | D-Nar | Cys | D-aMeO [symbol] | Gln | D-Phe | Arg | Trp(6-F) | Cys | Ac | NH₂ | Disulfide |
| 1149 | | Lys [symbol] | Gly | D-Arg | D-Nar | Cys | D-aMeO [symbol] | Gln | D-Phe | Arg | Trp(6-F) | Cys | Ac | NH₂ | Disulfide |
| 1137 | | Lys [symbol] | PEG1 | PEG1 | D-Nar | Cys | D-aMeO [symbol] | Gln | D-Phe | Arg | Trp(6-F) | Cys | Ac | NH₂ | Disulfide |
| 1136 | Lys [symbol] | D-Arg | PEG1 | D-Arg | Beta-homoArg | Cys | D-aMeO [symbol] | Gln | D-Phe | Arg | Trp(6-F) | Cys | Ac | NH₂ | Disulfide |
| 1150 | Lys [symbol] | D-Arg | Gly | D-Arg | D-Nar | Cys | A [symbol] (O-cyclic) | Gln | D-Phe(4-F) | Arg | Trp(6-F) | Cys | Ac | NH₂ | Disulfide |
| 1142 | | Lys [symbol] | Gly | D-Arg | D-Nar | Cys | A [symbol] (O-cyclic) | Gln | D-Phe(4-F) | Arg | Trp(6-F) | Cys | Ac | NH₂ | Disulfide |
| 1144 | | Lys [symbol] | PEG1 | PEG1 | D-Nar | Cys | A [symbol] (O-cyclic) | Gln | D-Phe(4-F) | Arg | Trp(6-F) | Cys | Ac | NH₂ | Disulfide |
| 1151 | Lys [symbol] | D-Arg | PEG1 | D-Arg | Beta-homoArg | Cys | A [symbol] (O-cyclic) | Gln | D-Phe(4-F) | Arg | Trp(6-F) | Cys | Ac | NH₂ | Disulfide |
| 1152 | Lys [symbol] | D-Arg | Gly | D-Arg | D-Nar | Cys | A [symbol] (O-cyclic) | [symbol] | D-Phe(4-F) | Arg | Trp(6-F) | Cys | Ac | NH₂ | Disulfide |
| 1153 | Lys [symbol] | D-Arg | Gly | D-Arg | D-Nar | Cys | A [symbol] (O-cyclic) | O [symbol] | D-Phe(4-F) | Arg | Trp(6-F) | Cys | Ac | NH₂ | Disulfide |
| 1154 | Lys [symbol] | D-Arg | Gly | D-Arg | D-Nar | Cys | D-aMeO [symbol] | [symbol] | D-Phe(4-F) | Arg | Trp( [symbol] -F) | Cys | Ac | NH₂ | Disulfide |
| 1155 | Lys [symbol] | D-Arg | Gly | D-Arg | D-Nar | Cys | D-aMeO [symbol] | O [symbol] | D-Phe(4-F) | Arg | Trp(6-F) | Cys | Ac | NH₂ | Disulfide |
| 1156 | Lys [symbol] | D-Arg | Gly | D-Arg | D-Nar | Cys | D-aMeO [symbol] | [symbol] | D-Phe | Arg | Trp(6-F) | Cys | Ac | NH₂ | Disulfide |
| 1157 | Lys [symbol] | D-Arg | Gly | D-Arg | D-Nar | Cys | D-aMeO [symbol] | O [symbol] | D-Phe | Arg | Trp(6-F) | Cys | Ac | NH₂ | Disulfide |
| 1122 | | | | | D-Nar | Cys | A [symbol] b(O-cyclic) | [symbol] | D-Phe(4-F) | Arg | Trp(6-F) | Cys | Ac | NH₂ | Disulfide |
| 1123 | | | | | D-Nar | Cys | A [symbol] b(O-cyclic) | O [symbol] | D-Phe(4-F) | Arg | Trp(6-F) | Cys | Ac | NH₂ | Disulfide |
| 1124 | | | | | D-Nar | Cys | D-aMe [symbol] | [symbol] | D-Phe(4-F) | Arg | Trp(6-F) | Cys | Ac | NH₂ | Disulfide |
| 1125 | | | | | D-Nar | Cys | D-aMeO [symbol] | O [symbol] | D-Phe(4-F) | Arg | Trp(6-F) | Cys | Ac | NH₂ | Disulfide |
| 1126 | | | | | D-Nar | Cys | D-aMeO [symbol] | [symbol] | D-Phe | Arg | Trp(6-F) | Cys | Ac | NH₂ | Disulfide |
| 1127 | | | | | D-Nar | Cys | D-aMeO [symbol] | [symbol] | D-Phe | Arg | Trp(6-F) | Cys | Ac | NH₂ | Disulfide |
| 25 | | | | | D-Nar | Glu | A [symbol] b(O-cyclic) | Gln | D-Phe(4-F) | Arg | Trp(6-F) | Dap | Ac | NH₂ | Disulfide |
| 26 | | | | | D-Nar | Glu | D-aMe [symbol] | Gln | D-Phe | Arg | Trp(6-F) | Dap | Ac | NH₂ | Disulfide |
| 27 | | | | | D-Nar | Glu | Phg | [symbol] | D-Phe(4-F) | Arg | Trp(6-F) | Dap | Ac | NH₂ | Disulfide |
| 28 | | | | | Beta-homoArg | Cys | A [symbol] (O-cyclic) | Gln | D-Phe(4-F) | Arg | Trp(6-F) | Cys | Ac | NH₂ | Disulfide |
| 29 | | | | | Beta-homoArg | Cys | D-aMeO [symbol] | Gln | D-Phe | Arg | Trp(6-F) | Cys | Ac | NH₂ | Disulfide |
| 30 | | | | | Beta-homoArg | Cys | Phg | [symbol] | D-Phe(4-F) | Arg | Trp(6-F) | Cys | Ac | NH₂ | Disulfide |
| 31 | | | | | Beta-homoArg | Cys | Phg | Gln | D-Phe(4-F) | Arg | Trp(6-F) | Cys | Ac | NH₂ | Disulfide |
| 32 | | | | | D-Nar | Cys | Phg | Gln | D-Phe(4-F) | Arg | Trp(6-F) | Cys | Ac | NH₂ | Disulfide |
| 33 | | | | | D-Nar | Cys | Phg | [symbol] | D-Phe(4-F) | Arg | Trp(6-F) | Cys | Ac | NH₂ | Disulfide |
| 34 | | | | | D-Nar | Cys | Phg | [symbol] | D-Phe(4-F) | Arg | Trp(6-F) | Cys | Ac | NH₂ | Disulfide |
| 35 | | | | | D-Nar | Cys | Phe | [symbol] | D-Phe(4-F) | Arg | Trp(6-F) | Cys | Ac | NH₂ | Disulfide |
| 36 | | | | | D-Nar | Cys | Tyr | [symbol] | D-Phe(4-F) | Arg | Trp(6-F) | Cys | Ac | NH₂ | Disulfide |
| 37 | | | | | D-Nar | Cys | Phe | Gln | D-Phe(4-F) | Arg | Trp(6-F) | Cys | Ac | NH₂ | Disulfide |
| 38 | | | | | D-Nar | Cys | Tyr | Gln | D-Phe(4-F) | Arg | Trp(6-F) | Cys | Ac | NH₂ | Disulfide |
| 39 | | | | | D-Nar | Cys | D-Phe | Gln | D-Phe(4-F) | Arg | Trp(6-F) | Cys | Ac | NH₂ | Disulfide |
| 1158 | | | | | D-Nar | Cys | A [symbol] b(O-cyclic) | Gln | D-Phe(4-F) | Arg | Trp(6-F) | Pen | Ac | NH₂ | Disulfide |
| 41 | | | | | D-Nar | Cys | D-aMeO [symbol] | Gln | D-Phe | Arg | Trp(6-F) | Pen | Ac | NH₂ | Disulfide |
| 42 | | | | | D-Nar | Cys | Phg | [symbol] | D-Phe(4-F) | Arg | Trp(6-F) | Pen | Ac | NH₂ | Disulfide |
| 43 | | | | | D-Nar | hCys | A [symbol] (O-cyclic) | Gln | D-Phe(4-F) | Arg | Trp(6-F) | Pen | Ac | NH₂ | Disulfide |
| 44 | | | | | D-Nar | hCys | D-aMeO [symbol] | Gln | D-Phe | Arg | Trp(6-F) | Pen | Ac | NH₂ | Disulfide |
| 45 | | | | | D-Nar | hCys | Phg | [symbol] | D-Phe(4-F) | Arg | Trp(6-F) | Pen | Ac | NH₂ | Disulfide |
| 46 | | | | | D-Nar | Cys | A [symbol] b(O-cyclic) | Gln | D-Phe | Arg | Trp | Cys | Ac | NH₂ | Disulfide |
| 47 | | | | | D-Nar | Cys | D-aMeO [symbol] | Gln | D-Phe | Arg | Trp | Cys | Ac | NH₂ | Disulfide |
| 48 | | | | | D-Nar | Cys | Phg | [symbol] | D-Phe | Arg | Trp | Cys | Ac | NH₂ | Disulfide |
| 49 | | | | | Arg | Cys | Phg | [symbol] | D-Phe | Arg | Trp | Cys | Ac | NH₂ | Disulfide |
| 50 | | | | | Arg | Cys | Phg | Gln | D-Phe | Arg | Trp | Cys | Ac | NH₂ | Disulfide |
| 51 | | | | | Arg | Cys | A [symbol] b(O-cyclic) | Gln | D-Phe | Arg | Trp | Cys | Ac | NH₂ | Disulfide |
| 52 | | | | | Arg | Cys | D-aMeO [symbol] | Gln | D-Phe | Arg | Trp | Cys | Ac | NH₂ | Disulfide |
| 53 | | | | | Arg | Cys | D-aMeO [symbol] | Gln | D-Phe | Arg | Trp | Pen | Ac | NH₂ | Disulfide |
| 54 | | | | | D-Nar | Cys | Phg | [symbol] | D-Phe(4-F) | Arg | Trp(6-F) | Cys | Ac | NH₂ | Disulfide |
| 55 | | | | | D-Nar | Cys | D-Phg | [symbol] | D-Phe(4-F) | Arg | Trp(6-F) | Cys | Ac | NH₂ | Disulfide |
| 56 | | | | | D-Nar | Cys | D-Phg | Gln | D-Phe(4-F) | Arg | Trp(6-F) | Cys | Ac | NH₂ | Disulfide |
| 57 | | | | | D-Nar | Cys | [symbol] | [symbol] | D-Phe(4-F) | Arg | Trp(6-F) | Cys | Ac | NH₂ | Disulfide |
| 58 | | | | | D-Nar | Cys | [symbol] | Gln | D-Phe(4-F) | Arg | Trp(6-F) | Cys | Ac | NH₂ | Disulfide |
| 59 | | | | | D-Nar | Cys | bAc5c | [symbol] | D-Phe(4-F) | Arg | Trp(6-F) | Cys | Ac | NH₂ | Disulfide |
| 60 | | | | | D-Nar | Cys | bAc5c | Gln | D-Phe(4-F) | Arg | Trp(6-F) | Cys | Ac | NH₂ | Disulfide |
| 61 | | | | | Beta-homoArg | Cys | bAc5c | [symbol] | D-Phe(4-F) | Arg | Trp(6-F) | Cys | Ac | NH₂ | Disulfide |
| 62 | | | | | Beta homoArg | Cys | bAc5c | Gln | D-Phe(4-F) | Arg | Trp(6-F) | Cys | Ac | NH₂ | Disulfide |
| 63 | | | | | D-Nar | Cys | bAc4c | [symbol] | D-Phe(4-F) | Arg | Trp(6-F) | Cys | Ac | NH₂ | Disulfide |
| 64 | | | | | D-Nar | Cys | bAc4c | Gln | D-Phe(4-F) | Arg | Trp(6-F) | Cys | Ac | NH₂ | Disulfide |
| 65 | | | | | Beta-homoArg | Cys | bAc4c | [symbol] | D-Phe(4-F) | Arg | Trp(6-F) | Cys | Ac | NH₂ | Disulfide |
| 66 | | | | | Beta-homoArg | Cys | bAc4c | Gln | D-Phe(4-F) | Arg | Trp(6-F) | Cys | Ac | NH₂ | Disulfide |
| 67 | | | | | D-Nar | Cys | bAc3c | [symbol] | D-Phe(4-F) | Arg | Trp(6-F) | Cys | Ac | NH₂ | Disulfide |
| 68 | | | | | D-Nar | Cys | bAc3c | Gln | D-Phe(4-F) | Arg | Trp(6-F) | Cys | Ac | NH₂ | Disulfide |
| 69 | | | | | Beta-homoArg | Cys | bAc3c | [symbol] | D-Phe(4-F) | Arg | Trp(6-F) | Cys | Ac | NH₂ | Disulfide |
| 70 | | | | | Beta-homoArg | Cys | bAc3c | Gln | D-Phe(4-F) | Arg | Trp(6-F) | Cys | Ac | NH₂ | Disulfide |
| 71 | | | | | D-Nar | Cys | Ac3c | [symbol] | D-Phe(4-F) | Arg | Trp(6-F) | Cys | Ac | NH₂ | Disulfide |
| 72 | | | | | D-Nar | Cys | Ac4c | [symbol] | D-Phe(4-F) | Arg | Trp(6-F) | Cys | Ac | NH₂ | Disulfide |
| 73 | | | | | D-Nar | Cys | Ac6c | [symbol] | D-Phe(4-F) | Arg | Trp(6-F) | Cys | Ac | NH₂ | Disulfide |
| 74 | | | | | Beta-homoArg | Cys | Ac3c | [symbol] | D-Phe(4-F) | Arg | Trp(6-F) | Cys | Ac | NH₂ | Disulfide |
| 75 | | | | | Beta-homoArg | Cys | Ac4c | [symbol] | D-Phe(4-F) | Arg | Trp(6-F) | Cys | Ac | NH₂ | Disulfide |
| 76 | | | | | Beta-homoArg | Cys | Ac5c | [symbol] | D-Phe(4-F) | Arg | Trp(6-F) | Cys | Ac | NH₂ | Disulfide |
| 77 | | | | | D-Nar | Cys | Cyclo-Leu | [symbol] | D-Phe(4-F) | Arg | Trp(6-F) | Cys | Ac | NH₂ | Disulfide |
| 78 | | | | | D-Nar | Cys | Cyclo-Leu | Gln | D-Phe(4-F) | Arg | Trp(6-F) | Cys | Ac | NH₂ | Disulfide |
| 79 | | | | | Beta-homoArg | Cys | Cyclo-Leu | [symbol] | D-Phe(4-F) | Arg | Trp(6-F) | Cys | Ac | NH₂ | Disulfide |
| 80 | | | | | Beta-homoArg | Cys | Cyclo-Leu | Gln | D-Phe(4-F) | Arg | Trp(6-F) | Cys | Ac | NH₂ | Disulfide |
| 81 | | | | | Beta-homoArg | Cys | Cyclo-Leu | [symbol] | D-Phe(4-F) | Arg | Trp(6-F) | Cys | Ac | NH₂ | Disulfide |
| 82 | | | | | Beta-homoArg | Cys | Cyclo-Leu | [symbol] | D-Phe(4-F) | Arg | Trp(6-F) | Cys | Ac | NH₂ | Disulfide |
| 83 | | | | | D-Nar | Cys | Alb(O-cyclic) | Thr | D-Phe(4-F) | Arg | Trp(6-F) | Cys | Ac | NH₂ | Disulfide |
| 84 | | | | | D-Nar | Cys | D-aMe [symbol] | Thr | D-Phe | Arg | Trp(6-F) | Cys | Ac | NH₂ | Disulfide |
| 85 | | | | | D-Nar | Cys | Phg | Thr | D-Phe(4-F) | Arg | Trp(6-F) | Cys | Ac | NH₂ | Disulfide |
| 86 | | | | | D-Nar | Cys | A [symbol] b(O-cyclic) | Thr | D-Phe(4-F) | Arg | Trp(6-F) | Pen | Ac | NH₂ | Disulfide |
| 87 | | | | | D-Nar | Cyc | D-aMeO [symbol] | Thr | D-Phe | Arg | Trp(6-F) | Pen | Ac | NH₂ | Disulfide |
| 88 | | | | | D-Nar | Cys | Phg | Thr | D-Phe(4-F) | Arg | Trp(6-F) | Pen | Ac | NH₂ | Disulfide |
| 89 | | | | | D-Nar | Cys | A [symbol] b(O-cyclic) | Ser | D-Phe(4-F) | Arg | Trp(6-F) | Cys | Ac | NH₂ | Disulfide |
| 90 | | | | | D-Nar | Cys | D-aMeO [symbol] | Ser | D-Phe | Arg | Trp(6-F) | Cys | Ac | NH₂ | Disulfide |
| 91 | | | | | D-Nar | Cys | Phg | Ser | D-Phe(4-F) | Arg | Trp(6-F) | Cys | Ac | NH₂ | Disulfide |
| 92 | | | | | D-Nar | Cys | A [symbol] b(O-ycylic) | Ser | D-Phe(4-F) | Arg | Trp(6-F) | Pen | Ac | NH₂ | Disulfide |
| 93 | | | | | D-Nar | Cys | D-aMeO [symbol] | Ser | D-Phe | Arg | Trp(6-F) | Pen | Ac | NH₂ | Disulfide |
| 94 | | | | | D-Nar | Cys | Phg | Ser | D-Phe(4-F) | Arg | Trp(6-F) | Cys | Ac | NH₂ | Disulfide |
| 95 | | | | | D-Nar | Cys | D-aMeSer | [symbol] | D-Phe(4-F) | Arg | Trp(6-F) | Cys | Ac | NH₂ | Disulfide |
| 96 | | | | | D-Nar | Cys | D-aMeSer | Thr | D-Phe(4-F) | Arg | Trp(6-F) | Cys | Ac | NH₂ | Disulfide |
| 97 | | | | | D-Nar | Cys | D-aMeSer | Ser | D-Phe(4-F) | Arg | Trp(6-F) | Cys | Ac | NH₂ | Disulfide |
| 98 | | | | | D-Nar | Cys | D-aMeSer | [symbol] | D-Phe(4-F) | Arg | Trp(6-F) | Cys | Ac | NH₂ | Disulfide |
| 99 | | | | | D-Nar | Cys | D-aMeSer | [symbol] | D-Phe | Arg | Trp(6-F) | Cys | Ac | NH₂ | Disulfide |
| 100 | | | | | Beta-homoArg | Cys | D-aMeSer | [symbol] | D-Phe | Arg | Trp(6-F) | Cys | Ac | NH₂ | Disulfide |
| 101 | | | | | Beta-homoArg | Cys | D-aMeSer | Gln | D-Phe | Arg | Trp(6-F) | Cys | Ac | NH₂ | Disulfide |
| 102 | | | | | D-Nar | Cys | L-aMeSer | [symbol] | D-Phe(4-F) | Arg | Trp(6-F) | Cys | Ac | NH₂ | Disulfide |
| 103 | | | | | D-Nar | Cys | L-aMeSer | Thr | D-Phe(4-F) | Arg | Trp(6-F) | Cys | Ac | NH₂ | Disulfide |
| 104 | | | | | D-Nar | Cys | L-aMeSer | Ser | D-Phe(4-F) | Arg | Trp(6-F) | Cys | Ac | NH₂ | Disulfide |
| 105 | | | | | D-Nar | Cys | L-aMeSer | [symbol] | D-Phe(4-F) | Arg | Trp(6-4) | Cys | Ac | NH₂ | Disulfide |
| 106 | | | | | D-Nar | Cys | L-aMeSer | [symbol] | D-Phe | Arg | Trp(6-F) | Cys | Ac | NH₂ | Disulfide |
| 107 | | | | | Beta-homoArg | Cys | L-aMeSer | [symbol] | D-Phe | Arg | Trp(6-F) | Cys | Ac | NH₂ | Disulfide |
| 108 | | | | | Beta-homoArg | Cys | L-aMeSer | Gln | D-Phe | Arg | Trp(6-F) | Cys | Ac | NH₂ | Disulfide |
| 109 | | | | | D-Nar | Cys | D-aMeAsp | [symbol] | D-Phe(4-F) | Arg | Trp(6-F) | Cys | Ac | NH₂ | Disulfide |
| 110 | | | | | D-Nar | Cys | D-aMeSer | Gln | D-Phe(4-F) | Arg | Trp(5-Me) | Cys | Ac | NH₂ | Disulfide |
| 111 | Lys [symbol] | Gly | Gly | Gly | D-Nar | Cys | A [symbol] (O-cyclic) | Gln | D-Phe(4-F) | Arg | Trp(6-F) | Cys | Ac | NH₂ | Disulfide |
| 112 | | Lys [symbol] | Gly | Gly | D-Nar | Cys | A [symbol] (O-cyclic) | Gln | D-Phe(4-F) | Arg | Trp(6-F) | Cys | Ac | NH₂ | Disulfide |
| 113 | | | Lys [symbol] | Gly | D-Nar | Cys | A [symbol] (O-cyclic) | Gln | D-Phe(4-F) | Arg | Trp(6-F) | Cys | Ac | NH₂ | Disulfide |
| 114 | | | | Lys [symbol] | D-Nar | Cys | A [symbol] (O-cyclic) | Gln | D-Phe(4-F) | Arg | Trp(6-F) | Cys | Ac | NH₂ | Disulfide |
| 123 | | Lys [symbol] | Gly | Gly | D-Nar | Cys | Phg | [symbol] | D-Phe(4-F) | Arg | Trp(6-F) | Cys | Ac | NH₂ | Disulfide |
| 124 | | | Lys [symbol] | Gly | D-Nar | Cys | Phg | [symbol] | D-Phe(4-F) | Arg | Trp(6-F) | Cys | Ac | NH₂ | Disulfide |
| 125 | | | Lys [symbol] | Gly | D-Nar | Cys | Phg | [symbol] | D-Phe(4-F) | Arg | Trp(6-F) | Cys | Ac | NH₂ | Disulfide |
| 130 | | Lys [symbol] | Gly | Gly | D-Nar | Cys | D-aMeO [symbol] | Gln | D-Phe | Arg | Trp(6-F) | Cys | Ac | NH₂ | Disulfide |
| 131 | | | Lys [symbol] | Gly | D-Nar | Cys | D-aMeO [symbol] | Gln | D-Phe | Arg | Trp(6-F) | Cys | Ac | NH₂ | Disulfide |
| 132 | | | | Lys [symbol] | D-Nar | Cys | D-aMeO [symbol] | Gln | D-Phe | Arg | Trp(6-F) | Cys | Ac | NH₂ | Disulfide |
| 137 | | | | | Beta-homoArg | Cys | A [symbol] (O-cyclic) | [symbol] | D-Phe(4-F) | Arg | Trp(6-F) | Cys | Ac | NH₂ | Disulfide |
| 138 | | | Lys [symbol] | Gly | D-Nar | Cys | Cyclo-Leu | [symbol] | D-Phe | Arg | Trp(6-F) | Cys | Ac | NH₂ | Disulfide |
| 139 | | | Lys [symbol] | Gly | D-Nar | Cys | A [symbol] (O-cyclic) | [symbol] | D-Phe | Arg | Trp(6-F) | Cys | Ac | NH₂ | Disulfide |
| 140 | | | Lys [symbol] | Lys [symbol] | Arg | Cys | A [symbol] (O-cyclic) | Gln | D-Phe(4-F) | Arg | Trp(6-F) | Pen | Ac | NH₂ | Disulfide |
| 141 | | | | Lys [symbol] | D-Nar | Cys | A [symbol] (O-cyclic) | Gln | D-Phe(4-F) | Arg | Trp(6-F) | Pen | Ac | NH₂ | Disulfide |
| 142 | | | | Lys [symbol] | BetahomoArg | Cys | A [symbol] (O-cyclic) | Gln | D-Phe(4-F) | Arg | Trp(6-F) | Pen | Ac | NH₂ | Disulfide |
| 143 | | | | Lys [symbol] | Arg | Cys | A [symbol] (O-cyclic) | Gln | D-Phe | Arg | Trp | Pen | Ac | NH₂ | Disulfide |
| 144 | | | | Lys [symbol] | D-Nar | Cys | A [symbol] (O-cyclic) | Gln | D-Phe | Arg | Trp | Pen | Ac | NH₂ | Disulfide |
| 145 | | | | Lys [symbol] | BetahomoArg | Cys | A [symbol] (O-cyclic) | Gln | D-Phe | Arg | Trp | Pen | Ac | NH₂ | Disulfide |
| 146 | | | | Lys [symbol] | Arg | Cys | A [symbol] (O-cyclic) | Gln | D-Phe | Arg | Trp(6-F) | Pen | Ac | NH₂ | Disulfide |
| 147 | | | | Lys [symbol] | D-Nar | Cys | A [symbol] (O-cyclic) | Gln | D-Phe | Arg | Trp(6-F) | Pen | Ac | NH₂ | Disulfide |
| 148 | | | | Lys [symbol] | BetahomoArg | Cys | A [symbol] (O-cyclic) | Gln | D-Phe | Arg | Trp(6-F) | Pen | Ac | NH₂ | Disulfide |
| 149 | | | | Lys [symbol] | Arg | Cys | A [symbol] (O-cyclic) | Gln | D-Phe(4-F) | Arg | Trp | Pen | Ac | NH₂ | Disulfide |
| 150 | | | | Lys [symbol] | D-Nar | Cys | A [symbol] (O-cyclic) | Gln | D-Phe(4-F) | Arg | Trp | Pen | Ac | NH₂ | Disulfide |
| 151 | | | | Lys [symbol] | BetahomoArg | Cys | A [symbol] (O-cyclic) | Gln | D-Phe(4-F) | Arg | Trp | Pen | Ac | NH₂ | Disulfide |
| 152 | | | | | D-Nar | Cys | A [symbol] (O-cyclic) | Gln | D-Phe | Arg | Trp(6-F) | Pen | Ac | NH₂ | Disulfide |
| 153 | | | | | D-Nar | Cys | A [symbol] (O-cyclic) | Gln | D-Phe(4-F) | Arg | Trp | Pen | Ac | NH₂ | Disulfide |
| 154 | | | | | Beta-homoArg | Cys | D-aMeO [symbol] | Gln | D-Phe | Arg | Trp(6-F) | Pen | Ac | NH₂ | Disulfide |
| 155 | | | | | Beta-homoArg | Cys | D-aMeO [symbol] | Gln | D-Phe(4-F) | Arg | Trp(6-F) | Pen | Ac | NH₂ | Disulfide |
| 156 | | | | | Beta-homoArg | Cys | D-aMeO [symbol] | Gln | D-Phe(4-F) | Arg | Trp | Pen | Ac | NH₂ | Disulfide |
| 157 | | | | | Beta-homoArg | Cys | Cyclo-Leu | Gln | D-Phe(4-F) | Arg | Trp(6-F) | Pen | Ac | NH₂ | Disulfide |
| 158 | | | | | D-Nar | Cys | Cyclo-Leu | Gln | D-Phe(4-F) | Arg | Trp(6-F) | Pen | Ac | NH₂ | Disulfide |
| 159 | | | | | D-Nar | Cys | D- [symbol] | [symbol] | D-Phe(4-F) | Arg | Trp(6-F) | Pen | Ac | NH₂ | Disulfide |
| 160 | | | | | D-Nar | Cys | D- [symbol] | Gln | D-Phe(4-F) | Arg | Trp(6-F) | Pen | Ac | NH₂ | Disulfide |
| 161 | | | | | Beta-homoArg | Cys | Cyclo-Leu | Gln | D-Phe | Arg | Trp(6-F) | Pen | Ac | NH₂ | Disulfide |
| 162 | | | | | D-Nar | Cys | Cyclo-Leu | Gln | D-Phe | Arg | Trp(6-F) | Pen | Ac | NH₂ | Disulfide |
| 163 | | | | | D-Nar | Cys | D- [symbol] | [symbol] | D-Phe | Arg | Trp(6-F) | Pen | Ac | NH₂ | Disulfide |
| 164 | | | | | D-Nar | Cys | D- [symbol] | Gln | D-Phe | Arg | Trp(6-F) | Pen | Ac | NH₂ | Disulfide |
| 165 | | | | Lys [symbol] | Arg | Cys | A [symbol] | Gln | D-Phe(4-F) | Arg | Trp(6-F) | Pen | Ac | NH₂ | Disulfide |
| 166 | | | | Lys [symbol] | D-Nar | Pen | A [symbol] (O-cyclic) | Gln | D-Phe(4-F) | Arg | Trp(6-F) | Pen | Ac | NH₂ | Disulfide |
| 167 | | | | Lys [symbol] | D-Nar | Pen | A [symbol] (O-cyclic) | Gln | D-Phe(4-F) | Arg | Trp(6-F) | Cys | Ac | NH₂ | Disulfide |
| 168 | | | | | D-Nar | Pen | A [symbol] (O-cyclic) | Gln | D-Phe(4-F) | Arg | Trp(6-F) | Pen | Ac | NH₂ | Disulfide |
| 169 | | | | | D-Nar | Pen | A [symbol] (O-cyclic) | Gln | D-Phe(4-F) | Arg | Trp(6-F) | Cys | Ac | NH₂ | Disulfide |
| 170 | | | | | D-Nar | Cys | A [symbol] (O-cyclic) | Lys | D-Phe(4-F) | Arg | Trp(6-F) | Pen | Ac | NH₂ | Disulfide |
| 171 | | | | | D-Nar | Cys | (3S)-3- | Gln | D-Phe(4-F) | Arg | Trp(6-F) | Pen | Ac | NH₂ | Disulfide |
| | | | | | | | Aminotetrahydro- | | | | | | | | |
| | | | | | | | 3-furancarbocyclic acid | | | | | | | | |
| 172 | | | | | D-Nar | Cys | (3S)-3- | Gln | D-Phe(4-F) | Arg | Trp(6-F) | Pen | Ac | NH₂ | Disulfide |
| | | | | | | | Aminotetrahydro- | | | | | | | | |
| | | | | | | | 3-furancarbocyclic acid | | | | | | | | |
| 173 | | | | | D-Nar | Cys | (3S)-3- | Gln | D-Phe(4-F) | Arg | Trp(6-F) | Pen | Ac | NH₂ | Disulfide |
| | | | | | | | Aminotetrahydro- | | | | | | | | |
| | | | | | | | 3- [symbol] acid | | | | | | | | |
| 174 | | | | | D-Nar | Cys | (3R)-3- | Gln | D-Phe(4-F) | Arg | Trp(6-F) | Pen | Ac | NH₂ | Disulfide |
| | | | | | | | Aminotetrahydro- | | | | | | | | |
| | | | | | | | 3- [symbol] acid | | | | | | | | |
| 175 | | | | | D-Nar | Cys | N-Boc-(3S)-3-amino- | Gln | D-Phe(4-F) | Arg | Trp(6-F) | Pen | Ac | NH₂ | Disulfide |
| | | | | | | | 1,3- | | | | | | | | |
| | | | | | | | pyro [symbol] carboxylate | | | | | | | | |
| 176 | | | | | D-Nar | Cys | N-Boc-(3R)-3-amino- | Gln | D-Phe(4-F) | Arg | Trp(6-F) | Pen | Ac | NH₂ | Disulfide |
| | | | | | | | 1,3- | | | | | | | | |
| | | | | | | | pyro [symbol] carboxylate | | | | | | | | |
| 177 | | | | | D-Nar | Cys | 3-Amino-3- [symbol] - | Gln | D-Phe(4-F) | Arg | Trp(6-F) | Pen | Ac | NH₂ | Disulfide |
| | | | | | | | [symbol] acid | | | | | | | | |
| 178 | | | | | D-Nar | Cys | 3-Amino [symbol] -3- | Gln | D-Phe(4-F) | Arg | Trp(6-F) | Pen | Ac | NH₂ | Disulfide |
| | | | | | | | carb [symbol] acid 1,1- | | | | | | | | |
| | | | | | | | dioxide | | | | | | | | |
| 179 | | | | | D-Nar | Cys | N-Boc-3-amino-1,3- | Gln | D-Phe(4-F) | Arg | Trp(6-F) | Pen | Ac | NH₂ | Disulfide |
| | | | | | | | [symbol] | | | | | | | | |
| 180 | | | | | D-Nar | Cys | 1-Amino-3,3- | Gln | D-Phe(4-F) | Arg | Trp(6-F) | Pen | Ac | NH₂ | Disulfide |
| | | | | | | | dimethylcyclo [symbol] | | | | | | | | |
| | | | | | | | carboxylic acid | | | | | | | | |
| 181 | | | | | D-Nar | Cys | [symbol] - | Gln | D-Phe(4-F) | Arg | Trp(6-F) | Pen | Ac | NH₂ | Disulfide |
| | | | | | | | Amino [symbol] [2.3]hexane- | | | | | | | | |
| | | | | | | | 5-carboxylic acid | | | | | | | | |
| 182 | | | | | D-Nar | Cys | 6-Amino-2- | Gln | D-Phe(4-F) | Arg | Trp(6-F) | Pen | Ac | NH₂ | Disulfide |
| | | | | | | | [symbol] [3.3]heptane- | | | | | | | | |
| | | | | | | | 6-carboxylic acid | | | | | | | | |
| 183 | | | | | D-Nar | Cys | 2-amino-2- | Gln | D-Phe(4-F) | Arg | Trp(6-F) | Pen | Ac | NH₂ | Disulfide |
| | | | | | | | ethylbutanoic acid | | | | | | | | |
| 184 | | | | | D-Nar | Cys | (1S)-1-Amino-2,3- | Gln | D-Phe(4-F) | Arg | Trp(6-F) | Pen | Ac | NH₂ | Disulfide |
| | | | | | | | dihydro- | | | | | | | | |
| | | | | | | | [symbol] -1-carboxylic | | | | | | | | |
| | | | | | | | acid | | | | | | | | |
| 185 | | | | | D-Nar | Cys | (1R)-1-Amino-2,3- | Gln | D-Phe(4-F) | Arg | Trp(6-F) | Pen | Ac | NH₂ | Disulfide |
| | | | | | | | dihydro- | | | | | | | | |
| | | | | | | | [symbol] -1-carboxylic | | | | | | | | |
| | | | | | | | acid | | | | | | | | |
| 186 | | | | | D-Nar | Cys | A [symbol] (O-cyclic) | Gln | L-Meth [symbol] | Arg | Trp(6-F) | Pen | Ac | NH₂ | Disulfide |
| | | | | | | | | | sulfoxide | | | | | | |
| 187 | | | | | D-Nar | Cys | A [symbol] (O-cyclic) | Gln | L-Meth [symbol] | Arg | Trp(6-F) | Pen | Ac | NH₂ | Disulfide |
| | | | | | | | | | sulfoxide | | | | | | |
| 188 | | | | | D-Nar | Cys | A [symbol] (O-cyclic) | Gln | (2S)-2-Amino-4- | Arg | Trp(6-F) | Pen | Ac | NH₂ | Disulfide |
| | | | | | | | | | cyano [symbol] acid | | | | | | |
| 189 | | | | | D-Nar | Cys | A [symbol] (O-cyclic) | Gln | 3-(Acetylamino)-L- | Arg | Trp(6-F) | Pen | Ac | NH₂ | Disulfide |
| | | | | | | | | | alanine | | | | | | |
| 190 | | | | | D-Nar | Cys | A [symbol] (O-cyclic) | Gln | O-Carb [symbol] -L- | Arg | Trp(6-F) | Pen | Ac | NH₂ | Disulfide |
| | | | | | | | | | [symbol] | | | | | | |
| 191 | | | | | D-Nar | Cys | A [symbol] (O-cyclic) | Gln | 2-Hydroxy-L- | Arg | Trp(6-F) | Pen | Ac | NH₂ | Disulfide |
| | | | | | | | | | tryptophan | | | | | | |
| 192 | | | | | D-Nar | Cys | A [symbol] (O-cyclic) | Gln | 3-(Trimethyl [symbol] )-D- | Arg | Trp(6-F) | Pen | Ac | NH₂ | Disulfide |
| | | | | | | | | | alanine | | | | | | |
| 193 | | | | | D-Nar | Cys | A [symbol] (O-cyclic) | Gln | 5,5,5-Tri [symbol] -D- | Arg | Trp(6-F) | Pen | Ac | NH₂ | Disulfide |
| | | | | | | | | | [symbol] | | | | | | |
| 194 | | | | | D-Nar | Cys | A [symbol] (O-cyclic) | Gln | 3-( [symbol] )- | Arg | Trp(6-F) | Pen | Ac | NH₂ | Disulfide |
| | | | | | | | | | D-alanine | | | | | | |
| 195 | | | | | D-Nar | Cys | A [symbol] (O-cyclic) | Gln | 3-Cyano-D-alanine | Arg | Trp(6-F) | Pen | Ac | NH₂ | Disulfide |
| 196 | | | | | D-Nar | Cys | A [symbol] (O-cyclic) | Gln | 3-Cyclopropyl-D- | Arg | Trp(6-F) | Pen | Ac | NH₂ | Disulfide |
| | | | | | | | | | alanine | | | | | | |
| 197 | | | | | D-Nar | Cys | A [symbol] (O-cyclic) | Gln | (R)-2-Amino-4- | Arg | Trp(6-F) | Pen | Ac | NH₂ | Disulfide |
| | | | | | | | | | cyclopropyl [symbol] | | | | | | |
| | | | | | | | | | acid | | | | | | |
| 198 | | | | | D-Nar | Cys | A [symbol] (O-cyclic) | Gln | (αR)-α-Amino-2- | Arg | Trp(6-F) | Pen | Ac | NH₂ | Disulfide |
| | | | | | | | | | [symbol] | | | | | | |
| | | | | | | | | | propanoic acid | | | | | | |
| 199 | | | | | D-Nar | Cys | A [symbol] (O-cyclic) | Gln | (αR)-α-Amino-3- | Arg | Trp(6-F) | Pen | Ac | NH₂ | Disulfide |
| | | | | | | | | | [symbol] - | | | | | | |
| | | | | | | | | | propanoic acid | | | | | | |
| 200 | | | | | D-Nar | Cys | A [symbol] (O-cyclic) | Gln | (αR)-α-Amino-4- | Arg | Trp(6-F) | Pen | Ac | NH₂ | Disulfide |
| | | | | | | | | | [symbol] | | | | | | |
| | | | | | | | | | propanoic acid | | | | | | |
| 201 | | | | | N-4-aminobutyl- | Cys | D-Ala | His | D-Phe | Arg | Trp | Cys | Ac | NH₂ | Disulfide |
| | | | | | Gly | | | | | | | | | | |
| 202 | | | | | Arg | Cys | D-Asp | His | D-Phe | Arg | Trp | Cys | Ac | NH₂ | Disulfide |
| 203 | | | | | Arg | Cys | D-Glu | His | D-Phe | Arg | Trp | Cys | Ac | NH₂ | Disulfide |
| 204 | | | | | Arg | Cys | D-Dab | His | D-Phe | Arg | Trp | Cys | Ac | NH₂ | Disulfide |
| 205 | | | | | Arg | Cys | D-Ala | His | (aMe) [symbol] -Phe | Arg | Trp | Cys | Ac | NH₂ | Disulfide |
| 206 | | | | | Arg | Cys | Glu | His | D-Phe | Arg | Trp | Cys | Ac | NH₂ | Disulfide |
| 207 | | | | | Arg | Cys | D-Ser | His | D-Phe | Arg | Trp | Cys | Ac | NH₂ | Disulfide |
| 208 | | | | | Arg | Cys | D-Abu | His | D-Phe | Arg | Trp | Cys | Ac | NH₂ | Disulfide |
| 209 | | | | | Arg | Cys | Glu | Pro(4OH) | D-Phe | Arg | Trp | Cys | Ac | NH₂ | Disulfide |
| 210 | | | | | Arg | Cys | D-Ala | Pro(4OH) | D-Phe | Arg | Trp | Cys | Ac | NH₂ | Disulfide |
| 211 | | | | | Arg | Cys | D-Ala | His | D-Phe(4-Me) | Arg | Trp | Cys | Ac | NH₂ | Disulfide |
| 212 | | | | | Arg | Cys | D-Ala | His | D-Phe(3-Me) | Arg | Trp | Cys | Ac | NH₂ | Disulfide |
| 213 | | | | | Arg | Cys | D-Ala | His | D-homoPhe | Arg | Trp | Cys | Ac | NH₂ | Disulfide |
| 214 | | | | | Arg | Cys | D-Ala | His | D-phenylGly | Arg | Trp | Cys | Ac | NH₂ | Disulfide |
| 215 | | | | | Beta-homoArg | Cys | D-Ala | His | D-Phe | Arg | Trp | Cys | Ac | NH₂ | Disulfide |
| 216 | | | | | Arg | Cys | Ala(2-Me) | His | D-Phe | Arg | Trp | Cys | Ac | NH₂ | Disulfide |
| 1001 | | | | | Arg | Cys | Cyclo-Leu | His | D-Phe | Arg | Trp | Cys | Ac | NH₂ | Disulfide |
| 218 | | | | | Arg | Cys | D-Ala | N—Me-His | D-Phe | Arg | Trp | Cys | Ac | NH₂ | Disulfide |
| 219 | | | | | Arg | Cys | D-Ala | His | homoPhe | Arg | Trp | Cys | Ac | NH₂ | Disulfide |
| 220 | | | | | Arg | Cys | D-Ala | His | Pro(4-phenyl) | Arg | Trp | Cys | Ac | NH₂ | Disulfide |
| 221 | | | | | Arg | Cys | D-Ala | His | Phe(4-Me) | Arg | Trp | Cys | Ac | NH₂ | Disulfide |
| 222 | | | | | Arg | Cys | D-Ala | His | Phe(3-Me) | Arg | Trp | Cys | Ac | NH₂ | Disulfide |
| 223 | | | | | Arg | Cys | D-Ala | His | Phe(3,4-diMe) | Arg | Trp | Cys | Ac | NH₂ | Disulfide |
| 224 | | | | | Arg | Cys | D-Ala | indoine- | D-Phe | Arg | Trp | Cys | Ac | NH₂ | Disulfide |
| | | | | | | | | COOH | | | | | | | |
| 1002 | | | | | Arg | Cys | D-Ala | His | D-Phe(3,4-diMe) | Arg | Trp | Cys | Ac | NH₂ | Disulfide |
| 226 | | | | | Arg | Cys | D-Ala | His | D-Phe | Arg | Trp(6-Me) | Cys | Ac | NH₂ | Disulfide |
| 227 | | | | | D-hArg | Cys | D-Ala | His | D-Phe | Arg | Trp | Cys | Ac | NH₂ | Disulfide |
| 228 | | | | | L-hArg | Cys | D-Ala | His | D-Phe | Arg | Trp | Cys | Ac | NH₂ | Disulfide |
| 229 | | | | | [delta- | Cys | D-Ala | His | D-Phe | Arg | Trp | Cys | Ac | NH₂ | Disulfide |
| | | | | | [symbol] acid | | | | | | | | | | |
| 230 | | | | | [4- | Cys | D-Ala | His | D-Phe | Arg | Trp | Cys | Ac | NH₂ | Disulfide |
| | | | | | [symbol] acid [symbol] | | | | | | | | | | |
| 1003 | | | | | Arg | Cys | L-aMeGlu | His | D-Phe | Arg | Trp | Cys | Ac | NH₂ | Disulfide |
| 232 | | | | | Arg | Cys | D-aMeAsp | His | D-Phe | Arg | Trp | Cys | Ac | NH₂ | Disulfide |
| 1004 | | | | | Arg | Cys | L-aMeAsp | His | D-Phe | Arg | Trp | Cys | Ac | NH₂ | Disulfide |
| 234 | | | | | Arg | Cys | D-aMeSer | His | D-Phe | Arg | Trp | Cys | Ac | NH₂ | Disulfide |
| 235 | | | | | Arg | Cys | L-aMeSer | His | D-Phe | Arg | Trp | Cys | Ac | NH₂ | Disulfide |
| 236 | | | | | Arg | Cys | Ac4c | His | D-Phe | Arg | Trp | Cys | Ac | NH₂ | Disulfide |
| 237 | | | | | Arg | Cys | Ac6c | His | D-Phe | Arg | Trp | Cys | Ac | NH₂ | Disulfide |
| 238 | | | | | Arg | Cys | 4-aminooxane-4- | His | D-Phe | Arg | Trp | Cys | Ac | NH₂ | Disulfide |
| | | | | | | | carboxylic acid | | | | | | | | |
| 239 | | | | | Arg | Cys | D-hSer | His | D-Phe | Arg | Trp | Cys | Ac | NH₂ | Disulfide |
| 240 | | | | | Arg | Cys | D-Nva | His | D-Phe | Arg | Trp | Cys | Ac | NH₂ | Disulfide |
| 241 | | | | | Arg | Cys | D-Ala | D-His | D-Phe | Arg | Trp | Cys | Ac | NH₂ | Disulfide |
| 242 | | | | | D-Arg | Cys | Ala(2-Me) | His | D-Phe | Arg | Trp | Cys | Ac | NH₂ | Disulfide |
| 1005 | | | | | D-Arg | Cys | Cyclo-Leu | His | D-Phe | Arg | Trp | Cys | Ac | NH₂ | Disulfide |
| 244 | | | | | Arg | Cys | Glu | His | D-Phe(4-Me) | Arg | Trp | Cys | Ac | NH₂ | Disulfide |
| 245 | | | | | Arg | Cys | Ala(2-Me) | His | D-Phe(4-Me) | Arg | Trp | Cys | Ac | NH₂ | Disulfide |
| 246 | | | | | D-Arg | Cys | D-Asp | His | D-Phe | Arg | Trp | Cys | Ac | NH₂ | Disulfide |
| 247 | | | | | Beta-homoArg | Cys | Glu | His | D-Phe | Arg | Trp | Cys | Ac | NH₂ | Disulfide |
| 248 | | | | | D-Arg | Cys | D-Ser | His | D-Phe | Arg | Trp | Cys | Ac | NH₂ | Disulfide |
| 249 | | | | | Arg | Cys | D-Asp | His | D-Phe | Arg | Trp(6-Me) | Cys | Ac | NH₂ | Disulfide |
| 250 | | | | | D-Arg | Cys | D-Ala | His | D-Phe | Arg | Trp(6-Me) | Cys | Ac | NH₂ | Disulfide |
| 251 | | | | | Arg | Cys | Ala(2-Me) | His | D-Phe | Arg | Trp(6-Me) | Cys | Ac | NH₂ | Disulfide |
| 252 | | | | | Arg | Cys | Glu | His | D-Phe | Arg | Trp(6-Me) | Cys | Ac | NH₂ | Disulfide |
| 253 | | | | | Beta-homoArg | Cys | Ala(2-Me) | His | D-Phe | Arg | Trp | Cys | Ac | NH₂ | Disulfide |
| 1006 | | | | | Beta-homoArg | Cys | Cyclo-Leu | His | D-Phe | Arg | Trp | Cys | Ac | NH₂ | Disulfide |
| 255 | | | | | D-Arg | Cys | D-Dab | His | D-Phe | Arg | Trp | Cys | Ac | NH₂ | Disulfide |
| 1007 | | | | | D-Arg | Cys | L-aMeGlu | His | D-Phe | Arg | Trp | Cys | Ac | NH₂ | Disulfide |
| 257 | | | | | [symbol] | Cys | D-Ala | His | D-Phe | Arg | Trp | Cys | Ac | NH₂ | Disulfide |
| 258 | | | | | D-Nar | Cys | D-Ala | His | D-Phe | Arg | Trp | Cys | Ac | NH₂ | Disulfide |
| 259 | | | | | Arg | Cys | 3-aminoazetidine-3- | His | D-Phe | Arg | Trp | Cys | Ac | NH₂ | Disulfide |
| | | | | | | | carboxylic acid | | | | | | | | |
| 260 | | | | | Arg | Cys | D-Lys | His | D-Phe | Arg | Trp | Cys | Ac | NH₂ | Disulfide |
| 261 | | | | | Arg | Cys | D-Orn | His | D-Phe | Arg | Trp | Cys | Ac | NH₂ | Disulfide |
| 262 | | | | | Arg | Cys | Ame-L-Abu | His | D-Phe | Arg | Trp | Cys | Ac | NH₂ | Disulfide |
| 263 | | | | | Arg | Cys | D-aMeLeu | His | D-Phe | Arg | Trp | Cys | Ac | NH₂ | Disulfide |
| 264 | | | | | Arg | Cys | Gln | His | D-Phe | Arg | Trp | Cys | Ac | NH₂ | Disulfide |
| 265 | | | | | Arg | Cys | D-Leu | His | D-Phe | Arg | Trp | Cys | Ac | NH₂ | Disulfide |
| 266 | | | | | Arg | Cys | D-Ala | 3-Me-His | D-Phe | Arg | Trp | Cys | Ac | NH₂ | Disulfide |
| 267 | | | | | Arg | Cys | D-Ala | Ala(2-furyl) | D-Phe | Arg | Trp | Cys | Ac | NH₂ | Disulfide |
| 268 | | | | | Arg | Cys | D-Ala | His | D-Phe(4-Br) | Arg | Trp | Cys | Ac | NH₂ | Disulfide |
| 269 | | | | | Arg | Cys | D-Ala | His | D-Phe(4-F) | Arg | Trp | Cys | Ac | NH₂ | Disulfide |
| 270 | | | | | Arg | Cys | D-Ala | His | D-Phe(4-Cl) | Arg | Trp | Cys | Ac | NH₂ | Disulfide |
| 271 | | | | | Arg | Cys | D-Ala | His | D-Phe | Arg | Trp(6-F) | Cys | Ac | NH₂ | Disulfide |
| 272 | | | | | Beta-homoArg | Cys | D-Dab | His | D-Phe | Arg | Trp | Cys | Ac | NH₂ | Disulfide |
| 273 | | | | | Arg | Cys | Ala(2-Me) | His | D-Phe(3-Me) | Arg | Trp | Cys | Ac | NH₂ | Disulfide |
| 274 | | | | | Beta-homoArg | Cys | D-Dap | His | D-Phe | Arg | Trp | Cys | Ac | NH₂ | Disulfide |
| 275 | | | | | Beta-homoArg | Cys | D-Dab | His | D-Phe | Arg | Trp(6-Me) | Cys | Ac | NH₂ | Disulfide |
| 276 | | | | | Beta-homoArg | Cys | Ala(2-Me) | His | D-Phe | Arg | Trp(6-Me) | Cys | Ac | NH₂ | Disulfide |
| 1008 | | | | | Beta-homoArg | Cys | Cyclo-Leu | His | D-Phe | Arg | Trp(6-Me) | Cys | Ac | NH₂ | Disulfide |
| 278 | | | | | D-Arg | Cys | D-Dab | His | D-Phe | Arg | Trp(6-Me) | Cys | Ac | NH₂ | Disulfide |
| 279 | | | | | D-Arg | Cys | Ala(2-Me) | His | D-Phe | Arg | Trp(6-Me) | Cys | Ac | NH₂ | Disulfide |
| 1009 | | | | | D-Arg | Cys | Cyclo-Leu | His | D-Phe | Arg | Trp(6-Me) | Cys | Ac | NH₂ | Disulfide |
| 287 | | | | gGlu | Arg | Cys | D-Ala | His | D-Phe | Arg | Trp | Cys | Ac | NH₂ | Disulfide |
| 288 | | | | gGlu | D-Arg | Cys | D-Ala | His | D-Phe | Arg | Trp | Cys | Ac | NH₂ | Disulfide |
| 289 | | | | gGlu | Beta-homoArg | Cys | D-Ala | His | D-Phe | Arg | Trp | Cys | Ac | NH₂ | Disulfide |
| 290 | | | Arg | Gly | Arg | Cys | D-Ala | His | D-Phe | Arg | Trp | Cys | Ac | NH₂ | Disulfide |
| 291 | | | Glu | Pro | Arg | Cys | D-Ala | His | D-Phe | Arg | Trp | Cys | Ac | NH₂ | Disulfide |
| 292 | | | | Inp | D-Arg | Cys | D-Ala | His | D-Phe | Arg | Trp | Cys | Ac | NH₂ | Disulfide |
| 293 | | | | Tyr | Arg | Cys | D-Ala | His | D-Phe | Arg | Trp | Cys | Ac | NH₂ | Disulfide |
| 294 | | | | D-homoPhe | Arg | Cys | D-Ala | His | D-Phe | Arg | Trp | Cys | Ac | NH₂ | Disulfide |
| 295 | | | | Beta-homoArg | Arg | Cys | D-Ala | His | D-Phe | Arg | Trp | Cys | Ac | NH₂ | Disulfide |
| 296 | | | Leu | Ala | Arg | Cys | D-Ala | His | D-Phe | Arg | Trp | Cys | Ac | NH₂ | Disulfide |
| 297 | | | Glu | Ala | Beta-homoArg | Cys | D-Ala | His | D-Phe | Arg | Trp | Cys | Ac | NH₂ | Disulfide |
| 298 | | | Arg | Gly | Beta-homoArg | Cys | D-Ala | His | D-Phe | Arg | Trp | Cys | Ac | NH₂ | Disulfide |
| 299 | | | Leu | Ala | Beta-homoArg | Cys | D-Ala | His | D-Phe | Arg | Trp | Cys | Ac | NH₂ | Disulfide |
| 300 | | | Glu | Pro | Beta-homoArg | Cys | D-Ala | His | D-Phe | Arg | Trp | Cys | Ac | NH₂ | Disulfide |
| 301 | | | Phe | Gly | Beta-homoArg | Cys | D-Ala | His | D-Phe | Arg | Trp | Cys | Ac | NH₂ | Disulfide |
| 345 | | | | | Arg | Cys | D-homoPhe | His | D-Phe | Arg | Trp | Cys | Ac | NH₂ | Disulfide |
| 346 | | | | | Arg | Cys | D-Ala | His | D-Phe(3-F) | Arg | Trp | Cys | Ac | NH₂ | Disulfide |
| 347 | | | | D-Phe | Arg | Cys | D-Ala | His | D-Phe | Arg | Trp | Cys | Ac | NH₂ | Disulfide |
| 348 | | | | D-Tyr | Arg | Cys | D-Ala | His | D-Phe | Arg | Trp | Cys | Ac | NH₂ | Disulfide |
| 349 | | | Ser | Tyr | Arg | Cys | D-Ala | His | D-Phe | Arg | Trp | Cys | Ac | NH₂ | Disulfide |
| 350 | | | | | Lys | Cys | D-Ala | His | D-Phe | Arg | Trp | Cys | Ac | NH₂ | Disulfide |
| 351 | | | | | Ser | Cys | D-Ala | His | D-Phe | Arg | Trp | Cys | Ac | NH₂ | Disulfide |
| 357 | | | | | Arg | Cys | D-Ala | His | D-Phe | Arg | Trp | hCys | Ac | NH₂ | Disulfide |
| 358 | | | | | Arg | Cys | D-Ala | His | D-Phe | Arg | Trp(5-Me) | Cys | Ac | NH₂ | Disulfide |
| 359 | | | | | Arg | Cys | D-Ala | His | D-Bpa | Arg | Trp | Cys | Ac | NH₂ | Disulfide |
| 360 | | | | | Arg | Cys | D-Ala | Dap | D-Phe | Arg | Trp | Cys | Ac | NH₂ | Disulfide |
| 361 | | | | | Arg | Cys | D-Tyr | His | D-Phe | Arg | Trp | Cys | Ac | NH₂ | Disulfide |
| 362 | | | | | Arg | Cys | D-Ala | His | D-Tyr | Arg | Trp | Cys | Ac | NH₂ | Disulfide |
| 363 | | | | Arg(Me) | Arg | Cys | D-Ala | His | D-Phe | Arg | Trp | Cys | Ac | NH₂ | Disulfide |
| 364 | | | | | Arg | Cys | D-Ala | His | D-Tyr | Arg | Trp(5-OH) | Cys | Ac | NH₂ | Disulfide |
| 365 | | | | PEG1 | Arg | Cys | D-Ala | His | D-Phe | Arg | Trp | Cys | Ac | NH₂ | Disulfide |
| 366 | | | | | Nar | Cys | D-Ala | His | D-Phe | Arg | Trp | Cys | Ac | NH₂ | Disulfide |
| 367 | | | | | Arg | Cys | L-aMeOrn | His | D-Phe | Arg | Trp | Cys | Ac | NH₂ | Disulfide |
| 1010 | | | | | Arg | Cys | D-aMeOrn | His | D-Phe | Arg | Trp | Cys | Ac | NH₂ | Disulfide |
| 369 | | | | | Arg | Cys | beta-Ala(2Me) | His | D-Phe | Arg | Trp | Cys | Ac | NH₂ | Disulfide |
| 370 | | | | | Arg | Cys | D-Ala | His | D-Phe(3-Ph) | Arg | Trp | Cys | Ac | NH₂ | Disulfide |
| 374 | | | | | Arg | Cys | L-aMeGly(Allyl) | His | D-Phe | Arg | Trp | Cys | Ac | NH₂ | Disulfide |
| 375 | | | | | Arg | Cys | D-Ala | 2Pal | D-Phe | Arg | Trp | Cys | Ac | NH₂ | Disulfide |
| 376 | | | | | Arg | Cys | D-Ala | His | D-Phe | Arg | Trp(7-Me) | Cys | Ac | NH₂ | Disulfide |
| 377 | | | | | Arg | Cys | Nip(4-NH2) | His | D-Phe | Arg | Trp | Cys | Ac | NH₂ | Disulfide |
| 378 | | | | | D-Arg | Cys | Nip(4-NH2) | His | D-Phe | Arg | Trp | Cys | Ac | NH₂ | Disulfide |
| 379 | | | | | D-Arg | Cys | D-Dap | His | D-Phe | Arg | Trp | Cys | Ac | NH₂ | Disulfide |
| 380 | | | | Gaba | D-Arg | Cys | D-Ala | His | D-Phe | Arg | Trp | Cys | Ac | NH₂ | Disulfide |
| 381 | | | | | Arg | Cys | L-Dab | His | D-Phe | Arg | Trp(6-Me) | Cys | Ac | NH₂ | Disulfide |
| 382 | | | | | D-Arg | Cys | L-Dab | His | D-Phe(3-Me) | Arg | Trp | Cys | Ac | NH₂ | Disulfide |
| 383 | | | | | Arg | Cys | Orn | His | D-Phe | Arg | Trp(6-Me) | Cys | Ac | NH₂ | Disulfide |
| 385 | | | | | D-Arg | Cys | L-aMeVal | His | D-Phe | Arg | Trp | Cys | Ac | NH₂ | Disulfide |
| 386 | | | | | D-Arg | Cys | D-aMeVal | His | D-Phe | Arg | Trp | Cys | Ac | NH₂ | Disulfide |
| 387 | | | | gGlu | Me-D-Arg | Cys | D-Ala | His | D-Phe | Arg | Trp | Cys | Ac | NH₂ | Disulfide |
| 388 | | | Glu | Gly | Beta-homoArg | Cys | D-Ala | His | D-Phe | Arg | Trp | Cys | Ac | NH₂ | Disulfide |
| 390 | | | | Inp | Arg | Cys | D-Ala | His | D-Phe | Arg | Trp | Cys | Ac | NH₂ | Disulfide |
| 391 | | | | D-Arg | Arg | Cys | D-Ala | His | D-Phe | Arg | Trp | Cys | Ac | NH₂ | Disulfide |
| 392 | | | | Tranexamic | Arg | Cys | D-Ala | His | D-Phe | Arg | Trp | Cys | Ac | NH₂ | Disulfide |
| | | | | acid | | | | | | | | | | | |
| 393 | | | | homoPhe | Arg | Cys | D-Ala | His | D-Phe | Arg | Trp | Cys | Ac | NH₂ | Disulfide |
| 394 | | | | D-hArg | Arg | Cys | D-Ala | His | D-Phe | Arg | Trp | Cys | Ac | NH₂ | Disulfide |
| 395 | | | | Gaba | Arg | Cys | D-Ala | His | D-Phe | Arg | Trp | Cys | Ac | NH₂ | Disulfide |
| 396 | | | | Gaba | Beta-homoArg | Cys | D-Ala | His | D-Phe | Arg | Trp | Cys | Ac | NH₂ | Disulfide |
| 397 | | | Gln | Gly | Arg | Cys | D-Ala | His | D-Phe | Arg | Trp | Cys | Ac | NH₂ | Disulfide |
| 399 | | | Lys | Gly | Me-Arg | Cys | D-Ala | His | D-Phe | Arg | Trp | Cys | Ac | NH₂ | Disulfide |
| 400 | | | | gGlu | Me-Arg | Cys | D-Ala | His | D-Phe | Arg | Trp | Cys | Ac | NH₂ | Disulfide |
| 401 | | | | 2Nal | Arg | Cys | D-Ala | His | D-Phe | Arg | Trp | Cys | Ac | NH₂ | Disulfide |
| 1011 | | | | | Arg | Cys | D-Ala | Gln | D-Phe | Arg | Trp | Cys | Ac | NH₂ | Disulfide |
| 1012 | | | | | Arg | Cys | Cyclo-Leu | Gln | D-Phe | Arg | Trp | Cys | Ac | NH₂ | Disulfide |
| 407 | | | | | Arg | Cys | Ala(2-Me) | His | D-Phe(4-F) | Arg | Trp | Cys | Ac | NH₂ | Disulfide |
| 1013 | | | | | Arg | Cys | Ala(2-Me) | Gln | D-Phe | Arg | Trp | Cys | Ac | NH₂ | Disulfide |
| 409 | | | | | Arg | Cys | D-Glu | 3-Me-His | D-Phe | Arg | D-Trp | Cys | Ac | NH₂ | Disulfide |
| 410 | | | | | Arg | Cys | D-Ala | Ala( [symbol] ) | D-Phe | Arg | Trp | Cys | Ac | NH₂ | Disulfide |
| 411 | | | | Tyr | Beta-homoArg | Cys | D-Ala | His | D-Phe | Arg | Trp | Cys | Ac | NH₂ | Disulfide |
| 412 | | | | | Arg | Cys | Nip(4-NH2) | His | D-Phe | Arg | Trp(6-Me) | Cys | Ac | NH₂ | Disulfide |
| 413 | | | | | Beta-homoArg | Cys | Nip(4-NH2) | His | D-Phe | Arg | Trp | Cys | Ac | NH₂ | Disulfide |
| 414 | | | | | Arg | Cys | Ala(2-Me) | His | D-Phe(3-F) | Arg | Trp | Cys | Ac | NH₂ | Disulfide |
| 1014 | | | | | Beta-homoArg | Cys | D-Dab | Gln | D-Phe | Arg | Trp | Cys | Ac | NH₂ | Disulfide |
| 419 | | | | | Arg | Cys | D-Dab | His | D-Phe(4-Me) | Arg | Trp | Cys | Ac | NH₂ | Disulfide |
| 1015 | | | | | D-Arg | Cys | Cyclo-Leu | His | D-Phe(4-Me) | Arg | Trp(6-Me) | Cys | Ac | NH₂ | Disulfide |
| 421 | | | Lys | Gly | Me-D-Arg | Cys | D-Ala | His | D-Phe | Arg | Trp | Cys | Ac | NH₂ | Disulfide |
| 422 | | | | | Beta-homoArg | Cys | Ala(2-Me) | His | D-Phe | Arg | Trp(6-F) | Cys | Ac | NH₂ | Disulfide |
| 424 | | | | PEG2 | Arg | Cys | D-Ala | His | D-Phe | Arg | Trp | Cys | Ac | NH₂ | Disulfide |
| 1016 | | | | | D-Arg | Cys | L-aMeGlu | His | D-Phe | Arg | Trp(6-Me) | Cys | Ac | NH₂ | Disulfide |
| 1017 | | | | | D-Arg | Cys | L-aMeGlu | His | D-Phe(4-Me) | Arg | Trp | Cys | Ac | NH₂ | Disulfide |
| 1018 | | | | Gly | D-Arg | Cys | L-aMeGlu | His | D-Phe | Arg | Trp | Cys | Ac | NH₂ | Disulfide |
| 428 | | | Lys* | Gly | D-Arg | Cys | L-aMeGlu | His | D-Phe | Arg | Trp | Cys | Ac | NH₂ | Disulfide |
| 1019 | | | | | D-Nar | Cys | L-aMeGlu | His | D-Phe | Arg | Trp | Cys | Ac | NH₂ | Disulfide |
| 1020 | | | | | D-Nar | Cys | L-aMeGlu | His | D-Phe | Arg | Trp(6-Me) | Cys | Ac | NH₂ | Disulfide |
| 1021 | | | | | D-Nar | Cys | L-aMeGlu | His | D-Phe(4-Me) | Arg | Trp | Cys | Ac | NH₂ | Disulfide |
| 1022 | | | | | Beta-homoArg | Cys | L-aMeGlu | His | D-Phe | Arg | Trp | Cys | Ac | NH₂ | Disulfide |
| 1023 | | | | | Beta-homoArg | Cys | L-aMeGlu | His | D-Phe(4-Me) | Arg | Trp | Cys | Ac | NH₂ | Disulfide |
| 1024 | | | | | Beta-homoArg | Cys | L-aMeGlu | His | D-Phe | Arg | Trp(6-Me) | Cys | Ac | NH₂ | Disulfide |
| 1025 | | | | Gly | Beta-homoArg | Cys | L-aMeGlu | His | D-Phe | Arg | Trp | Cys | Ac | NH₂ | Disulfide |
| 436 | | | Lys* | Gly | Beta-homoArg | Cys | L-aMeGlu | His | D-Phe | Arg | Trp | Cys | Ac | NH₂ | Disulfide |
| 437 | | | | | Beta-homoArg | Cys | D-hSer | His | D-Phe(4-Cl) | Arg | Trp(6-Me) | Cys | Ac | NH₂ | Disulfide |
| 1026 | | | | | Beta-homoArg | Cys | L-aMeGlu | His | D-Phe(4-Cl) | Arg | Trp(6-Me) | Cys | Ac | NH₂ | Disulfide |
| 439 | | | | | Beta-homoArg | Cys | 4-aminooxane-4- | His | D-Phe | Arg | Trp(6-Me) | Cys | Ac | NH₂ | Disulfide |
| | | | | | | | carboxylic acid | | | | | | | | |
| 1027 | | | | | Beta-homoArg | Cys | Cyclo-Leu | His | D-Phe(4-Cl) | Arg | TRP | Cys | Ac | NH₂ | Disulfide |
| 441 | | | | | Beta-homoArg | Cys | D-aMeSer | His | D-Phe | Arg | Trp(6-F) | Cys | Ac | NH₂ | Disulfide |
| 1028 | | | | | Beta-homoArg | Cys | L-aMeGlu | His | D-Phe | Arg | Trp(6-F) | Cys | Ac | NH₂ | Disulfide |
| 1029 | | | | | L-hArg | Cys | L-aMeGlu | His | D-Phe | Arg | Trp(6-Me) | Cys | Ac | NH₂ | Disulfide |
| 444 | | | | | L-hArg | Cys | D-hSer | His | D-Phe | Arg | Trp(6-Me) | Cys | Ac | NH₂ | Disulfide |
| 1030 | | | | | Beta-homoArg | Cys | L-aMeGlu | His | D-Phe(3-CF3) | Arg | TRP | Cys | Ac | NH₂ | Disulfide |
| 1031 | | | | | Beta-homoArg | Cys | L-aMeGlu | His | D-Phe(3-Cl) | Arg | TRP | Cys | Ac | NH₂ | Disulfide |
| 1032 | | | | | Beta-homoArg | Cys | L-aMeGlu | His | D-Phe | Arg | Trp(6-Cl) | Cys | Ac | NH₂ | Disulfide |
| 1033 | | | | | Beta-homoArg | Cys | bhGlu | His | D-Phe | Arg | TRP | Cys | Ac | NH₂ | Disulfide |
| 449 | | | | | Beta-homoArg | Cys | hGlu | His | D-Phe | Arg | TRP | Cys | Ac | NH₂ | Disulfide |
| 450 | | | | | Beta-homoArg | Cys | D-3Thi | His | D-Phe | Arg | TRP | Cys | Ac | NH₂ | Disulfide |
| 451 | | | | | Beta-homoArg | Cys | D-Iva | His | D-Phe | Arg | TRP | Cys | Ac | NH₂ | Disulfide |
| 452 | | | | | Beta-homoArg | Cys | bAc5c | His | D-Phe | Arg | TRP | Cys | Ac | NH₂ | Disulfide |
| 1034 | | | | | Beta-homoArg | Cys | Phg | His | D-Phe | Arg | TRP | Cys | Ac | NH₂ | Disulfide |
| 454 | | | | | Beta-homoArg | Cys | D-Phg | His | D-Phe | Arg | TRP | Cys | Ac | NH₂ | Disulfide |
| 455 | | | | | D-Nar | Cys | Cyclo-Leu(3-ene) | His | D-Phe | Arg | Trp(6-Me) | Cys | Ac | NH₂ | Disulfide |
| 456 | | | | | D-Nar | Cys | L-Apm | His | D-Phe | Arg | Trp(6-Me) | Cys | Ac | NH₂ | Disulfide |
| 1035 | | | | | D-Nar | Cys | L-aMeGlu | His | D-Phe | Arg | TRP(6-F) | Cys | Ac | NH₂ | Disulfide |
| 1036 | | | | | D-Nar | Cys | D-aMeOrn | His | D-Phe | Arg | TRP(6-F) | Cys | Ac | NH₂ | Disulfide |
| 1037 | | | | | D-Nar | Cys | Ala(2-Me) | His | D-Phe | Arg | TRP(6-F) | Cys | Ac | NH₂ | Disulfide |
| 1038 | | | | | D-Nar | Cys | L-aMeGlu | His | D-Phe(4-F) | Arg | TRP(6-F) | Cys | Ac | NH₂ | Disulfide |
| 1039 | | | | | D-Nar | Cys | D-aMeSer | His | D-Phe | Arg | Trp(6-Me) | Cys | Ac | NH₂ | Disulfide |
| 1040 | | | | | D-Nar | Cys | L-aMeGlu | His | D-Phe(3-F) | Arg | TRP(6-F) | Cys | Ac | NH₂ | Disulfide |
| 463 | | | | | D-Nar | Cys | Ac3c | His | D-Phe | Arg | Trp(6-Me) | Cys | Ac | NH₂ | Disulfide |
| 1041 | | | | | D-Nar | Cys | L-aMeGlu | His | D-Phe(4-F) | Arg | Trp(6-Me) | Cys | Ac | NH₂ | Disulfide |
| 1042 | | | | | D-Nar | Cys | L-aMeAsp | His | D-Phe | Arg | Trp(6-F) | Cys | Ac | NH₂ | Disulfide |
| 1043 | | | | | D-Nar | Cys | L-aMeGlu | His | D-Phe | Arg | Trp(5-Me) | Cys | Ac | NH₂ | Disulfide |
| 467 | | | | | D-Nar | Cys | bAc4c | His | D-Phe | Arg | Trp(6-F) | Cys | Ac | NH₂ | Disulfide |
| 468 | | | | | D-Nar | Cys | 4-aminooxane-4- | His | D-Phe | Arg | Trp(6-Me) | Cys | Ac | NH₂ | Disulfide |
| | | | | | | | carboxylic acid | | | | | | | | |
| 469 | | | | | D-Nar | Cys | Ala(2-Me) | His | D-Phe | Arg | Trp(6-Me) | Cys | Ac | NH₂ | Disulfide |
| 1044 | | | | | D-Nar | Cys | D-bhGlu | His | D-Phe | Arg | Trp(6-Me) | Cys | Ac | NH₂ | Disulfide |
| 1045 | | | | | D-Nar | Cys | L-aMeGlu | His | D-Phe(4-F) | Arg | Trp(6-Cl) | Cys | Ac | NH₂ | Disulfide |
| 1046 | | | | | D-Nar | Cys | L-aMeGlu | His | D-Phe(3,4,5-triF) | Arg | Trp(6-Me) | Cys | Ac | NH₂ | Disulfide |
| 1047 | | | | | D-Nar | Cys | L-aMeGlu | His | D-Phe(3,4,5-triF) | Arg | Trp(6-F) | Cys | Ac | NH₂ | Disulfide |
| 1048 | | | | | D-Nar | Cys | L-aMeGlu | His | D-Phe(3-Cl) | Arg | Trp(6-Me) | Cys | Ac | NH₂ | Disulfide |
| 475 | | | | | D-Nar | Cys | bhGlu | His | D-Phe | Arg | Trp(6-Me) | Cys | Ac | NH₂ | Disulfide |
| 1049 | | | | | D-Nar | Cys | hGlu | His | D-Phe | Arg | Trp(6-Me) | Cys | Ac | NH₂ | Disulfide |
| 1050 | | | | | D-Nar | Cys | L-aMeGlu | His | D-Phe(3-CF3) | Arg | Trp(6-Me) | Cys | Ac | NH₂ | Disulfide |
| 1051 | | | | | D-Nar | Cys | L-aMeGlu | His | D-Phe(4-Cl) | Arg | Trp(6-F) | Cys | Ac | NH₂ | Disulfide |
| 1052 | | | | | Beta-homoArg | Cys | cyclo-Leu | His | D-Phe | Arg | Trp(6-F) | Cys | Ac | NH₂ | Disulfide |
| 1053 | | | | | Arg | Cys | L-aMeGlu | His | D-Phe(4-F) | Arg | Trp(6-F) | Cys | Ac | NH₂ | Disulfide |
| 1054 | | | | | L-hArg | Cys | L-aMeGlu | His | D-Phe(4-F) | Arg | Trp(6-F) | Cys | Ac | NH₂ | Disulfide |
| 482 | | | | | D-Nar | Cys | Ac3c | His | D-Phe | Arg | Trp(6-F) | Cys | Ac | NH₂ | Disulfide |
| 1055 | | | | | D-Arg | Cys | D-aMeOrn | His | D-Phe | Arg | Trp(6-Me) | Cys | Ac | NH₂ | Disulfide |
| 1056 | | | | | Arg | Cys | L-aMeGlu | His | D-Phe(4-F) | Arg | Trp(6-Me) | Cys | Ac | NH₂ | Disulfide |
| 1057 | | | | | Arg | Cys | L-aMeGlu | His | D-Phe(3-F) | Arg | Trp(6-F) | Cys | Ac | NH₂ | Disulfide |
| 1058 | | | | | D-Nar | Cys | Aib(O-cyclic) | His | D-Phe | Arg | Trp(6-F) | Cys | Ac | NH₂ | Disulfide |
| 487 | | | | | D-Nar | Cys | D-hSer | His | D-Phe | Arg | Trp(6-F) | Cys | Ac | NH₂ | Disulfide |
| 1059 | | | | | Beta-homoArg | Cys | L-aMeGlu | His | D-Phe(4-F) | Arg | Trp(6-Me) | Cys | Ac | NH₂ | Disulfide |
| 489 | | | | | Beta-homoArg | Cys | D-hSer | His | D-Phe | Arg | Trp(6-F) | Cys | Ac | NH₂ | Disulfide |
| 1060 | | | | | D-Arg | Cys | L-aMeGlu | His | D-Phe(4-F) | Arg | Trp(6-Me) | Cys | Ac | NH₂ | Disulfide |
| 1061 | | | | | L-hArg | Cys | L-aMeGlu | His | D-Phe(4-F) | Arg | Trp(6-Me) | Cys | Ac | NH₂ | Disulfide |
| 1062 | | | | | D-Nar | Cys | L-aMeAsp | His | D-Phe(4-F) | Arg | Trp(6-Me) | Cys | Ac | NH₂ | Disulfide |
| 1063 | | | | | D-Arg | Cys | L-aMeAsp | His | D-Phe(4-F) | Arg | Trp(6-Me) | Cys | Ac | NH₂ | Disulfide |
| 1129 | | Lys* | Gly | D-Arg | D-Nar | Cys | L-aMeGlu | His | D-Phe | Arg | Trp(6-Me) | Cys | Ac | NH₂ | Disulfide |
| 1128 | | Lys* | Glu | PRO | D-Nar | Cys | L-aMeGlu | His | D-Phe | Arg | Trp(6-Me) | Cys | Ac | NH₂ | Disulfide |
| 1133 | | Lys* | Gly | gGlu | D-Nar | Cys | L-aMeGlu | His | D-Phe | Arg | Trp(6-Me) | Cys | Ac | NH₂ | Disulfide |
| 1064 | | | | | D-Nar | Cys | L-aMeAsp | His | D-Phe | Arg | Trp(6-Me) | Cys | Ac | NH₂ | Disulfide |
| 499 | | Lys* | Gly | Gly | D-Nar | Cys | L-aMeAsp | His | D-Phe | Arg | Trp(6-Me) | Cys | Ac | NH₂ | Disulfide |
| 1131 | | Lys* | PEG1 | PEG1 | D-Nar | Cys | L-aMeAsp | His | D-Phe | Arg | Trp(6-Me) | Cys | Ac | NH₂ | Disulfide |
| 1065 | | | | | beta-homoArg | Cys | L-aMeAsp | His | D-Phe | Arg | Trp(6-Me) | Cys | Ac | NH₂ | Disulfide |
| 1130 | | Lys* | Gly | Gly | beta-homoArg | Cys | L-aMeAsp | His | D-Phe | Arg | Trp(6-Me) | Cys | Ac | NH₂ | Disulfide |
| 504 | | Lys* | PEG1 | PEG1 | beta-homoArg | Cys | L-aMeAsp | His | D-Phe | Arg | Trp(6-Me) | Cys | Ac | NH₂ | Disulfide |
| 1135 | | Lys* | Gly | Gly | D-Arg | Cys | L-aMeAsp | His | D-Phe | Arg | Trp(6-Me) | Cys | Ac | NH₂ | Disulfide |
| 1066 | | | | | D-Nar | Glu | L-aMeGlu | His | D-Phe | Arg | Trp(6-Me) | Dap | Ac | NH₂ | Disulfide |
| 1067 | | | | | D-Nar | Asp | L-aMeGlu | His | D-Phe | Arg | Trp(6-Me) | Dap | Ac | NH₂ | Disulfide |
| 1068 | | | | | D-Nar | Glu | L-aMeAsp | His | D-Phe | Arg | Trp(6-Me) | Dap | Ac | NH₂ | Disulfide |
| 1069 | | | | | D-Nar | Asp | L-aMeAsp | His | D-Phe | Arg | Trp(6-Me) | Dap | Ac | NH₂ | Disulfide |
| 1070 | | | | | Arg | Cys | D-Ala | Gln | D-Phe | Arg | Trp | Cys | Ac | NH₂ | Disulfide |
| 1071 | | | | | D-Nar | Cys | L-aMeGlu | His | D-Phe(3-F,4-Me) | Arg | Trp(6-F) | Cys | Ac | NH₂ | Disulfide |
| 1072 | | | | | D-Nar | Cys | L-aMeGlu | His | D-Phe(4-CF3) | Arg | Trp(6-F) | Cys | Ac | NH₂ | Disulfide |
| 1073 | | | | | D-Nar | Cys | L-aMeGlu | His | D-Phe(2-F,4-Cl) | Arg | Trp(6-F) | Cys | Ac | NH₂ | Disulfide |
| 1074 | | | | | D-Nar | Cys | L-aMeGlu | His | D-Phe(3,4-diF) | Arg | Trp(6-F) | Cys | Ac | NH₂ | Disulfide |
| 1075 | | | | | D-Nar | Cys | L-aMeGlu | His | D-Phe | Arg | Trp(6-CF3) | Cys | Ac | NH₂ | Disulfide |
| 1076 | | | | | D-Nar | Cys | L-aMeGlu | His | D-Phe | Arg | Trp(4-F) | Cys | Ac | NH₂ | Disulfide |
| 1077 | | | | | D-Nar | Cys | L-aMeGlu | His | D-Phe | Arg | Trp(5-F) | Cys | Ac | NH₂ | Disulfide |
| 1078 | | | | | D-Nar | Cys | L-aMeGlu | His | D-Phe | Arg | Trp(7-F) | Cys | Ac | NH₂ | Disulfide |
| 1079 | | | | | D-Nar | Cys | L-aMeGlu | His | D-Phe | Arg | Trp(5-Cl) | Cys | Ac | NH₂ | Disulfide |
| 1080 | | | | | D-Nar | Cys | L-aMeGlu | His | D-Phe | Arg | Trp(6-Br) | Cys | Ac | NH₂ | Disulfide |
| 1081 | | | | | D-Nar | Cys | L-aMeGlu | His | D-Phe(3-F) | Arg | Trp(5-F) | Cys | Ac | NH₂ | Disulfide |
| 1082 | | | | | D-Nar | Cys | L-aMeGlu | His | D-Phe(2,4-diCl) | Arg | Trp(6-F) | Cys | Ac | NH₂ | Disulfide |
| 1083 | | | | | D-Nar | Cys | L-aMeGlu | His | D-Phe(2,3-diF) | Arg | Trp(6-F) | Cys | Ac | NH₂ | Disulfide |
| 1084 | | | | | D-Nar | Cys | L-aMeGlu | His | D-Phe(3-Cl) | Arg | Trp(6-F) | Cys | Ac | NH₂ | Disulfide |
| 1085 | | | | | D-Nar | Cys | L-aMeGlu | His | D-Phe(3-F) | Arg | Trp(6-Me) | Cys | Ac | NH₂ | Disulfide |
| 1086 | | | | | D-Nar | Cys | L-aMeGlu | His | D-Phe(3-Me) | Arg | Trp(6-F) | Cys | Ac | NH₂ | Disulfide |
| 1087 | | | | | D-Nar | Cys | L-aMeGlu | His | D-Phe(2,4-diF) | Arg | Trp(6-F) | Cys | Ac | NH₂ | Disulfide |
| 1088 | | | | | D-Nar | Cys | L-aMeGlu | His | D-Phe(2,4,5-TriF) | Arg | Trp(6-F) | Cys | Ac | NH₂ | Disulfide |
| 1089 | | | | | D-Nar | Cys | L-aMeAsp | His | D-Phe(3-CF3) | Arg | Trp(6-F) | Cys | Ac | NH₂ | Disulfide |
| 532 | | | | | D-Nar | Cys | D-bhGlu | His | D-Phe | Arg | Trp(6-F) | Cys | Ac | NH₂ | Disulfide |
| 533 | | | | | D-Nar | Cys | D-bhGlu | His | D-Phe(3-F) | Arg | Trp(6-F) | Cys | Ac | NH₂ | Disulfide |
| 534 | | | | | D-Nar | Cys | D-bhGlu | His | D-Phe | Arg | Trp(6-CF3) | Cys | Ac | NH₂ | Disulfide |
| 535 | | | | | D-Nar | Cys | hGlu | His | D-Phe | Arg | Trp(6-F) | Cys | Ac | NH₂ | Disulfide |
| 536 | | | | | D-Nar | Cys | hGlu | His | D-Phe(4-F) | Arg | Trp(6-F) | Cys | Ac | NH₂ | Disulfide |
| 537 | | | | | D-Nar | Cys | hGlu | His | D-Phe(3,4,5-TriF) | Arg | Trp(6-F) | Cys | Ac | NH₂ | Disulfide |
| 538 | | | | | D-Nar | Cys | hGlu | His | D-Phe(3-F) | Arg | Trp(6-F) | Cys | Ac | NH₂ | Disulfide |
| 539 | | | | | D-Nar | Cys | Apm | His | D-Phe | Arg | Trp(6-F) | Cys | Ac | NH₂ | Disulfide |
| 540 | | | | | D-Nar | Cys | Apm | His | D-Phe | Arg | Trp(5-F) | Cys | Ac | NH₂ | Disulfide |
| 541 | | | | | D-Nar | Cys | Apm | His | D-Phe(3,4,5-TriF) | Arg | Trp(6-F) | Cys | Ac | NH₂ | Disulfide |
| 542 | | | | | D-Nar | Cys | Apm | His | D-Phe(4-F) | Arg | Trp(6-F) | Cys | Ac | NH₂ | Disulfide |
| 1090 | | | | | D-Nar | Cys | L-aMeGlu | Gln | D-Phe | Arg | Trp(6-F) | Cys | Ac | NH₂ | Disulfide |
| 1091 | | | | | D-Nar | Cys | hGlu | Gln | D-Phe | Arg | Trp(6-F) | Cys | Ac | NH₂ | Disulfide |
| 1092 | | | | | D-Nar | Cys | Aib(O-cyclic) | Gln | D-Phe | Arg | Trp(6-F) | Cys | Ac | NH₂ | Disulfide |
| 1093 | | | | | D-Nar | Cys | Aib(O-cyclic) | Gln | D-Phe(3-F) | Arg | Trp(6-F) | Cys | Ac | NH₂ | Disulfide |
| 1094 | | | | | D-Nar | Cys | D-aMeOrn | Gln | D-Phe | Arg | Trp(6-F) | Cys | Ac | NH₂ | Disulfide |
| 1096 | | | | | D-Nar | Cys | D-aMeSer | Gln | D-Phe | Arg | Trp(6-F) | Cys | Ac | NH₂ | Disulfide |
| 1097 | | | | | D-Nar | Cys | D-aMeSer | Gln | D-Phe(4-F) | Arg | Trp(6-F) | Cys | Ac | NH₂ | Disulfide |
| 1098 | | | | | D-Nar | Cys | bhGlu | Gln | D-Phe | Arg | Trp(6-F) | Cys | Ac | NH₂ | Disulfide |
| 1099 | | | | | D-Nar | Cys | Aib | Gln | D-Phe(4-F) | Arg | Trp(6-F) | Cys | Ac | NH₂ | Disulfide |
| 1100 | | | | | D-Nar | Cys | D-aMeOrn | hGln | D-Phe | Arg | Trp(6-F) | Cys | Ac | NH₂ | Disulfide |
| 1101 | | | | | D-Nar | Cys | D-aMeOrn | hGln | D-Phe(4-F) | Arg | Trp(6-F) | Cys | Ac | NH₂ | Disulfide |
| 1102 | | | | | D-Nar | Cys | Aib(O-cyclic) | hGln | D-Phe | Arg | Trp(6-F) | Cys | Ac | NH₂ | Disulfide |
| 1103 | | | | | D-Nar | Cys | Aib(O-cyclic) | Cit | D-Phe | Arg | Trp(6-F) | Cys | Ac | NH₂ | Disulfide |
| 1104 | | | | | D-Nar | Cys | L-aMeGlu | Cit | D-Phe | Arg | Trp(6-F) | Cys | Ac | NH₂ | Disulfide |
| 1106 | | | | | D-Nar | Cys | Aib(O-cyclic) | Cit | D-Phe(4-F) | Arg | Trp(6-F) | Cys | Ac | NH₂ | Disulfide |
| 1107 | | | | | D-Nar | Cys | Aib(O-cyclic) | hCit | D-Phe(4-F) | Arg | Trp(6-F) | Cys | Ac | NH₂ | Disulfide |
| 1108 | | | | | D-Nar | Cys | Cyclo-Leu | 3Pal | D-Phe | Arg | Trp(6-F) | Cys | Ac | NH₂ | Disulfide |
| 1109 | | | | | D-Nar | Cys | D-aMeOrn | 4Pal | D-Phe | Arg | Trp(6-F) | Cys | Ac | NH₂ | Disulfide |
| 561 | Lys* | D-Arg | PEG1 | PEG1 | D-Nar | Cys | L-aMeGlu | His | D-Phe | Arg | Trp(6-F) | Cys | Ac | NH₂ | Disulfide |
| 562 | Lys* | PEG1 | PEG1 | PEG1 | D-Nar | Cys | L-aMeGlu | His | D-Phe | Arg | Trp(6-F) | Cys | Ac | NH₂ | Disulfide |
| 563 | Lys* | Glu | PEG1 | PEG1 | D-Nar | Cys | L-aMeGlu | His | D-Phe | Arg | Trp(6-F) | Cys | Ac | NH₂ | Disulfide |
| 564 | Lys* | Glu | Glu | Pro | D-Nar | Cys | L-aMeGlu | His | D-Phe | Arg | Trp(6-F) | Cys | Ac | NH₂ | Disulfide |
| 1141 | Lys* | Ser | Glu | Pro | D-Nar | Cys | L-aMeGlu | His | D-Phe | Arg | Trp(6-F) | Cys | Ac | NH₂ | Disulfide |
| 566 | Lys* | Ser | Gly | D-Arg | D-Nar | Cys | L-aMeGlu | His | D-Phe | Arg | Trp(6-F) | Cys | Ac | NH₂ | Disulfide |
| 567 | Lys* | D-Arg | Gly | D-Arg | D-Nar | Cys | L-aMeGlu | His | D-Phe | Arg | Trp(6-F) | Cys | Ac | NH₂ | Disulfide |
| 568 | Lys* | D-Arg | Ser | γ-Glu | D-Nar | Cys | L-aMeGlu | His | D-Phe | Arg | Trp(6-F) | Cys | Ac | NH₂ | Disulfide |
| 569 | Lys* | Glu | Gly | γ-Glu | D-Nar | Cys | L-aMeGlu | His | D-Phe | Arg | Trp(6-F) | Cys | Ac | NH₂ | Disulfide |
| 1143 | Lys* | Gly | Gly | γ-Glu | D-Nar | Cys | L-aMeGlu | His | D-Phe | Arg | Trp(6-F) | Cys | Ac | NH₂ | Disulfide |
| 571 | | Lys* | Gly | γ-Glu | Me-D-Arg | Cys | L-aMeGlu | His | D-Phe | Arg | Trp(6-F) | Cys | Ac | NH₂ | Disulfide |
| 572 | | Lys* | Gly | D-Arg | D-Nar | Cys | L-aMeGlu | His | D-Phe | Arg | Trp(6-F) | Cys | Ac | NH₂ | Disulfide |
| 573 | | Lys* | Glu | Pro | D-Nar | Cys | L-aMeGlu | His | D-Phe | Arg | Trp(6-F) | Cys | Ac | NH₂ | Disulfide |
| 574 | | Lys* | Glu | γ-Glu | D-Nar | Cys | L-aMeGlu | His | D-Phe | Arg | Trp(6-F) | Cys | Ac | NH₂ | Disulfide |
| 575 | | Lys* | D-Arg | γ-Glu | D-Nar | Cys | L-aMeGlu | His | D-Phe | Arg | Trp(6-F) | Cys | Ac | NH₂ | Disulfide |
| 1110 | | | | | D-Nar | Cys | Phg | His | D-Phe | Arg | Trp(6-F) | Cys | Ac | NH₂ | Disulfide |
| 1111 | | | | | D-Nar | Cys | Phg | His | D-Phe(4-F) | Arg | Trp(6-F) | Cys | Ac | NH₂ | Disulfide |
| 1112 | | | | | D-Nar | Cys | Phg | His | D-Phe(4-F) | Arg | Trp(6-Me) | Cys | Ac | NH₂ | Disulfide |
| 1113 | | | | | D-Nar | Cys | Phg | His | D-Phe(4-F) | Arg | Trp(6-Me) | Cys | Ac | NH₂ | Disulfide |
| 1114 | | | | | Nar | Cys | L-aMeGlu | His | D-Phe | Arg | Trp(6-F) | Cys | Ac | NH₂ | Disulfide |
| 1115 | | | | | Nar | Cys | L-aMeGlu | His | D-Phe(4-F) | Arg | Trp(6-F) | Cys | Ac | NH₂ | Disulfide |
| 1116 | | | | | D-Nar | Cys | L-aMeGlu | 3Pal | D-Phe | Arg | Trp(6-Me) | Cys | Ac | NH₂ | Disulfide |
| 1118 | | | | | D-Nar | Cys | L-aMeGlu | 4Pal | D-Phe | Arg | Trp(6-Me) | Cys | Ac | NH₂ | Disulfide |
| 1119 | | | | | D-Nar | Cys | Phg | 3Pal | D-Phe(4-F) | Arg | Trp(6-F) | Cys | Ac | NH₂ | Disulfide |
| 1120 | | | | | D-Nar | Cys | L-aMeAsp | His | D-Phe | Arg | Trp(6-F) | Cys | Ac | NH₂ | Disulfide |
| 1121 | | | | | D-Nar | Cys | L-aMeAsp | His | D-Phe(4-F) | Arg | Trp(6-F) | Cys | Ac | NH₂ | Disulfide |
| 1134 | Lys* | Glu | Glu | Pro | D-Nar | Cys | L-aMeGlu | His | D-Phe(3,4,5-triF) | Arg | Trp(6-Me) | Cys | Ac | NH₂ | Disulfide |
| 1132 | Lys* | Glu | PEG1 | PEG1 | D-Nar | Cys | L-aMeGlu | His | D-Phe(3,4,5-triF) | Arg | Trp(6-Me) | Cys | Ac | NH₂ | Disulfide |
| 589 | Lys* | Glu | Glu | Pro | D-Nar | Cys | D-bhGlu | His | D-Phe | Arg | Trp(6-Me) | Cys | Ac | NH₂ | Disulfide |
| 590 | Lys* | Glu | PEG1 | PEG1 | D-Nar | Cys | D-bhGlu | His | D-Phe | Arg | Trp(6-Me) | Cys | Ac | NH₂ | Disulfide |

[symbol]

indicates data missing or illegible when filed

In embodiments, the peptide of formula (I) is selected from Table AMA

In embodiments, the peptide of formula (I) is selected from Table A1A, wherein the N-terminal, C-terminal and/or cyclic structure are optional structure.

TABLE A1A
Exemplary peptides.

| Molecule | | | | | | | | | N- | C- | |
|---|---|---|---|---|---|---|---|---|---|---|---|
| Name | X₁ | X₂ | X₃ | X₄ | X₅ | X₆ | X₇ | X₈ | term | term | Cyclic |
| 1093 | D-Nar | Cys | Aib(O-cyclic) | Gln | D-Phe(4-F) | Arg | Trp(6-F) | Cys | Ac | NH₂ | Disulfide |
| 1092 | D-Nar | Cys | Aib(O-cyclic) | Gln | D-Phe | Arg | Trp(6-F) | Cys | Ac | NH₂ | Disulfide |
| 1107 | D-Nar | Cys | Aib(O-cyclic) | hCit | D-Phe(4-F) | Arg | Trp(6-F) | Cys | Ac | NH₂ | Disulfide |
| 1106 | D-Nar | Cys | Aib(O-cyclic) | Cit | D-Phe(4-F) | Arg | Trp(6-F) | Cys | Ac | NH₂ | Disulfide |
| 1103 | D-Nar | Cys | Aib(O-cyclic) | Cit | D-Phe | Arg | Trp(6-F) | Cys | Ac | NH₂ | Disulfide |
| 1105 | D-Nar | Cys | Aib(O-cyclic) | Cit | D-Phe(4-Me) | Arg | Trp(6-F) | Cys | Ac | NH₂ | Disulfide |
| 1095 | D-Nar | Cys | Aib(O-cyclic) | Gln | D-Phe(4-Me) | Arg | Trp(6-F) | Cys | Ac | NH₂ | Disulfide |
| 1122 | D-Nar | Cys | Aib(O-cyclic) | 3-Pal | D-Phe(4-F) | Arg | Trp(6-F) | Cys | Ac | NH₂ | Disulfide |
| 1102 | D-Nar | Cys | Aib(O-cyclic) | hGln | D-Phe | Arg | Trp(6-F) | Cys | Ac | NH₂ | Disulfide |
| 1058 | D-Nar | Cys | Aib(O-cyclic) | His | D-Phe | Arg | Trp(6-F) | Cys | Ac | NH₂ | Disulfide |
| 1123 | D-Nar | Cys | Aib(O-cyclic) | Orn | D-Phe(4-F) | Arg | Trp(6-F) | Cys | Ac | NH₂ | Disulfide |
| 1158 | D-Nar | Cys | Aib(O-cyclic) | Gln | D-Phe(4-F) | Arg | Trp(6-F) | Pen | Ac | NH₂ | Disulfide |

In embodiments, the peptide of formula (II) is selected from Table A2.

TABLE A2
Exemplary peptides.

| Molecule | | | | | | | | | | | | |
|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ |
| 1150-2 | Lys* | D-Arg | Gly | D-Arg | D-Nar | Cys | Aib(O-cyclic) | Gln | D-Phe(4-F) | Arg | Trp(6-F) | Cys |
| 1142-2 | | Lys* | Gly | D-Arg | D-Nar | Cys | Aib(O-cyclic) | Gln | D-Phe(4-F) | Arg | Trp(6-F) | Cys |
| 1144-2 | | Lys* | PEG1 | PEG1 | D-Nar | Cys | Aib(O-cyclic) | Gln | D-Phe(4-F) | Arg | Trp(6-F) | Cys |
| 1151-2 | Lys* | D-Arg | PEG1 | D-Arg | Beta- | Cys | Aib(O-cyclic) | Gln | D-Phe(4-F) | Arg | Trp(6-F) | Cys |
| | | | | | homoArg | | | | | | | |
| 1152-2 | Lys* | D-Arg | Gly | D-Arg | D-Nar | Cys | Aib(O-cyclic) | 3Pal | D-Phe(4-F) | Arg | Trp(6-F) | Cys |
| 1153-2 | Lys* | D-Arg | Gly | D-Arg | D-Nar | Cys | Aib(O-cyclic) | Orn | D-Phe(4-F) | Arg | Trp(6-F) | Cys |

In embodiments, the peptide of formula (II) is selected from Table A2A.

In embodiments, the peptide of formula (II) is selected from Table A2A, wherein the N-terminal, C-terminal and/or cyclic structure are optional feature.

TABLE A2A
Exemplary lipidated molecules.

| Mole- | | | | | | | | | | | | | | | |
|---|---|---|---|---|---|---|---|---|---|---|---|---|---|---|---|
| cule | | | | | | | | | | | | | N- | C- | |
| Name | X₋₄ | X₋₃ | X₋₂ | X₋₁ | X₁ | X₂ | X₃ | X₄ | X₅ | X₆ | X₇ | X₈ | term | term | Cyclic |
| 1150 | Lys* | D-Arg | Gly | D-Arg | D-Nar | Cys | Aib | Gln | D- | Arg | Trp(6-F) | Cys | Ac | NH₂ | Disulfide |
| | | | | | | | (O-cyclic) | | Phe(4-F) | | | | | | |
| 1142 | | Lys* | Gly | D-Arg | D-Nar | Cys | Aib | Gln | D- | Arg | Trp(6-F) | Cys | Ac | NH₂ | Disulfide |
| | | | | | | | (O-cyclic) | | Phe(4-F) | | | | | | |
| 1144 | | Lys* | PEG1 | PEG1 | D-Nar | Cys | Aib | Gln | D- | Arg | Trp(6-F) | Cys | Ac | NH₂ | Disulfide |
| | | | | | | | (O-cyclic) | | Phe(4-F) | | | | | | |
| 1151 | Lys* | D-Arg | PEG1 | D-Arg | Beta- | Cys | Aib | Gln | D- | Arg | Trp(6-F) | Cys | Ac | NH₂ | Disulfide |
| | | | | | homoArg | | (O-cyclic) | | Phe(4-F) | | | | | | |
| 1152 | Lys* | D-Arg | Gly | D-Arg | D-Nar | Cys | Aib | 3Pal | D- | Arg | Trp(6-F) | Cys | Ac | NH₂ | Disulfide |
| | | | | | | | (O-cyclic) | | Phe(4-F) | | | | | | |
| 1153 | Lys* | D-Arg | Gly | D-Arg | D-Nar | Cys | Aib | Orn | D- | Arg | Trp(6-F) | Cys | Ac | NH₂ | Disulfide |
| | | | | | | | (O-cyclic) | | Phe(4-F) | | | | | | |

TABLE 2
Exemplary lipidated molecules. Cyclic peptides include bridge (e.g. disulfide) between X₂ and X₃.

| | | | | | | | | | | | | | N- | C- | |
|---|---|---|---|---|---|---|---|---|---|---|---|---|---|---|---|
| [symbol] | [symbol] | X [symbol] | X₋₂ | X₋₁ | X₁ | X₂ | X₃ | X₄ | X₅ | X₆ | X₇ | X₈ | term | term | Cyclic |
| [symbol] | [symbol] | D-Arg | Gly | D-Arg | D-Nar | Cys | Phg | 3Pal | D-Phe(4-F) | Arg | Trp(6-F) | Cys | Ac | NH2 | Disulfide |
| [symbol] | [symbol] | Lys* | Gly | D-Arg | D-Nar | Cys | Phg | 3Pal | D-Phe(4-F) | Arg | Trp(6-F) | Cys | Ac | NH2 | Disulfide |
| [symbol] | [symbol] | Lys* | PEG1 | PEG1 | D-Nar | Cys | Phg | 3Pal | D-Phe(4-F) | Arg | Trp(6-F) | Cys | Ac | NH2 | Disulfide |
| [symbol] | Lys* | D-Arg | PEG1 | D-Arg | Beta- | Cys | Phg | 3Pal | D-Phe(4-F) | Arg | Trp(6-F) | Cys | Ac | NH2 | Disulfide |
| | | | | | homoArg | | | | | | | | | | |
| 1148 | Lys* | D-Arg | Gly | D-Arg | D-Nar | Cys | [symbol] | [symbol] | D-Phe | Arg | Trp(6-F) | Cys | Ac | NH2 | Disulfide |
| 1149 | | Lys* | Gly | D-Arg | D-Nar | Cys | [symbol] | [symbol] | D-Phe | Arg | Trp(6-F) | Cys | Ac | NH2 | Disulfide |
| 1137 | | Lys* | PEG1 | PEG1 | D-Nar | Cys | [symbol] | [symbol] | D-Phe | Arg | Trp(6-F) | Cys | Ac | NH2 | Disulfide |
| 1136 | Lys* | D-Arg | PEG1 | D-Arg | Beta- | Cys | [symbol] | [symbol] | D-Phe | Arg | Trp(6-F) | Cys | Ac | NH2 | Disulfide |
| | | | | | homoArg | | | | | | | | | | |
| 1150 | Lys* | D-Arg | Gly | D-Arg | D-Nar | Cys | Aib(O- | [symbol] | D-Phe(4-F) | Arg | Trp(6-F) | Cys | Ac | NH2 | Disulfide |
| | | | | | | | cyclic) | | | | | | | | |
| 1142 | | Lys* | Gly | D-Arg | D-Nar | Cys | Aib(O- | [symbol] | D-Phe(4-F) | Arg | Trp(6-F) | Cys | Ac | NH2 | Disulfide |
| | | | | | | | cyclic) | | | | | | | | |
| 1144 | | Lys* | PEG1 | PEG1 | D-Nar | Cys | Aib(O- | [symbol] | D-Phe(4-F) | Arg | Trp(6-F) | Cys | Ac | NH2 | Disulfide |
| | | | | | | | cyclic) | | | | | | | | |
| 1151 | Lys* | D-Arg | PEG1 | D-Arg | Beta- | Cys | Aib(O- | [symbol] | D-Phe(4-F) | Arg | Trp(6-F) | Cys | Ac | NH2 | Disulfide |
| | | | | | homoArg | | cyclic) | | | | | | | | |
| 1152 | Lys* | D-Arg | Gly | D-Arg | D-Nar | Cys | Aib(O- | 3Pal | D-Phe(4-F) | Arg | Trp(6-F) | Cys | Ac | NH2 | Disulfide |
| | | | | | | | cyclic) | | | | | | | | |
| 1153 | Lys* | D-Arg | Gly | D-Arg | D-Nar | Cys | Aib(O- | Orn | D-Phe(4-F) | Arg | Trp(6-F) | Cys | Ac | NH2 | Disulfide |
| | | | | | | | cyclic) | | | | | | | | |
| 1154 | Lys* | D-Arg | Gly | D-Arg | D-Nar | Cys | [symbol] | 3Pal | D-Phe(4-F) | Arg | Trp(6-F) | Cys | Ac | NH2 | Disulfide |
| 1155 | Lys* | D-Arg | Gly | D-Arg | D-Nar | Cys | [symbol] | Orn | D-Phe(4-F) | Arg | Trp(6-F) | Cys | Ac | NH2 | Disulfide |
| 1156 | Lys* | D-Arg | Gly | D-Arg | D-Nar | Cys | [symbol] | 3Pal | D-Phe | Arg | Trp(6-F) | Cys | Ac | NH2 | Disulfide |
| 1157 | Lys* | D-Arg | Gly | D-Arg | D-Nar | Cys | [symbol] | Orn | D-Phe | Arg | Trp(6-F) | Cys | Ac | NH2 | Disulfide |
| 111 | Lys* | Gly | Gly | Gly | D-Nar | Cys | Aib(O- | [symbol] | D-Phe(4-F) | Arg | Trp(6-F) | Cys | Ac | NH2 | Disulfide |
| | | | | | | | cyclic) | | | | | | | | |
| 112 | | Lys* | Gly | Gly | D-Nar | Cys | Aib(O- | [symbol] | D-Phe(4-F) | Arg | Trp(6-F) | Cys | Ac | NH2 | Disulfide |
| | | | | | | | cyclic) | | | | | | | | |
| 113 | | | Lys* | Gly | D-Nar | Cys | Aib(O- | [symbol] | D-Phe(4-F) | Arg | Trp(6-F) | Cys | Ac | NH2 | Disulfide |
| | | | | | | | cyclic) | | | | | | | | |
| 114 | | | | Lys* | D-Nar | Cys | Aib(O- | [symbol] | D-Phe(4-F) | Arg | Trp(6-F) | Cys | Ac | NH2 | Disulfide |
| | | | | | | | cyclic) | | | | | | | | |
| 123 | | Lys* | Gly | Gly | D-Nar | Cys | Phg | 3Pal | D-Phe(4-F) | Arg | Trp(6-F) | Cys | Ac | NH2 | Disulfide |
| 124 | | | Lys* | Gly | D-Nar | Cys | Phg | 3Pal | D-Phe(4-F) | Arg | Trp(6-F) | Cys | Ac | NH2 | Disulfide |
| 125 | | | | Lys* | D-Nar | Cys | Phg | 3Pal | D-Phe(4-F) | Arg | Trp(6-F) | Cys | Ac | NH2 | Disulfide |
| 130 | | Lys* | Gly | Gly | D-Nar | Cys | [symbol] | [symbol] | D-Phe | Arg | Trp(6-F) | Cys | Ac | NH2 | Disulfide |
| 131 | | | Lys* | Gly | D-Nar | Cys | [symbol] | [symbol] | D-Phe | Arg | Trp(6-F) | Cys | Ac | NH2 | Disulfide |
| 132 | | | | Lys* | D-Nar | Cys | [symbol] | [symbol] | D-Phe | Arg | Trp(6-F) | Cys | Ac | NH2 | Disulfide |
| 138 | | | Lys* | Gly | D-Nar | Cys | Cyclo- | 3Pal | D-Phe | Arg | Trp(6-F) | Cys | Ac | NH2 | Disulfide |
| | | | | | | | Leu | | | | | | | | |
| 139 | | | Lys* | Gly | D-Nar | Cys | Aib(O- | [symbol] | D-Phe | Arg | Trp(6-F) | Cys | Ac | NH2 | Disulfide |
| | | | | | | | cyclic) | | | | | | | | |
| 140 | | | | Lys* | Arg | Cys | Aib(O- | [symbol] | D-Phe(4-F) | Arg | Trp(6-F) | Pen | Ac | NH2 | Disulfide |
| | | | | | | | cyclic) | | | | | | | | |
| 141 | | | | Lys* | D-Nar | Cys | Aib(O- | [symbol] | D-Phe(4-F) | Arg | Trp(6-F) | Pen | Ac | NH2 | Disulfide |
| | | | | | | | cyclic) | | | | | | | | |
| 142 | | | | Lys* | Beta- | Cys | Aib(O- | [symbol] | D-Phe(4-F) | Arg | Trp(6-F) | Pen | Ac | NH2 | Disulfide |
| | | | | | homoArg | | cyclic) | | | | | | | | |
| 143 | | | | Lys* | Arg | Cys | Aib(O- | [symbol] | D-Phe | Arg | Trp | Pen | Ac | NH2 | Disulfide |
| | | | | | | | cyclic) | | | | | | | | |
| 144 | | | | Lys* | D-Nar | Cys | Aib(O- | [symbol] | D-Phe | Arg | Trp | Pen | Ac | NH2 | Disulfide |
| | | | | | | | cyclic) | | | | | | | | |
| 145 | | | | Lys* | Beta- | Cys | Aib(O- | [symbol] | D-Phe | Arg | Trp | Pen | Ac | NH2 | Disulfide |
| | | | | | homoArg | | cyclic) | | | | | | | | |
| 146 | | | | Lys* | Arg | Cys | Aib(O- | [symbol] | D-Phe | Arg | Trp(6-F) | Pen | Ac | NH2 | Disulfide |
| | | | | | | | cyclic) | | | | | | | | |
| 147 | | | | Lys* | D-Nar | Cys | Aib(O- | [symbol] | D-Phe | Arg | Trp(6-F) | Pen | Ac | NH2 | Disulfide |
| | | | | | | | cyclic) | | | | | | | | |
| 148 | | | | Lys* | Beta- | Cys | Aib(O- | [symbol] | D-Phe | Arg | Trp(6-F) | Pen | Ac | NH2 | Disulfide |
| | | | | | homoArg | | cyclic) | | | | | | | | |
| 149 | | | | Lys* | Arg | Cys | Aib(O- | [symbol] | D-Phe(4-F) | Arg | Trp | Pen | Ac | NH2 | Disulfide |
| | | | | | | | cyclic) | | | | | | | | |
| 150 | | | | Lys* | D-Nar | Cys | Aib(O- | [symbol] | D-Phe(4-F) | Arg | Trp | Pen | Ac | NH2 | Disulfide |
| | | | | | | | cyclic) | | | | | | | | |
| 151 | | | | Lys* | Beta- | Cys | Aib(O- | [symbol] | D-Phe(4-F) | Arg | Trp | Pen | Ac | NH2 | Disulfide |
| | | | | | homoArg | | cyclic) | | | | | | | | |
| 165 | | | | Lys* | Arg | Cys | Aib | [symbol] | D-Phe(4-F) | Arg | Trp(6-F) | Pen | Ac | NH2 | Disulfide |
| 166 | | | | Lys* | D-Nar | Pen | Aib(O- | [symbol] | D-Phe(4-F) | Arg | Trp(6-F) | Pen | Ac | NH2 | Disulfide |
| | | | | | | | cyclic) | | | | | | | | |
| 167 | | | | Lys* | D-Nar | Pen | Aib(O- | [symbol] | D-Phe(4-F) | Arg | Trp(6-F) | Cys | Ac | NH2 | Disulfide |
| | | | | | | | cyclic) | | | | | | | | |
| [symbol] | | | Lys* | Gly | D-Arg | Cys | L-aMeGlu | His | D-Phe | Arg | Trp | Cys | Ac | NH2 | Disulfide |
| 436 | | | Lys* | Gly | Beta- | Cys | L-aMeGlu | His | D-Phe | Arg | Trp | Cys | Ac | NH2 | Disulfide |
| | | | | | homoArg | | | | | | | | | | |
| 1129 | | Lys* | Gly | D-Arg | D-Nar | Cys | L-aMeGlu | His | D-Phe | Arg | Trp(6-Me) | Cys | Ac | NH2 | Disulfide |
| 1128 | | Lys* | Glu | PRO | D-Nar | Cys | L-aMeGlu | His | D-Phe | Arg | Trp(6-Me) | Cys | Ac | NH2 | Disulfide |
| 1133 | | Lys* | Gly | gGlu | D-Nar | Cys | L-aMeGlu | His | D-Phe | Arg | Trp(6-Me) | Cys | Ac | NH2 | Disulfide |
| 499 | | Lys* | Gly | Gly | D-Nar | Cys | L-aMeAsp | His | D-Phe | Arg | Trp(6-Me) | Cys | Ac | NH2 | Disulfide |
| 1131 | | Lys* | PEG1 | PEG1 | D-Nar | Cys | L-aMeAsp | His | D-Phe | Arg | Trp(6-Me) | Cys | Ac | NH2 | Disulfide |
| 1130 | | Lys* | Gly | Gly | beta- | Cys | L-aMeAsp | His | D-Phe | Arg | Trp(6-Me) | Cys | Ac | NH2 | Disulfide |
| | | | | | homoArg | | | | | | | | | | |
| 504 | | Lys* | PEG1 | PEG1 | beta- | Cys | L-aMeAsp | His | D-Phe | Arg | Trp(6-Me) | Cys | Ac | NH2 | Disulfide |
| | | | | | homoArg | | | | | | | | | | |
| 1135 | | Lys* | Gly | Gly | D-Arg | Cys | L-aMeAsp | His | D-Phe | Arg | Trp(6-Me) | Cys | Ac | NH2 | Disulfide |
| [symbol] | Lys* | D-Arg | PEG1 | FEG1 | D-Nar | Cys | L-aMeGlu | His | D-Phe | Arg | Trp(6-F) | Cys | Ac | NH2 | Disulfide |
| [symbol] | Lys* | PEG1 | PEG1 | PEG1 | D-Nar | Cys | L-aMeGlu | His | D-Phe | Arg | Trp(6-F) | Cys | Ac | NH2 | Disulfide |
| [symbol] | Lys* | Glu | PEG1 | PEG1 | D-Nar | Cys | L-aMeGlu | His | D-Phe | Arg | Trp(6-F) | Cys | Ac | NH2 | Disulfide |
| [symbol] | Lys* | Glu | Glu | Pro | D-Nar | Cys | L-aMeGlu | His | D-Phe | Arg | Trp(6-F) | Cys | Ac | NH2 | Disulfide |
| 1141 | Lys* | Ser | Glu | Pro | D-Nar | Cys | L-aMeGlu | His | D-Phe | Arg | Trp(6-F) | Cys | Ac | NH2 | Disulfide |
| 566 | Lys* | Ser | Gly | D-Arg | D-Nar | Cys | L-aMeGlu | His | D-Phe | Arg | Trp(6-F) | Cys | Ac | NH2 | Disulfide |
| 567 | Lys* | D-Arg | Gly | D-Arg | D-Nar | Cys | L-aMeGlu | His | D-Phe | Arg | Trp(6-F) | Cys | Ac | NH2 | Disulfide |
| 568 | Lys* | D-Arg | Ser | γ-Glu | D-Nar | Cys | L-aMeGlu | His | D-Phe | Arg | Trp(6-F) | Cys | Ac | NH2 | Disulfide |
| 569 | Lys* | Glu | Gly | γ-Glu | D-Nar | Cys | L-aMeGlu | His | D-Phe | Arg | Trp(6-F) | Cys | Ac | NH2 | Disulfide |
| 1143 | Lys* | Gly | Gly | γ-Glu | D-Nar | Cys | L-aMeGlu | His | D-Phe | Arg | Trp(6-F) | Cys | Ac | NH2 | Disulfide |
| 571 | | Lys* | Gly | γ-Glu | Me-D- | Cys | L-aMeGlu | His | D-Phe | Arg | Trp(6-F) | Cys | Ac | NH2 | Disulfide |
| | | | | | Arg | | | | | | | | | | |
| 572 | | Lys* | Gly | D-Arg | D-Nar | Cys | L-aMeGlu | His | D-Phe | Arg | Trp(6-F) | Cys | Ac | NH2 | Disulfide |
| 573 | | Lys* | Glu | Pro | D-Nar | Cys | L-aMeGlu | His | D-Phe | Arg | Trp(6-F) | Cys | Ac | NH2 | Disulfide |
| 574 | | Lys* | Glu | γ-Glu | D-Nar | Cys | L-aMeGlu | His | D-Phe | Arg | Trp(6-F) | Cys | Ac | NH2 | Disulfide |
| 575 | | Lys* | D-Arg | γ-Glu | D-Nar | Cys | L-aMeGlu | His | D-Phe | Arg | Trp(6-F) | Cys | Ac | NH2 | Disulfide |
| 1134 | Lys* | Glu | Glu | Pro | D-Nar | Cys | L-aMeGlu | His | D-Phe(3,4,5- | Arg | Trp(6-Me) | Cys | Ac | NH2 | Disulfide |
| | | | | | | | | | [symbol] | | | | | | |
| 1132 | Lys* | Glu | PEG1 | PEG1 | D-Nar | Cys | L-aMeGlu | His | D-Phe(3,4,5- | Arg | Trp(6-Me) | Cys | Ac | NH2 | Disulfide |
| | | | | | | | | | [symbol] | | | | | | |
| 589 | Lys* | Glu | Glu | Pro | D-Nar | Cys | [symbol] | His | D-Phe | Arg | Trp(6-Me) | Cys | Ac | NH2 | Disulfide |
| 590 | Lys* | Glu | PEG1 | PEG1 | D-Nar | Cys | [symbol] | His | D-Phe | Arg | Trp(6-Me) | Cys | Ac | NH2 | Disulfide |
| 1146 | Lys* | D-Arg | Gly | D-Arg | D-Nar | Cys | Phg | 3Pal | D-Phe(4-F) | Arg | Trp(6-F) | Cys | Ac | NH2 | Disulfide |

[symbol]

indicates data missing or illegible when filed

In embodiments, one or more amino acid residues and/or linkers are conjugated to X₁ of any one of the peptides of formula (I) or formula (II). In embodiments, at least 1, or at least 2, or at least 3, or at least 4, or at least 5 amino acid residues are conjugated to X₁ of any one of the peptides of formula (I) or formula (II). In embodiments, at least 1, or at least 2, or at least 3, or at least 4, or at least 5 linkers are conjugated to X₁ of any one of the peptides of formula (I) or formula (II). In embodiments, the one or more amino acid residues and/or linkers and/or spacers conjugated to X₁ have the sequential designation of X₋₁, X₋₂, X₋₃, X₋₄, X₋₅, and so forth. Non-limiting examples of linkers include lipids and PEG linkers (e.g. PEG-1). In embodiments, the one or more amino acid residues and/or linkers are selected from Table 2. In embodiments, the linker is a PEG linker or lipid selected from Table 2. In embodiments, one or more amino acid residues and/or linkers are conjugated to X₈ of any one of the peptides of formula (I) or formula (II). In embodiments, at least 1, or at least 2, or at least 3, or at least 4, or at least 5 amino acid residues are conjugated to X₈ of any one of the peptides of formula (I) or formula (II). In embodiments, at least 1, or at least 2, or at least 3, or at least 4, or at least 5 linkers and/or spacers are conjugated to X₁ of any one of the peptides of formula (I) or formula (II). In embodiments, the one or more amino acid residues and/or linkers conjugated to X₈ have the sequential designation of X₉, X₁₀, X₁₁, X₁₂, X₁₃, X₁₄ and so forth. Non-limiting examples of linkers include lipids and PEG linkers (e.g. PEG-1). In embodiments, the one or more amino acid residues and/or linkers are selected from Table 2. In embodiments, the linker is a PEG linker or lipid selected from Table 2.

In embodiments, the one or more amino acid residues and/or linkers are selected from Table 3. In embodiments, the linker is a PEG linker or lipid selected from Table 3.

In embodiments, a linker is an amino acid, including but not limited to modified amino acids. Non-limiting examples of modified amino acids include PEG groups (e.g., PEG-2), and lipids. In embodiments, the modified amino acid is Lys(AEEAc-AEEAc-L-γ-Glu-17-carboxyheptadecanoyl) (Lys*). In embodiments, the linker is Lys*.

In embodiments, the peptide further comprises one or more lipids conjugated to X₁ and/or X₈.

In embodiments, the peptide further comprises one or more PEG linkers conjugated to X₁ and/or X₈.

In embodiments, the peptide of formula (I) is a peptide of formula (Ia):

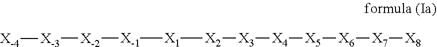

wherein in formula (Ia): X₁, X₋₂, X₋₃, X₋₄, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the peptide of formula (I) is a peptide of formula (Ib):

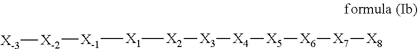

wherein in formula (Ib): X₋₁, X₋₂, X₋₃, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the peptide of formula (I) is a peptide of formula (Ic):

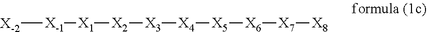

wherein in formula (Ic): X₋₁, X₋₂, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the peptide of formula (I) is a peptide of formula (Id):

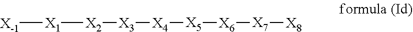

wherein in formula (Id): X₋₁, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the peptide of formula (I) is a peptide of formula (Ie):

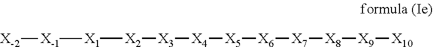

wherein in formula (Ie): X₋₁, X₋₂, X₁, X₂, X₃, X₄, X₅, X₆, X₇, X₈, X₉, and X₁₀ are each independently an amino acid selected from Table 1 or a linker. In embodiments, the linker is a PEG linker (e.g. PEG-1).

In embodiments, the peptide of formula (I) is a peptide of formula (If):

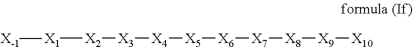

wherein in formula (Ie): X₋₁, X₁, X₂, X₃, X₄, X₅, X₆, X₇, X₈, X₉, and X₁₀ are each independently an amino acid selected from Table 1 or a linker. In embodiments, the linker is a PEG linker (e.g. PEG-1).

In embodiments, the cyclic peptide of formula (I) is a cyclic peptide of any one of formula (Ia), formula (Ib), formula (Ic), formula (Id), formula (Ie), or formula (If), wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide consists of the amino acid sequence as set forth in formula (Ia). In embodiments, the peptide consists of the amino acid sequence as set forth in formula (Ib). In embodiments, the peptide consists of the amino acid sequence as set forth in formula (Ic). In embodiments, the peptide consists of the amino acid sequence as set forth in formula (Id). In embodiments, the peptide consists of the amino acid sequence as set forth in formula (Ie). In embodiments, the peptide consists of the amino acid sequence as set forth in formula (If).

In aspects and embodiments, there is provided a peptide comprising the amino acid sequence of formula (BI), formula (CI), formula (DI), formula (EI), or formula (FI).

In embodiments, in formula (BI), X₁, X₂, X₃, X₄, X₅, X₆, X₇, and X₈ are each independently an amino acid selected from Table 1, Table 2, Table 3, Table B1, Table B1A, Table B2, and Table B2A.

In embodiments, in formula (CI), X₁, X₂, X₃, X₄, X₅, X₆, X₇, and X₈ are each independently an amino acid selected from Table 1, Table 2, Table 3, Table C1, Table C1A, Table C2, and Table C2A.

In embodiments, in formula (DI), X₁, X₂, X₃, X₄, X₅, X₆, X₇, and X₈ are each independently an amino acid selected from Table 1, Table 2, Table 3, Table D1, Table D1A, Table D2, and Table D2A.

In embodiments, in formula (EI), X₁, X₂, X₃, X₄, X₅, X₆, X₇, and X₈ are each independently an amino acid selected from Table 1, Table 2, Table 3, Table E1, Table E1A, Table E2, and Table E2A.

In embodiments, in formula (FI), X₁, X₂, X₃, X₄, X₅, X₆, X₇, and X₈ are each independently an amino acid selected from Table 1, Table 2, Table 3, Table F1, Table F1A, Table F2, and Table F2A.

In embodiments, the peptide of formula (I) is a peptide of any one of formula (Ia), formula (Ib), formula (Ic), formula (Id), formula (Ie), or formula (If).

In embodiments, the peptide of formula (BI) is a peptide of anyone of formula (BIa), formula (BIb), formula (BIc), formula (BId), formula (BIe), or formula (BIf).

In embodiments, in formula (BIa), formula (BIb), formula (BIc), formula (BId), formula (BIe), or formula (BIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (CI) is a peptide of anyone of formula (CIa), formula (CIb), formula (CIc), formula (CId), formula (CIe), or formula (CIf).

In embodiments, in formula (CIa), formula (CIb), formula (CIc), formula (CId), formula (CIe), or formula (CIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (DI) is a peptide of any one of formula (DIa), formula (DIb), formula (DIc), formula (DId), formula (DIe), or formula (DIf).

In embodiments, in formula (DIa), formula (DIb), formula (DIc), formula (DId), formula (DIe), or formula (DIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (EI) is a peptide of anyone of formula (EIa), formula (EIb), formula (EIc), formula (EId), formula (EIe), or formula (EIf).

In embodiments, in formula (EIa), formula (EIb), formula (EIc), formula (EId), formula (EIe), or formula (EIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide of formula (FI) is a peptide of any one of formula (FIa), formula (FIb), formula (FIc), formula (FId), formula (FIe), or formula (FIf).

In embodiments, in formula (FIa), formula (FIb), formula (FIc), formula (FId), formula (FIe), or formula (FIf), X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

In embodiments, the peptide consists of the amino acid sequence as set forth in formula (BI). In embodiments, the peptide consists of the amino acid sequence as set forth in formula (BIa). In embodiments, the peptide consists of the amino acid sequence as set forth in formula (BIb). In embodiments, the peptide consists of the amino acid sequence as set forth in formula (BIc). In embodiments, the peptide consists of the amino acid sequence as set forth in formula (BId). In embodiments, the peptide consists of the amino acid sequence as set forth in formula (BIe). In embodiments, the peptide consists of the amino acid sequence as set forth in formula (BIf).

In embodiments, the peptide consists of the amino acid sequence as set forth in formula (CI). In embodiments, the peptide consists of the amino acid sequence as set forth in formula (CIa). In embodiments, the peptide consists of the amino acid sequence as set forth in formula (CIb). In embodiments, the peptide consists of the amino acid sequence as set forth in formula (CIc). In embodiments, the peptide consists of the amino acid sequence as set forth in formula (CId). In embodiments, the peptide consists of the amino acid sequence as set forth in formula (CIe). In embodiments, the peptide consists of the amino acid sequence as set forth in formula (CIf).

In embodiments, the peptide consists of the amino acid sequence as set forth in formula (DI). In embodiments, the peptide consists of the amino acid sequence as set forth in formula (DIa). In embodiments, the peptide consists of the amino acid sequence as set forth in formula (DIb). In embodiments, the peptide consists of the amino acid sequence as set forth in formula (DIc). In embodiments, the peptide consists of the amino acid sequence as set forth in formula (DId). In embodiments, the peptide consists of the amino acid sequence as set forth in formula (DIe). In embodiments, the peptide consists of the amino acid sequence as set forth in formula (DIf).

In embodiments, the peptide consists of the amino acid sequence as set forth in formula (EI). In embodiments, the peptide consists of the amino acid sequence as set forth in formula (EIa). In embodiments, the peptide consists of the amino acid sequence as set forth in formula (EIb). In embodiments, the peptide consists of the amino acid sequence as set forth in formula (EIc). In embodiments, the peptide consists of the amino acid sequence as set forth in formula (EId). In embodiments, the peptide consists of the amino acid sequence as set forth in formula (EIe). In embodiments, the peptide consists of the amino acid sequence as set forth in formula (EIf).

In embodiments, the peptide consists of the amino acid sequence as set forth in formula (FI). In embodiments, the peptide consists of the amino acid sequence as set forth in formula (FIa). In embodiments, the peptide consists of the amino acid sequence as set forth in formula (FIb). In embodiments, the peptide consists of the amino acid sequence as set forth in formula (FIc). In embodiments, the peptide consists of the amino acid sequence as set forth in formula (FId). In embodiments, the peptide consists of the amino acid sequence as set forth in formula (FIe). In embodiments, the peptide consists of the amino acid sequence as set forth in formula (FIf).

Amino acids described herein are construed as L-amino acids unless specified otherwise (e.g., D-amino acids, such as D-arginine (D-Arg)).

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 linkers and/or spacers at the amino and/or carboxy terminus.

In embodiments, the peptides of formula (I) or formula (II) comprises an N-terminal and/or C-terminal modification. In embodiments, the N-terminal modification comprises acetylation, propionylation, methylation, myristoylation, palmitoylation, or ubiquitylation. In embodiments, the N-terminal modification comprises acyl group. Non-limiting examples of acyl group include acetyl, propionyl, butyryl, formyl, propenyl, crotyl, butenyl, and benzyl. In embodiments, C-terminal modifications include neutralization of the negative charge that the carboxylic acid derivative displays at physiological pH. In embodiments, C-terminal modifications include NR1R2, wherein R1 and R2 are selected from H or alkyl.

In embodiments, the peptides of formula (I) or formula (II) comprises an N-terminal acetyl. In embodiments, the peptides of formula (I) or formula (II) comprises a C-terminal amide. In embodiments, the peptides of formula (I) or formula (II) comprises an N-terminal acetyl and C-terminal amide.

In embodiments, the peptide of formula (I) is selected from Table 1 and Table 2.

In embodiments, the peptide of formula (II) is selected from Table 1 and Table 2.

In embodiments, X₄ is Gln. In embodiments, X₄ is Cit. In embodiments, X₄ is hCit. In embodiments, X₄ is 3-Pal. In embodiments, X₄ is hGln. In embodiments, X₄ is His. In embodiments, X₄ is Orn.

In embodiments, X₃-X₄ is selected from Aib(O-cyclic)-Gln, Aib(O-cyclic)-hCit, Aib(O-cyclic)-Cit, Aib(O-cyclic)-3-Pal, Aib(O-cyclic)-hGln, Aib(O-cyclic)-His, and Aib(O-cyclic)-Orn.

In embodiments, X₅ is selected from 4-fluoro-D-phenylalanine (D-Phe(4-F)), D-phenylalanine (D-Phe), and 4-methyl-D-phenylalanine (D-Phe(4-Me)).

In embodiments, X₅ is D-Phe(4-F).

In embodiments, X₅ is D-Phe.

In embodiments, X₅ is D-Phe(4-Me).

In embodiments, X₆ is arginine (Arg).

In embodiments, X₇ is 6-fluoro-L-tryptophan (Trp(6-F)).

In embodiments, X₈ is cysteine (Cys).

In embodiments, X₈ is penicillamine (Pen).

In embodiments, when X₂ and X₈ are Cys, X₂ and X₈ are linked by a disulfide bridge (-S-S-). In embodiments, when one of X₂ and X₈ is Cys and the other of X₂ and X₈ is Pen, X₂ and X₈ are linked by a disulfide bridge. In embodiments, when X₂ is Cys and X₈ is Pen, X₂ and X₈ are linked by a disulfide bridge.

In embodiments, X₁ is selected from D-norarginine (D-Nar) and beta-homo-L-arginine (Beta-homoArg).

In embodiments, X₁ is D-Nar.

In embodiments, X₁ is beta-homoArg.

In embodiments, X₂ is Cys.

In embodiments, X₃ is selected from X₃ column in Table 9 and X₄ is selected from X₄ column in Table 9. In embodiments, X₃ is selected from X₃ column in Table 1 and X₄ is selected from X₄ column in Table 1.

In embodiments, X₃-X₄ is selected from Phg-3-Pal, D-aMeOrn-Gln, Aib(O-cyclic)-Gln, Aib(O-cyclic)-hCit, Aib(O-cyclic)-Cit, Cyclo-Leu-3-Pal, L-aMeGlu-His, hGlu-Gln, D-aMeSer-Gln, Cyclo-Leu-Gln, hGlu-His, Ala(2-Me)-Gln, L-aMeAsp-His, Ala(2-Me)-His, D-bhGlu-His, D-aMeSer-His, bhGlu-His, D-aMeOrn-3-Pal, and Aib(O-cyclic)-3-Pal.

In embodiments, X₃-X₄ is selected from Phg-3-Pal, D-aMeOrn-Gln, Cyclo-Leu-3-Pal, and Aib(O-cyclic)-Gln.

In embodiments, X₃-X₄ is Phg-3-Pal.

In embodiments, X₃-X₄ is D-aMeOrn-Gln.

In embodiments, X₃-X₄ is Aib(O-cyclic)-Gln.

In embodiments, X₃-X₄ is Cyclo-Leu-3-Pal.

TABLE 9
Exemplary combinations of X₃ and X₄ positions.

| X₃ | X₄ |
|---|---|
| Phg | 3-Pal |
| D-aMeOrn | Gln |
| Aib(O-cyclic) | Gln |
| Aib(O-cyclic) | hCit |
| Aib(O-cyclic) | Cit |
| L-aMeGlu | His |
| hGlu | Gln |
| D-aMeSer | Gln |
| Cyclo-Leu | Gln |
| hGlu | His |
| Ala(2-Me) | Gln |
| L-aMeAsp | His |
| Ala(2-Me) | His |
| Cyclo-Leu | 3-Pal |
| D-bhGlu | His |
| D-aMeSer | His |
| bhGlu | His |
| D-aMeOrn | 3-Pal |
| Aib(O-cyclic) | 3-Pal |

In embodiments, X₅ is selected from 4-fluoro-D-phenylalanine (D-Phe(4-F)), D-phenylalanine (D-Phe), 4-methyl-D-phenylalanine (D-Phe(4-Me)), 3-trifluoromethyl-D-phenylalanine (D-Phe(3-CF3)), 3-fluoro-D-phenylalanine (D-Phe(3-F)), 2,3-difluoro-D-phenylalanine (D-Phe(2,3-diF)), 2,4,5-trifluoro-D-phenylalanine (D-Phe(2,4,5-triF)), 2,4-dichloro-D-phenylalanine (D-Phe(2,4-diCl)), 2,4-difluoro-D-phenylalanine (D-Phe(2,4-diF)), 4-chloro-2-fluoro-D-phenylalanine (D-Phe(2-F,4-Cl)), 3,4,5-trifluoro-D-phenylalanine (D-Phe(3,4,5-triF)), 3,4-difluoro-D-phenylalanine (D-Phe(3,4-diF)), 3,4-dimethyl-D-phenylalanine (D-Phe(3,4-diMe)), 3-chloro-D-phenylalanine (D-Phe(3-Cl)), 4-methyl-3-fluoro-D-phenylalanine (D-Phe(3-F,4-Me)), 3-methyl-D-phenylalanine (D-Phe(3-Me)), 4-(trifluoromethyl)-D-phenylalanine (D-Phe(4-CF3)), and 4-chloro-D-phenylalanine (D-Phe(4-Cl)).

In embodiments, X₅ is 4-fluoro-D-phenylalanine (D-Phe(4-F)) or D-phenylalanine (D-Phe). In embodiments, X₅ is 4-Fluoro-D-phenylalanine (D-Phe(4-F)). In embodiments, X₅ is D-phenylalanine (D-Phe).

In embodiments, X₆ is arginine (Arg).

In embodiments, X₇ is selected from 4-fluoro-L-tryptophan (Trp(4-F)), 5-chloro-L-tryptophan (Trp(5-Cl)), 5-fluoro-L-tryptophan (Trp(5-F)), 5-methyl-L-tryptophan (Trp(5-Me)), 6-bromo-L-tryptophan (Trp(6-Br)), 6-(trifluoromethyl)-L-tryptophan (Trp(6-CF3)), 6-chloro-L-tryptophan (Trp(6-Cl)), 6-fluoro-L-tryptophan (Trp(6-F)), 6-methyl-L-tryptophan (Trp(6-Me)), 7-fluoro-L-tryptophan (Trp(7-F)), and tryptophan (Trp).

In embodiments, X₇ is 4-fluoro-L-tryptophan (Trp(4-F)). In embodiments, X₇ is 6-fluoro-L-tryptophan (Trp(6-F)).

In embodiments, X₈ is selected from Cysteine (Cys), 3-amino-L-alanine (Dap), and penicillamine (Pen).

In embodiments, X₈ is cysteine (Cys). In embodiments, X₈ is penicillamine (Pen).

In embodiments, X₁ is selected from Arg, beta-homo-L-arginine (Beta-homoArg), D-arginine (D-Arg), D-norarginine (D-Nar), homo-L-arginine (L-hArg), and L-norarginine (Nar).

In embodiments, X₁ is selected from D-arginine (D-Arg).

In embodiments, X₂ is selected from aspartic acid (Asp), Cys, and L-glutamate (Glu).

In embodiments, X₂ is Cysteine (Cys).

In embodiments, when X₂ and X₈ are Cys, the amino acids are linked by a disulfide bridge. In embodiments, when one of X₂ and X₈ is Cys and the other of X₂ and X₈ is Pen, the amino acids are linked by a disulfide bridge. In embodiments, when X₂ is Cys and X₈ is Pen, the amino acids are linked by a disulfide bridge. In embodiments, when X₂ is Asp or Glu, and X₈ is Dap, the amino acids are linked by an amide bond.

In embodiments, X₁ is selected from D-Nar, Arg, Beta-homoArg, D-Arg, X₅ is selected from D-Phe(4-F), D-Phe, D-Phe(3-CF3), D-Phe(3-F), and X₇ is selected from Trp(6-F), Trp(6-Me), Trp(5-Me) and Trp.

In embodiments, X₁ is selected from X₁ column of Table 10. In embodiments, X₅ is selected from X₅ column of Table 10. In embodiments, X₇ is selected from X₇ column of Table 10.

TABLE 10
Exemplary X₁, X₅, and X₇ Residues

| X₁ | X₅ | X₇ |
|---|---|---|
| D-Nar | D-Phe(4-F) | Trp(6-F) |
| Arg | D-Phe | Trp(6-Me) |
| Beta-homoArg | D-Phe(3-CF3) | Trp(5-Me) |
| D-Arg | D-Phe(3-F) | Trp |

In embodiments, X₁, X₅, and X₇ are D-Nar, D-Phe(4-F), and Trp(6-F), respectively. In embodiments, X₁, X₅, and X₇ are D-Nar, D-Phe, and Trp(6-F), respectively.

In embodiments, X₁, X₅, and X₇ are D-Nar, D-Phe(4-F), and Trp(6-F), respectively, and X₈ is penicillamine (Pen). In embodiments, X₁, X₅, and X₇ are D-Nar, D-Phe, and Trp(6-F), respectively, and X₈ is penicillamine (Pen).

In embodiments, the peptide is a peptide consisting of the amino acid sequence as set forth in formula (III):

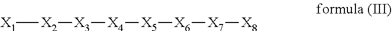

wherein in formula (III):
X₁ is D-norarginine (D-Nar);
X₂ is cysteine (Cys);
X₃ is 3-Aminooxetane-3-carboxylic acid (Aib(O-cyclic));
X₄ is selected from glutamine (Gln), homocitrulline (hCit), and citrulline (Cit);
X₅ is selected from 4-fluoro-D-phenylalanine (D-Phe(4-F)), D-phenylalanine (D-Phe), and 4-methyl-D-phenylalanine (D-Phe(4-Me));
X₆ is arginine (Arg);
X₇ is 6-fluoro-L-tryptophan (Trp(6-F)); and
X₈ is penicillamine (Pen).

In embodiments of formula (III), X₄ is glutamine (Gln). In embodiments of formula (III), X₄ is homocitrulline (hCit). In embodiments of formula (III), X₄ is citrulline (Cit). In embodiments of formula (III), X₅ is 4-fluoro-D-phenylalanine (D-Phe(4-F)). In embodiments of formula (III), X₅ is D-phenylalanine (D-Phe). In embodiments of formula (III), X₅ is 4-methyl-D-phenylalanine (D-Phe(4-Me)). In embodiments, the peptide of formula (III) is capped with N-terminal acetyl.

In embodiments, the peptide is a peptide consisting of the amino acid sequence as set forth in formula (III):

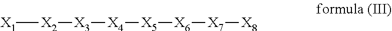

wherein in formula (III):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen).

In embodiments, the peptide of formula (III) is capped with N-terminal acetyl.

In embodiments, the cyclic peptide is a peptide consisting of the amino acid sequence as set forth in formula (IV):

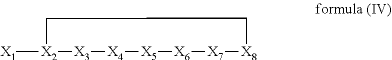

wherein in formula (IV):
X₁ is D-norarginine (D-Nar);
X₂ is cysteine (Cys);
X₃ is 3-Aminooxetane-3-carboxylic acid (Aib(O-cyclic));
X₄ is selected from glutamine (Gln), homocitrulline (hCit), and citrulline (Cit);
X₅ is selected from 4-fluoro-D-phenylalanine (D-Phe(4-F)), D-phenylalanine (D-Phe), and 4-methyl-D-phenylalanine (D-Phe(4-Me));
X₆ is arginine (Arg);
X₇ is 6-fluoro-L-tryptophan (Trp(6-F)); and
X₈ is penicillamine (Pen), wherein

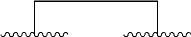 [SMILES: *CC*]

represents a disulfide bridge, and the peptide is capped with N-terminal acetyl.

In embodiments of formula (IV), X₄ is glutamine (Gln). In embodiments of formula (IV), X₄ is homocitrulline (hCit). In embodiments of formula (IV), X₄ is citrulline (Cit). In embodiments of formula (IV), X₅ is 4-fluoro-D-phenylalanine (D-Phe(4-F)). In embodiments of formula (IV), X₅ is D-phenylalanine (D-Phe). In embodiments of formula (IV), X₅ is 4-methyl-D-phenylalanine (D-Phe(4-Me)). In embodiments, the peptide of formula (IV) is capped with N-terminal acetyl.

In embodiments, the cyclic peptide is a peptide consisting of the amino acid sequence as set forth in formula (IV):

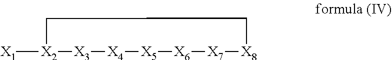

wherein in formula (IV):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen), wherein

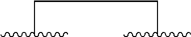 [SMILES: *CC*]

represents a disulfide bridge, and the peptide is capped with N-terminal acetyl.

### Cyclic Peptides

In embodiments, the peptide of the present disclosure is a cyclic peptide. In the embodiments, cyclic peptides include polypeptide chains taking cyclic ring structure. In embodiments, the ring structure is formed by linking one end of the peptide to its other end with an amide bond, or other chemically stable bonds such as lactam, ether, thioether, disulfide or via a stapled linkage. In embodiments, N-to-C(or head-to-tail) cyclization is amide bond formation between amino and carboxyl termini. In embodiments, the cyclic peptide comprises a bridge between amino acid at position X₂ and amino acid at position X₈ (e.g. as exemplified in formula II or formula IV).

In embodiments, the cyclic peptide comprises a disulfide bridge or a lactam bridge. In embodiments, the cyclic peptide comprises a disulfide bridge. In embodiments, the cyclic peptide comprises a lactam bridge (—NH—C(═O)—). In embodiments, the cyclic peptide comprises a bridge comprising one or more selected from amide, ether, disulfide, lactam, head-tail amidation, and Asp-Lys.

In embodiments, cyclic peptide comprises a bridge.

As used herein, the notation

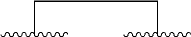 [SMILES: *CC*]

represents the linkage between two amino acids to form a cyclic peptide. In embodiments, the linkage is a bridge (e.g. a disulfide bridge -S-S-).

In embodiments, the cyclic peptide has the formula (II):

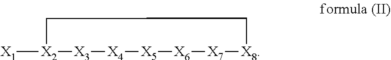

In embodiments, the peptide of formula (I) is a cyclic peptide of formula (II). In embodiments, the peptide of formula (I) is a peptide of formula (II) comprising a disulfide bridge. In embodiments, the peptide of formula (I) is a peptide of formula (II) comprising a lactam bridge. In embodiments, the peptide of molecules 1150, 1142, 1144, 1151, or 1158 is a cyclic peptide comprising a disulfide bridge. In embodiments, the peptide of molecules 1150, 1142, 1144, 1151, or 1158 is a cyclic peptide comprising a lactam bridge.

In embodiments, the peptide further comprises one or more lipids conjugated to X₁ and/or X₈. In embodiments, the peptide further comprises one or more amino acids conjugated to X₁ and/or X₈.

In embodiments, the one or more lipids are directly conjugated to X₁ and/or X₈. In embodiments, the one or more lipids are directly conjugated to the one or more amino acids, and the one or more amino acids are directly conjugated to X₁ and/or X₈.

In embodiments, the peptide further comprises one or more PEG linkers conjugated to X₁ and/or X₈.

In embodiments, the one or more PEG linkers are directly conjugated to X₁ and/or X₈. In embodiments, the one or more PEG linkers are directly conjugated to the one or more amino acids, and the one or more amino acids are directly conjugated to X₁ and/or X₈.

In embodiments, one or more amino acid residues and/or linkers are conjugated to X₁ of any one of the peptides of formula (I) or formula (II). In embodiments, at least 1, or at least 2, or at least 3, or at least 4, or at least 5 amino acid residues are conjugated to X₁ of any one of the peptides of formula (I) or formula (II). In embodiments, at least 1, or at least 2, or at least 3, or at least 4, or at least 5 linkers are conjugated to X₁ of any one of the peptides of formula (I) or formula (II). In embodiments, the one or more amino acid residues and/or linkers and/or spacers conjugated to X₁ have the sequential designation of X₋₁, X₂, X₃, X₄, X₋₅, and so forth. Non-limiting examples of linkers include lipids and PEG linkers (e.g. PEG-1). In embodiments, the one or more amino acid residues and/or linkers are selected from Table 2. In embodiments, the linker is a PEG linker or lipid selected from Table 2.

In embodiments, one or more amino acid residues and/or linkers are conjugated to X₈ of any one of the peptides of formula (I) or formula (II). In embodiments, at least 1, or at least 2, or at least 3, or at least 4, or at least 5 amino acid residues are conjugated to X₈ of any one of the peptides of formula (I) or formula (II). In embodiments, at least 1, or at least 2, or at least 3, or at least 4, or at least 5 linkers are conjugated to X₁ of any one of the peptides of formula (I) or formula (II). In embodiments, the one or more amino acid residues and/or linkers and/or spacers conjugated to X₈ have the sequential designation of X₉, X₁₀, X₁₁, X₁₂, X₁₃, X₁₄ and so forth. Non-limiting examples of linkers include lipids and PEG linkers (e.g. PEG-1). In embodiments, the one or more amino acid residues and/or linkers are selected from Table 2. In embodiments, the linker is a PEG linker or lipid selected from Table 2.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 linkers and/or spacers at the amino and/or carboxy terminus.

In embodiments, the cyclic peptide of formula (II) is a cyclic peptide of formula (IIa):

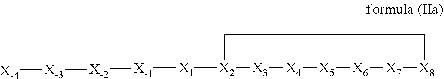

wherein in formula (IIa): X₋₁, X₋₂, X₋₃, X₋₄, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the cyclic peptide of formula (II) is a cyclic peptide of formula (IIb):

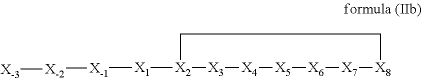

wherein in formula (IIb): X₋₁, X₋₂, X₋₃, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the cyclic peptide of formula (II) is a cyclic peptide of formula (IIc):

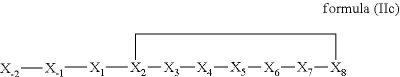

wherein in formula (IIc): X₋₁, X₋₂, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the cyclic peptide of formula (II) is a cyclic peptide of formula (IId):

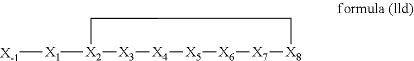

wherein in formula (IId): X₋₁, X₁, X₂, X₃, X₄, X₅, X₆, X₇, and X₈ are each independently an amino acid selected from Table 1 or a linker. In embodiments, the linker is a PEG linker (e.g. PEG-1).

In embodiments, the cyclic peptide of formula (II) is a cyclic peptide of formula (IIe):

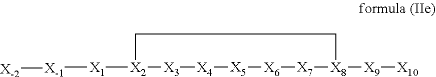

wherein in formula (IIe): X₋₁, X₋₂, X₁, X₂, X₃, X₄, X₅, X₆, X₇, X₈, X₉, and X₁₀ are each independently an amino acid selected from Table 1 or a linker. In embodiments, the linker is a PEG linker (e.g. PEG-1).

In embodiments, the cyclic peptide of formula (II) is a cyclic peptide of formula (IIf):

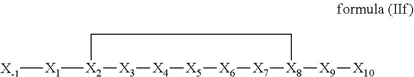

wherein in formula (IIf): X₋₁, X₋₂, X₁, X₂, X₃, X₄, X₅, X₆, X₇, X₈, X₉, and X₁₀ are each independently an amino acid selected from Table 1 or a linker. In embodiments, the linker is a PEG linker (e.g. PEG-1).

In embodiments, the cyclic peptide of formula (II) is a cyclic peptide of any one of formula (IIa), formula (IIb), formula (IIc), formula (IId), formula (IIe), or formula (IIf), wherein X₋₁, X₋₂, X₋₃, X₋₄, X₁, X₂, X₃, X₄, X₅, X₆, X₇, X₈, X₉, and X₁₀ are each independently an amino acid selected from Table 1, Table 2, and Table 3 or a linker.

In embodiments, the peptides of formula (I) or formula (II) comprises an N-terminal acetyl (-C(═O)CH₃). In embodiments, the peptides of formula (I) or formula (II) comprises a C-terminal amide. In embodiments, the peptides of formula (I) or formula (II) comprises an N-terminal acetyl and C-terminal amide.

In embodiments, the peptide is capped with N-terminal acetyl and/or C-terminal amide groups.

In embodiments, the peptides of formula (I) or formula (II) comprises an N-terminal and/or C-terminal modification. In embodiments, the N-terminal modification comprises acetylation, propionylation, methylation, myristoylation, palmitoylation or ubiquitylation. In embodiments, the N-terminal modification comprises acyl group. Non-limiting examples of acyl group include acetyl, propionyl, butyryl, formyl, propenyl, crotyl, butenyl, and benzyl. In embodiments, C-terminal modifications include neutralization of the negative charge that the carboxylic acid derivative displays at physiological pH. In embodiments, C-terminal modifications include NR1R2, wherein R1 and R2 are selected from H or alkyl.

In embodiments, the peptide of formula (I) or formula (II) is selected from Table 1 and Table 2.

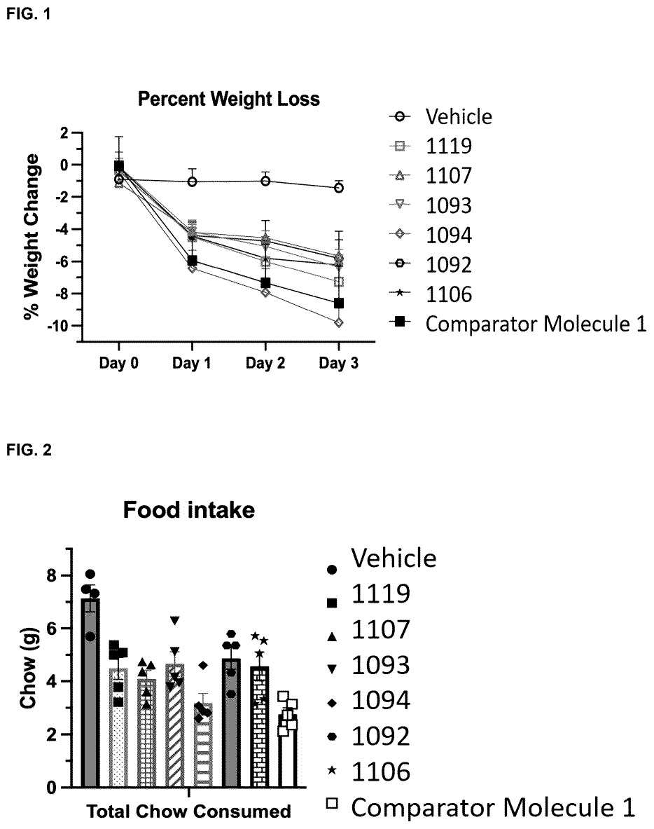

In embodiments, the peptide of the disclosure is a peptide described in FIG. 1.

In embodiments, the peptide of the disclosure is a peptide described in FIG. 2.

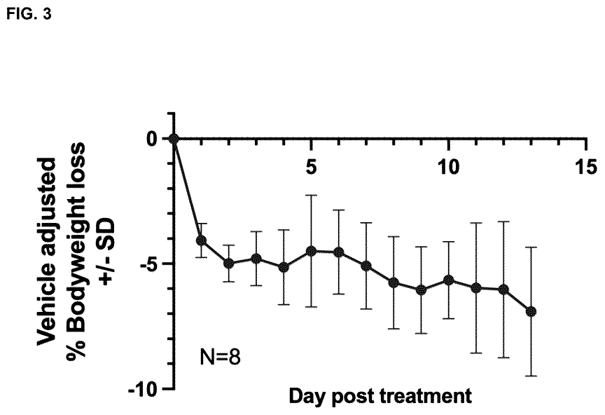

In embodiments, the peptide of the disclosure is a peptide described in FIG. 3.

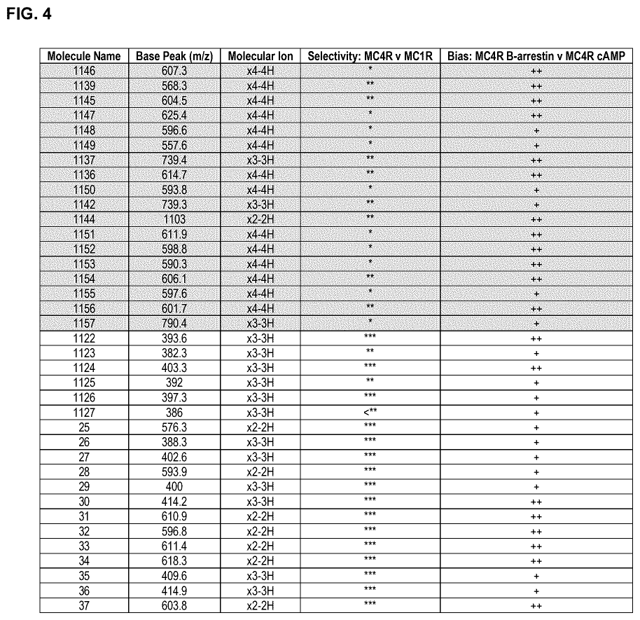

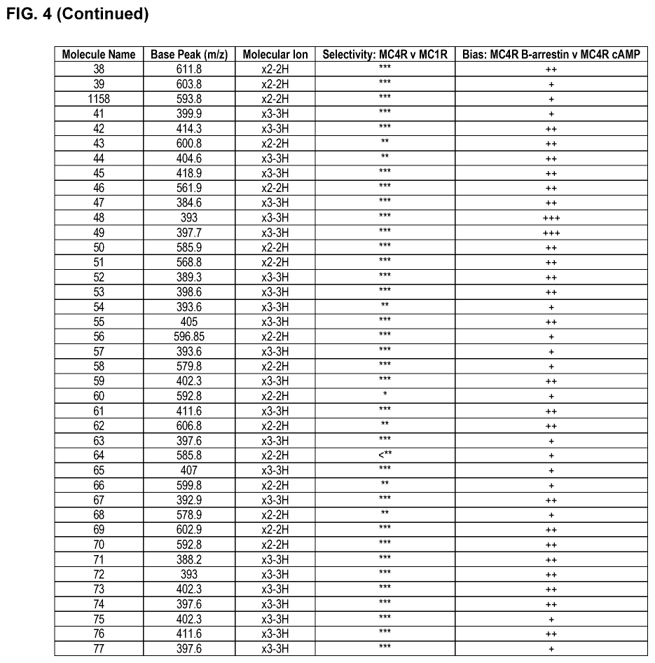

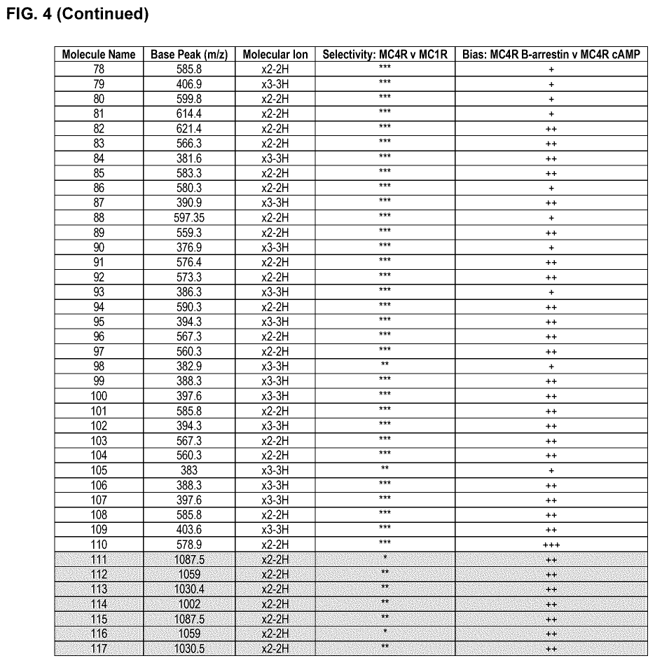

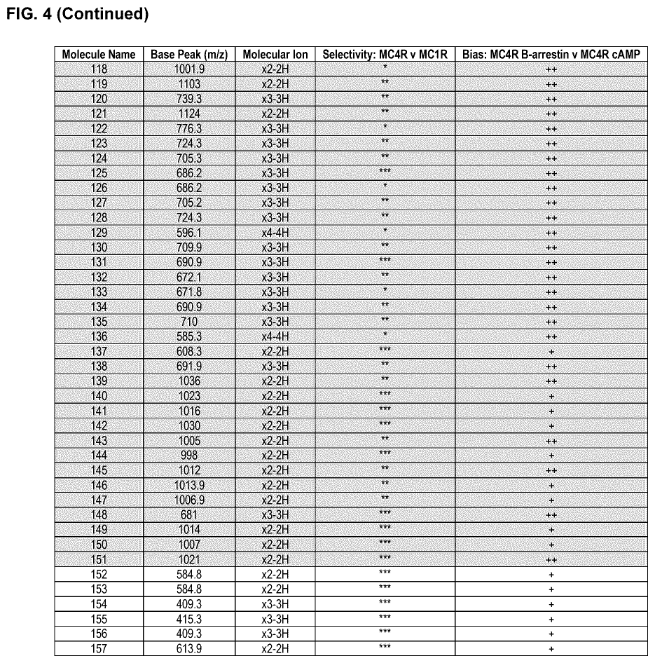

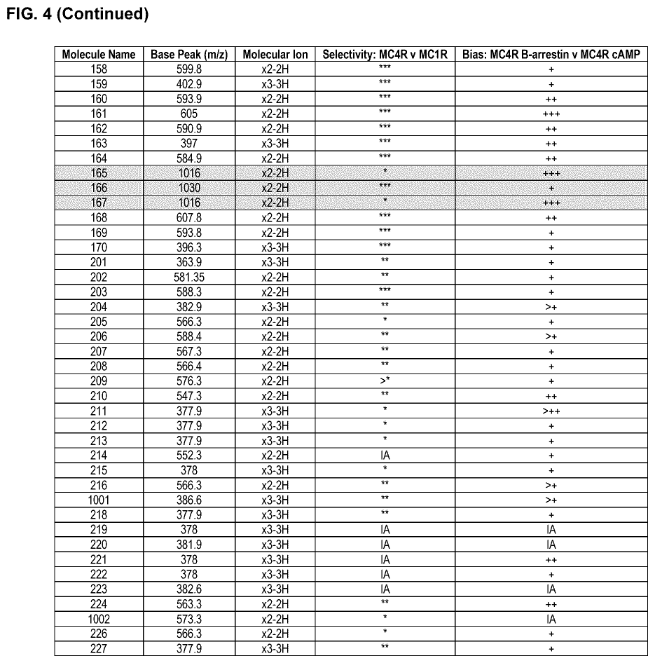

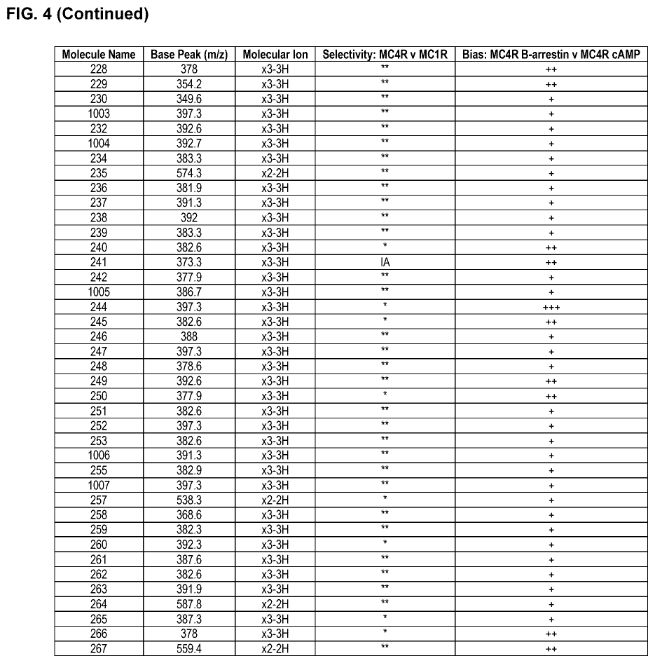

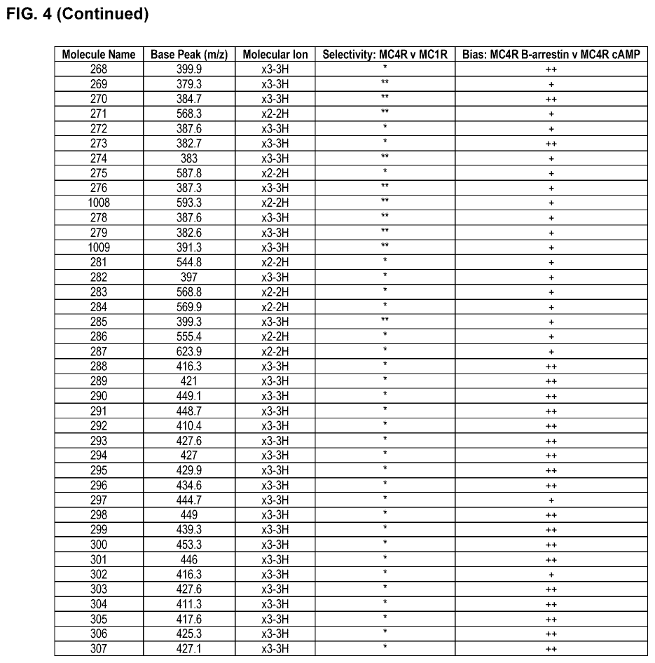

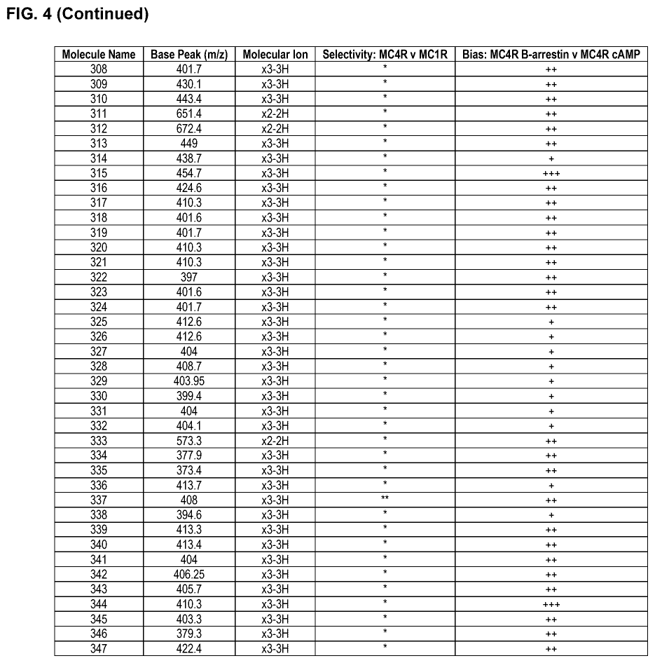

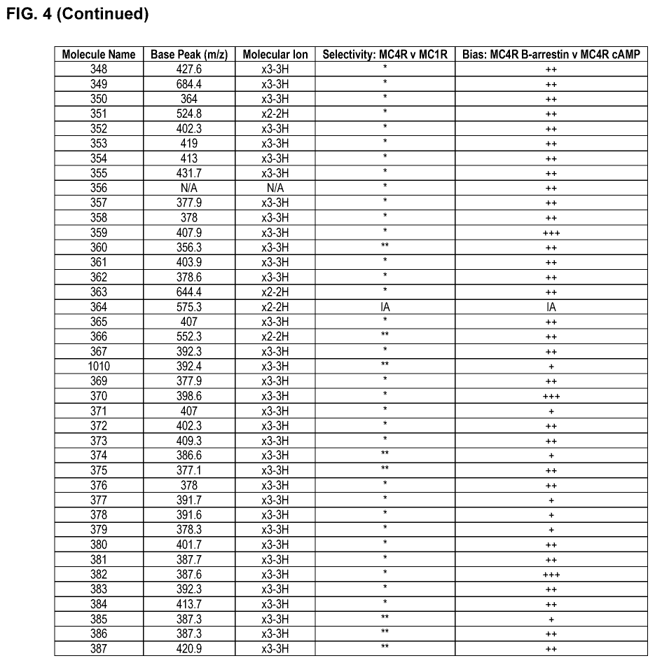

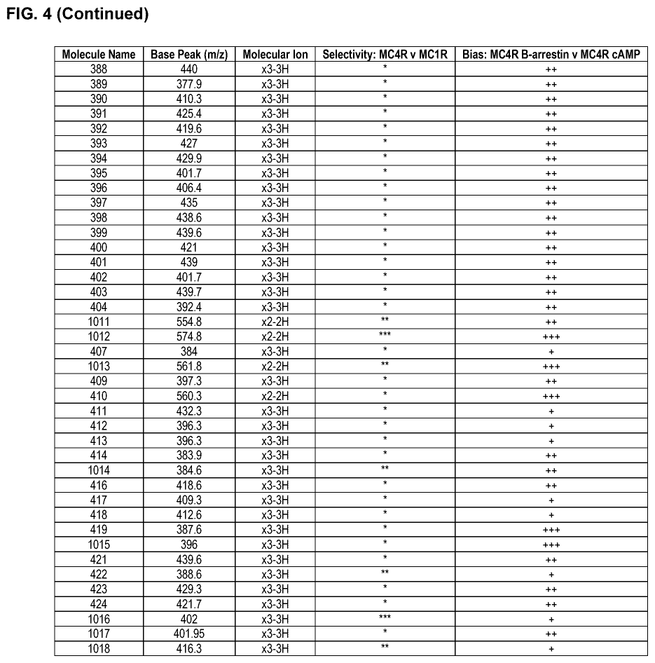

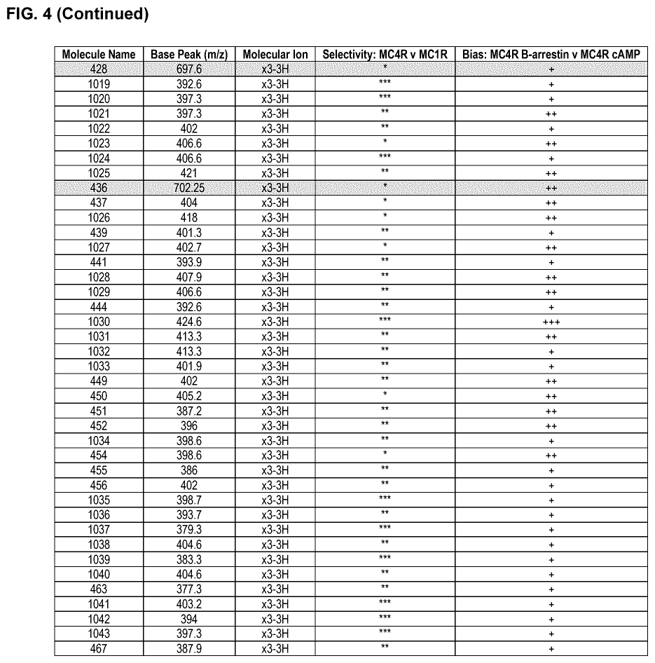

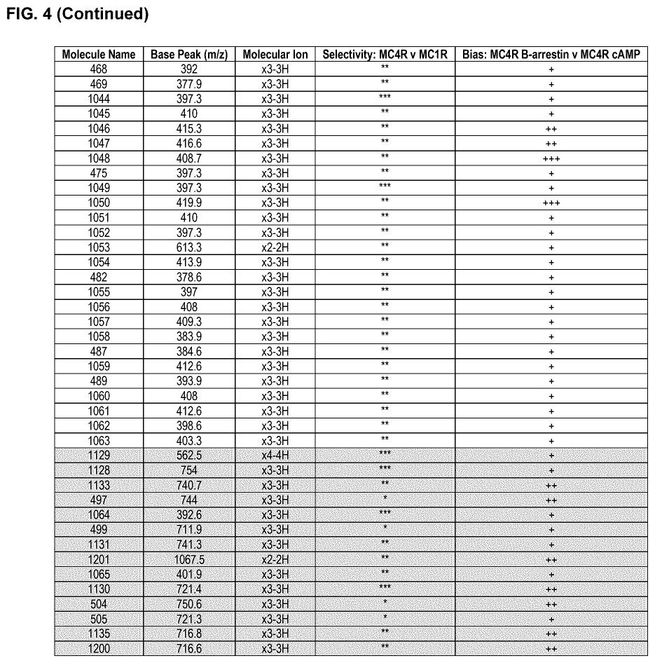

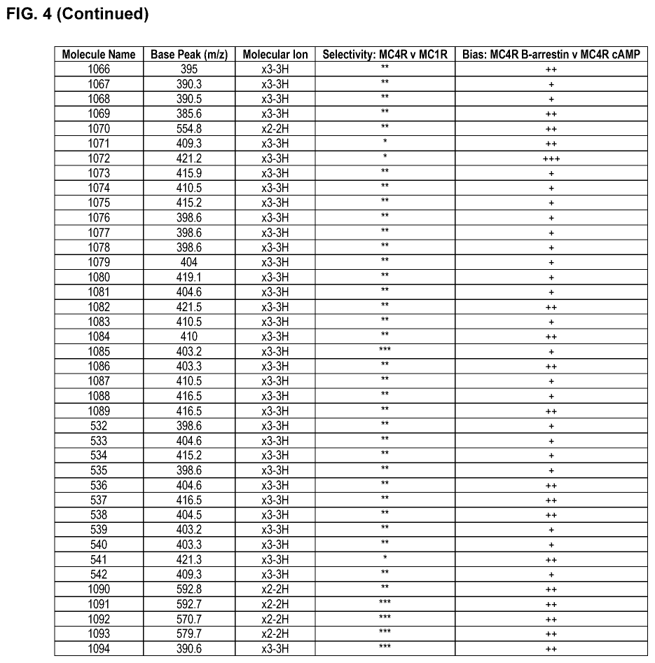

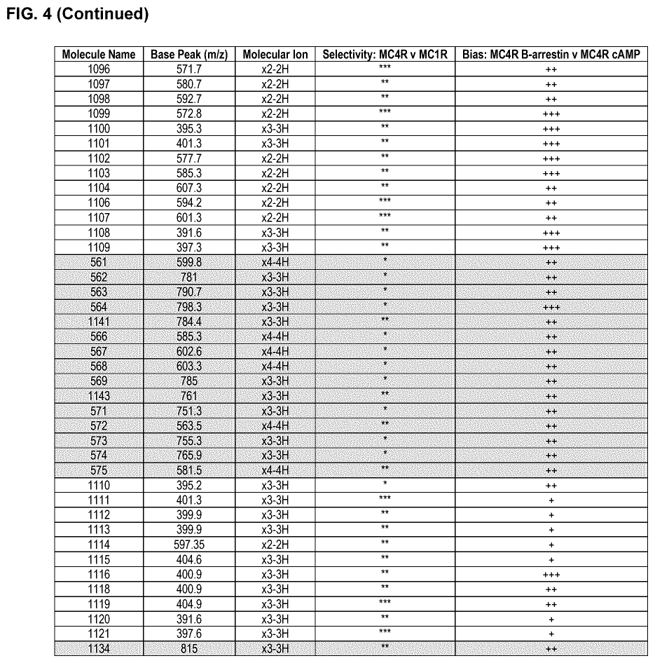

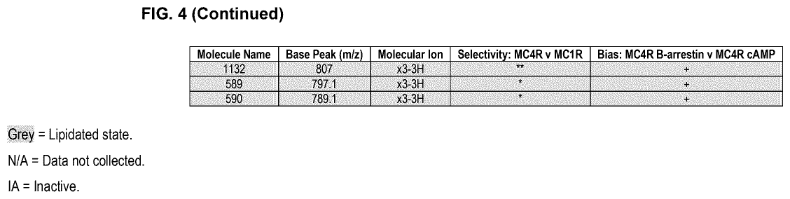

In embodiments, the peptide of the disclosure is a peptide described in FIG. 4.

In embodiments, the peptide of the disclosure is molecule 1092:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1092 | D-Nar | Cys | Aib(O- | Gln | D-Phe | Arg | Trp(6-F) | Cys | Ac | NH2 | Disulfide |
| | | | cyclic) | | | | | | | | |

In embodiments, the peptide of the disclosure is molecule 1093:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1093 | D-Nar | Cys | Aib(O- | Gln | D-Phe(4-F) | Arg | Trp(6-F) | Cys | Ac | NH2 | Disulfide |
| | | | cyclic) | | | | | | | | |

In embodiments, the peptide of the disclosure is molecule 1094:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1094 | D-Nar | Cys | D-aMeOrn | Gln | D-Phe | Arg | Trp(6-F) | Cys | Ac | NH2 | Disulfide |

In embodiments, the peptide of the disclosure is molecule 1106:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1106 | D-Nar | Cys | Aib(O- | Cit | D-Phe(4-F) | Arg | Trp(6-F) | Cys | Ac | NH2 | Disulfide |
| | | | cyclic) | | | | | | | | |

In embodiments, the peptide of the disclosure is molecule 1107:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1107 | D-Nar | Cys | Aib(O- | hCit | D-Phe(4-F) | Arg | Trp(6-F) | Cys | Ac | NH2 | Disulfide |
| | | | cyclic) | | | | | | | | |

In embodiments, the peptide of the disclosure is molecule 1119:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1119 | D-Nar | Cys | Phg | 3Pal | D-Phe(4-F) | Arg | Trp(6-F) | Cys | Ac | NH2 | Disulfide |

In embodiments, the peptide of the disclosure is molecule 1158:

| | | | | | | | | | N- | C- | |
|---|---|---|---|---|---|---|---|---|---|---|---|
| Molecule | X1 | X2 | X3 | X4 | X5 | X6 | X7 | X8 | term | term | Cyclic |
| 1158 | D-Nar | Cys | Aib(O- | Gln | D-Phe(4-F) | Arg | Trp(6-F) | Pen | Ac | NH2 | Disulfide |
| | | | cyclic) | | | | | | | | |

### Modified Peptides

In aspects, a method for achieving half-life extension is through chemical modification of molecules to extend their residence time in the serum. In embodiments, the peptides of the present disclosure are modified, for example, to achieve half-life extension. In embodiments, the peptides of the present disclosure comprise a lipid-containing moiety grafted with linker fragments onto a peptide (e.g., without limitation the peptides of Table 1 and Table 2). In embodiments, the half-life of the peptides of the present disclosure can be extended without substantially affecting the selectivity and efficacy parameters. In embodiments, the peptides of the present disclosure are highly selective, B-arrestin biased MC4R agonists that can be modified for extended half-life in vivo.

In embodiments, the peptides of the disclosure are lipidated. In a non-limiting example, a lipidated peptide further comprises one or more lipids conjugated to an amino acid of the peptide. In embodiments, the peptide of formula (I) comprises one or more lipids conjugated to one or more of X₁-X₈. In embodiments, the peptide of formula (I) comprises one or more lipids conjugated to X₁ and/or X₈. In embodiments, the one or more lipids comprise cysteine prenylation, N-terminal glycine myristylation, cysteine palmitoylation, and serine and/or lysine fatty acylation.

In embodiments, the peptide further comprises one or more amino acids conjugated to X₁ and/or X₈. In embodiments the one or more amino acids are selected from D-arginine (D-Arg), glycine (Gly), and L-Lys(AEEAc-AEEAc-L-γ-Glu-17-carboxyheptadecanoyl) (Lys*). In embodiments, the one or more amino acids are selected from Table 1 and Table 2. In embodiments, the one or more amino acid is modified. Non-limiting examples of modified amino acids include PEG groups (e.g., PEG-2), and lipids. In embodiments, the modified amino acid is Lys(AEEAc-AEEAc-L-γ-Glu-17-carboxyheptadecanoyl) (Lys*).

In embodiments, the peptide further comprises one or more lipids conjugated to X₁ and/or X₈. In embodiments, the peptide further comprises one or more lipids conjugated to the one or more amino acids conjugated to X₁ and/or X₈.

In embodiments, the peptide further comprises one or more PEG linkers conjugated to X₁ and/or X₈.

In embodiments, the peptide further comprises one or more dicarboxylic acid, e.g., a C12-C24 dicarboxylic acid.

In embodiments, one or more amino acid residues are conjugated to X₁ of any one of the peptides of formula (I) or formula (II). In embodiments, at least 1, or at least 2, or at least 3, or at least 4, or at least 5 amino acid residues are conjugated to X₁ of any one of the peptides of formula (I) or formula (II). In embodiments, the one or more amino acid residues conjugated to X₁ have the sequential designation of X₋₁, X₋₂, X₋₃, X₋₄, X₋₅, and so forth.

In embodiments, one or more amino acid residues are conjugated to X₈ of any one of the peptides of formula (I) or formula (II). In embodiments, at least 1, or at least 2, or at least 3, or at least 4, or at least 5 amino acid residues are conjugated to X₈ of any one of the peptides of formula (I) or formula (II). In embodiments, the one or more amino acid residues conjugated to X₈ have the sequential designation of X₉, X₁₀, X₁₁, X₁₂, X₁₃, X₁₄ and so forth.

In embodiments, the peptides of formula (I) or formula (II) comprises an N-terminal acetyl. In embodiments, the peptides of formula (I) or formula (II) comprises a C-terminal amide. In embodiments, the peptides of formula (I) or formula (II) comprises an N-terminal acetyl and C-terminal amide.

In embodiments, the peptide is capped with N-terminal acetyl and/or C-terminal amide groups.

In embodiments, the peptides of formula (I) or formula (II) comprises an N-terminal and/or C-terminal modification. In embodiments, the N-terminal modification comprises acetylation, propionylation, methylation, myristoylation, palmitoylation or ubiquitylation. In embodiments, the N-terminal modification comprises acyl group. Non-limiting examples of acyl group include acetyl, propionyl, butyryl, formyl, propenyl, crotyl, butenyl, and benzyl. In embodiments, C-terminal modifications include neutralization of the negative charge that the carboxylic acid derivative displays at physiological pH. In embodiments, C-terminal modifications include NR1R2, wherein R1 and R2 are selected from H or alkyl.

In embodiments, the peptide of formula (I) or formula (II) is selected from Table 1 and Table 2.

In embodiments, one or more amino acid residues are conjugated to X₈. In embodiments, the one or more amino acid residues conjugated to X₈ have the sequential designation of X₉, X₁₀, X₁₁, and X₁₂ as exemplified in Table 3, and/or cyclic structure.

In embodiments, the one or more amino acid residues conjugated to X₈ comprise an N-terminal acetyl and C-terminal amide and/or a cyclic structure as exemplified in Table 3.

TABLE 3
Exemplary peptides with of one or more amino acid residues conjugated to X₃. Cyclic peptides include bridge (e.g. disulfide) between X₂ and X₃.

| | | | | | | | | | | | | | | | Cyclic |
|---|---|---|---|---|---|---|---|---|---|---|---|---|---|---|---|
| | | | | | | | | | | | | | N- | C- | mole- |
| [symbol] | [symbol] | X [symbol] | X [symbol] | X [symbol] | X [symbol] | X [symbol] | X [symbol] | X₈ | X₉ | X₁₀ | X₁₁ | X₁₂ | term | term | cule |
| [symbol] | [symbol] | Cys | Aib(O- | [symbol] | D- | Arg | Trp(6-F) | Cys | Gly | Gly | Gly | Lys* | Ac | NH2 | Disul- |
| | | | cyclic) | | Phe(4-F) | | | | | | | | | | fide |
| 116 | D-Nar | Cys | Aib(O- | [symbol] | D- | Arg | Trp(6-F) | Cys | Gly | Gly | Lys* | | Ac | NH2 | Disul- |
| | | | cyclic) | | Phe(4-F) | | | | | | | | | | fide |
| 117 | D-Nar | Cys | Aib(O- | [symbol] | D- | Arg | Trp(6-F) | Cys | Gly | Lys* | | | Ac | NH2 | Disul- |
| | | | cyclic) | | Phe(4-F) | | | | | | | | | | fide |
| 118 | D-Nar | Cys | Aib(O- | [symbol] | D- | Arg | Trp(6-F) | Cys | Lys* | | | | Ac | NH2 | Disul- |
| | | | cyclic) | | Phe(4-F) | | | | | | | | | | fide |
| 119 | D-Nar | Cys | Aib(O- | [symbol] | D- | Arg | Trp(6-F) | Cys | PEG1 | PEG1 | Lys* | | Ac | NH2 | Disul- |
| | | | cyclic) | | Phe(4-F) | | | | | | | | | | fide |
| 120 | D-Nar | Cys | Aib(O- | [symbol] | D- | Arg | Trp(6-F) | Cys | D-Arg | Gly | Lys* | | Ac | NH2 | Disul- |
| | | | cyclic) | | Phe(4-F) | | | | | | | | | | fide |
| 121 | D-Nar | Cys | Aib(O- | [symbol] | D- | Arg | Trp(6-F) | Cys | Pro | Phe | Lys* | | Ac | NH2 | Disul- |
| | | | cyclic) | | Phe(4-F) | | | | | | | | | | fide |
| 122 | D-Nar | Cys | Aib(O- | [symbol] | D- | Arg | Trp(6-F) | Cys | Lys | Pro | Val | Lys* | Ac | NH2 | Disul- |
| | | | cyclic) | | Phe(4-F) | | | | | | | | | | fide |
| 126 | D-Nar | Cys | Phg | 3Pal | D- | Arg | Trp(6-F) | Cys | Lys* | | | | Ac | NH2 | Disul- |
| | | | | | Phe(4-F) | | | | | | | | | | fide |
| 127 | D-Nar | Cys | Phg | 3Pal | D- | Arg | Trp(6-F) | Cys | Gly | Lys* | | | Ac | NH2 | Disul- |
| | | | | | Phe(4-F) | | | | | | | | | | fide |
| 128 | D-Nar | Cys | Phg | 3Pal | D- | Arg | Trp(6-F) | Cys | Gly | Gly | Lys* | | Ac | NH2 | Disul- |
| | | | | | Phe(4-F) | | | | | | | | | | fide |
| 129 | D-Nar | Cys | Phg | 3Pal | D- | Arg | Trp(6-F) | Cys | Lys | Pro | Val | Lys* | Ac | NH2 | Disul- |
| | | | | | Phe(4-F) | | | | | | | | | | fide |
| 133 | D-Nar | Cys | D- | [symbol] | D-Phe | Arg | Trp(6-F) | Cys | Lys* | | | | Ac | NH2 | Disul- |
| | | | aMeOrn | | | | | | | | | | | | fide |
| 134 | D-Nar | Cys | D- | [symbol] | D-Phe | Arg | Trp(6-F) | Cys | Gly | Lys* | | | Ac | NH2 | Disul- |
| | | | aMeOrn | | | | | | | | | | | | fide |
| 135 | D-Nar | Cys | D- | [symbol] | D-Phe | Arg | Trp(6-F) | Cys | Gly | Gly | Lys* | | Ac | NH2 | Disul- |
| | | | aMeOrn | | | | | | | | | | | | fide |
| 136 | D-Nar | Cys | D- | [symbol] | D-Phe | Arg | Trp(6-F) | Cys | Lys | Pro | Val | Lys* | Ac | NH2 | Disul- |
| | | | aMeOrn | | | | | | | | | | | | fide |
| 281 | | [symbol] | Cys | His | D-Phe | Arg | Trp | Gaba | Cys | | | | Ac | NH2 | Disul- |
| | | | | | | | | | | | | | | | fide |
| 282 | Arg | Gly | Cys | His | D-Phe | Arg | Trp | Gaba | Cys | | | | Ac | NH2 | Disul- |
| | | | | | | | | | | | | | | | fide |
| 283 | | D-Phe | Cys | His | D-Phe | Arg | Trp | 5- [symbol] | Cys | | | | Ac | NH2 | Disul- |
| | | | | | | | | | | | | | | | fide |
| 284 | | Arg | Asp | His | D-Phe | Arg | Trp | Ala | Lys | | | | Ac | NH2 | [symbol] |
| 285 | Arg | Gly | Asp | His | D-Phe | Arg | Trp | Ala | Lys | | | | Ac | NH2 | [symbol] |
| 286 | | [symbol] | Asp | His | D-Phe | Arg | Trp | [symbol] | Lys | | | | Ac | NH2 | [symbol] |
| 302 | Arg | Cys | D-Ala | His | D-Phe | Arg | Trp | Cys | [symbol] | | | | Ac | NH2 | Disul- |
| | | | | | | | | | | | | | | | fide |
| 303 | Arg | Cys | D-Ala | His | D-Phe | Arg | Trp | Cys | Tyr | | | | Ac | NH2 | Disul- |
| | | | | | | | | | | | | | | | fide |
| 304 | Arg | Cys | D-Ala | His | D-Phe | Arg | Trp | Cys | D-Orn | | | | Ac | NH2 | Disul- |
| | | | | | | | | | | | | | | | fide |
| 305 | Arg | Cys | D-Ala | His | D-Phe | Arg | Trp | Cys | Phg | | | | Ac | NH2 | Disul- |
| | | | | | | | | | | | | | | | fide |
| 306 | Arg | Cys | D-Ala | His | D-Phe | Arg | Trp | Cys | Arg | | | | Ac | NH2 | Disul- |
| | | | | | | | | | | | | | | | fide |
| 307 | Arg | Cys | D-Ala | His | D-Phe | Arg | Trp | Cys | homoPhe | | | | Ac | NH2 | Disul- |
| | | | | | | | | | | | | | | | fide |
| 308 | Arg | Cys | D-Ala | His | D-Phe | Arg | Trp | Cys | [symbol] | | | | Ac | NH2 | Disul- |
| | | | | | | | | | | | | | | | fide |
| 309 | Arg | Cys | D-Ala | His | D-Phe | Arg | Trp | Cys | Beta- | | | | Ac | NH2 | Disul- |
| | | | | | | | | | homo- | | | | | | fide |
| | | | | | | | | | Arg | | | | | | |
| 310 | Arg | Cys | D-Ala | His | D-Phe | Arg | Trp | Cys | Ala | [symbol] | | | Ac | NH2 | Disul- |
| | | | | | | | | | | | | | | | fide |
| 311 | Arg | Cys | D-Ala | His | D-Phe | Arg | Trp | Cys | Ala | Leu | | | Ac | NH2 | Disul- |
| | | | | | | | | | | | | | | | fide |
| 312 | Arg | Cys | D-Ala | His | D-Phe | Arg | Trp | Cys | Pro | Glu | | | Ac | NH2 | Disul- |
| | | | | | | | | | | | | | | | fide |
| 313 | Arg | Cys | D-Ala | His | D-Phe | Arg | Trp | Cys | Ala | Arg | | | Ac | NH2 | Disul- |
| | | | | | | | | | | | | | | | fide |
| 314 | Arg | Cys | D-Ala | His | D-Phe | Arg | Trp | Cys | Gly | [symbol] | | | Ac | NH2 | Disul- |
| | | | | | | | | | | | | | | | fide |
| 315 | Arg | Cys | D-Ala | His | D-Phe | Arg | Trp | Cys | Pro | Phe | | | Ac | NH2 | Disul- |
| | | | | | | | | | | | | | | | fide |
| 316 | Arg | Cys | D-Ala | His | D-Phe | Arg | Trp | Cys | Gly | Pro | | | Ac | NH2 | Disul- |
| | | | | | | | | | | | | | | | fide |
| 317 | Arg | Pro | Cys | His | D-Phe | Arg | Trp | Gaba | Cys | | | | Ac | NH2 | Disul- |
| | | | | | | | | | | | | | | | fide |
| 318 | Arg | Ala | Cys | His | D-Phe | Arg | Trp | Gaba | Cys | | | | Ac | NH2 | Disul- |
| | | | | | | | | | | | | | | | fide |
| 319 | Arg | [symbol] | Cys | His | D-Phe | Arg | Trp | Gaba | Cys | | | | Ac | NH2 | Disul- |
| | | | | | | | | | | | | | | | fide |
| 320 | Arg | D-Pro | Cys | His | D-Phe | Arg | Trp | Gaba | Cys | | | | Ac | NH2 | Disul- |
| | | | | | | | | | | | | | | | fide |
| 321 | D-Arg | Pro | Cys | His | D-Phe | Arg | Trp | Gaba | Cys | | | | Ac | NH2 | Disul- |
| | | | | | | | | | | | | | | | fide |
| 322 | D-Arg | [symbol] | Cys | His | D-Phe | Arg | Trp | Gaba | Cys | | | | Ac | NH2 | Disul- |
| | | | | | | | | | | | | | | | fide |
| 323 | D-Arg | D-Ala | Cys | His | D-Phe | Arg | Trp | Gaba | Cys | | | | Ac | NH2 | Disul- |
| | | | | | | | | | | | | | | | fide |
| 324 | D-Arg | Ala | Cys | His | D-Phe | Arg | Trp | Gaba | Cys | | | | Ac | NH2 | Disul- |
| | | | | | | | | | | | | | | | fide |
| 325 | Arg | D-Pro | Asp | His | D-Phe | Arg | Trp | Ala | Lys | | | | Ac | NH2 | [symbol] |
| 326 | Arg | Pro | Asp | His | D-Phe | Arg | Trp | Ala | Lys | | | | Ac | NH2 | [symbol] |
| 327 | Arg | Ala | Asp | His | D-Phe | Arg | Trp | Ala | Lys | | | | Ac | NH2 | [symbol] |
| 328 | Arg | Ala(2Me) | Asp | His | D-Phe | Arg | Trp | Ala | Lys | | | | Ac | NH2 | [symbol] |
| 329 | Arg | D-Ala | Asp | His | D-Phe | Arg | Trp | Ala | Lys | | | | Ac | NH2 | [symbol] |
| 330 | D-Arg | Gly | Asp | His | D-Phe | Arg | Trp | Ala | Lys | | | | Ac | NH2 | [symbol] |
| 331 | D-Arg | Ala | Asp | His | D-Phe | Arg | Trp | Ala | Lys | | | | Ac | NH2 | [symbol] |
| 332 | D-Arg | D-Ala | Asp | His | D-Phe | Arg | Trp | Ala | Lys | | | | Ac | NH2 | [symbol] |
| 333 | | Arg | Cys | His | D-Phe | Arg | Trp | 5-Ava | Cys | | | | Ac | NH2 | Disul- |
| | | | | | | | | | | | | | | | fide |
| 334 | | Arg | Cys | His | D-Phe | Arg | Trp | Gaba | Cys | | | | Ac | NH2 | Disul- |
| | | | | | | | | | | | | | | | fide |
| 335 | | Arg | Cys | His | D-Phe | Arg | Trp | bAla | Cys | | | | Ac | NH2 | Disul- |
| | | | | | | | | | | | | | | | fide |
| 336 | Arg | Gly | Asp | His | D-Phe | Arg | Trp | [symbol] | Lys | | | | Ac | NH2 | [symbol] |
| 337 | Arg | Gly | Asp | His | D-Phe | Arg | Trp | Pro | Lys | | | | Ac | NH2 | [symbol] |
| 338 | Arg | Gly | Asp | His | D-Phe | Arg | Trp | [symbol] | Lys | | | | Ac | NH2 | [symbol] |
| 339 | Arg | Gly | Asp | His | D-Phe | Arg | Trp | Leu | Lys | | | | Ac | NH2 | [symbol] |
| 340 | Arg | Gly | Asp | His | D-Phe | Arg | Trp | D-Leu | Lys | | | | Ac | NH2 | [symbol] |
| 341 | Arg | Ala | Asp | His | D-Phe | Arg | Trp | Ala(2-Me) | Lys | | | | Ac | NH2 | [symbol] |
| 342 | Arg | Gly | Cys | His | D-Phe | Arg | Trp | 4-amino- | Cys | | | | Ac | NH2 | Disul- |
| | | | | | | | | 4- [symbol] | | | | | | | fide |
| | | | | | | | | acid | | | | | | | |
| 343 | Arg | Gly | Cys | His | D-Phe | Arg | Trp | [symbol] | Cys | | | | Ac | NH2 | Disul- |
| | | | | | | | | | | | | | | | fide |
| 344 | Arg | Gly | Cys | His | D-Phe | Arg | Trp | [symbol] | Cys | | | | Ac | NH2 | Disul- |
| | | | | | | | | | | | | | | | fide |
| 352 | Arg | Cys | D-Ala | His | D-Phe | Arg | Trp | Cys | Ser | | | | Ac | NH2 | Disul- |
| | | | | | | | | | | | | | | | fide |
| 353 | Arg | Cys | D-Ala | His | D-Phe | Arg | Trp | Cys | D-His | | | | Ac | NH2 | Disul- |
| | | | | | | | | | | | | | | | fide |
| 354 | Arg | Cys | D-Ala | His | D-Phe | Arg | Trp | Cys | 0FU | | | | Ac | NH2 | Disul- |
| | | | | | | | | | | | | | | | fide |
| 355 | Arg | Cys | D-Ala | His | D-Phe | Arg | Trp | Cys | [symbol] | | | | Ac | NH2 | Disul- |
| | | | | | | | | | | | | | | | fide |
| 356 | Arg | Cys | D-Ala | His | D-Phe | Arg | Trp | Cys | [symbol] | | | | Ac | NH2 | Disul- |
| | | | | | | | | | | | | | | | fide |
| 371 | Arg | Cys | D-Ala | His | D-Phe | Arg | Trp | Cys | PEG1 | | | | Ac | NH2 | Disul- |
| | | | | | | | | | | | | | | | fide |
| 372 | Arg | Gly | Cys | His | D-Phe | Arg | Trp | PEG1 | Cys | | | | Ac | NH2 | Disul- |
| | | | | | | | | | | | | | | | fide |
| 373 | Arg | Gly | Lys | His | D-Phe | Arg | Trp | PEG1 | Asp | | | | Ac | NH2 | [symbol] |
| 384 | Arg | Gly | Asp | His | D-Phe | Arg | Trp | Orn | Lys | | | | Ac | NH2 | [symbol] |
| 389 | | D-Arg | Cys | His | D-Phe | Arg | Trp | Gaba | Cys | | | | Ac | NH2 | Disul- |
| | | | | | | | | | | | | | | | fide |
| 398 | Arg | Cys | D-Ala | His | D-Phe | Arg | Trp | Cys | Gly | [symbol] | | | Ac | NH2 | Disul- |
| | | | | | | | | | | | | | | | fide |
| 402 | Arg | Gly | Cys | His | D-Phe | Arg | Trp | Me-Gaba | Cys | | | | Ac | NH2 | Disul- |
| | | | | | | | | | | | | | | | fide |
| 403 | Arg | Cys | D-Ala | His | D-Phe | Arg | Trp | Cys | Gly | [symbol] | | | Ac | NH2 | Disul- |
| | | | | | | | | | | | | | | | fide |
| 404 | Arg | Gly | Cys | His | D-Phe | Arg | Trp | bAla | Cys | | | | Ac | NH2 | Disul- |
| | | | | | | | | | | | | | | | fide |
| 416 | Arg | Gly | Asp | His | D-Phe | Arg | Trp | D-Glu | Lys | | | | Ac | NH2 | [symbol] |
| 417 | Arg | Ala | Asp | His | D-Phe | Arg | Trp | Ser | Lys | | | | Ac | NH2 | [symbol] |
| 418 | Arg | D-Pro | Asp | His | D-Phe | Arg | Trp | Ala | Lys | | | | Ac | NH2 | [symbol] |
| 423 | Arg | Cys | D-Ala | His | D-Phe | Arg | Trp | Cys | D-Ala | Pro | | | Ac | NH2 | Disul- |
| | | | | | | | | | | | | | | | fide |
| 497 | D-Nar | Cys | L- | His | D-Phe | Arg | Trp(6-Me) | Cys | Gly | [symbol] | Lys* | | Ac | NH2 | Disul- |
| | | | aMeGlu | | | | | | | | | | | | fide |
| 1201 | D-Nar | Cys | L- | His | D-Phe | Arg | Trp(6-Me) | Cys | Gly | Gly | Lys* | | Ac | NH2 | Disul- |
| | | | aMeAsp | | | | | | | | | | | | fide |
| 505 | beta- | Cys | L- | His | D-Phe | Arg | Trp(6-Me) | Cys | Gly | Gly | Lys* | | Ac | NH2 | Disul- |
| | homo- | | aMeAsp | | | | | | | | | | | | fide |
| | Arg | | | | | | | | | | | | | | |
| 1200 | D-Arg | Cys | L- | His | D-Phe | Arg | Trp(6-Me) | Cys | Gly | Gly | Lys* | | Ac | NH2 | Disul- |
| | | | aMeAsp | | | | | | | | | | | | fide |

[symbol]

indicates data missing or illegible when filed

In aspects, the peptides of the disclosure (e.g., without limitation, peptides of formula (I)) or formula (II) comprise additional substituents and/or functional groups that further modify the properties and/or function of the peptides.

In embodiments, the peptide comprises a half-life extending moiety. Non-limiting examples of half-life extending moieties include polyethylene glycol (PEG), recombinant PEG mimetics, glycosylation of carbohydrates, Fc-fusion proteins or conjugates, albumin fusion proteins or conjugates (including fusions with albumin-binding proteins and peptides that, in turn, recruit albumin molecules), polypropylene glycol (PPG), XTEN fusion protein or conjugates, or a combination thereof.

In embodiments, the peptides of the present disclosure are pegylated. In some embodiments, attachment of the PEG moiety increases the half-life and/or reduces the immunogenicity of the peptide. In some embodiments, wherein the PEG-based moiety comprises one or more of poly(ethylene glycol) (PEG), an amine reactive PEG-based linker (e.g. Bis-PEG-acid, Bis-PEG-NHS, Boc-PEG, Fmoc-PEG, PEG Acid, PEG Aldehyde, PEG NHS ester, PEG Phosphonate, PEG PFP ester, PEG Tosylate), a biotinylated PEG-based linker (e.g. PEG-biotin), a branched PEG-based linker; a reactive carbonyl PEG-based linker (e.g. aminooxy-PEG), a carboxyl and/or active ester reactive PEG-based linker (e.g. amine PEG), a click-reagent PEG-based linker (e.g. alkyne PEG, azide-PEG); a copper-free click chemistry PEG-based linker (e.g. DBCO-PEG, BCN-PEG); a Cu-catalyzed click chemistry PEG-based linker (e.g. propargyl-PEG); a fluorescent PEG labeling and homobifunctional PEG-based linker (e.g. bis-PEG-acid, bis-PEG-NHS, bis-PEG-PFP, bis-propargyl-PEG, amine-PEG-amine, azido-PEG-azide, bromo-PEG-bromide), a lipid PEG, a metal surface binding and thiol reactive PEG-based linker (e.g. Thiol PEG, Bromo-PEG, Mal PEG), PEG-pAsp, PEG-pGlu, PEG-b-PMPMC, and PEG-PGLA.

In embodiments, the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

In embodiments, the peptide further comprises a therapeutic, diagnostic, and/or imaging moiety. Non-limiting examples of therapeutic, diagnostic, and/or imaging moiety include a small molecule, a biological (e.g., without limitation, a biopolymer, a protein, a nucleic acid, a polysaccharide), or a radionuclide.

In embodiments, the therapeutic, diagnostic, and/or imaging moiety is the functional and clinically significant part of the active ingredient substance(s) present in a medicinal product. In embodiments, the medicinal product comprises orlistat, phentermine-topiramate, naltrexone-bupropion, liraglutide, retatrutide, tirzepatide, semaglutide, and setmelanotide.

In embodiments, the therapeutic, diagnostic, and/or imaging moiety comprises an antibody or antigen-binding fragment thereof, an aptamer, a peptide, a biological ligand (e.g., including a glycoconjugate), lipid, sterol, cholesterol or derivative thereof, integrin, RGD peptide, or cell-penetrating peptide (CPP). In embodiments, the moiety is selected from a single-domain antibody, a single chain antibody, a bi-specific antibody, a recombinant heavy-chain-only antibody (VHH), a single-chain antibody (scFv), a shark heavy-chain-only antibody (VNAR), a microprotein (cysteine knot protein, knottin), a DARPin, a tetranectin, an affibody, a transbody, an anticalin, an AdNectin, an affilin, a microbody, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, and a peptide mimetic molecule. Various non-limiting examples of ligand-binding platforms are described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties.

In embodiments, the disclosure provides conjugates comprising a peptide or pharmaceutical composition of the present disclosure (e.g., without limitation, a peptide of formula (I) or formula (II)) conjugated to or co-formulated with a therapeutic agent or therapeutic moiety).

In embodiments, the therapeutic moiety or therapeutic agent comprises incretin, an incretin analogue, or a modulator of an incretin receptor.

In embodiments, the modulator is an agonist. In embodiments, the agonist is an agonist of GLP-1, GIP, and/or glucagon receptor. In embodiments, the agonist is a GLP-1 analogue. In embodiments, the GLP-1 analogue comprises a non-canonical amino acid. In embodiments, the GLP-1 analogue comprises tirzepatide, liraglutide, retatrutide, exenatide, lixisenatide, semaglutide or a semaglutide derivative.

In embodiments, the agonist is an agonist of GIP receptor. In embodiments, the agonist is a dual GLP-1-GIP receptor co-agonist. In embodiments, the agonist is a triple hormone receptor agonist. In embodiments, the agonist comprises retatrutide.

In embodiments, the agonist is an agonist of the amylin receptor. In embodiments, the agonist is an amylin analogue. In embodiments, the amylin analogue is cagrlintide. In embodiments, the agonist is selected from a PYY (3-36) analogue, an orexin analogue, and a leptin analog.

In embodiments, the therapeutic agent is a clinically significant part of the active ingredient substance(s) present in a medicinal product. In embodiments, the medicinal product comprises orlistat, phentermine-topiramate, naltrexone-bupropion, liraglutide, retatrutide, semaglutide, tirzepatide, and setmelanotide.

In embodiments, the peptide or pharmaceutical composition of the present disclosure is labeled with a fluorescent compound. In a non-limiting example, when the fluorescently labeled peptide is exposed to light of the proper wavelength, its presence and/or amount can then be detected. In embodiments, fluorescent labeling compounds are selected from fluorescein isothiocyanate, rhodamine, phycoerythrin, phycocyanin, allophycocyanin, o-phthaldehyde and fluorescamine. In embodiments, the peptide can also be detectably labeled using fluorescence emitting metals such as 152Eu, or others of the lanthanide series. In embodiments, the metal is attached to the peptide using such metal chelating groups as diethylenetriaminepentacetic acid (DTPA) or ethylenediaminetetraacetic acid (EDTA). In embodiments, the peptide is detectably labeled by coupling to a chemiluminescent compound. In a non-limiting example, the presence of the chemiluminescent-tagged antibody coupled to the peptide is determined by detecting luminescence that arises during the course of a chemical reaction. In embodiments, chemiluminescent labeling compounds are selected from luminol, isoluminol, theromatic acridinium ester, imidazole, acridinium salt and oxalate ester. In embodiments, a bioluminescent compound may be used to label the peptide. In a non-limiting example, the presence of a bioluminescent protein is determined by detecting the presence of luminescence. In embodiments, bioluminescent compounds for purposes of labeling are luciferin, luciferase and aequorin.

In embodiments, the disclosure provides a protein comprising a peptide or pharmaceutical composition of the present disclosure (e.g., without limitation, a peptide of formula (I) or a peptide of formula (II)).

In embodiments, the protein has a size of at least about 10 amino acid residues, or at least about 15 resides, or at least about 20 residues, or at least about 25 residues, or at least about 30 residues, or at least about 35 residues, or at least about 40 residues, or at least about 45 residues, or at least about 50 residues, or at least about 60 residues, or at least about 70 residues, or at least about 80 residues, or at least about 90 residues, or at least about 100 residues, or at least about 250 residues, or at least about 500 residues, at least about 750 residues, at least about 1,000 residues, at least about 1,250 residues, at least about 1,500 residues, at least about 1,750 residues, at least about 2,000 residues, at least about 3,000 residues, at least about 4,000 residues, or at least about 5,000 residues.

In embodiments, the disclosure provides a nucleic acid encoding the peptide of the present disclosure, or the protein of the present disclosure. In embodiments, the disclosure provides a system for encoding one or more non-canonical amino acids. Site-specific incorporation of unnatural amino acids with orthogonal chemical reactivity into proteins enables the synthesis of structurally defined protein conjugates. Amino acids containing ketone, azide, alkyne, alkene, and tetrazine side chains can be genetically encoded in response to nonsense and frameshift codons. Kim, Chan Hyuk et al. “Protein conjugation with genetically encoded unnatural amino acids.” Current opinion in chemical biology vol. 17, 3 (2013): 412-9.

In embodiments, the disclosure provides a solid synthesis device conjugated to a peptide of the present disclosure (e.g., without limitation, a peptide of formula (I) or formula (II)), a protein of the present disclosure, a nucleic acid of the present disclosure, and/or an amino acid therein.

In embodiments, the solid synthesis device is conjugated to one or more amino acids, wherein the one or more amino acids are intermediates in the synthesis of a peptide of the present disclosure (e.g., without limitation, a peptide of formula (I) or formula (II)), a protein of the present disclosure, and/or a nucleic acid of the present disclosure. In embodiments, a non-limiting example of a solid synthesis device comprises covalently binding the peptide of the present disclosure onto a solid support material and synthesized step-by-step in a single reaction vessel utilizing selective protecting group chemistry. In embodiments, the peptides of the present disclosure are synthesized from the carbonyl group side (C-terminus) to amino group side (N-terminus) of the amino acid chain in the solid-phase peptide synthesis method.

The peptides of the present disclosure are described from N-terminus to C-terminus unless stated otherwise.

In embodiments, the solid synthesis device is conjugated to about 1, about 2, about 3, about 4, about 5, about 6, about 7, about 8, or about 9, about 10, or about 10 or more amino acids, wherein the one or more amino acids are intermediates in the synthesis of a peptide of the present disclosure (e.g., without limitation, a peptide of formula (I) or formula (II)), a protein of the present disclosure, and/or a nucleic acid of the present disclosure.

In embodiments, the solid synthesis device comprises a solid phase material or resin. In embodiments, the resin is selected from core resin, Merrifield resin, hydroxymethyl resin, and amino core resin. In embodiments, the core resin comprises material selected from polystyrene polyacrylate, polyacrylamide, and polyethylene glycol. In embodiments, the polystyrene resin is crosslinked, uncrosslinked, or linear.

In embodiments, the solid synthesis device comprises aminomethyl resin or 4-methylbenzhydryl amine resin.

In embodiments, the solid synthesis device comprises polystyrene beads.

In embodiments, the size of the bead is about or at least about 1 micron, or about 5 microns, or about 10 microns, or about 20 microns, or about 30 microns, or about 40 microns, or about 50 microns, or about 75 microns, or about 100 microns, or about 200 microns, or about 300 microns, or about 400 microns, or about 500 microns, or about 600 microns, or about 700 microns, or about 800 microns in diameter.

In embodiments, the solid synthesis device comprises dosage forms such as pre-filled syringes, (including auto-injectors), patches, containers, solutions, suspensions, emulsion, drops, tablets, pills, pellets, capsules, capsules containing liquids, powders, sustained-release formulations, emulsions, aerosols, sprays, and suspensions.

### Peptide Selectivity

In embodiments, the peptides of the disclosure (e.g., a peptide of formula (I) or formula (II)) demonstrates increased selectivity for MC4R over MC1R when administered to a subject compared to a control.

In embodiments, the peptide demonstrates increased selectivity for MC4R over MC1R as measured by an in vitro, ex vivo, or in vivo assay when compared to a control.

In embodiments, the peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

In embodiments, the peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

In embodiments, the peptide demonstrates an increased ratio of MC4R intracellular signaling to MC1R intracellular signaling in a subject when administered to a subject compared to a control.

In embodiments, the peptide demonstrates an increased ratio of MC4R intracellular signaling to MC1R intracellular signaling as measured by an in vitro, ex vivo, or in vivo assay when compared to a control.

In embodiments, the peptide demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

In embodiments, the peptide demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

In embodiments, the peptide demonstrates enhanced melanocortin 4 receptor (MC4R) function in a subject when compared to before the peptide is administered or to a pre-treatment or non-treatment state, or a subject treated with control.

In embodiments, the peptide demonstrates decreased melanocortin 1 receptor (MC1R) function in a subject when compared to before the peptide is administered or to a pre-treatment or non-treatment state, or a subject treated with control.

In embodiments, the peptide demonstrates enhanced melanocortin 4 receptor (MC4R) function as measured by an in vitro, ex vivo, or in vivo assay when compared to a control.

In embodiments, the peptide demonstrates decreased melanocortin 1 receptor (MC1R) function as measured by an in vitro, ex vivo, or in vivo assay when compared to a control.

In embodiments, the peptide demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

In embodiments, the peptide demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

In embodiments, the cyclic peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

In embodiments, the cyclic peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

In embodiments, the control is selected from LY2112688, setmelanotide, ring peptide compound from KR2002-0038400, NDP-aMSH, melanotan-II, a-MSH, and bremelanotide.

In embodiments, the control comprises a vehicle control such as saline or mineral oil.

In embodiments, the control comprises a subject that has not been administered the peptide or pharmaceutical composition of the present disclosure. In embodiments, the control comprises a subject that has been administered LY2112688, setmelanotide, Ring Peptide Compound from KR2002-0038400, NDP-aMSH, Melanotan-II, a-MSH, and/or Bremelanotide. In embodiments, the control is a subject that is in a pre-treatment or non-treatment state.

In embodiments, the in vitro, ex vivo, or in vivo assay that demonstrates increased selectivity for MC4R over MC1R is selected from, one or more of: cAMP assay, B-arrestin assay, ELISA, electro-chemiluminescence assays, radioimmunoassay (RIA), fluorescence immunoassay (FIA), thermal shift assay, LC-MS detection, surface plasmon resonance (SPR), and bio-layer interferometry (BLI), including combinations of the foregoing. In embodiments, other assays to test the functionality of the protein include chromatographic, electrophoretic and chemical techniques. In embodiments, exemplary assays and technique include, but are not limited to, luciferase assays, bimolecular fluorescence complementation, affinity electrophoresis, pull down assays, ELISA, western blots, immunoblotting, high-through screening of protein interaction, in vivo crosslinking of protein complexes, chemical cross linking, chemical cross linking followed by high mass MALDI mass spectrometry, quantitative immunoprecipitation combined with knock-down (QUICK), proximity ligation assay in situ, surface plasmon resonance (SPR), dual polarization interferometry (DPI), static light scattering (SLS), dynamic light scattering (DLS), flow-induced dispersion analysis (FIDA), fluorescence polarization/anisotropy, fluorescence resonance energy transfer (FRET), bio-layer interferometry (BLI), rotating cell-based ligand binding assay using radioactivity or fluorescence, single color reflectometry (SCORE), and protein NMR, including combinations of the foregoing.

### Pharmaceutical Compositions and Formulations

In aspects, the disclosure provides a pharmaceutical composition comprising a peptide of the present disclosure, or a protein of the present disclosure, or a nucleic acid of the present disclosure, and a pharmaceutically acceptable excipient or carrier.

In embodiments, the pharmaceutical composition further comprises an additional therapeutic, diagnostic, and/or imaging moiety.

In one aspect, the composition is a pharmaceutical composition. In some embodiments, the pharmaceutical compositions of the present disclosure are formulated to provide a therapeutically effective amount of the peptides, as described herein, as the active ingredient. In embodiments, the pharmaceutical compositions further comprise one or more pharmaceutically acceptable excipients, carriers, including inert solid diluents and fillers, diluents, including sterile aqueous solution and various organic solvents, permeation enhancers, solubilizers and adjuvants.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In some embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any composition disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19^{th} ed. 1995), incorporated herein by reference.

In embodiments, the compositions, e.g., pharmaceutical compositions, disclosed herein are suspended in a saline buffer (including, without limitation TBS, PBS, and the like).

In embodiments, the disclosure includes the disclosed compositions and/or peptides in various formulations of pharmaceutical compositions. Any compositions and/or peptides disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Compositions of the present disclosure may be administered orally, parenterally, by inhalation spray, topically, rectally, nasally, buccally, sub-lingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this disclosure are aqueous or oleaginous suspension. These suspensions are formulated according to techniques known in the art using suitable dispersing or wetting agents and suspending agents. The sterile injectable preparation is a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

In embodiments, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. In embodiments, the pharmaceutical composition of the present disclosure are topically-transdermal patches. In embodiments, the pharmaceutical composition of the present disclosure are smart pills designed for targeted drug delivery to the GI tract and systemic, needle-free delivery.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

In certain embodiments, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

Pharmaceutically acceptable compositions of this disclosure can be administered to humans and other animals orally, rectally, parenterally, intracisternally, intravaginally, intraperitoneally, topically (as by powders, ointments, or drops), buccally, as an oral or nasal spray, or the like, depending on the severity of the disease being treated. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In embodiments, pharmaceutically acceptable compositions of the present disclosure further comprise preservatives. Non-limiting examples of preservatives include m-cresol, phenol, benzyl alcohol, tonicifiers, such as glycerin, mannitol, sucrose, and buffering agents such as phosphate, Tris, acetate, citrate.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. In embodiments, the capsules, tablets, and pills can be enterically coated.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

One or more peptides can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Methods of Treatment

In one aspect, the present disclosure includes methods for treating, preventing, or ameliorating a disease or disorder in a subject in need thereof, comprising administering a composition comprising an effective amount of the peptide or pharmaceutical composition of the present disclosure to the subject.

In embodiments, there is provided a method of selectively increasing the activity of MC4R over MC1R comprising administering a peptide or pharmaceutical composition of the present disclosure (e.g., without limitation, a peptide of formula (I) or a peptide of formula (II)) or a pharmaceutical composition of the present disclosure to a subject in need thereof.

In embodiments, the method prevents, reduces, or eliminates hyperpigmentation in the subject when compared to an untreated or pre-treatment subject, or subject treated with a control.

In embodiments, the hyperpigmentation is reduced by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% when compared to an untreated or pre-treatment subject, or subject treated with a control.

In embodiments, the hyperpigmentation is reduced by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold when compared to an untreated or pre-treatment subject, or subject treated with a control.

In embodiments, there is provided a method of treating a disease or disorder associated with insufficiency of MC4R signaling comprising administering a peptide of the present disclosure or the pharmaceutical composition of the present disclosure to a subject in need thereof. In embodiments, there is provided a method of treating a disease or disorder associated with MC4R function comprising administering a peptide of the present disclosure or the pharmaceutical composition of the present disclosure to a subject in need thereof.

In embodiments, there is provided a method of treating a disease or disorder associated with MC4R function comprising administering a peptide of the present disclosure or the pharmaceutical composition of the present disclosure to a subject receiving and/or previously received incretin.

In embodiments, there is provided a method of treating a disease or disorder associated with MC4R function comprising administering a peptide of the present disclosure or the pharmaceutical composition of the present disclosure to a subject not receiving incretin.

In embodiments, the method of the present disclosure further comprises administration of an additional therapeutic agent. In embodiments, the additional therapeutic agent is administered sequentially or concurrently (whether as a fixed dose combination, individually, or conjugate). In embodiments, the therapeutic agent comprises an incretin, an incretin analogue, or a modulator of an incretin receptor.

In embodiments, the modulator of an incretin receptor is an agonist. In embodiments, the agonist is an agonist of GLP-1, GIP, and/or glucagon receptor. In embodiments, the agonist is a GLP-1 analogue. In embodiments, the GLP-1 analogue comprises a non-canonical amino acid. In embodiments, the GLP-1 analogue comprises a semaglutide, tirzepatide liragutide, exenatide, lixisenatide, or a semaglutide derivative.

In embodiments, the agonist is an agonist of GIP receptor. In embodiments, the agonist is a dual GLP-1-GIP receptor co-agonist. In embodiments, the agonist is a triple hormone receptor agonist. In embodiments, the agonist comprises retatrutide.

In embodiments, the therapeutic agent is a clinically significant part of the active ingredient substance(s) present in a medicinal product. In embodiments, the medicinal product comprises orlistat, phentermine-topiramate, naltrexone-bupropion, tirzepatide, exenatide, lixisenatide liraglutide, retatrutide, semaglutide, and setmelanotide.

In embodiments, the therapeutic agent is an agonist of the amylin receptor. In embodiments, the agonist is an amylin analogue. In embodiments, the amylin analogue is cagrlintide. In embodiments, the therapeutic agent is an agonist selected from one or more of a PYY (3-36) analogue, an orexin analogue, and a leptin analog.

In embodiments, the subject is receiving or previously received treatment comprising incretin, an incretin analogue, or a modulator of an incretin receptor. In embodiments, the subject is not receiving treatment comprising incretin, an incretin analogue, or a modulator of an incretin receptor.

For example, including, but not limited to, embodiments pertaining to obesity and/or weight reduction/loss, the additional therapeutic agents include one or more of orlistat (e.g. ALLI, XENICAL), loracaserin (e.g. BELVIQ), phentermine-topiramate (e.g. QSYMIA), sibutramine (e.g. REDUCTIL or MERIDIA), rimonabant (ACOMPLIA), exenatide (e.g. BYETTA), pramlintide (e.g. SYMLIN) phentermine, benzphetamine, diethylpropion, phendimetrazine, bupropion, and metformin.

In embodiments, the therapeutic agents comprise GLP-1, GIP, and/or glucagon agonists. In embodiments, the therapeutic agent is selected from one or more of zepbound, wegovy, ozempic, rybelsus, maridebart cafraglutide (AMG133), retatrutude, bimagrumab, and amycretin.

In embodiments, the therapeutic agent comprises agents that interfere with the body's ability to absorb specific nutrients in food, e.g. orlistat (e.g. ALU, XENICAL), glucomannan, and guar gum. In embodiments, agents that suppress appetite are also among the therapeutic agents, e.g. catecholamines and their derivatives (such as phenteimine and other amphetamine-based drugs), various antidepressants and mood stabilizers (e.g. bupropion and topiramate), anorectics (e.g. dexedrine, digoxin). In embodiments, agents that increase the body's metabolism are also among the therapeutic agents.

In some embodiments, additional agents are selected from among appetite suppressants, neurotransmitter reuptake inhibitors, dopaminergic agonists, serotonergic agonists, modulators of GABAergic signaling, anticonvulsants, antidepressants, monoamine oxidase inhibitors, substance P (NK1) receptor antagonists, melanocortin receptor agonists and antagonists, lipase inhibitors, inhibitors of fat absorption, regulators of energy intake or metabolism, cannabinoid receptor modulators, agents for treating addiction, agents for treating metabolic syndrome, peroxisome proliferator-activated receptor (PPAR) modulators; dipeptidyl peptidase 4 (DPP-4) inhibitors, agents for treating cardiovascular disease, agents for treating elevated triglyceride levels, agents for treating low HDL, agents for treating hypercholesterolemia, and agents for treating hypertension. Some agents for cardiovascular disease include statins (e.g. lovastatin, atorvastatin, fluvastatin, rosuvastatin, simvastatin and pravastatin) and omega-3 agents (e.g. LOVAZA, EPANQVA, VASCEPA, esterified omega-3's in general, fish oils, krill oils, algal oils). In some embodiments, additional agents are selected from among amphetamines, benzodiazepines, suifonyl ureas, meglitinides, thiazolidinediones, biguanides, beta-blockers, XCE inhibitors, diuretics, nitrates, calcium channel blockers, phentermine, sibutramine, iorcaserin, cetilistat, rimonabant, taranabant, topiramate, gabapentin, valproate, vigabatrin, bupropion, tiagabine, sertraline, fluoxetine, trazodone, zonisamide, methylphenidate, varenicline, naltrexone, diethylpropion, phendimetrazine, rapaglinide, nateglinide, glimepiride, metformin, pioglitazone, rosiglilazone, and sitagliptin.

In embodiments, the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency or downregulation, and Prader-Willi syndrome.

In embodiments, MC4R allele variant or deficiency or downregulation in a subject is exemplified by at least about 5%, or at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% decrease in MC4R activity compared to a healthy subject.

In embodiments, there is provided a method of treating obesity or abnormal or excessive fat accumulation comprising administering a peptide of the present disclosure or the pharmaceutical composition of the present disclosure to a subject in need thereof.

In embodiments, the peptide or pharmaceutical composition of the present disclosure treats and/or prevents eating disorders such as overeating, metabolic disorders typified by positive energy imbalance, emotional or mental disorders, and dietary or syndromic obesity.

Diseases or disorders of particular interest are obesity, morbid obesity, morbid obesity prior to surgery, obesity linked inflammation, obesity linked gallbladder disease, obesity induced sleep apnea, hyperlipidemia, dyslipidemia, hypercholesterolemia, atherogenic dyslipidemia, fatty liver disease (FLD), insulin resistance, glucose intolerance, pre-diabetes, increased fasting glucose, diabetes mellitus, including type 2 diabetes mellitus, inflammation, including vascular inflammation, hypertension, endothelial dysfunction, dyslipidemia (e.g. high triglycerides, low HDL cholesterol and/or high LDL cholesterol), Prader-Willi Syndrome, insulin resistance, glucose intolerance, pre-diabetes, increased fasting glucose, diabetes mellitus, type 2 diabetes mellitus, inflammation, metabolic syndrome (insulin resistance, hypertension, hyperlipidemia), a metabolic disorder, excess body weight, and a combination of any two or more thereof.

In some embodiments, the metabolic disorder is excess body weight (e.g., excess body fat, such as visceral fat, subcutaneous fat, intramuscular fat, and combinations of the foregoing. In embodiments the at least one peptide selected from Table 1 and Table 2, or a variant thereof, is used to promote weight loss, e.g., by reducing excess body fat, such as visceral fat, subcutaneous fat, intramuscular fat, and combinations of the foregoing).

In some embodiments, the metabolic disorder is selected from the group consisting of obesity, diabetes, hypercholesterolemia, metabolic dysfunction-associated steatohepatitis (MASH), which includes but is not limited to nonalcoholic steatohepatitis, hyperlipidemia, and fatty liver.

In some embodiments, the present disclosure relates to methods for treating obesity. Obesity is a chronic disease that is highly prevalent in modern society and is associated not only with a social stigma, but also with decreased life span and numerous medical problems, including diabetes mellitus, insulin resistance, hypertension, hypercholesterolemia, cholelithiasis, osteoarthritis, orthopedic injury, thromboembolic disease, cancer, and coronary heart disease. Rissanen et al, British Medical Journal, 301: 835-837 (1990). In some embodiments, Obesity can be calculated using the body mass index (BMI: body weight per height in meters squared). In some embodiments, obesity is defined as an otherwise healthy subject that has a BMI greater than or equal to 30 kg/m², or a condition whereby a subject with at least one co-morbidity has a BMI greater than or equal to 27 kg/m². In some embodiments of the method of the disclosure, the subject is obese and has a body mass index of greater than about 30. In some embodiments, the subject is overweight and has a body mass index of about 25-29.9. In some embodiments, the method induces weight loss. In some embodiments, the method prevents weight gain. In some embodiments, the method prevents the growth of adipose tissue and impair adipocyte differentiation.

In this aspect, the patient may not substantially change caloric intake. In some embodiments, the caloric intake is high, relative to guidelines, such as the USDA tables. In some embodiments, the patient's caloric intake is 2000-10000 calories/day, or greater than about 2000 calories/day, or about 2200 calories/day, or about 2400 calories/day, or about 2600 calories/day, or about 2800 calories/day, or about 3000 calories/day, or about 3200 calories/day, or about 3400 calories/day, or about 3600 calories/day, or about 3800 calories/day, or about 4000 calories/day, or about 5000 calories/day, or about 6000 calories/day. In some embodiments, the patient has a high caloric intake and does not gain weight or even loses weight. Therefore, the present disclosure provides for an effect without lifestyle changes that often reduce patient adherence (e.g., failed dieting). In some embodiments, the patient's caloric intake is not restricted by more than about 20%, or not by more than about 10%, or not by more than about 5% of the patient's caloric intake at the start of treatment. In some embodiments, a high proportion of the patient's caloric intake is “empty calories,” i.e. calories from solid fats and/or added sugars. In some embodiments, greater than about 15%, or 20%, or 25%, or 30%, or 35%, or 50% of the patient's caloric intake is empty calories. Even in these embodiments, a patient may not gain weight or even lose weight.

In some embodiments, the patient of the present disclosure is overweight or obese. In some embodiments, the patient of the present disclosure suffers from central obesity. In some embodiments, the obesity of one of simple obesity (alimentary obesity; usually resulting from consumption of more calories than the body can utilize), secondary obesity (usually resulting from an underlying medical condition, such as, for example, Cushing's syndrome and polycystic ovary syndrome), and childhood obesity. In some embodiments, the obesity is classified as: Class I, which includes a BMI between 30 and 34.99; Class II, which includes BMIs of 35 to 39.99; and Class III, which includes a BMI of over 40. Further, the present disclosure provides for obesity of any of classes I, II, or III that is further classified as severe, morbid, and super obesity. In some embodiments, the patient is at risk of further weight gain, as assessed by, for example, daily caloric intake.

In some embodiments, simple circumferential measurement of the body may be used. In some embodiments, a patient of the present disclosure has a waist circumference exceeding about 35 inches, or about 36 inches, or about 37 inches, or about 38 inches, or about 39 inches, or about 40 inches, or about 41 inches, or about 42 inches, or about 43 inches, or about 44 inches, or about 45 inches, or about 46 inches, or about 47 inches, or about 48 inches, or about 50 inches, or about 55 inches, or about 60 inches. In some embodiments, the patient is male human with a waist circumference exceeding 40 inches. In some embodiments, the patient is a female human with a waist circumference exceeding 35 inches.

In embodiments, the methods of the disclosure may be used to treat humans having a body fat percentage above the recommended body fat percentage, i.e., at least in the “overweight” range, or at least in the “obese” range. The body fat percentage will differ between women and men. Specifically, for women, the methods of the disclosure may be used to treat a female human having a body fat percentage of at least about 25%, above 25%, at least about 32%, or above 32%. For men, the methods of the disclosure may be used to treat a male human having a body fat percentage of at least about 14%, above 14%, at least about 18%, above 18%, at least about 25%, or above 25%. Body fat percentage may be estimated using any method accepted in the art, including, for example, near infrared interactance, dual energy X-ray absorptiometry, body density measurement, bioelectrical impedance analysis, and the like.

The methods of the disclosure may be used to treat a patient who is a man that is greater than 100 pounds overweight and/or has waist circumference exceeding 40 inches. The methods of the disclosure may be used to treat a patient who is a woman that is greater than 80 pounds' overweight and/or waist circumference exceeding 35 inches.

In some embodiments, the present disclosure relates to glucose tolerance. The term “glucose tolerance” refers to the ability and time required for the body to respond to the administration of glucose by clearing excess glucose from the circulation. A common test for measuring glucose tolerance is to perform a glucose tolerance test (GTT) on an individual, which typically involves orally administering 75 g of a glucose solution to a fasted individual and measuring blood glucose levels at intervals between 0 and 2 hours after administration. An individual who is “glucose intolerant”, as defined by the World Health Organization, has a 2-hour blood glucose level of between 140 to 199 mg per dL (7.8 to 11.0 mmol/l). An individual who has a 2-hour blood glucose level above 199 mg per dL is also glucose intolerant, but is additionally considered to have type II diabetes. Several different parameters of the GTT may be considered to evaluate the glucose tolerance of an individual, such as the 2-hour blood glucose level, fasting blood glucose level, GTT area under the curve, the max/peak blood glucose concentration obtained by an individual and mean blood glucose level. In some embodiments, improved glucose tolerance is an increase in the amount of glucose tolerance in an individual by at least about 0.5%, and preferably an increase of about 1% or more, e.g. by 5%, 10%, 30%, 50%, 70% or greater of the glucose tolerance of an individual prior to treatment with a compound of the disclosure, or a reduction in the rate that an individual develops glucose intolerance by at least about 1% or more, such as a reduction of glucose tolerance loss by about 5% or more, e.g. by 10%, 30%, 50%, 70%, 90% or greater than the rate of glucose tolerance loss of the individual prior to treatment. Lean mass, blood cholesterol level, blood triglyceride level and glucose tolerance are commonly measured in the fasted state in order to minimize the amount of nutrients which are newly entering circulation from the digestion of food and minimize the amount of nutrients which have yet to be been cleared from the blood following ingestion of food.

The term “impaired glucose tolerance” (IGT) or “pre-diabetes” refers to a condition in a person who, when given a glucose tolerance test, has a blood glucose level that falls between normal and hyperglycemic, i.e., has abnormal glucose tolerance, e.g., pathologically abnormal glucose tolerance. Such a person is at a higher risk of developing diabetes although not clinically characterized as having diabetes. In a non-limiting example, impaired glucose tolerance refers to a condition in which a patient has a fasting blood glucose concentration or fasting serum glucose concentration greater than 110 mg/dl and less than 126 mg/dl (7.00 mmol/L), or a 2 hour postprandial blood glucose or serum glucose concentration greater than 140 mg/dl (7.78 mmol/L) and less than 200 mg/dl (11.11 mmol/L). Prediabetes, also referred to as impaired glucose tolerance or impaired fasting glucose is a major risk factor for the development of type 2 diabetes mellitus, cardiovascular disease and mortality.

In some embodiments, the present disclosure provides for methods of treating impaired glucose tolerance in a patient in need thereof. In some embodiments, the present disclosure provides for methods of promoting improved glucose tolerance in a patient in need thereof.

Subjects and/or Animals

In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal is a human. In some embodiments, the human is a pediatric human. In some embodiments, the human is an infant or child. In some embodiments, the human is an adult human. In some embodiments, the human is a geriatric human. In other embodiments, the human may be referred to as a patient.

In certain some embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the disclosure pertains to veterinary use. In a specific embodiment, the non-human animal is a household pet. In another specific embodiment, the non-human animal is a livestock animal.

In some embodiments, such a subject and/or animal is administered an agent of the disclosure or is not administered an agent of the disclosure.

### Definitions

The following definitions are used in connection with the disclosure disclosed herein. Unless defined otherwise, all technical and scientific terms used herein have the same meaning as commonly understood to one of skill in the art to which this disclosure belongs.

An “effective amount” is an amount that is effective for treating, preventing, or ameliorating a disease or disorder such as those described herein.

An agent is “useful for the treatment of a disease or disorder” if the agent provides a measurable treatment, prevention, or reduction in the rate of pathogenesis of a disease or disorder.

As used herein, “a,” “an,” or “the” can mean one or more than one.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

Table 22 provides a list of residue shorthands and associated full residue name.

TABLE 22
Amino Acid Residues

| Residue Shorthand | Full Residue Name |
|---|---|
| (1S,3R)-3-aminocyclohexane-1-carboxylic acid | (1S,3R)-3-aminocyclohexane-1-carboxylic acid |
| (aMe)D-Phe | alpha-methyl-D-Phenylalanine |
| 0FU | 1,2-Phenylenedimethanamine |
| 2Nal | 3-(2-naphthyl)-L-Alanine |
| 2Pal | 3-(2-Pyridyl)-L-Alanine |
| 3-aminoazetidine-3-carboxylic acid | 3-aminoazetidine-3-carboxylic acid |
| 3-Pal | 3-(3-Pyridyl)-L-alanine |
| 4-amino-4-methylpentanoic acid | 4-amino-4-methylpentanoic acid |
| 4-aminooxane-4-carboxylic acid | 4-aminooxane-4-carboxylic acid |
| 4-Guanidinobutyric acid | 4-Guanidinobutyric acid |
| 4-Pal | 3-(4-pyridyl)-L-alanine |
| 5-Ava | 5-aminovaleric acid |
| Abu | L-2-Aminobutyric acid |
| Ac3c | 1-aminocyclopropane-1-carboxylic acid |
| Ac4c | 1-Aminocyclobutanecarboxylic acid |
| Ac6c | 1-aminocyclohexane-1-carboxylic acid |
| Aib | 2-Aminoisobutyric acid |
| Aib(O-cyclic) | 3-Aminooxetane-3-carboxylic acid |
| Ala(2-furyl) | 3-(2-Furyl)-L-Alanine |
| Ala(2Me) | 2-Methyl-Alanine |
| Ala(4-piperidyl) | 3-(4-Piperidinyl)-L-alanine |
| Ala(cPent) | 3-Cyclopentane-L-Alanine |
| Ame-L-Abu | Isovaline |
| aMeGly(allyl) | (S)-2-Amino-2-methylpent-4-enoic acid |
| aMeOrn | alpha-Methyl-L-Ornithine |
| Arg | Arginine |
| Arg(Me) | N-Monomethyl-L-Arginine, Tilarginine |
| Asp | Aspartic acid |
| bAc4c | 1-(Aminomethyl)cyclobutanecarboxylic acid |
| bAc5c | 1-(Aminomethyl)cyclopentanecarboxylic acid |
| bAla | beta-alanine |
| bDGLN | beta-D-Glutamine |
| beta-Ala(2-Me) | 3-amino-2,2-dimethyl-propionic acid |
| Beta-homoArg | beta-homo-L-arginine |
| bhDSER | beta-homo-D-Serine |
| bhGlu | beta-L-homoglutamic acid |
| Chg | L-Cyclohexylglycine |
| Cit | Citrulline |
| Cyclo-Leu | Cycloleucine |
| Cyclo-Leu(3-ene) | 1-Aminocyclopent-3-enecarboxylic acid |
| Cys | Cysteine |
| D-2Nal | 3-(2-Naphthyl)-D-Alanine |
| D-3Thi | 3-(3-Thienyl)-D-Alanine |
| D-Abu | D-2-Aminobutyric acid |
| D-Ala | D-Alanine |
| D-aMeAsp | alpha-methyl-D-aspartic acid |
| D-aMeLeu | Alpha-methyl-D-Leucine |
| D-aMeOrn | alpha-Methyl-D-Ornithine |
| D-aMeSer | Alpha-methyl-D-serine |
| D-aMeVal | alpha-Methyl-D-Valine |
| D-Arg | D-Arginine |
| D-Asp | D-Aspartic acid |
| D-bhGlu | beta-D-homoglutamic acid |
| D-Bpa | 4-Benzoyl-D-Phenylalanine |
| D-Dab | D-2,4-Diaminobutyric acid |
| D-Dap | 3-Amino-L-Alanine |
| D-Glu | D-Glutamic acid |
| D-hArg | Homo-D-Arginine |
| D-His | D-Histidine |
| D-homoPhe | Homo-D-Phenylalanine |
| D-hSer | Homo-D-Serine |
| D-Iva | D-Isovaline |
| D-Leu | D-Leucine |
| D-Lys | D-Lysine |
| D-Nar | D-Norarginine |
| D-Nva | D-Norvaline |
| D-Orn | D-Ornithine |
| D-Phe | D-Phenylalanine |
| D-Phe(2-F,4-Cl) | 4-Chloro-2-fluoro-D-phenylalanine |
| D-Phe(2,3-diF) | 2,3-difluoro-D-phenylalanine |
| D-Phe(2,4-diCl) | 2,4-dichloro-D-phenylalanine |
| D-Phe(2,4-diF) | 2,4-difluoro-D-phenylalanine |
| D-Phe(2,4,5-triF) | 2,4,5-trifluoro-D-phenylalanine |
| D-Phe(3-CF3) | 3-Trifluoromethyl-D-Phenylalanine |
| D-Phe(3-Cl) | 3-Chloro-D-Phenylalanine |
| D-Phe(3-F,4-Me) | 4-methyl-3-fluoro-D-phenylalanine |
| D-Phe(3-F) | 3-fluoro-D-phenylalanine |
| D-Phe(3-Me) | 3-methyl-D-phenylalanine |
| D-Phe(3-Ph) | 3-phenyl-D-phenylalanine |
| D-Phe(3,4-diF) | 3,4-difluoro-D-phenylalanine |
| D-Phe(3,4-diMe) | 3,4-Dimethyl-D-Phenylalanine |
| D-Phe(3,4,5-triF) | 3,4,5-trifluoro-D-phenylalanine |
| D-Phe(4-Br) | 4-Bromo-D-phenylalanine |
| D-Phe(4-CF3) | 4-(trifluoromethyl)-D-phenylalanine |
| D-Phe(4-Cl) | 4-Chloro-D-Phenylalanine |
| D-Phe(4-F) | 4-Fluoro-D-phenylalanine |
| D-Phe(4-Me) | 4-Methyl-D-Phenylalanine |
| D-Phg | D-Phenylglycine |
| D-Pro | D-Proline |
| D-Ser | D-Serine |
| D-Trp | D-Tryptophan |
| D-Tyr | D-Tyrosine |
| Dap | 3-Amino-L-Alanine |
| delta-Guanidinovaleric acid | 5-guanidinopentanoic acid |
| delta-Guanidinovaleric acid | delta-Guanidinovaleric acid |
| Gaba | gamma-aminobutyric-acid |
| Gln | Glutamine |
| Glu | L-glutamate |
| Gly | Glycine |
| Gly(thien-3-yl) | L-alpha-(3-Thienyl)glycine |
| hCit | Homocitrulline |
| hCys | Homo-L-Cysteine |
| hGln | L-Homoglutamine |
| hGlu | L-homoglutamic acid |
| His | Histidine |
| His(3-Me) | 3-Methyl-L-Histidine |
| homoPhe | Homo-L-Phenylalanine |
| Indoline-COOH | L-Indoline-2-carboxylic acid |
| Inp | Isonipecotic acid |
| L-aMeAsp | alpha-methyl-L-aspartic acid |
| L-aMeGlu | alpha-methyl-L-glutamic acid |
| L-aMeSer | Alpha-methyl-L-serine |
| L-aMeVal | alpha-Methyl-L-Valine |
| L-Apm | L-Aminopimelic acid |
| L-Dab | L-2,4-Diaminobutanoic acid |
| L-hArg | Homo-L-Arginine |
| Lys | Lysine |
| Lys* | L-Lys(AEEAc-AEEAc-L-γ-Glu-17-carboxyheptadecanoyl) |
| Me-Arg | N2-Methyl-L-Arginine |
| Me-D-Arg | N2-Methyl-D-Arginine |
| Me-Gaba | 4-(methylamino)butanoic acid |
| N-4-aminobutyl-Gly | N-(4-aminobutyl)-Glycine |
| N-Me-His | N-Methyl-L-Histidine |
| Nar | L-Norarginine |
| Nip(4-NH₂) | 4-Amino-4-piperidinecarboxylic acid |
| Nle | L-Norleucine |
| Nva(Ph) | 5-Phenyl-L-Norvaline |
| Orn | L-Ornithine |
| PEG1 | 2-(2-aminoethoxy)acetic Acid |
| PEG2 | PEG2 |
| Pen | Penicillamine |
| Phe(3-Me) | 3-Methyl-L-Phenylalanine |
| Phe(3,4-diMe) | 3,4-Dimethyl-L-Phenylalanine |
| Phe(4-Me) | 4-Methyl-L-Phenylalanine |
| Phg | L-Phenylglycine |
| Pro(4-OH) | 4-Hydroxy-L-Proline |
| Pro(4-phenyl) | trans-4-Phenyl-L-Proline |
| Sar | N-methyl-glycine |
| Ser | Serine |
| Thz | L-Thioproline |
| Tranexamic acid | Tranexamic acid |
| Trp | Tryptophan |
| Trp(4-F) | 4-Fluoro-L-tryptophan |
| Trp(5-Cl) | 5-Chloro-L-tryptophan |
| Trp(5-F) | 5-Fluoro-L-tryptophan |
| Trp(5-Me) | 5-Methyl-L-Tryptophan |
| Trp(5-OH) | 5-Hydroxy-L-Tryptophan |
| Trp(6-Br) | 6-Bromo-L-tryptophan |
| Trp(6-CF3) | 6-(trifluoromethyl)-L-tryptophan |
| Trp(6-Cl) | 6-Chloro-L-Tryptophan |
| Trp(6-F) | 6-Fluoro-L-Tryptophan |
| Trp(6-Me) | 6-Methyl-L-Tryptophan |
| Trp(7-F) | 7-Fluoro-L-tryptophan |
| Trp(7-Me) | 7-methyl-L-Tryptophan |

### NUMBERED EMBODIMENTS

The disclosure will be further described in the following series of embodiments (i.e., series A, B, C, D, E, and F), which do not limit the scope of the disclosure described in the claims.

### Embodiments—Series A

Embodiment 1A. A peptide comprising the amino acid sequence of formula (I):

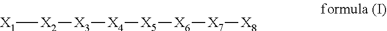

wherein in formula (I):
X₃ is 3-Aminooxetane-3-carboxylic acid (Aib(O-cyclic));
X₄ is glutamine (Gln), homocitrulline (hCit), citrulline (Cit), 3-(3-pyridyl)-L-alanine (3-Pal), L-homoglutamine (hGln), histidine (His), or L-ornithine (Orn); and
X₁, X₂, X₅, X₆, X₇, and X₈ are each independently a canonical or non-canonical amino acid.

Embodiment 2A. The peptide of Embodiment 1A, wherein the peptide of formula (I) is a peptide of any one of formula (Ia), formula (Ib), formula (Ic), formula (Id), formula (Ie), or formula (If):

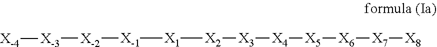

wherein in formula (Ia):
X₋₁, X₋₂, X₋₃, X₋₄, X₁, X₂, X₃, X₄, X₅, X₆, X₇, and X₈ are each independently an amino acid selected from Table A1; or Table A1 or a linker;

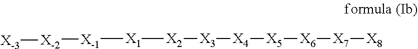

wherein in formula (Ib):
X₋₁, X₋₂, X₋₃, X₁, X₂, X₃, X₄, X₅, X₆, X₇, and X₈ are each independently an amino acid selected from Table A1; or Table A1 or a linker;

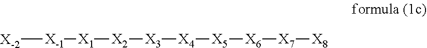

wherein in formula (Ic):
X₋₁, X₋₂, X₁, X₂, X₃, X₄, X₅, X₆, X₇, and X₈ are each independently an amino acid selected from Table A1; or Table A1 or a linker;

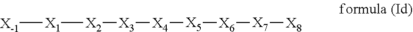

wherein in formula (Id):
X₋₁, X₁, X₂, X₃, X₄, X₅, X₆, X₇, and X₈ are each independently an amino acid selected from Table A1; or Table A1 or a linker;

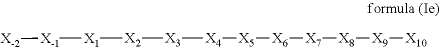

wherein in formula (Ie):
X₋₁, X₋₂, X₁, X₂, X₃, X₄, X₅, X₆, X₇, X₈, X₉, and X₁₀ are each independently an amino acid selected from Table A1; or Table A1 or a linker;

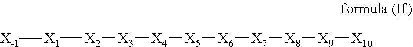

wherein in formula (If):
X₋₁, X₁, X₂, X₃, X₄, X₅, X₆, X₇, X₈, X₉, and X₁₀ are each independently an amino acid selected from Table A1; or Table A1 or a linker.

Embodiment 3A. The peptide of Embodiment 1A or 2A, wherein the peptide of formula (I) is a peptide of formula (Ia).

Embodiment 4A. The peptide of Embodiment 1A or 2A, wherein the peptide of formula (I) is a peptide of formula (Ib).

Embodiment 5A. The peptide of Embodiment 1A or 2A, the peptide of formula (I) is a peptide of formula (Ic).

Embodiment 6A. The peptide of Embodiment 1A or 2A, the peptide of formula (I) is a peptide of formula (Id).

Embodiment 7A. The peptide of Embodiment 1A or 2A, the peptide of formula (I) is a peptide of formula (Ie).

Embodiment 8A. The peptide of Embodiment 1A or 2A, the peptide of formula (I) is a peptide of formula (If).

Embodiment 9A. The peptide of any one of Embodiments 1A-8A, wherein X₄ is Gln.

Embodiment 10A. The peptide of any one of Embodiments 1A-8A, wherein X₄ is Cit.

Embodiment 11A. The peptide of any one of Embodiments 1A-8A, wherein X₄ is hCit.

Embodiment 12A. The peptide of any one of Embodiments 1A-8A, wherein X₄ is 3-Pal.

Embodiment 13A. The peptide of any one of Embodiments 1A-8A, wherein X₄ is hGln.

Embodiment 14A. The peptide of any one of Embodiments 1A-8A, wherein X₄ is His.

Embodiment 15A. The peptide of any one of Embodiments 1A-8A, wherein X₄ is Orn.

Embodiment 16A. The peptide of any one of Embodiments 1A-15A, wherein X₅ is selected from 4-fluoro-D-phenylalanine (D-Phe(4-F)), D-phenylalanine (D-Phe), and 4-methyl-D-phenylalanine (D-Phe(4-Me)).

Embodiment 17A. The peptide of Embodiment 16A, wherein X₅ is D-Phe(4-F).

Embodiment 18A. The peptide of Embodiment 16A, wherein X₅ is D-Phe.

Embodiment 19A. The peptide of Embodiment 16A, wherein X₅ is D-Phe(4-Me).

Embodiment 20A. The peptide of any one of Embodiments 1A-19A, wherein X₆ is arginine (Arg).

Embodiment 21A. The peptide of any one of Embodiments 1A-20A, wherein X₇ is 6-fluoro-L-tryptophan (Trp(6-F)).

Embodiment 22A. The peptide of any one of Embodiments 1A-21A, wherein X₈ is penicillamine (Pen) or cysteine (Cys).

Embodiment 23A. The peptide of any one of Embodiments 1A-22A, wherein X₁ is selected from D-norarginine (D-Nar) and beta-homo-L-arginine (Beta-homoArg).

Embodiment 24A. The peptide of any one of Embodiments 1A-23A, wherein X₁ is D-Nar.

Embodiment 25A. The peptide of any one of Embodiments 1A-24A, wherein X₂ is Cys.

Embodiment 26A. The peptide of any one of Embodiments 1A-25A, wherein the peptide of formula (I) is selected from Table A1, Table A1A, Table A2 and Table A2A.

Embodiment 27A. The peptide of any one of Embodiments 1A-26A, wherein the peptide is a cyclic peptide.

Embodiment 28A. The peptide of Embodiment 27A, wherein the cyclic peptide comprises a disulfide bridge or a lactam bridge.

Embodiment 29A. The peptide of Embodiment 27A or 28A, wherein the cyclic peptide has the formula (II):

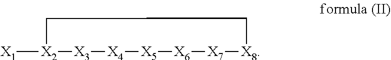

Embodiment 30A. The peptide of any one of Embodiments 1A-29A, wherein the cyclic peptide of formula (II) is a cyclic peptide of any one of formula (IIa), formula (IIb), formula (IIc), formula (IId), formula (IIe), or formula (IIf):

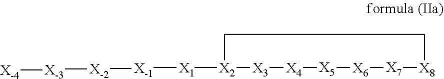

wherein in formula (IIa): X₋₁, X₋₂, X₋₃, X₋₄, X₁, X₂, X₃, X₄, X₅, X₆, X₇, and X₈ are each independently an amino acid selected from Table A1; or Table A1 or a linker;

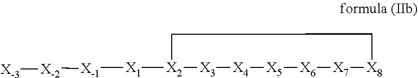

wherein in formula (IIb): X₋₁, X₋₂, X₋₃, X₁, X₂, X₃, X₄, X₅, X₆, X₇, and X₈ are each independently an amino acid selected from Table A1; or Table A1 or a linker;

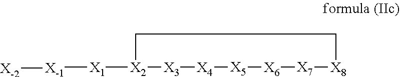

wherein in formula (IIc): X₋₁, X₋₂, X₁, X₂, X₃, X₄, X₅, X₆, X₇, and X₈ are each independently an amino acid selected from Table A1; or Table A1 or a linker;

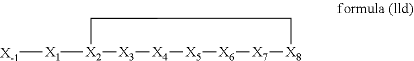

wherein in formula (IId): X₋₁, X₁, X₂, X₃, X₄, X₅, X₆, X₇, and X₈ are each independently an amino acid selected from Table A1; or Table A1 or a linker;

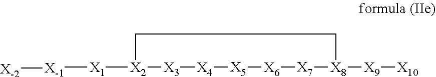

wherein in formula (IIe): X₋₁, X₋₂, X₁, X₂, X₃, X₄, X₅, X&, X₇, X₈, X₉, and X₁₀ are each independently an amino acid selected from Table A1; or Table A1 or a linker;

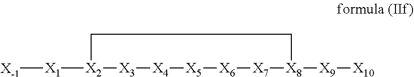

wherein in formula (IIf): X₋₁, X₋₂, X₁, X₂, X₃, X₄, X₅, X₆, X₇, X₈, X₉, and X₁₀ are each independently an amino acid selected from Table A1; or Table A1 or a linker.

Embodiment 31A. The peptide of Embodiments 29A or 30A, wherein the cyclic peptide of formula (II) is a cyclic peptide of formula (IIa).

Embodiment 32A. The peptide of Embodiments 29A or 30A, wherein the cyclic peptide of formula (II) is a cyclic peptide of formula (IIb).

Embodiment 33A. The peptide of Embodiments 29A or 30A, wherein the cyclic peptide of formula (II) is a cyclic peptide of formula (IIc).

Embodiment 34A. The peptide of Embodiments 29A or 30A, wherein the cyclic peptide of formula (II) is a cyclic peptide of formula (IId).

Embodiment 35A. The peptide of Embodiments 29A or 30A, wherein the cyclic peptide of formula (II) is a cyclic peptide of formula (IIe).

Embodiment 36A. The peptide of Embodiments 29A or 30A, wherein the cyclic peptide of formula (II) is a cyclic peptide of formula (IIf).

Embodiment 37A. The peptide of any one of Embodiments 1A-36A, wherein the peptide further comprises one or more amino acids conjugated to X₁ and/or X₈, optionally wherein the one or more amino acids are selected from D-Arginine (D-Arg), glycine (Gly), and L-Lys(AEEAc-AEEAc-L-γ-Glu-17-carboxyheptadecanoyl) (Lys*).

Embodiment 38A. The peptide of any one of Embodiments 1A-37A, wherein the peptide further comprises one or more lipids conjugated to X₁ and/or X₈.

Embodiment 39A. The peptide of any one of Embodiments 1A-38A, wherein the peptide is capped with N-terminal acetyl and/or C-terminal amide groups.

Embodiment 40A. The peptide of any one of Embodiments 1A-39A, wherein the peptide is selected from Table A1, Table A1A, Table A2 and Table A2A.

Embodiment 41A. A peptide consisting of the amino acid sequence as set forth in formula (III):

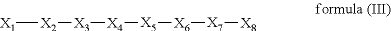

wherein in formula (III):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen).

Embodiment 42A. The peptide of Embodiment 41A, wherein the peptide is a cyclic peptide.

Embodiment 43A. The peptide of Embodiment 42A, wherein the cyclic peptide comprises a disulfide bridge or a lactam bridge.

Embodiment 44A. The peptide of Embodiment 42A or 43A, wherein the cyclic peptide is a peptide consisting of the amino acid sequence as set forth in formula (IV):

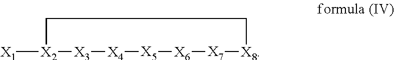

wherein in formula (IV):
X₁ is D-norarginine (D-Nar);
X₂ is cysteine (Cys);
X₃ is 3-Aminooxetane-3-carboxylic acid (Aib(O-cyclic));
X₄ is selected from glutamine (Gln), homocitrulline (hCit), and citrulline (Cit);
X₅ is selected from 4-fluoro-D-phenylalanine (D-Phe(4-F)), D-phenylalanine (D-Phe), and 4-methyl-D-phenylalanine (D-Phe(4-Me));
X₆ is arginine (Arg);
X₇ is 6-fluoro-L-tryptophan (Trp(6-F)); and
X₈ is penicillamine (Pen).

Embodiment 45A. The peptide of any one of Embodiments 41A-44A, wherein the peptide is capped with N-terminal acetyl and/or C-terminal amide groups.

Embodiment 46A. The peptide of any one of Embodiments 41A-45A, wherein the peptide is capped with N-terminal acetyl.

Embodiment 47A. The peptide of any one of Embodiments 1A-46A, wherein the peptide demonstrates increased selectivity for MC4R over MC1R when administered to a subject compared to a control.

Embodiment 48A. The peptide of any one of Embodiments 1A-47A, wherein the peptide demonstrates increased selectivity for MC4R over MC1R as measured by an in vitro, ex vivo, or in vivo assay when compared to a control.

Embodiment 49A. The peptide of Embodiment 47A or 48A, wherein the peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 50A. The peptide of Embodiment 47A or 48A, wherein the peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 51A. The peptide of any one of Embodiments 1A-46A, wherein the peptide demonstrates an increased ratio of MC4R intracellular signaling to MC1R intracellular signaling when administered to a subject compared to a control.

Embodiment 52A. The peptide of any one of Embodiments 1A-47A, wherein the peptide demonstrates increased selectivity for MC4R intracellular signaling to MC1R intracellular signaling as measured by an in vitro, ex vivo, or in vivo assay when compared to a control.

Embodiment 53A. The peptide of Embodiment 51A or 52A, wherein the peptide demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when administered to a subject compared to a control.

Embodiment 54A. The peptide of Embodiment 51A or 52A, wherein the peptide demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when administered to a subject compared to a control.

Embodiment 55A. The peptide of any one of Embodiments 1A-54A, wherein the peptide demonstrates enhanced melanocortin 4 receptor (MC4R) function in a subject when compared to before the peptide is administered or to a pre-treatment or non-treatment state, or a subject treated with control.

Embodiment 56A. The peptide of any one of Embodiments 1A-55A, wherein the peptide demonstrates decreased melanocortin 1 receptor (MC1R) function in a subject when compared to before the peptide is administered or to a pre-treatment or non-treatment state, or a subject treated with control.

Embodiment 57A. The peptide of any one of Embodiments 1A-56A, wherein the peptide demonstrates enhanced melanocortin 4 receptor (MC4R) function as measured by an in vitro, ex vivo, or in vivo assay when compared to a control.

Embodiment 58A. The peptide of any one of Embodiments 1A-57A, wherein the peptide demonstrates decreased melanocortin 1 receptor (MC1R) function as measured by an in vitro, ex vivo, or in vivo assay when compared to a control.

Embodiment 59A. The peptide of any one of Embodiments 55A-58A, wherein the peptide demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 60A. The peptide of any one of Embodiments 55A-58A, wherein the peptide demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 61A. The peptide of any one of Embodiments 33A-60A, wherein the cyclic peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 62A. The peptide of any one of Embodiments 33A-60A, wherein the cyclic peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 63A. The peptide of any one of Embodiments 1A-62A, wherein the peptide comprises a half-life extending moiety, optionally wherein the half-life extending moiety comprises polyethylene glycol (PEG), recombinant PEG mimetics, glycosylation of carbohydrates, Fc-fusion proteins or conjugates, albumin fusion proteins or conjugates, albumin-binding fusion proteins or conjugates, polypropylene glycol (PPG), XTEN fusion protein or conjugates, or a combination thereof.

Embodiment 64A. The peptide of any one of Embodiments 1A-63A, wherein the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

Embodiment 65A. The peptide of any one of Embodiments 1A-64A, wherein the peptide further comprises a therapeutic, diagnostic, and/or imaging moiety, optionally wherein the therapeutic, diagnostic, and/or imaging moiety comprises a small molecule, a biological (e.g., a biopolymer, a protein, a nucleic acid, a polysaccharide), or a radionuclide.

Embodiment 66A. The peptide of any one of Embodiments 1A-65A, wherein the peptide of formula (I) or formula (III) is conjugated to or co-formulated with an additional therapeutic agent or therapeutic moiety.

Embodiment 67A. The peptide of Embodiments 65A or 66A, wherein the additional therapeutic agent or therapeutic moiety comprises incretin, an incretin analogue, or a modulator of an incretin receptor.

Embodiment 68A. The peptide of Embodiment 67A, wherein the modulator is an agonist.

Embodiment 69A. The peptide of Embodiment 68A, wherein the agonist is an agonist of GLP-1, GIP, and/or glucagon receptor.

Embodiment 70A. The peptide of Embodiment 68A, wherein the agonist is a GLP-1 analogue.

Embodiment 71A. The peptide of Embodiment 70A, wherein the GLP-1 analogue comprises a non-canonical amino acid.

Embodiment 72A. The peptide of Embodiment 70A, wherein the GLP-1 analogue comprises tirzepatide, tirzepatide analogue, liraglutide, retatrutide, exenatide, lixisenatide, semaglutide or semaglutide derivative.

Embodiment 73A. A protein comprising the peptide of any one of Embodiments 1A to 72A.

Embodiment 74A. The protein of Embodiment 73A, wherein the protein has a size of at least about 10 amino acid residues, or at least about 15 resides, or at least about 20 residues, or at least about 25 residues, or at least about 30 residues, or at least about 35 residues, or at least about 40 residues, or at least about 45 residues, or at least about 50 residues, or at least about 60 residues, or at least about 70 residues, or at least about 80 residues, or at least about 90 residues, or at least about 100 residues, or at least about 250 residues, or at least about 500 residues, at least about 750 residues, at least about 1,000 residues, at least about 1,250 residues, at least about 1,500 residues, at least about 1,750 residues, at least about 2,000 residues, at least about 3,000 residues, at least about 4,000 residues, or at least about 5,000 residues.

Embodiment 75A. A nucleic acid encoding the peptide of any one of Embodiments 1A to 72A, or the protein of Embodiment 73A or 74A.

Embodiment 76A. A solid synthesis device conjugated to the peptide of any one of Embodiments 1A to 72A, or the protein of Embodiment 73 or 74, or the nucleic acid of Embodiment 75A.

Embodiment 77A. A pharmaceutical composition comprising a peptide of any one of Embodiments 1A to 72A, or the protein of Embodiment 73A or 74A, or the nucleic acid of Embodiment 75A, and a pharmaceutically acceptable excipient or carrier.

Embodiment 78A. The pharmaceutical composition of Embodiment 77A, wherein the pharmaceutical composition further comprises an additional therapeutic, diagnostic, and/or imaging moiety.

Embodiment 79A. The pharmaceutical composition of Embodiment 77A or 78A, wherein the pharmaceutical composition is conjugated to or co-formulated with an additional therapeutic agent or therapeutic moiety.

Embodiment 80. The pharmaceutical composition of any one of Embodiments 77A or 78A, wherein the pharmaceutical composition is administered orally, parenterally, by inhalation spray, topically, rectally, nasally, buccally, sub-lingually, vaginally or via an implanted reservoir.

Embodiment 81. The pharmaceutical composition of any one of Embodiments 77A or 78A, wherein the pharmaceutical composition is administered via intravenous injection, intramuscular injection, subcutaneous injection, or depot injection.

Embodiment 82A. A method of selectively increasing the activity of MC4R over MC1R comprising administering a peptide of any one of Embodiments 1A to 72A or the pharmaceutical composition of Embodiments 77A to 81A to a subject in need thereof.

Embodiment 83. The method of Embodiment 82A, wherein the method prevents, reduces, or eliminates hyperpigmentation in the subject when compared to an untreated or pre-treatment subject, or subject treated with a control.

Embodiment 84A. The method of Embodiment 83A, wherein the hyperpigmentation is reduced by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% when compared to an untreated or pre-treatment subject, or subject treated with a control.

Embodiment 85A. The method of Embodiment 83A, wherein the hyperpigmentation is reduced by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold when compared to an untreated or pre-treatment subject, or subject treated with a control.

Embodiment 86A. A method of treating a disease or disorder associated with MC4R function comprising administering a peptide of any one of Embodiments 1A to 72A or the pharmaceutical composition of Embodiments 77A to 81A to a subject in need thereof.

Embodiment 87A. The method of Embodiment 86A, wherein the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency, and Prader-Willi syndrome.

Embodiment 88A. The method of Embodiment 86A or 87A, wherein the method further comprises co-administration of an additional therapeutic agent.

Embodiment 89A. The method of any one of Embodiments 86A-88A, wherein the additional therapeutic agent comprises incretin, an incretin analogue, or a modulator of an incretin receptor.

Embodiment 90A. The method of Embodiment 89A, wherein the modulator is an agonist.

Embodiment 91A. The method of Embodiment 90A, wherein the agonist is an agonist of GLP-1, GIP, and/or glucagon receptor.

Embodiment 92A. The method of Embodiment 90A, wherein the agonist is a GLP-1 analogue.

Embodiment 93. The method of Embodiment 92A, wherein the GLP-1 analogue comprises a non-canonical amino acid.

Embodiment 94. The method of Embodiment 92A, wherein the GLP-1 analogue comprises tirzepatide, tirzepatide analogue, liraglutide, retatrutide, exenatide, lixisenatide, semaglutide or semaglutide derivative.

Embodiment 95A. The method of Embodiment 89A, wherein the subject is receiving and/or previously received treatment comprising incretin, an incretin analogue, or a modulator of an incretin receptor.

Embodiment 96A. The method of Embodiment 89A, wherein the subject has not or is not receiving treatment comprising incretin, an incretin analogue, or a modulator of an incretin receptor.

Embodiment 97A. A method of treating obesity or abnormal or excessive fat accumulation comprising administering a peptide of any one of Embodiments 1A to 72A or the pharmaceutical composition of Embodiments 77A to 81A to a subject in need thereof.

Embodiment 98A. A method of treating a disease or disorder associated with MC4R function comprising administering a peptide of any one of Embodiments 1A to 72A or the pharmaceutical composition of Embodiments 77A to 81A to a subject receiving and/or previously received incretin.

Embodiment 99A. A method of treating a disease or disorder associated with MC4R function comprising administering a peptide of any one of Embodiments 1A to 72A or the pharmaceutical composition of Embodiments 77A to 81A to a subject not receiving incretin.

Embodiment 100A. The method of any one of Embodiments 82A to 99A, wherein the peptide or pharmaceutical composition is administered from a prefilled dosage form device, such as syringe or autoinjector.

Embodiment 101A. A peptide consisting of the amino acid sequence as set forth in formula (IV):

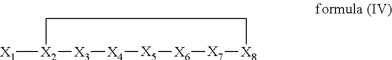

wherein in formula (IV):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen),
wherein

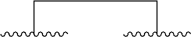 [SMILES: *CC*]

represents a disulfide bridge, and the peptide is capped with a N-terminal acetyl.

### Embodiments—Series B

Embodiment 1B. A peptide comprising the amino acid sequence of formula (BI):

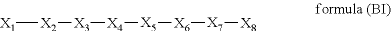

wherein in formula (BI):
X₃ is L-Phenylglycine (Phg);
X₄ is 3-(3-Pyridyl)-L-alanine (3-Pal); and
X₁, X₂, X₅, X₆, X₇, and X₈ are each independently a canonical or non-canonical amino acid.

Embodiment 2B. The peptide of Embodiment 1B, wherein the peptide of formula (BI) is a peptide of any one of formula (BIa), formula (BIb), formula (BIc), formula (BId), formula (BIe), or formula (BIf).

Embodiment 3B. The peptide of Embodiment 1B or 2B, wherein the peptide of formula (BI) is a peptide of formula (BIa):

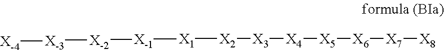

wherein in formula (BIa):
X₋₁, X₋₂, X₋₃, X₋₄, X₁, X₂, X₃, X₄, X₅, X₆, X₇, and X₈ are each independently an amino acid selected from Table B1; or Table B1 or a linker.

Embodiment 4B. The peptide of Embodiment 1B or 2B, wherein the peptide of formula (BI) is a peptide of formula (BIb):

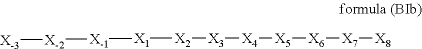

wherein in formula (Ib): X₋₁, X₋₂, X₋₃, X₁, X₂, X₃, X₄, X₅, X₆, X₇, and X₈ are each independently an amino acid selected from Table B1; or Table B1 or a linker.

Embodiment 5B. The peptide of Embodiment 1B or 2B, the peptide of formula (BI) is a peptide of formula (BIc):

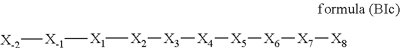

wherein in formula (Ic): X₋₁, X₋₂, X₁, X₂, X₃, X₄, X₅, X₆, X₇, and X₈ are each independently an amino acid selected from Table B1; or Table B1 or a linker.

Embodiment 6B. The peptide of Embodiment 1B or 2B, the peptide of formula (BI) is a peptide of formula (BId):

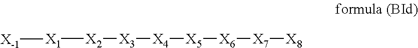

wherein in formula (Id): X₋₁, X₁, X₂, X₃, X₄, X₅, X₆, X₇, and X₈ are each independently an amino acid selected from Table B1; or Table B1 or a linker.

Embodiment 7B. The peptide of Embodiment 1B or 2B, the peptide of formula (BI) is a peptide of formula (BIe):

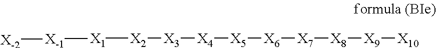

wherein in formula (Ie): X₋₁, X₋₂, X₁, X₂, X₃, X₄, X₅, X₆, X₇, X₈, X₉, and X₁₀ are each independently an amino acid selected from Table B1; or Table B1 or a linker.

Embodiment 8B. The peptide of Embodiment 1B or 2B, the peptide of formula (BI) is a peptide of formula (BIf):

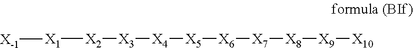

wherein in formula (BIf): X₋₁, X₁, X₂, X₃, X₄, X₅, X₆, X₇, X₈, X₉, and X₁₀ are each independently an amino acid selected from Table B1; or Table B1 or a linker.

Embodiment 9B. The peptide of any one of Embodiments 1B-8B, wherein X₅ is selected from 4-Fluoro-D-phenylalanine (D-Phe(4-F)).

Embodiment 10B. The peptide of any one of Embodiments 1B-9B, wherein X₆ is Arginine (Arg).

Embodiment 11B. The peptide of any one of Embodiments 1B-10B, wherein X₇ is 6-Fluoro-L-Tryptophan (Trp(6-F)).

Embodiment 12B. The peptide of any one of Embodiments 1B-11B, wherein X₈ is Cysteine (Cys).

Embodiment 13B. The peptide of any one of Embodiments 1B-12B, wherein X₁ is selected from D-Norarginine (D-Nar) and beta-homo-L-arginine (Beta-homoArg).

Embodiment 14B. The peptide of any one of Embodiments 1B-13B, wherein X₁ is Beta-homoArg.

Embodiment 15B. The peptide of any one of Embodiments 1B-14B, wherein X₂ is Cys.

Embodiment 16B. The peptide of any one of Embodiments 1B-15B, wherein the peptide of formula (I) is selected from Table B1, Table B1A, Table B2, and Table B2A.

Embodiment 17B. The peptide of any one of Embodiments 1B-16B, wherein the peptide is a cyclic peptide.

Embodiment 18B. The peptide of Embodiment 17B, wherein the cyclic peptide comprises a disulfide bridge or a lactam bridge.

Embodiment 19B. The peptide of Embodiment 17B or 18B, wherein the cyclic peptide has the formula (II):

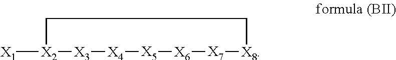

Embodiment 20B. The peptide of any one of Embodiments 1B-19B, wherein the cyclic peptide of formula (BII) is a cyclic peptide of any one of formula (BIIa), formula (BIIb), formula (BIIc), formula (BIId), formula (BIIe), or formula (BIIf).

Embodiment 21B. The peptide of Embodiments 19B or 20B, wherein the cyclic peptide of formula (BII) is a cyclic peptide of formula (BIIa):

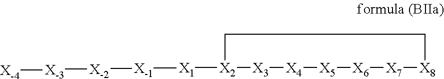

wherein in formula (BIIa): X₋₁, X₋₂, X₋₃, X₋₄, X₁, X₂, X₃, X₄, X₅, X₆, X₇, and X₈ are each independently an amino acid selected from Table B1; or Table B1 or a linker.

Embodiment 22B. The peptide of Embodiments 19B or 20B, wherein the cyclic peptide of formula (B1) is a cyclic peptide of formula (BIIb):

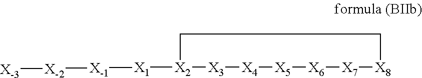

wherein in formula (IIb): X₋₁, X₋₂, X₋₃, X₁, X₂, X₃, X₄, X₅, X₆, X₇, and X₈ are each independently an amino acid selected from Table B1; or Table B1 or a linker.

Embodiment 23B. The peptide of Embodiments 19B or 20B, wherein the cyclic peptide of formula (BII) is a cyclic peptide of formula (BIIc):

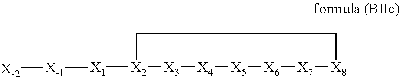

wherein in formula (BIIc): X₋₁, X₋₂, X₁, X₂, X₃, X₄, X₅, X₆, X₇, and X₈ are each independently an amino acid selected from Table B1; or Table B1 or a linker.

Embodiment 24B. The peptide of Embodiments 19B or 20B, wherein the cyclic peptide of formula (II) is a cyclic peptide of formula (BIId):

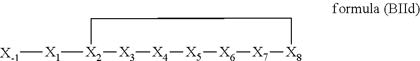

wherein in formula (IId): X₋₁, X₁, X₂, X₃, X₄, X₅, X₆, X₇, and X₈ are each independently an amino acid selected from Table B1; or Table B1 or a linker.

Embodiment 25B. The peptide of Embodiments 19B or 20B, wherein the cyclic peptide of formula (II) is a cyclic peptide of formula (BIIe):

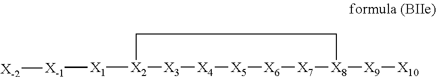

wherein in formula (IIe): X₋₁, X₋₂, X₁, X₂, X₃, X₄, X₅, X₆, X₇, X₈, X₉, and X₁₀ are each independently an amino acid selected from Table B1; or Table B1 or a linker.

Embodiment 26B. The peptide of Embodiments 19B or 20B, wherein the cyclic peptide of formula (II) is a cyclic peptide of formula (BIIf):

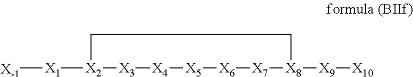

wherein in formula (IIf): X₋₁, X₋₂, X₁, X₂, X₃, X₄, X₅, X₆, X₇, X₈, X₉, and X₁₀ are each independently an amino acid selected from Table B1; or Table B1 or a linker.

Embodiment 27B. The peptide of any one of Embodiments 1B-26B, wherein the peptide further comprises one or more amino acids conjugated to X₁ and/or X₈, optionally wherein the one or more amino acids are selected from D-Arginine (D-Arg), glycine (Gly), and L-Lys(AEEAc-AEEAc-L-γ-Glu-17-carboxyheptadecanoyl) (Lys*).

Embodiment 28B. The peptide of any one of Embodiments 1B-27B, wherein the peptide further comprises one or more lipids conjugated to X₁ and/or X₈.

Embodiment 29B. The peptide of any one of Embodiments 1B-28B, wherein the peptide is capped with N-terminal acetyl and/or C-terminal amide groups.

Embodiment 30B. The peptide of any one of Embodiments 1B-29B, wherein the peptide is selected from Table B1, Table B1A, Table B2, and Table B2A.

Embodiment 31B. The peptide of any one of Embodiments 1B-30B, wherein the peptide demonstrates increased selectivity for MC4R over MC1R when administered to a subject compared to a control.

Embodiment 32B. The peptide of any one of Embodiments 1B-31B, wherein the peptide demonstrates increased selectivity for MC4R over MC1R as measured by an in vitro, ex vivo, or in vivo assay when compared to a control.

Embodiment 33B. The peptide of Embodiment 31B or 32B, wherein the peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 34B. The peptide of Embodiment 31B or 32B, wherein the peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 35B. The peptide of any one of Embodiments 1B-30B, wherein the peptide demonstrates an increased ratio of MC4R intracellular signaling to MC1R intracellular signaling when administered to a subject compared to a control.

Embodiment 36B. The peptide of any one of Embodiments 1B-31B, wherein the peptide demonstrates increased selectivity for MC4R intracellular signaling to MC1R intracellular signaling as measured by an in vitro, ex vivo, or in vivo assay when compared to a control.

Embodiment 37B. The peptide of Embodiment 35B or 36B, wherein the peptide demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when administered to a subject compared to a control.

Embodiment 38B. The peptide of Embodiment 35B or 36B, wherein the peptide demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when administered to a subject compared to a control.

Embodiment 39B. The peptide of any one of Embodiments 1B-38B, wherein the peptide demonstrates enhanced melanocortin 4 receptor (MC4R) function in a subject when compared to before the peptide is administered or to a pre-treatment or non-treatment state, or a subject treated with control.

Embodiment 40B. The peptide of any one of Embodiments 1B-39B, wherein the peptide demonstrates decreased melanocortin 1 receptor (MC1R) function in a subject when compared to before the peptide is administered or to a pre-treatment or non-treatment state, or a subject treated with control.

Embodiment 41B. The peptide of any one of Embodiments 1B-40B, wherein the peptide demonstrates enhanced melanocortin 4 receptor (MC4R) function as measured by an in vitro, ex vivo, or in vivo assay when compared to a control.

Embodiment 42B. The peptide of any one of Embodiments 1B-41B, wherein the peptide demonstrates decreased melanocortin 1 receptor (MC1R) function as measured by an in vitro, ex vivo, or in vivo assay when compared to a control.

Embodiment 43B. The peptide of any one of Embodiments 39B-42B, wherein the peptide demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 44B. The peptide of any one of Embodiments 39B-42B, wherein the peptide demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 45B. The peptide of any one of Embodiments 17B-44B, wherein the cyclic peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 46B. The peptide of any one of Embodiments 17B-44B, wherein the cyclic peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 47B. The peptide of any one of Embodiments 1B-46B, wherein the peptide comprises a half-life extending moiety, optionally wherein the half-life extending moiety comprises polyethylene glycol (PEG), recombinant PEG mimetics, glycosylation of carbohydrates, Fc-fusion proteins or conjugates, albumin fusion proteins or conjugates, albumin-binding fusion proteins or conjugates, polypropylene glycol (PPG), XTEN fusion protein or conjugates, or a combination thereof.

Embodiment 48B. The peptide of any one of Embodiments 1B-47B, wherein the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

Embodiment 49B. The peptide of any one of Embodiments 1B-48B, wherein the peptide further comprises a therapeutic, diagnostic, and/or imaging moiety, optionally wherein the therapeutic, diagnostic, and/or imaging moiety comprises a small molecule, a biological (e.g., a biopolymer, a protein, a nucleic acid, a polysaccharide), or a radionuclide.

Embodiment 50B. The peptide of any one of Embodiments 1B-49B, wherein the peptide of formula (BI) is conjugated to or co-formulated with an additional therapeutic agent or therapeutic moiety.

Embodiment 51B. The peptide of Embodiments 49B or 50B, wherein the additional therapeutic agent or therapeutic moiety comprises incretin, an incretin analogue, or a modulator of an incretin receptor.

Embodiment 52B. The peptide of Embodiment 51B, wherein the modulator is an agonist.

Embodiment 53B. The peptide of Embodiment 52B, wherein the agonist is an agonist of GLP-1, GIP, and/or glucagon receptor.

Embodiment 54B. The peptide of Embodiment 52B, wherein the agonist is a GLP-1 analogue.

Embodiment 55B. The peptide of Embodiment 54B, wherein the GLP-1 analogue comprises a non-canonical amino acid.

Embodiment 56B. The peptide of Embodiment 54B, wherein the GLP-1 analogue comprises tirzepatide, tirzepatide analogue, liraglutide, retatrutide, exenatide, lixisenatide, semaglutide or semaglutide derivative.

Embodiment 57B. A protein comprising the peptide of any one of Embodiments 1B to 56B.

Embodiment 58B. The protein of Embodiment 57B, wherein the protein has a size of at least about 10 amino acid residues, or at least about 15 resides, or at least about 20 residues, or at least about 25 residues, or at least about 30 residues, or at least about 35 residues, or at least about 40 residues, or at least about 45 residues, or at least about 50 residues, or at least about 60 residues, or at least about 70 residues, or at least about 80 residues, or at least about 90 residues, or at least about 100 residues, or at least about 250 residues, or at least about 500 residues, at least about 750 residues, at least about 1,000 residues, at least about 1,250 residues, at least about 1,500 residues, at least about 1,750 residues, at least about 2,000 residues, at least about 3,000 residues, at least about 4,000 residues, or at least about 5,000 residues.

Embodiment 59B. A nucleic acid encoding the peptide of any one of Embodiments 1B to 56B, or the protein of Embodiment 57B or 58B.

Embodiment 60B. A solid synthesis device conjugated to the peptide of any one of Embodiments 1B to 56B, the protein of Embodiment 57B or 58B, or the nucleic acid of Embodiment 59B.

Embodiment 61B. A pharmaceutical composition comprising a peptide of any one of Embodiments 1B to 56B, or the protein of Embodiment 57B or 58B, or the nucleic acid of Embodiment 59B, and a pharmaceutically acceptable excipient or carrier.

Embodiment 62B. The pharmaceutical composition of Embodiment 61B, wherein the pharmaceutical composition further comprises an additional therapeutic, diagnostic, and/or imaging moiety.

Embodiment 63B. The pharmaceutical composition of Embodiment 61B or 62B, wherein the pharmaceutical composition is conjugated to or co-formulated with an additional therapeutic agent or therapeutic moiety.

Embodiment 64B. The pharmaceutical composition of any one of Embodiments 61B or 62B, wherein the pharmaceutical composition is administered orally, parenterally, by inhalation spray, topically, rectally, nasally, buccally, sub-lingually, vaginally or via an implanted reservoir.

Embodiment 65B. The pharmaceutical composition of any one of Embodiments 61B or 62B, wherein the pharmaceutical composition is administered via intravenous injection, intramuscular injection, subcutaneous injection, or depot injection.

Embodiment 66B. A method of selectively increasing the activity of MC4R over MC1R comprising administering a peptide of any one of Embodiments 1B to 56B or the pharmaceutical composition of Embodiments 61B to 65B to a subject in need thereof.

Embodiment 67B. The method of Embodiment 66B, wherein the method prevents, reduces, or eliminates hyperpigmentation in the subject when compared to an untreated or pre-treatment subject, or subject treated with a control.

Embodiment 68B. The method of Embodiment 67B, wherein the hyperpigmentation is reduced by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% when compared to an untreated or pre-treatment subject, or subject treated with a control.

Embodiment 69B. The method of Embodiment 67B, wherein the hyperpigmentation is reduced by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold when compared to an untreated or pre-treatment subject, or subject treated with a control.

Embodiment 70B. A method of treating a disease or disorder associated with MC4R, function comprising administering a peptide of any one of Embodiments 1B to 56B or the pharmaceutical composition of Embodiments 61B to 6B5 to a subject in need thereof.

Embodiment 71B. The method of Embodiment 70B, wherein the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency, and Prader-Willi syndrome.

Embodiment 72B. The method of Embodiment 70B or 71B, wherein the method further comprises co-administration of an additional therapeutic agent.

Embodiment 73B. The method of any one of Embodiments 70B to 72B, wherein the additional therapeutic agent comprises incretin, an incretin analogue, or a modulator of an incretin receptor.

Embodiment 74B. The method of Embodiment 73B, wherein the modulator is an agonist.

Embodiment 75B. The method of Embodiment 74B, wherein the agonist is an agonist of GLP-1, GIP, and/or glucagon receptor.

Embodiment 76B. The method of Embodiment 74B, wherein the agonist is a GLP-1 analogue.

Embodiment 77B. The method of Embodiment 76B, wherein the GLP-1 analogue comprises a non-canonical amino acid.

Embodiment 78B. The method of Embodiment 76B, wherein the GLP-1 analogue comprises tirzepatide, tirzepatide analogue, liraglutide, retatrutide, exenatide, lixisenatide, semaglutide or semaglutide derivative.

Embodiment 79B. The method of Embodiment 73B, wherein the subject is receiving and/or previously received treatment comprising incretin, an incretin analogue, or a modulator of an incretin receptor.

Embodiment 80B. The method of Embodiment 73B, wherein the subject has not or is not receiving treatment comprising incretin, an incretin analogue, or a modulator of an incretin receptor.

Embodiment 81B. A method of treating obesity or abnormal or excessive fat accumulation comprising administering a peptide of any one of Embodiments 1B to 56B or the pharmaceutical composition of Embodiments 61B to 65B to a subject in need thereof.

Embodiment 82B. A method of treating a disease or disorder associated with MC4R function comprising administering a peptide of any one of Embodiments 1B to 56B or the pharmaceutical composition of Embodiments 61B to 65B to a subject receiving and/or previously received incretin.

Embodiment 83B. A method of treating a disease or disorder associated with MC4R function comprising administering a peptide of any one of Embodiments 1B to 56B or the pharmaceutical composition of Embodiments 61B to 65B to a subject not receiving incretin.

Embodiment 84B. The method of any one of Embodiments 65B to 83B, wherein the peptide or pharmaceutical composition is administered from a prefilled dosage form device, such as syringe or autoinjector.

### Embodiments—Series C

Embodiment 1C. A peptide comprising the amino acid sequence of formula (I):

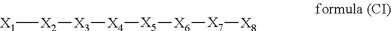

wherein in formula (CI):
X₃ is alpha-Methyl-D-Ornithine (D-aMeOrn);
X₄ is Glutamine (Gln); and
X₁, X₂, X₅, X₆, X₇, and X₈ are each independently a canonical or non-canonical amino acid.

Embodiment 2C. The peptide of Embodiment 1C, wherein the peptide of formula (CI) is a peptide of any one of formula (CIa), formula (CIb), formula (CIc), formula (CId), formula (CIe), or formula (CIf).

Embodiment 3C. The peptide of Embodiment 1C or 2C, wherein the peptide of formula (CI) is a peptide of formula (CIa):

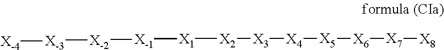

wherein in formula (CIa):
X₋₁, X₋₂, X₋₃, X₋₄, X₁, X₂, X₃, X₄, X₅, X₆, X₇, and X₈ are each independently an amino acid selected from Table C1; or Table C1 or a linker.

Embodiment 4C. The peptide of Embodiment 1C or 2C, wherein the peptide of formula (I) is a peptide of formula (Ib):

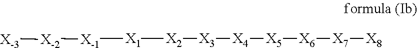

wherein in formula (Ib): X₋₁, X₋₂, X₋₃, X₁, X₂, X₃, X₄, X₅, X₆, X₇, and X₈ are each independently an amino acid selected from Table C1; or Table C1 or a linker.

Embodiment 5C. The peptide of Embodiment 1C or 2C, the peptide of formula (CI) is a peptide of formula (CIc):

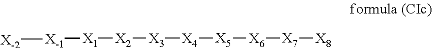

wherein in formula (Ic): X₋₁, X₋₂, X₁, X₂, X₃, X₄, X₅, X₆, X₇, and X₈ are each independently an amino acid selected from Table C1; or Table C1 or a linker.

Embodiment 6C. The peptide of Embodiment 1C or 2C, the peptide of formula (CI) is a peptide of formula (Id):

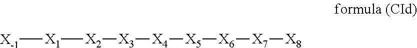

wherein in formula (Id): X₋₁, X₁, X₂, X₃, X₄, X₅, X₆, X₇, and X₈ are each independently an amino acid selected from Table C1; or Table C1 or a linker.

Embodiment 7C. The peptide of Embodiment 1C or 2C, the peptide of formula (CI) is a peptide of formula (CIe):

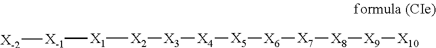

wherein in formula (Ie): X₋₁, X₋₂, X₁, X₂, X₃, X₄, X₅, X₆, X₇, X₈, X₉, and X₁₀ are each independently an amino acid selected from Table C1; or Table C1 or a linker.

Embodiment 8C. The peptide of Embodiment 1C or 2C, the peptide of formula (CI) is a peptide of formula (CIf):

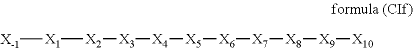

wherein in formula (If): X₋₁, X₁, X₂, X₃, X₄, X₅, X₆, X₇, X₈, X₉, and X₁₀ are each independently an amino acid selected from Table C1; or Table C1 or a linker.

Embodiment 9C. The peptide of any one of Embodiments 1C-8C, wherein X₅ is D-Phenylalanine (D-Phe).

Embodiment 10C. The peptide of any one of Embodiments 1C-9C, wherein X₆ is Arginine (Arg).

Embodiment 11C. The peptide of any one of Embodiments 1C-10C, wherein X₇ is 6-Fluoro-L-Tryptophan (Trp(6-F)).

Embodiment 12C. The peptide of any one of Embodiments 1C-11C, wherein X₈ is Cysteine (Cys).

Embodiment 13C. The peptide of any one of Embodiments 1C-12C, wherein X₁ is selected from D-Norarginine (D-Nar) and beta-homo-L-arginine (Beta-homoArg).

Embodiment 14C. The peptide of any one of Embodiments 1C-13C, wherein X₁ is D-Nar.

Embodiment 15C. The peptide of any one of Embodiments 1C-14C, wherein X₂ is Cys.

Embodiment 16C. The peptide of any one of Embodiments 1C-15C, wherein the peptide of formula (I) is selected from Table C1, Table C1A, Table C2, and Table C2A.

Embodiment 17C. The peptide of any one of Embodiments 1C-16C, wherein the peptide is a cyclic peptide.

Embodiment 18C. The peptide of Embodiment 17C, wherein the cyclic peptide comprises a disulfide bridge or a lactam bridge.

Embodiment 19C. The peptide of Embodiment 17C or 18C, wherein the cyclic peptide has the formula (CII):

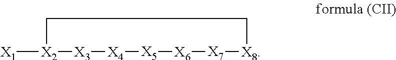

Embodiment 20C. The peptide of any one of Embodiments 1C-19C, wherein the cyclic peptide of formula (CII) is a cyclic peptide of any one of formula (CIIa), formula (CIIb), formula (CIIc), formula (CIId), formula (CIIe), or formula (CIIf).

Embodiment 21C. The peptide of Embodiments 19C or 20C, wherein the cyclic peptide of formula (CII) is a cyclic peptide of formula (CIIa):

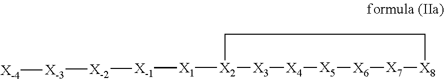

wherein in formula (IIa): X₋₁, X₋₂, X₋₃, X₋₄, X₁, X₂, X₃, X₄, X₅, X₆, X₇, and X₈ are each independently an amino acid selected from Table C1; or Table C1 or a linker.

Embodiment 22C. The peptide of Embodiments 19C or 20C, wherein the cyclic peptide of formula (CII) is a cyclic peptide of formula (CIIb):

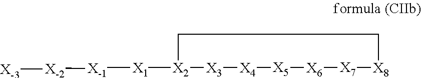

wherein in formula (IIb): X₋₁, X₋₂, X₋₃, X₁, X₂, X₃, X₄, X₅, X₆, X₇, and X₈ are each independently an amino acid selected from Table C1; or Table C1 or a linker.

Embodiment 23C. The peptide of Embodiments 19C or 20C, wherein the cyclic peptide of formula (CII) is a cyclic peptide of formula (CIIc):

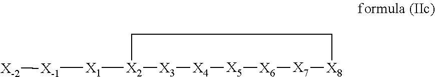

wherein in formula (IIc): X₋₁, X₋₂, X₁, X₂, X₃, X₄, X₅, X₆, X₇, and X₈ are each independently an amino acid selected from Table C1; or Table C1 or a linker.

Embodiment 24C. The peptide of Embodiments 19C or 20C, wherein the cyclic peptide of formula (CII) is a cyclic peptide of formula (CIId):

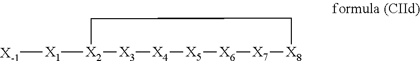

wherein in formula (IId): X₋₁, X₁, X₂, X₃, X₄, X₅, X₆, X₇, and X₈ are each independently an amino acid selected from Table C1; or Table C1 or a linker.

Embodiment 25C. The peptide of Embodiments 19C or 20C, wherein the cyclic peptide of formula (CII) is a cyclic peptide of formula (CIIe):

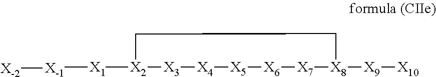

wherein in formula (IIe): X₋₁, X₋₂, X₁, X₂, X₃, X₄, X₅, X₆, X₇, X₈, X₉, and X₁₀ are each independently an amino acid selected from Table C1; or Table C1 or a linker.

Embodiment 26C. The peptide of Embodiments 19C or 20C, wherein the cyclic peptide of formula (CII) is a cyclic peptide of formula (CIIf):

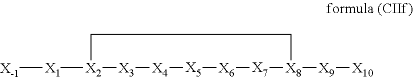

wherein in formula (IIf): X₋₁, X₋₂, X₁, X₂, X₃, X₄, X₅, X₆, X₇, X₈, X₉, and X₁₀ are each independently an amino acid selected from Table C1; or Table C1 or a linker.

Embodiment 27C. The peptide of any one of Embodiments 1C-26C, wherein the peptide further comprises one or more amino acids conjugated to X₁ and/or X₈, optionally wherein the one or more amino acids are selected from D-Arginine (D-Arg), glycine (Gly), and L-Lys(AEEAc-AEEAc-L-γ-Glu-17-carboxyheptadecanoyl) (Lys*).

Embodiment 28C. The peptide of any one of Embodiments 1C-27C, wherein the peptide further comprises one or more lipids conjugated to X₁ and/or X_{8C}.

Embodiment 29C. The peptide of any one of Embodiments 1C-28C, wherein the peptide is capped with N-terminal acetyl and/or C-terminal amide groups.

Embodiment 30C. The peptide of any one of Embodiments 1C-29C, wherein the peptide is selected from Table C1, Table C1A, Table C2, and Table C2A.

Embodiment 31C. The peptide of any one of Embodiments 1C-30C, wherein the peptide demonstrates increased selectivity for MC4R over MC1R when administered to a subject compared to a control.

Embodiment 32C. The peptide of any one of Embodiments 1C-31C, wherein the peptide demonstrates increased selectivity for MC4R over MC1R as measured by an in vitro, ex vivo, or in vivo assay when compared to a control.

Embodiment 33C. The peptide of Embodiment 31C or 32C, wherein the peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 34C. The peptide of Embodiment 31C or 32C, wherein the peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 35C. The peptide of any one of Embodiments 1C-30C, wherein the peptide demonstrates an increased ratio of MC4R intracellular signaling to MC1R intracellular signaling when administered to a subject compared to a control.

Embodiment 36C. The peptide of any one of Embodiments 1C-31C, wherein the peptide demonstrates increased selectivity for MC4R intracellular signaling to MC1R intracellular signaling as measured by an in vitro, ex vivo, or in vivo assay when compared to a control.

Embodiment 37C. The peptide of Embodiment 35C or 36C, wherein the peptide demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when administered to a subject compared to a control.

Embodiment 38C. The peptide of Embodiment 35C or 36C, wherein the peptide demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when administered to a subject compared to a control.

Embodiment 39C. The peptide of any one of Embodiments 1C-38C, wherein the peptide demonstrates enhanced melanocortin 4 receptor (MC4R) function in a subject when compared to before the peptide is administered or to a pre-treatment or non-treatment state, or a subject treated with control.

Embodiment 40C. The peptide of any one of Embodiments 1C-39C, wherein the peptide demonstrates decreased melanocortin 1 receptor (MC1R) function in a subject when compared to before the peptide is administered or to a pre-treatment or non-treatment state, or a subject treated with control.

Embodiment 41C. The peptide of any one of Embodiments 1C-40C, wherein the peptide demonstrates enhanced melanocortin 4 receptor (MC4R) function as measured by an in vitro, ex vivo, or in vivo assay when compared to a control.

Embodiment 42C. The peptide of any one of Embodiments 1C-41C, wherein the peptide demonstrates decreased melanocortin 1 receptor (MC1R) function as measured by an in vitro, ex vivo, or in vivo assay when compared to a control.

Embodiment 43C. The peptide of any one of Embodiments 39C-42C, wherein the peptide demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 44C. The peptide of any one of Embodiments 39C-42C, wherein the peptide demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 45C. The peptide of any one of Embodiments 17C-44C, wherein the cyclic peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 46C. The peptide of any one of Embodiments 17C-44C, wherein the cyclic peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 47C. The peptide of any one of Embodiments 1C-46C, wherein the peptide comprises a half-life extending moiety, optionally wherein the half-life extending moiety comprises polyethylene glycol (PEG), recombinant PEG mimetics, glycosylation of carbohydrates, Fc-fusion proteins or conjugates, albumin fusion proteins or conjugates, albumin-binding fusion proteins or conjugates, polypropylene glycol (PPG), XTEN fusion protein or conjugates, or a combination thereof.

Embodiment 48C. The peptide of any one of Embodiments 1C-47C, wherein the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

Embodiment 49C. The peptide of any one of Embodiments 1C-48C, wherein the peptide further comprises a therapeutic, diagnostic, and/or imaging moiety, optionally wherein the therapeutic, diagnostic, and/or imaging moiety comprises a small molecule, a biological (e.g., a biopolymer, a protein, a nucleic acid, a polysaccharide), or a radionuclide.

Embodiment 50C. The peptide of any one of Embodiments 1C-49C, wherein the peptide of formula (I) is conjugated to or co-formulated with an additional therapeutic agent or therapeutic moiety.

Embodiment 51C. The peptide of Embodiments 49C or 50C, wherein the additional therapeutic agent or therapeutic moiety comprises incretin, an incretin analogue, or a modulator of an incretin receptor.

Embodiment 52C. The peptide of Embodiment 51C, wherein the modulator is an agonist.

Embodiment 53C. The peptide of Embodiment 52C, wherein the agonist is an agonist of GLP-1, GIP, and/or glucagon receptor.

Embodiment 54C. The peptide of Embodiment 52C, wherein the agonist is a GLP-1 analogue.

Embodiment 55C. The peptide of Embodiment 54C, wherein the GLP-1 analogue comprises a non-canonical amino acid.

Embodiment 56C. The peptide of Embodiment 54C, wherein the GLP-1 analogue comprises tirzepatide, tirzepatide analogue, liraglutide, retatrutide, exenatide, lixisenatide, semaglutide or semaglutide derivative.

Embodiment 57C. A protein comprising the peptide of any one of Embodiments 1C to 56C.

Embodiment 58C. The protein of Embodiment 57C, wherein the protein has a size of at least about 10 amino acid residues, or at least about 15 resides, or at least about 20 residues, or at least about 25 residues, or at least about 30 residues, or at least about 35 residues, or at least about 40 residues, or at least about 45 residues, or at least about 50 residues, or at least about 60 residues, or at least about 70 residues, or at least about 80 residues, or at least about 90 residues, or at least about 100 residues, or at least about 250 residues, or at least about 500 residues, at least about 750 residues, at least about 1,000 residues, at least about 1,250 residues, at least about 1,500 residues, at least about 1,750 residues, at least about 2,000 residues, at least about 3,000 residues, at least about 4,000 residues, or at least about 5,000 residues.

Embodiment 59C. A nucleic acid encoding the peptide of any one of Embodiments 1C to 56C, or the protein of Embodiment 57C or 58C.

Embodiment 60C. A solid synthesis device conjugated to the peptide of any one of Embodiments 1C to 56C, the protein of Embodiment 57C or 58C, or the nucleic acid of Embodiment 59C.

Embodiment 61C. A pharmaceutical composition comprising a peptide of any one of Embodiments 1C to 56C, or the protein of Embodiment 57C or 58C, or the nucleic acid of Embodiment 59C, and a pharmaceutically acceptable excipient or carrier.

Embodiment 62C. The pharmaceutical composition of Embodiment 61C, wherein the pharmaceutical composition further comprises an additional therapeutic, diagnostic, and/or imaging moiety.

Embodiment 63C. The pharmaceutical composition of Embodiment 61C or 62C, wherein the pharmaceutical composition is conjugated to or co-formulated with an additional therapeutic agent or therapeutic moiety.

Embodiment 64C. The pharmaceutical composition of any one of Embodiments 61C or 62C, wherein the pharmaceutical composition is administered orally, parenterally, by inhalation spray, topically, rectally, nasally, buccally, sub-lingually, vaginally or via an implanted reservoir.

Embodiment 65C. The pharmaceutical composition of any one of Embodiments 61C or 62C, wherein the pharmaceutical composition is administered via intravenous injection, intramuscular injection, subcutaneous injection, or depot injection.

Embodiment 66C. A method of selectively increasing the activity of MC4R over MC1R comprising administering a peptide of any one of Embodiments 1C to 56C or the pharmaceutical composition of Embodiments 61C to 65C to a subject in need thereof.

Embodiment 67C. The method of Embodiment 66C, wherein the method prevents, reduces, or eliminates hyperpigmentation in the subject when compared to an untreated or pre-treatment subject, or subject treated with a control.

Embodiment 68C. The method of Embodiment 67C, wherein the hyperpigmentation is reduced by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% when compared to an untreated or pre-treatment subject, or subject treated with a control.

Embodiment 69C. The method of Embodiment 67C, wherein the hyperpigmentation is reduced by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold when compared to an untreated or pre-treatment subject, or subject treated with a control.

Embodiment 70C. A method of treating a disease or disorder associated with MC4R function comprising administering a peptide of any one of Embodiments 1C to 56C or the pharmaceutical composition of Embodiments 61C to 65 to a subject in need thereof.

Embodiment 71C. The method of Embodiment 70C, wherein the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency, and Prader-Willi syndrome.

Embodiment 72C. The method of Embodiment 70C or 71C, wherein the method further comprises co-administration of an additional therapeutic agent.

Embodiment 73C. The method of any one of Embodiments 70C to 72C, wherein the additional therapeutic agent comprises incretin, an incretin analogue, or a modulator of an incretin receptor.

Embodiment 74C. The method of Embodiment 73C, wherein the modulator is an agonist.

Embodiment 75C. The method of Embodiment 74C, wherein the agonist is an agonist of GLP-1, GIP, and/or glucagon receptor.

Embodiment 76C. The method of Embodiment 74C, wherein the agonist is a GLP-1 analogue.

Embodiment 77C. The method of Embodiment 76C, wherein the GLP-1 analogue comprises a non-canonical amino acid.

Embodiment 78C. The method of Embodiment 76C, wherein the GLP-1 analogue comprises tirzepatide, tirzepatide analogue, liraglutide, retatrutide, exenatide, lixisenatide, semaglutide or semaglutide derivative.

Embodiment 79C. The method of Embodiment 73C, wherein the subject is receiving and/or previously received treatment comprising incretin, an incretin analogue, or a modulator of an incretin receptor.

Embodiment 80C. The method of Embodiment 73C, wherein the subject has not or is not receiving treatment comprising incretin, an incretin analogue, or a modulator of an incretin receptor.

Embodiment 81C. A method of treating obesity or abnormal or excessive fat accumulation comprising administering a peptide of any one of Embodiments 1C to 56C or the pharmaceutical composition of Embodiments 61C to 65 to a subject in need thereof.

Embodiment 82C. A method of treating a disease or disorder associated with MC4R function comprising administering a peptide of any one of Embodiments 1C to 56C or the pharmaceutical composition of Embodiments 61C to 65 to a subject receiving and/or previously received incretin.

Embodiment 83C. A method of treating a disease or disorder associated with MC4R function comprising administering a peptide of any one of Embodiments 1C to 56C or the pharmaceutical composition of Embodiments 61C to 65 to a subject not receiving incretin.

Embodiment 84C. The method of any one of Embodiments 65C to 83C, wherein the peptide or pharmaceutical composition is administered from a prefilled dosage form device, such as syringe or autoinjector.

### Embodiments—Series D

Embodiment 1D. A peptide comprising the amino acid sequence of formula (DI):

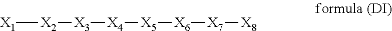

wherein in formula (DI):
X₁, X₂, X₃, X₄, X₅, X₆, X₇, and X₈ are each independently an amino acid selected from Table D1.

Embodiment 2D. The peptide of Embodiment 1D, wherein the peptide of formula (DI) is a peptide of any one of formula (DIa), formula (DIb), formula (DIc), formula (DId), formula (DIe), or formula (DIf).

Embodiment 3D. The peptide of Embodiment 1D or 2D, wherein the peptide of formula (DI) is a peptide of formula (DIa):

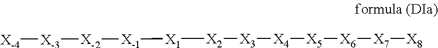

wherein in formula (DIa):
X₋₁, X₋₂, X₋₃, X₋₄, X₁, X₂, X₃, X₄, X₅, X₆, X₇, and X₈ are each independently an amino acid selected from Table D1; or Table D1 or a linker.

Embodiment 4D. The peptide of Embodiment 1D or 2D, wherein the peptide of formula (DI) is a peptide of formula (DIb):

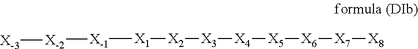

wherein in formula (Ib): X₋₁, X₋₂, X₋₃, X₁, X₂, X₃, X₄, X₅, X₆, X₇, and X₈ are each independently an amino acid selected from Table D1; or Table D1 or a linker.

Embodiment 5D. The peptide of Embodiment 1D or 2D, the peptide of formula (DI) is a peptide of formula (DIc):

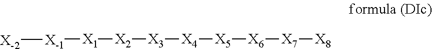

wherein in formula (Ic): X₋₁, X₋₂, X₁, X₂, X₃, X₄, X₅, X₆, X₇, and X₈ are each independently an amino acid selected from Table D1; or Table D1 or a linker.

Embodiment 6D. The peptide of Embodiment 1D or 2D, the peptide of formula (DI) is a peptide of formula (DId):

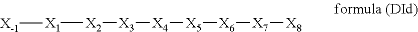

wherein in formula (Id): X₋₁, X₁, X₂, X₃, X₄, X₅, X₆, X₇, and X₈ are each independently an amino acid selected from Table D1; or Table D1 or a linker.

Embodiment 7D. The peptide of Embodiment 1D or 2D, the peptide of formula (DI) is a peptide of formula (DIe):

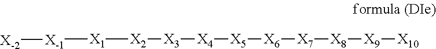

wherein in formula (Ie): X₋₁, X₋₂, X₁, X₂, X₃, X₄, X₅, X₆, X₇, X₈, X₉, and X₁₀ are each independently an amino acid selected from Table D1; or Table D1 or a linker.

Embodiment 8D. The peptide of Embodiment 1D or 2D, the peptide of formula (DI) is a peptide of formula (DIf):

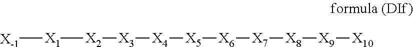

wherein in formula (DIf): X₋₁, X₁, X₂, X₃, X₄, X₅, X₆, X₇, X₈, X₉, and X₁₀ are each independently an amino acid selected from Table D1; or Table D1 or a linker.

Embodiment 9D. The peptide of any one of Embodiments 1D-8D, wherein X₃ is selected from 2-methyl-alanine (Ala(2-Me)), 3-aminooxetane-3-carboxylic acid (Aib(O-cyclic)), alpha-methyl-D-aspartic acid (L-aMeAsp), alpha-methyl-D-ornithine (D-aMeOrn), alpha-methyl-D-serine (D-aMeSer), alpha-methyl-L-glutamic acid (L-aMeGlu), cycloleucine (Cyclo-Leu), beta-D-homoglutamic acid (D-bhGlu), beta-L-homoglutamic acid (bhGlu), L-homoglutamic acid (hGlu), and L-phenylglycine (Phg).

Embodiment 10D. The peptide of any one of Embodiments 1D-9D, wherein X₄ is selected from 3-(3-pyridyl)-L-alanine (3-Pal), glutamine (Gln), homocitrulline (hCit), citrulline (Cit), histidine (His), and L-ornithine (Orn).

Embodiment 11D. The peptide of any one of Embodiments 1D-10D, wherein X₃ is selected from X₃ column in Table D1 and Table D1A and X₄ is selected from X₄ column in Table D1 and Table D1A.

Embodiment 12D. The peptide of any one of Embodiments 1D-10D, wherein X₃-X₄ is selected from Phg-3-Pal, D-aMeOrn-Gln, Aib(O-cyclic)-Gln, Aib(O-cyclic)-hCit, Aib(O-cyclic)-Cit, hGlu-Gln, D-aMeSer-Gln, Cyclo-Leu-Gln, hGlu-His, Ala(2-Me)-Gln, L-aMeGlu-His, L-aMeAsp-His, and Ala(2-Me)-His, Phg-His, Cyclo-Leu-3-Pal, D-bhGlu-His, D-aMeSer-His, bhGlu-His, D-aMeOrn-3-Pal, and Aib(O-cyclic)-3-Pal.

Embodiment 13D. The peptide of any one of Embodiments 1D-12D, wherein X₅ is selected from 4-fluoro-D-phenylalanine (D-Phe(4-F)), D-phenylalanine (D-Phe), 4-methyl-D-phenylalanine (D-Phe(4-Me)), 3-trifluoromethyl-D-phenylalanine (D-Phe(3-CF3)), and 3-fluoro-D-phenylalanine (D-Phe(3-F)).

Embodiment 14D. The peptide of any one of Embodiments 1D-13D, wherein X₆ is arginine (Arg).

Embodiment 15D. The peptide of any one of Embodiments 1D-14D, wherein X₇ is selected from 6-fluoro-L-tryptophan (Trp(6-F)), 6-methyl-L-tryptophan (Trp(6-Me)), tryptophan (Trp), and 5-methyl-L-tryptophan (Trp(5-Me)).

Embodiment 16D. The peptide of any one of Embodiments 1D-15D, wherein X₈ is cysteine (Cys) or penicillamine (Pen).

Embodiment 17D. The peptide of any one of Embodiments 1D-16D, wherein X₁ is selected from D-norarginine (D-Nar), Arg, and beta-homo-L-arginine (Beta-homoArg).

Embodiment 18D. The peptide of any one of Embodiments 1D-17D, wherein X₂ is Cys.

Embodiment 19D. The peptide of any one of Embodiments 1D-18D, wherein the peptide of formula (I) is selected from Table D1, Table D1A, Table D2, and Table D2A.

Embodiment 20D. The peptide of any one of Embodiments 1D-19D, wherein the peptide is a cyclic peptide.

Embodiment 21D. The peptide of Embodiment 20D, wherein the cyclic peptide comprises a disulfide bridge or a lactam bridge.

Embodiment 22D. The peptide of Embodiment 20D or 21D, wherein the cyclic peptide has the formula (DII):

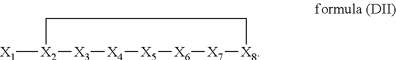

Embodiment 23D. The peptide of any one of Embodiments 1D-22D, wherein the cyclic peptide of formula (DII) is a cyclic peptide of any one of formula (DIIa), formula (DIIb), formula (DIIc), formula (DIId), formula (IIe), or formula (DIIf).

Embodiment 24D. The peptide of Embodiments 22D or 23D, wherein the cyclic peptide of formula (DII) is a cyclic peptide of formula (DIIa):

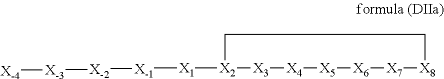

wherein in formula (DIIa): X₋₁, X₋₂, X₋₃, X₋₄, X₁, X₂, X₃, X₄, X₅, X₆, X₇, and X₈ are each independently an amino acid selected from Table D1; or Table D1 or a linker.

Embodiment 25D. The peptide of Embodiments 22D or 23D, wherein the cyclic peptide of formula (DII) is a cyclic peptide of formula (DIIb):

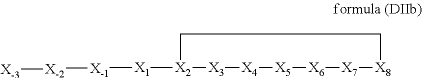

wherein in formula (DIIb): X₋₁, X₋₂, X₋₃, X₁, X₂, X₃, X₄, X₅, X₆, X₇, and X₈ are each independently an amino acid selected from Table D1; or Table D1 or a linker.

Embodiment 26D. The peptide of Embodiments 22D or 23D, wherein the cyclic peptide of formula (DII) is a cyclic peptide of formula (DIIc):

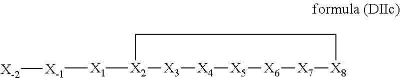

wherein in formula (DIIc): X₋₁, X₋₂, X₁, X₂, X₃, X₄, X₅, X₆, X₇, and X₈ are each independently an amino acid selected from Table D1; or Table D1 or a linker.

Embodiment 27D. The peptide of Embodiments 22D or 23D, wherein the cyclic peptide of formula (II) is a cyclic peptide of formula (DIId):

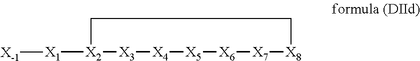

wherein in formula (DIId): X₋₁, X₁, X₂, X₃, X₄, X₅, X₆, X₇, and X₈ are each independently an amino acid selected from Table D1; or Table D1 or a linker.

Embodiment 28D. The peptide of Embodiments 22D or 23D, wherein the cyclic peptide of formula (II) is a cyclic peptide of formula (IIe):

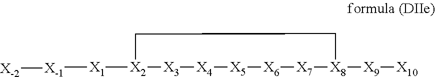

wherein in formula (DIIe): X₋₁, X₋₂, X₁, X₂, X₃, X₄, X₅, X₆, X₇, X₈, X₉, and X₁₀ are each independently an amino acid selected from Table D1; or Table D1 or a linker.

Embodiment 29D. The peptide of Embodiments 22D or 23D, wherein the cyclic peptide of formula (DII) is a cyclic peptide of formula (DIIf):

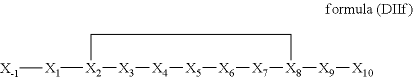

wherein in formula (DIIf): X₋₁, X₋₂, X₁, X₂, X₃, X₄, X₅, X₆, X₇, X₈, X₉, and X₁₀ are each independently an amino acid selected from Table D1; or Table D1 or a linker.

Embodiment 30D. The peptide of any one of Embodiments 1D-29D, wherein the peptide further comprises one or more amino acids conjugated to X₁ and/or X₈, optionally wherein the one or more amino acids are selected from D-arginine (D-Arg), glycine (Gly), and L-Lys(AEEAc-AEEAc-L-γ-Glu-17-carboxyheptadecanoyl) (Lys*).

Embodiment 31. The peptide of any one of Embodiments 1D-30D, wherein the peptide further comprises one or more lipids conjugated to X₁ and/or X₈.

Embodiment 32D. The peptide of any one of Embodiments 1D-31D, wherein the peptide is capped with N-terminal acetyl and/or C-terminal amide groups.

Embodiment 33D. The peptide of any one of Embodiments 1D-32D, wherein the peptide is selected from Table D1, Table D1A, Table D2, and Table 2.

Embodiment 34D. The peptide of any one of Embodiments 1D-33D, wherein the peptide demonstrates increased selectivity for MC4R over MC1R when administered to a subject compared to a control.

Embodiment 35D. The peptide of any one of Embodiments 1D-34D, wherein the peptide demonstrates increased selectivity for MC4R over MC1R as measured by an in vitro, ex vivo, or in vivo assay when compared to a control.

Embodiment 36D. The peptide of Embodiment 34D or 35D, wherein the peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 37D. The peptide of Embodiment 34D or 35D, wherein the peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 38D. The peptide of any one of Embodiments 1D-33D, wherein the peptide demonstrates an increased ratio of MC4R intracellular signaling to MC1R intracellular signaling when administered to a subject compared to a control.

Embodiment 39D. The peptide of any one of Embodiments 1D-34D, wherein the peptide demonstrates increased selectivity for MC4R intracellular signaling to MC1R intracellular signaling as measured by an in vitro, ex vivo, or in vivo assay when compared to a control.

Embodiment 40D. The peptide of Embodiment 38D or 39D, wherein the peptide demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when administered to a subject compared to a control.

Embodiment 41D. The peptide of Embodiment 38D or 39D, wherein the peptide demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when administered to a subject compared to a control.

Embodiment 42D. The peptide of any one of Embodiments 1D-41D, wherein the peptide demonstrates enhanced melanocortin 4 receptor (MC4R) function in a subject when compared to before the peptide is administered or to a pre-treatment or non-treatment state, or a subject treated with control.

Embodiment 43D. The peptide of any one of Embodiments 1D-42D, wherein the peptide demonstrates decreased melanocortin 1 receptor (MC1R) function in a subject when compared to before the peptide is administered or to a pre-treatment or non-treatment state, or a subject treated with control.

Embodiment 44D. The peptide of any one of Embodiments 1D-43D, wherein the peptide demonstrates enhanced melanocortin 4 receptor (MC4R) function as measured by an in vitro, ex vivo, or in vivo assay when compared to a control.

Embodiment 45D. The peptide of any one of Embodiments 1D-44D, wherein the peptide demonstrates decreased melanocortin 1 receptor (MC1R) function as measured by an in vitro, ex vivo, or in vivo assay when compared to a control.

Embodiment 46D. The peptide of any one of Embodiments 42D-45D, wherein the peptide demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 47D. The peptide of any one of Embodiments 42D-45D, wherein the peptide demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 48D. The peptide of any one of Embodiments 20D-47D, wherein the cyclic peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 49D. The peptide of any one of Embodiments 20D-47D, wherein the cyclic peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 50D. The peptide of any one of Embodiments 1D-49D, wherein the peptide comprises a half-life extending moiety, optionally wherein the half-life extending moiety comprises polyethylene glycol (PEG), recombinant PEG mimetics, glycosylation of carbohydrates, Fc-fusion proteins or conjugates, albumin fusion proteins or conjugates, albumin-binding fusion proteins or conjugates, polypropylene glycol (PPG), XTEN fusion protein or conjugates, or a combination thereof.

Embodiment 51D. The peptide of any one of Embodiments 1D-50D, wherein the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

Embodiment 52D. The peptide of any one of Embodiments 1D-51D, wherein the peptide further comprises a therapeutic, diagnostic, and/or imaging moiety, optionally wherein the therapeutic, diagnostic, and/or imaging moiety comprises a small molecule, a biological (e.g., a biopolymer, a protein, a nucleic acid, a polysaccharide), or a radionuclide.

Embodiment 53D. The peptide of any one of Embodiments 1D-52D, wherein the peptide of formula (I) is conjugated to or co-formulated with an additional therapeutic agent or therapeutic moiety.

Embodiment 54D. The peptide of Embodiments 52D or 53D, wherein the additional therapeutic agent or therapeutic moiety comprises incretin, an incretin analogue, or a modulator of an incretin receptor.

Embodiment 55D. The peptide of Embodiment 54D, wherein the modulator is an agonist.

Embodiment 56D. The peptide of Embodiment 55D, wherein the agonist is an agonist of GLP-1, GIP, and/or glucagon receptor.

Embodiment 57D. The peptide of Embodiment 55D, wherein the agonist is a GLP-1 analogue.

Embodiment 58D. The peptide of Embodiment 57D, wherein the GLP-1 analogue comprises a non-canonical amino acid.

Embodiment 59D. The peptide of Embodiment 57D, wherein the GLP-1 analogue comprises tirzepatide, tirzepatide analogue, liraglutide, retatrutide, exenatide, lixisenatide, semaglutide or semaglutide derivative.

Embodiment 60D. A protein comprising the peptide of any one of Embodiments 1D to 59D.

Embodiment 61D. The protein of Embodiment 60D, wherein the protein has a size of at least about 10 amino acid residues, or at least about 15 resides, or at least about 20 residues, or at least about 25 residues, or at least about 30 residues, or at least about 35 residues, or at least about 40 residues, or at least about 45 residues, or at least about 50 residues, or at least about 60 residues, or at least about 70 residues, or at least about 80 residues, or at least about 90 residues, or at least about 100 residues, or at least about 250 residues, or at least about 500 residues, at least about 750 residues, at least about 1,000 residues, at least about 1,250 residues, at least about 1,500 residues, at least about 1,750 residues, at least about 2,000 residues, at least about 3,000 residues, at least about 4,000 residues, or at least about 5,000 residues.

Embodiment 62D. A nucleic acid encoding the peptide of any one of Embodiments 1D to 59D, or the protein of Embodiment 60D or 61D.

Embodiment 63D. A solid synthesis device conjugated to the peptide of any one of Embodiments 1D to 59D, the protein of Embodiment 60D or 61D, or the nucleic acid of Embodiment 62D.

Embodiment 64D. A pharmaceutical composition comprising a peptide of any one of Embodiments 1D to 59D, or the protein of Embodiment 60D or 61D, or the nucleic acid of Embodiment 62D, and a pharmaceutically acceptable excipient or carrier.

Embodiment 65D. The pharmaceutical composition of Embodiment 64D, wherein the pharmaceutical composition further comprises an additional therapeutic, diagnostic, and/or imaging moiety.

Embodiment 66D. The pharmaceutical composition of Embodiment 64D or 65D, wherein the pharmaceutical composition is conjugated to or co-formulated with an additional therapeutic agent or therapeutic moiety.

Embodiment 67D. The pharmaceutical composition of any one of Embodiments 64D or 65D, wherein the pharmaceutical composition is administered orally, parenterally, by inhalation spray, topically, rectally, nasally, buccally, sub-lingually, vaginally or via an implanted reservoir.

Embodiment 68D. The pharmaceutical composition of any one of Embodiments 64D or 65D, wherein the pharmaceutical composition is administered via intravenous injection, intramuscular injection, subcutaneous injection, or depot injection.

Embodiment 69D. A method of selectively increasing the activity of MC4R over MC1R comprising administering a peptide of any one of Embodiments 1D to 59D or the pharmaceutical composition of Embodiments 64D to 68D to a subject in need thereof.

Embodiment 70D. The method of Embodiment 69D, wherein the method prevents, reduces, or eliminates hyperpigmentation in the subject when compared to an untreated or pre-treatment subject, or subject treated with a control.

Embodiment 71. The method of Embodiment 70D, wherein the hyperpigmentation is reduced by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% when compared to an untreated or pre-treatment subject, or subject treated with a control.

Embodiment 72D. The method of Embodiment 70D, wherein the hyperpigmentation is reduced by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold when compared to an untreated or pre-treatment subject, or subject treated with a control.

Embodiment 73D. A method of treating a disease or disorder associated with MC4R function comprising administering a peptide of any one of Embodiments 1D to 59D or the pharmaceutical composition of Embodiments 64D to 68D to a subject in need thereof.

Embodiment 74D. The method of Embodiment 73D, wherein the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency, and Prader-Willi syndrome.

Embodiment 75D. The method of Embodiment 73D or 74D, wherein the method further comprises co-administration of an additional therapeutic agent.

Embodiment 76D. The method of any one of Embodiments 73D-75D, wherein the additional therapeutic agent comprises incretin, an incretin analogue, or a modulator of an incretin receptor.

Embodiment 77D. The method of Embodiment 76D, wherein the modulator is an agonist.

Embodiment 78D. The method of Embodiment 77D, wherein the agonist is an agonist of GLP-1, GIP, and/or glucagon receptor.

Embodiment 79D. The method of Embodiment 77D, wherein the agonist is a GLP-1 analogue.

Embodiment 80D. The method of Embodiment 79D, wherein the GLP-1 analogue comprises a non-canonical amino acid.

Embodiment 81D. The method of Embodiment 79D, wherein the GLP-1 analogue comprises tirzepatide, tirzepatide analogue, liraglutide, retatrutide, exenatide, lixisenatide, semaglutide or semaglutide derivative.

Embodiment 82D. The method of Embodiment 76D, wherein the subject is receiving and/or previously received treatment comprising incretin, an incretin analogue, or a modulator of an incretin receptor.

Embodiment 83D. The method of Embodiment 76D, wherein the subject has not or is not receiving treatment comprising incretin, an incretin analogue, or a modulator of an incretin receptor.

Embodiment 84D. A method of treating obesity or abnormal or excessive fat accumulation comprising administering a peptide of any one of Embodiments 1D to 59D or the pharmaceutical composition of Embodiments 64D to 68D to a subject in need thereof.

Embodiment 85D. A method of treating a disease or disorder associated with MC4R function comprising administering a peptide of any one of Embodiments 1D to 59D or the pharmaceutical composition of Embodiments 64D to 68D to a subject receiving and/or previously received incretin.

Embodiment 86D. A method of treating a disease or disorder associated with MC4R function comprising administering a peptide of any one of Embodiments 1D to 59D or the pharmaceutical composition of Embodiments 64D to 68D to a subject not receiving incretin.

Embodiment 87D. The method of any one of Embodiments 69D to 86D, wherein the peptide or pharmaceutical composition is administered from a prefilled dosage form device, such as syringe or autoinjector.

### Embodiments—Series E

Embodiment 1E. A peptide comprising the amino acid sequence of formula (EI):

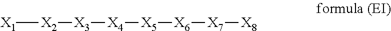

wherein in formula (EI):
X₁, X₂, X₃, X₄, X₅, X₆, X₇, and X₈ are each independently an amino acid selected from Table E1.

Embodiment 2E. The peptide of Embodiment 1E, wherein the peptide of formula (EI) is a peptide of any one of formula (EIa), formula (EIb), formula (EIc), formula (EId), formula (EIe), or formula (EIf).

Embodiment 3E. The peptide of Embodiment 1E or 2E, wherein the peptide of formula (EI) is a peptide of formula (EIa):

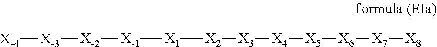

wherein in formula (EIa):
X₋₁, X₋₂, X₋₃, X₋₄, X₁, X₂, X₃, X₄, X₅, X₆, X₇, and X₈ are each independently an amino acid selected from Table E1; or Table E1 or a linker.

Embodiment 4E. The peptide of Embodiment 1E or 2E, wherein the peptide of formula (EI) is a peptide of formula (EIb):

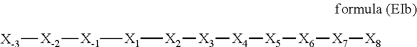

wherein in formula (Ib): X₋₁, X₋₂, X₋₃, X₁, X₂, X₃, X₄, X₅, X₆, X₇, and X₈ are each independently an amino acid selected from Table E1; or Table E1 or a linker.

Embodiment 5E. The peptide of Embodiment 1E or 2E, the peptide of formula (EI) is a peptide of formula (EIc):

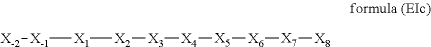

wherein in formula (Ic): X₋₁, X₋₂, X₁, X₂, X₃, X₄, X₅, X₆, X₇, and X₈ are each independently an amino acid selected from Table E1; or Table E1 or a linker.

Embodiment 6E. The peptide of Embodiment 1E or 2E, the peptide of formula (EI) is a peptide of formula (EId):

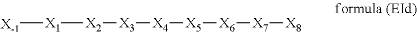

wherein in formula (Id): X₋₁, X₁, X₂, X₃, X₄, X₅, X₆, X₇, and X₈ are each independently an amino acid selected from Table E1; or Table E1 or a linker.

Embodiment 7E. The peptide of Embodiment 1E or 2E, the peptide of formula (EI) is a peptide of formula (Ie):

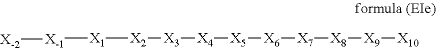

wherein in formula (Ie): X₋₁, X₋₂, X₁, X₂, X₃, X₄, X₅, X₆, X₇, X₈, X₉, and X₁₀ are each independently an amino acid selected from Table E1; or Table E1 or a linker.

Embodiment 8E. The peptide of Embodiment 1E or 2E, the peptide of formula (EI) is a peptide of formula (If):

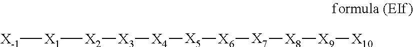

wherein in formula (EIf): X₋₁, X₁, X₂, X₃, X₄, X₅, X₆, X₇, X₈, X₉, and X₁₀ are each independently an amino acid selected from Table E1; or Table E1 or a linker.

Embodiment 9E. The peptide of any one of Embodiments 1E-8E, wherein X₃ is selected from 2-methyl-alanine (Ala(2-Me)), 3-aminooxetane-3-carboxylic acid (Aib(O-cyclic)), alpha-methyl-D-aspartic acid (L-aMeAsp), alpha-methyl-D-ornithine (D-aMeOrn), alpha-methyl-D-serine (D-aMeSer), alpha-methyl-L-glutamic acid (L-aMeGlu), cycloleucine (Cyclo-Leu), L-homoglutamic acid (hGlu), L-phenylglycine (Phg), beta-L-homoglutamic acid (bhGlu), D-alanine (D-ala), D-2,4-diaminobutyric acid (D-Dab), and L-homoglutamic acid (hGlu).

Embodiment 10E. The peptide of any one of Embodiments 1E-9E, wherein X₄ is selected from 3-(3-Pyridyl)-L-alanine (3-Pal), 3-(4-pyridyl)-L-alanine (4-Pal), glutamine (Gln), homocitrulline (hCit), citrulline (Cit), histidine (His), L-homoglutamine (hGln) and L-ornithine (Orn).

Embodiment 11E. The peptide of any one of Embodiments 1E-10E, wherein X₃ is selected from X₃ column in Table E1, and Table E1A and X₄ is selected from X₄ column in Table E1, and Table E1A.

Embodiment 12E. The peptide of any one of Embodiments 1E-11E, wherein X₅ is selected from 4-fluoro-D-phenylalanine (D-Phe(4-F)), D-phenylalanine (D-Phe), 4-methyl-D-phenylalanine (D-Phe(4-Me)), 3-trifluoromethyl-D-phenylalanine (D-Phe(3-CF3)), 3-fluoro-D-phenylalanine (D-Phe(3-F)), 2,3-difluoro-D-phenylalanine (D-Phe(2,3-diF)), 2,4,5-trifluoro-D-phenylalanine (D-Phe(2,4,5-triF)), 2,4-dichloro-D-phenylalanine (D-Phe(2,4-diCl)), 2,4-difluoro-D-phenylalanine (D-Phe(2,4-diF)), 4-Chloro-2-fluoro-D-phenylalanine (D-Phe(2-F,4-Cl)), 3,4,5-trifluoro-D-phenylalanine (D-Phe(3,4,5-triF)), 3,4-difluoro-D-phenylalanine (D-Phe(3,4-diF)), 3,4-dimethyl-D-phenylalanine (D-Phe(3,4-diMe)), 3-chloro-D-phenylalanine (D-Phe(3-Cl)), 4-methyl-3-fluoro-D-phenylalanine (D-Phe(3-F,4-Me)), 3-methyl-D-phenylalanine (D-Phe(3-Me)), 4-(trifluoromethyl)-D-phenylalanine (D-Phe(4-CF3)), and 4-chloro-D-phenylalanine (D-Phe(4-Cl)).

Embodiment 13E. The peptide of any one of Embodiments 1E-12E, wherein X₆ is Arginine (Arg).

Embodiment 14E. The peptide of any one of Embodiments 1E-13E, wherein X₇ is selected from 4-fluoro-L-tryptophan (Trp(4-F)), 5-chloro-L-tryptophan (Trp(5-Cl)), 5-fluoro-L-tryptophan (Trp(5-F)), 5-methyl-L-tryptophan (Trp(5-Me)), 6-bromo-L-tryptophan (Trp(6-Br)), 6-(trifluoromethyl)-L-tryptophan (Trp(6-CF3)), 6-chloro-L-tryptophan (Trp(6-Cl)), 6-fluoro-L-tryptophan (Trp(6-F)), 6-methyl-L-tryptophan (Trp(6-Me)), 7-fluoro-L-tryptophan (Trp(7-F)), and tryptophan (Trp).

Embodiment 15E. The peptide of any one of Embodiments 1E-14E, wherein X₈ is selected from cysteine (Cys), 3-amino-L-alanine (Dap), and penicillamine (Pen).

Embodiment 16E. The peptide of any one of Embodiments 1E-15E, wherein X₁ is selected from Arg, beta-homo-L-arginine (Beta-homoArg), D-arginine (D-Arg), D-norarginine (D-Nar), homo-L-arginine (L-hArg), and L-norarginine (Nar).

Embodiment 17E. The peptide of any one of Embodiments 1E-16E, wherein X₂ is selected from aspartic acid (Asp), Cys, and L-glutamate (Glu).

Embodiment 18E. The peptide of any one of Embodiments 1E-17E, wherein the peptide of formula (I) is selected from Table E1, Table E1A, Table E2 and Table E2A.

Embodiment 19E. The peptide of any one of Embodiments 1E-18E, wherein the peptide is a cyclic peptide.

Embodiment 20E. The peptide of Embodiment 19E, wherein the cyclic peptide comprises a disulfide bridge or a lactam bridge.

Embodiment 21. The peptide of Embodiment 19E or 20E, wherein the cyclic peptide has the formula (EII):

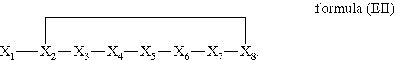

Embodiment 22E. The peptide of any one of Embodiments 1E-21E, wherein the cyclic peptide of formula (EII) is a cyclic peptide of any one of formula (EIIa), formula (EIIb), formula (EIIc), formula (EIId), formula (EIIe), or formula (EIIf).

Embodiment 23E. The peptide of Embodiments 21E or 22E, wherein the cyclic peptide of formula (EII) is a cyclic peptide of formula (EIIa):

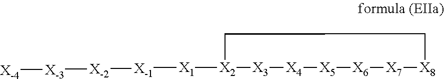

wherein in formula (EIIa): X₋₁, X₋₂, X₋₃, X₋₄, X₁, X₂, X₃, X₄, X₅, X₆, X₇, and X₈ are each independently an amino acid selected from Table E1; or Table E1 or a linker.

Embodiment 24E. The peptide of Embodiments 21E or 22E, wherein the cyclic peptide of formula (EII) is a cyclic peptide of formula (EIIb):

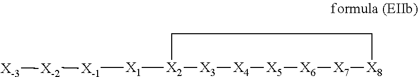

wherein in formula (EIIb): X₋₁, X₋₂, X₋₃, X₁, X₂, X₃, X₄, X₅, X₆, X₇, and X₈ are each independently an amino acid selected from Table E1; or Table E1 or a linker.

Embodiment 25E. The peptide of Embodiments 21E or 22E, wherein the cyclic peptide of formula (EII) is a cyclic peptide of formula (EIIc):

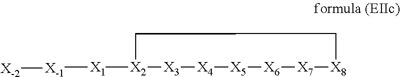

wherein in formula (EIIc): X₋₁, X₋₂, X₁, X₂, X₃, X₄, X₅, X₆, X₇, and X₈ are each independently an amino acid selected from Table E1; or Table E1 or a linker.

Embodiment 26E. The peptide of Embodiments 21E or 22E, wherein the cyclic peptide of formula (EII) is a cyclic peptide of formula (EIId):

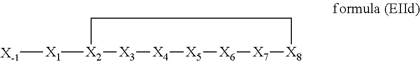

wherein in formula (IId): X₋₁, X₁, X₂, X₃, X₄, X₅, X₆, X₇, and X₈ are each independently an amino acid selected from Table E1; or Table E1 or a linker.

Embodiment 27E. The peptide of Embodiments 21E or 22E, wherein the cyclic peptide of formula (EII) is a cyclic peptide of formula (EIIe):

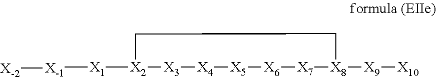

wherein in formula (EIIe): X₋₁, X₋₂, X₁, X₂, X₃, X₄, X₅, X₆, X₇, X₈, X₉, and X₁₀ are each independently an amino acid selected from Table E1; or Table E1 or a linker.

Embodiment 28E. The peptide of Embodiments 21E or 22E, wherein the cyclic peptide of formula (EII) is a cyclic peptide of formula (EIIf):

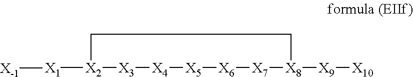

wherein in formula (EIIf): X₋₁, X₋₂, X₁, X₂, X₃, X₄, X₅, X₆, X₇, X₈, X₉, and X₁₀ are each independently an amino acid selected from Table E1; or Table E1 or a linker.

Embodiment 29E. The peptide of any one of Embodiments 1E-28E, wherein the peptide further comprises one or more amino acids conjugated to X₁ and/or X₈, optionally wherein the one or more amino acids are selected from D-Arginine (D-Arg), glycine (Gly), and L-Lys(AEEAc-AEEAc-L-γ-Glu-17-carboxyheptadecanoyl) (Lys*).

Embodiment 30E. The peptide of any one of Embodiments 1E-29E, wherein the peptide further comprises one or more lipids conjugated to X₁ and/or X₈.

Embodiment 31E. The peptide of any one of Embodiments 1E-30E, wherein the peptide is capped with N-terminal acetyl and/or C-terminal amide groups.

Embodiment 32E. The peptide of any one of Embodiments 1E-31E, wherein the peptide is selected from Table E1, Table E1A, Table E2 and Table E2A.

Embodiment 33E. The peptide of any one of Embodiments 1E-32E, wherein the peptide demonstrates increased selectivity for MC4R over MC1R when administered to a subject compared to a control.

Embodiment 34E. The peptide of any one of Embodiments 1E-33E, wherein the peptide demonstrates increased selectivity for MC4R over MC1R as measured by an in vitro, ex vivo, or in vivo assay when compared to a control.

Embodiment 35E. The peptide of Embodiment 33E or 34E, wherein the peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 36E. The peptide of Embodiment 33E or 34E, wherein the peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 37E. The peptide of any one of Embodiments 1E-32E, wherein the peptide demonstrates an increased ratio of MC4R intracellular signaling to MC1R intracellular signaling when administered to a subject compared to a control.

Embodiment 38E. The peptide of any one of Embodiments 1E-33E, wherein the peptide demonstrates increased selectivity for MC4R intracellular signaling to MC1R intracellular signaling as measured by an in vitro, ex vivo, or in vivo assay when compared to a control.

Embodiment 39E. The peptide of Embodiment 37E or 38E, wherein the peptide demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when administered to a subject compared to a control.

Embodiment 40E. The peptide of Embodiment 37E or 38E, wherein the peptide demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when administered to a subject compared to a control.

Embodiment 41E. The peptide of any one of Embodiments 1E-40E, wherein the peptide demonstrates enhanced melanocortin 4 receptor (MC4R) function in a subject when compared to before the peptide is administered or to a pre-treatment or non-treatment state, or a subject treated with control.

Embodiment 42E. The peptide of any one of Embodiments 1E-41E, wherein the peptide demonstrates decreased melanocortin 1 receptor (MC1R) function in a subject when compared to before the peptide is administered or to a pre-treatment or non-treatment state, or a subject treated with control.

Embodiment 43E. The peptide of any one of Embodiments 1E-42E, wherein the peptide demonstrates enhanced melanocortin 4 receptor (MC4R) function as measured by an in vitro, ex vivo, or in vivo assay when compared to a control.

Embodiment 44E. The peptide of any one of Embodiments 1E-43E, wherein the peptide demonstrates decreased melanocortin 1 receptor (MC1R) function as measured by an in vitro, ex vivo, or in vivo assay when compared to a control.

Embodiment 45E. The peptide of any one of Embodiments 41E-44E, wherein the peptide demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 46E. The peptide of any one of Embodiments 41E-44E, wherein the peptide demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 47E. The peptide of any one of Embodiments 19E-46E, wherein the cyclic peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 48E. The peptide of any one of Embodiments 19E-46E, wherein the cyclic peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 49E. The peptide of any one of Embodiments 1E-48E, wherein the peptide comprises a half-life extending moiety, optionally wherein the half-life extending moiety comprises polyethylene glycol (PEG), recombinant PEG mimetics, glycosylation of carbohydrates, Fc-fusion proteins or conjugates, albumin fusion proteins or conjugates, albumin-binding fusion proteins or conjugates, polypropylene glycol (PPG), XTEN fusion protein or conjugates, or a combination thereof.

Embodiment 50E. The peptide of any one of Embodiments 1E-49E, wherein the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

Embodiment 51E. The peptide of any one of Embodiments 1E-50E, wherein the peptide further comprises a therapeutic, diagnostic, and/or imaging moiety, optionally wherein the therapeutic, diagnostic, and/or imaging moiety comprises a small molecule, a biological (e.g., a biopolymer, a protein, a nucleic acid, a polysaccharide), or a radionuclide.

Embodiment 52E. The peptide of any one of Embodiments 1E-51E, wherein the peptide of formula (I) is conjugated to or co-formulated with an additional therapeutic agent or therapeutic moiety.

Embodiment 53E. The peptide of Embodiments 51E or 52E, wherein the additional therapeutic agent or therapeutic moiety comprises incretin, an incretin analogue, or a modulator of an incretin receptor.

Embodiment 54E. The peptide of Embodiment 53E, wherein the modulator is an agonist.

Embodiment 55E. The peptide of Embodiment 54E, wherein the agonist is an agonist of GLP-1, GIP, and/or glucagon receptor.

Embodiment 56E. The peptide of Embodiment 54E, wherein the agonist is a GLP-1 analogue.

Embodiment 57E. The peptide of Embodiment 56E, wherein the GLP-1 analogue comprises a non-canonical amino acid.

Embodiment 58E. The peptide of Embodiment 56E, wherein the GLP-1 analogue comprises tirzepatide, tirzepatide analogue, liraglutide, retatrutide, exenatide, lixisenatide, semaglutide or semaglutide derivative.

Embodiment 59E. A protein comprising the peptide of any one of Embodiments 1E to 58E.

Embodiment 60E. The protein of Embodiment 59E, wherein the protein has a size of at least about 10 amino acid residues, or at least about 15 resides, or at least about 20 residues, or at least about 25 residues, or at least about 30 residues, or at least about 35 residues, or at least about 40 residues, or at least about 45 residues, or at least about 50 residues, or at least about 60 residues, or at least about 70 residues, or at least about 80 residues, or at least about 90 residues, or at least about 100 residues, or at least about 250 residues, or at least about 500 residues, at least about 750 residues, at least about 1,000 residues, at least about 1,250 residues, at least about 1,500 residues, at least about 1,750 residues, at least about 2,000 residues, at least about 3,000 residues, at least about 4,000 residues, or at least about 5,000 residues.

Embodiment 61E. A nucleic acid encoding the peptide of any one of Embodiments 1E to 58E, or the protein of Embodiment 59E or 60E.

Embodiment 62E. A solid synthesis device conjugated to the peptide of any one of Embodiments 1E to 58E, the protein of Embodiment 59E or 60E, or the nucleic acid of Embodiment 61E.

Embodiment 63E. A pharmaceutical composition comprising a peptide of any one of Embodiments 1E to 58E, or the protein of Embodiment 59E or 60E, or the nucleic acid of Embodiment 61E, and a pharmaceutically acceptable excipient or carrier.

Embodiment 64E. The pharmaceutical composition of Embodiment 63E, wherein the pharmaceutical composition further comprises an additional therapeutic, diagnostic, and/or imaging moiety.

Embodiment 65E. The pharmaceutical composition of Embodiment 63E or 64E, wherein the pharmaceutical composition is conjugated to or co-formulated with an additional therapeutic agent or therapeutic moiety.

Embodiment 66E. The pharmaceutical composition of any one of Embodiments 63E or 64E, wherein the pharmaceutical composition is administered orally, parenterally, by inhalation spray, topically, rectally, nasally, buccally, sub-lingually, vaginally or via an implanted reservoir.

Embodiment 67E. The pharmaceutical composition of any one of Embodiments 63E or 64E, wherein the pharmaceutical composition is administered via intravenous injection, intramuscular injection, subcutaneous injection, or depot injection.

Embodiment 68E. A method of selectively increasing the activity of MC4R over MC1R comprising administering a peptide of any one of Embodiments 1E to 58E or the pharmaceutical composition of Embodiments 63E to 67E to a subject in need thereof.

Embodiment 69E. The method of Embodiment 68E, wherein the method prevents, reduces, or eliminates hyperpigmentation in the subject when compared to an untreated or pre-treatment subject, or subject treated with a control.

Embodiment 70E. The method of Embodiment 69E, wherein the hyperpigmentation is reduced by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% when compared to an untreated or pre-treatment subject, or subject treated with a control.

Embodiment 71E. The method of Embodiment 69E, wherein the hyperpigmentation is reduced by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold when compared to an untreated or pre-treatment subject, or subject treated with a control.

Embodiment 72E. A method of treating a disease or disorder associated with MC4R function comprising administering a peptide of any one of Embodiments 1E to 58E or the pharmaceutical composition of Embodiments 63E to 67E to a subject in need thereof.

Embodiment 73E. The method of Embodiment 72E, wherein the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency, and Prader-Willi syndrome.

Embodiment 74E. The method of Embodiment 72E or 73E, wherein the method further comprises co-administration of an additional therapeutic agent.

Embodiment 75E. The method of any one of Embodiments 72E to 74E, wherein the additional therapeutic agent comprises incretin, an incretin analogue, or a modulator of an incretin receptor.

Embodiment 76E. The method of Embodiment 75E, wherein the modulator is an agonist.

Embodiment 77E. The method of Embodiment 76E, wherein the agonist is an agonist of GLP-1, GIP, and/or glucagon receptor.

Embodiment 78E. The method of Embodiment 76E, wherein the agonist is a GLP-1 analogue.

Embodiment 79E. The method of Embodiment 78E, wherein the GLP-1 analogue comprises a non-canonical amino acid.

Embodiment 80E. The method of Embodiment 78E, wherein the GLP-1 analogue comprises tirzepatide, tirzepatide analogue, liraglutide, retatrutide, exenatide, lixisenatide, semaglutide or semaglutide derivative.

Embodiment 81E. The method of Embodiment 75E, wherein the subject is receiving and/or previously received treatment comprising incretin, an incretin analogue, or a modulator of an incretin receptor.

Embodiment 82E. The method of Embodiment 75E, wherein the subject has not or is not receiving treatment comprising incretin, an incretin analogue, or a modulator of an incretin receptor.

Embodiment 83E. A method of treating obesity or abnormal or excessive fat accumulation comprising administering a peptide of any one of Embodiments 1E to 58E or the pharmaceutical composition of Embodiments 63E to 67E to a subject in need thereof.

Embodiment 84E. A method of treating a disease or disorder associated with MC4R function comprising administering a peptide of any one of Embodiments 1E to 58E or the pharmaceutical composition of Embodiments 63E to 67E to a subject receiving and/or previously received incretin.

Embodiment 85E. A method of treating a disease or disorder associated with MC4R function comprising administering a peptide of any one of Embodiments 1E to 58E or the pharmaceutical composition of Embodiments 63E to 67E to a subject not receiving incretin.

Embodiment 86E. The method of any one of Embodiments 68E to 85E, wherein the peptide or pharmaceutical composition is administered from a prefilled dosage form device, such as syringe or autoinjector.

### Embodiments—Series F

Embodiment 1F. A peptide comprising the amino acid sequence of formula (FI):

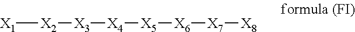

wherein in formula (FI):
X₃ is Cycloleucine (Cyclo-Leu);
X₄ is 3-(3-Pyridyl)-L-alanine (3Pal); and
X₁, X₂, X₅, X₆, X₇, and X₈ are each independently a canonical or non-canonical amino acid.

Embodiment 2F. The peptide of Embodiment 1, wherein the peptide of formula (FI) is a peptide of any one of formula (FIa), formula (FIb), formula (FIc), formula (FId), formula (FIe), or formula (FIf).

Embodiment 3F. The peptide of Embodiment 1F or 2F, wherein the peptide of formula (I) is a peptide of formula (FIa):

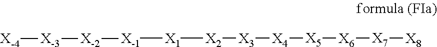

wherein in formula (FIa):
X₋₁, X₋₂, X₋₃, X₋₄, X₁, X₂, X₃, X₄, X₅, X₆, X₇, and X₈ are each independently an amino acid selected from Table F1; or Table F1 or a linker.

Embodiment 4F. The peptide of Embodiment 1F or 2F, wherein the peptide of formula (FI) is a peptide of formula (FIb):

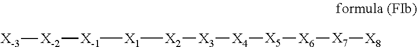

wherein in formula (Ib): X₋₁, X₋₂, X₋₃, X₁, X₂, X₃, X₄, X₅, X₆, X₇, and X₈ are each independently an amino acid selected from Table F1; or Table F1 or a linker.

Embodiment 5F. The peptide of Embodiment 1F or 2F, the peptide of formula (FI) is a peptide of formula (FIc):

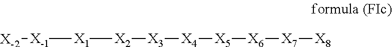

wherein in formula (Ic): X₋₁, X₁, X₂, X₃, X₄, X₅, X₆, X₇, and X₈ are each independently an amino acid selected from Table F1; or Table F1 or a linker.

Embodiment 6F. The peptide of Embodiment 1F or 2F, the peptide of formula (FI) is a peptide of formula (FId):

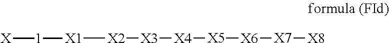

wherein in formula (Id): X₋₁, X₁, X₂, X₃, X₄, X₅, X₆, X₇, and X₈ are each independently an amino acid selected from Table F1; or Table F1 or a linker.

Embodiment 7F. The peptide of Embodiment 1F or 2F, the peptide of formula (FI) is a peptide of formula (FIe):

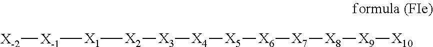

wherein in formula (Ie): X₋₁, X₋₂, X₁, X₂, X₃, X₄, X₅, X₆, X₇, X₈, X₉, and X₁₀ are each independently an amino acid selected from Table F1; or Table F1 or a linker.

Embodiment 8F. The peptide of Embodiment 1F or 2F, the peptide of formula (FI) is a peptide of formula (FIf):

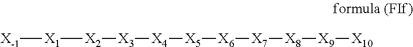

wherein in formula (Ie): X₋₁, X₁, X₂, X₃, X₄, X₅, X₆, X₇, X₈, X₉, and X₁₀ are each independently an amino acid selected from Table F1; or Table F1 or a linker.

Embodiment 9F. The peptide of any one of Embodiments 1F-8F, wherein X₅ is D-Phenylalanine (D-Phe).

Embodiment 10F. The peptide of any one of Embodiments 1F-9F, wherein X₆ is Arginine (Arg).

Embodiment 11F. The peptide of any one of Embodiments 1F-10F, wherein X₇ is 6-Fluoro-L-Tryptophan (Trp(6-F)).

Embodiment 12F. The peptide of any one of Embodiments 1F-11F, wherein X₈ is Cysteine (Cys).

Embodiment 13F. The peptide of any one of Embodiments 1F-12F, wherein X₁ is selected from D-Norarginine (D-Nar) and beta-homo-L-arginine (Beta-homoArg).

Embodiment 14F. The peptide of any one of Embodiments 1F-13F, wherein X₁ is D-Nar.

Embodiment 15F. The peptide of any one of Embodiments 1F-14F, wherein X₂ is Cys.

Embodiment 16F. The peptide of any one of Embodiments 1F-15F, wherein the peptide of formula (I) is selected from Table F1, Table F1A, Table F2, and Table F2A.

Embodiment 17F. The peptide of any one of Embodiments 1F-16F, wherein the peptide is a cyclic peptide.

Embodiment 18F. The peptide of Embodiment 17F, wherein the cyclic peptide comprises a disulfide bridge or a lactam bridge.

Embodiment 19F. The peptide of Embodiment 17F or 18F, wherein the cyclic peptide has the formula (FII):

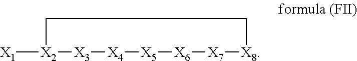

Embodiment 20F. The peptide of any one of Embodiments 1F-19F, wherein the cyclic peptide of formula (FII) is a cyclic peptide of any one of formula (FIIa), formula (FIIb), formula (FIIc), formula (FIId), formula (FIIe), or formula (IFIf).

Embodiment 21F. The peptide of Embodiments 19F or 20F, wherein the cyclic peptide of formula (FII) is a cyclic peptide of formula (FIIa):

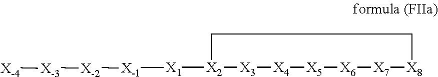

wherein in formula (FIIa): X₋₁, X₋₂, X₋₃, X₋₄, X₁, X₂, X₃, X₄, X₅, X₆, X₇, and X₈ are each independently an amino acid selected from Table F1; or Table F1 or a linker.

Embodiment 22F. The peptide of Embodiments 19F or 20F, wherein the cyclic peptide of formula (FII) is a cyclic peptide of formula (FIIb):

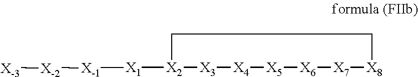

wherein in formula (FIIb): X₋₁, X₋₂, X₋₃, X₁, X₂, X₃, X₄, X₅, X₆, X₇, and X₈ are each independently an amino acid selected from Table F1; or Table F1 or a linker.

Embodiment 23F. The peptide of Embodiments 19F or 20F, wherein the cyclic peptide of formula (FII) is a cyclic peptide of formula (FIIc):

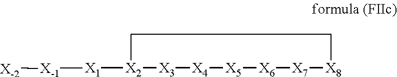

wherein in formula (FIIc): X₋₁, X₋₂, X₁, X₂, X₃, X₄, X₅, X₆, X₇, and X₈ are each independently an amino acid selected from Table F1; or Table F1 or a linker.

Embodiment 24F. The peptide of Embodiments 19F or 20F, wherein the cyclic peptide of formula (FII) is a cyclic peptide of formula (FIId):

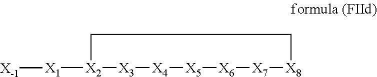

wherein in formula (IId): X₋₁, X₁, X₂, X₃, X₄, X₅, X₆, X₇, and X₈ are each independently an amino acid selected from Table F1; or Table F1 or a linker.

Embodiment 25F. The peptide of Embodiments 19F or 20F, wherein the cyclic peptide of formula (FII) is a cyclic peptide of formula (FIIe):

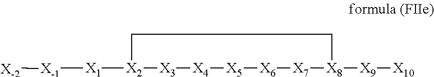

wherein in formula (FIIe): X₋₁, X₋₂, X₁, X₂, X₃, X₄, X₅, X&, X₇, X₈, X₉, and X₁₀ are each independently an amino acid selected from Table F1; or Table F1 or a linker.

Embodiment 26F. The peptide of Embodiments 19F or 20F, wherein the cyclic peptide of formula (FII) is a cyclic peptide of formula (FIIf):

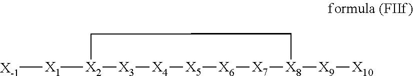

wherein in formula (FIIf): X₋₁, X₋₂, X₁, X₂, X₃, X₄, X₅, X₆, X₇, X₈, X₉, and X₁₀ are each independently an amino acid selected from Table F1; or Table F1 or a linker.

Embodiment 27F. The peptide of any one of Embodiments 1F-26F, wherein the peptide further comprises one or more amino acids conjugated to X₁ and/or X₈, optionally wherein the one or more amino acids are selected from D-Arginine (D-Arg), glycine (Gly), and L-Lys(AEEAc-AEEAc-L-γ-Glu-17-carboxyheptadecanoyl) (Lys*).

Embodiment 28F. The peptide of any one of Embodiments 1F-27F, wherein the peptide further comprises one or more lipids conjugated to X₁ and/or X₈.

Embodiment 29F. The peptide of any one of Embodiments 1F-28F, wherein the peptide is capped with N-terminal acetyl and/or C-terminal amide groups.

Embodiment 30F. The peptide of any one of Embodiments 1F-29F, wherein the peptide is selected from Table F1, Table F1A, Table F2 or Table F2A.

Embodiment 31F. The peptide of any one of Embodiments 1F-30F, wherein the peptide demonstrates increased selectivity for MC4R over MC1R when administered to a subject compared to a control.

Embodiment 32F. The peptide of any one of Embodiments 1F-31F, wherein the peptide demonstrates increased selectivity for MC4R over MC1R as measured by an in vitro, ex vivo, or in vivo assay when compared to a control.

Embodiment 33F. The peptide of Embodiment 31F or 32F, wherein the peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 34F. The peptide of Embodiment 31F or 32F, wherein the peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 35F. The peptide of any one of Embodiments 1F-30F, wherein the peptide demonstrates an increased ratio of MC4R intracellular signaling to MC1R intracellular signaling when administered to a subject compared to a control.

Embodiment 36F. The peptide of any one of Embodiments 1F-31F, wherein the peptide demonstrates increased selectivity for MC4R intracellular signaling to MC1R intracellular signaling as measured by an in vitro, ex vivo, or in vivo assay when compared to a control.

Embodiment 37F. The peptide of Embodiment 35F or 36F, wherein the peptide demonstrates an increased ratio of MC4R intracellular signaling to MC1R intracellular signaling by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when administered to a subject compared to a control.

Embodiment 38F. The peptide of Embodiment 35F or 36F, wherein the peptide demonstrates an increased ratio of MC4R intracellular signaling to MC1R intracellular signaling by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when administered to a subject compared to a control.

Embodiment 39F. The peptide of any one of Embodiments 1F-38F, wherein the peptide demonstrates enhanced melanocortin 4 receptor (MC4R) function in a subject when compared to before the peptide is administered or to a pre-treatment or non-treatment state, or a subject treated with control.

Embodiment 40F. The peptide of any one of Embodiments 1F-39F, wherein the peptide demonstrates decreased melanocortin 1 receptor (MC1R) function in a subject when compared to before the peptide is administered or to a pre-treatment or non-treatment state, or a subject treated with control.

Embodiment 41F. The peptide of any one of Embodiments 1F-40F, wherein the peptide demonstrates enhanced melanocortin 4 receptor (MC4R) function as measured by an in vitro, ex vivo, or in vivo assay when compared to a control.

Embodiment 42F. The peptide of any one of Embodiments 1F-41F, wherein the peptide demonstrates decreased melanocortin 1 receptor (MC1R) function as measured by an in vitro, ex vivo, or in vivo assay when compared to a control.

Embodiment 43F. The peptide of any one of Embodiments 39F-42F, wherein the peptide demonstrates enhanced MC4R function and/or decreased MC1R function by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 44F. The peptide of any one of Embodiments 39F-42F, wherein the peptide demonstrates enhanced MC4R function and/or decreased MC1R function by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 45F. The peptide of any one of Embodiments 17F-44F, wherein the cyclic peptide demonstrates increased selectivity for MC4R over MC1R by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% as measured by an in vitro, ex vivo, or in vivo assay when compared to a control, or before the peptide is administered, or to a pre-treatment or non-treatment state.

Embodiment 46F. The peptide of any one of Embodiments 17F-44F, wherein the cyclic peptide demonstrates increased selectivity for MC4R over MC1R by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold as measured by an in vitro, ex vivo, or in vivo assay when compared to a control, or before the peptide is administered, or to a pre-treatment or non-treatment state.

Embodiment 47F. The peptide of any one of Embodiments 1F-46F, wherein the peptide comprises a half-life extending moiety, optionally wherein the half-life extending moiety comprises polyethylene glycol (PEG), recombinant PEG mimetics, glycosylation of carbohydrates, Fc-fusion proteins or conjugates, albumin fusion proteins or conjugates, albumin-binding fusion proteins or conjugates, polypropylene glycol (PPG), XTEN fusion protein or conjugates, or a combination thereof.

Embodiment 48F. The peptide of any one of Embodiments 1F-47F, wherein the peptide further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids at the amino and/or carboxy terminus.

Embodiment 49F. The peptide of any one of Embodiments 1F-48F, wherein the peptide further comprises a therapeutic, diagnostic, and/or imaging moiety, optionally wherein the therapeutic, diagnostic, and/or imaging moiety comprises a small molecule, a biological (e.g., a biopolymer, a protein, a nucleic acid, a polysaccharide), or a radionuclide.

Embodiment 50F. The peptide of any one of Embodiments 1F-49F, wherein the peptide of formula (FI) is conjugated to or co-formulated with an additional therapeutic agent or therapeutic moiety.

Embodiment 51F. The peptide of Embodiments 49F or 50F, wherein the additional therapeutic agent or therapeutic moiety comprises incretin, an incretin analogue, or a modulator of an incretin receptor.

Embodiment 52F. The peptide of Embodiment 51F, wherein the modulator is an agonist.

Embodiment 53F. The peptide of Embodiment 52F, wherein the agonist is an agonist of GLP-1, GIP, and/or glucagon receptor.

Embodiment 54F. The peptide of Embodiment 52F, wherein the agonist is a GLP-1 analogue.

Embodiment 55F. The peptide of Embodiment 54F, wherein the GLP-1 analogue comprises a non-canonical amino acid.

Embodiment 56F. The peptide of Embodiment 54F, wherein the GLP-1 analogue comprises tirzepatide, tirzepatide analogue, liraglutide, retatrutide, exenatide, lixisenatide, semaglutide or semaglutide derivative.

Embodiment 57F. A protein comprising the peptide of any one of Embodiments 1F to 56F.

Embodiment 58F. The protein of Embodiment 57F, wherein the protein has a size of at least about 10 amino acid residues, or at least about 15 resides, or at least about 20 residues, or at least about 25 residues, or at least about 30 residues, or at least about 35 residues, or at least about 40 residues, or at least about 45 residues, or at least about 50 residues, or at least about 60 residues, or at least about 70 residues, or at least about 80 residues, or at least about 90 residues, or at least about 100 residues, or at least about 250 residues, or at least about 500 residues, at least about 750 residues, at least about 1,000 residues, at least about 1,250 residues, at least about 1,500 residues, at least about 1,750 residues, at least about 2,000 residues, at least about 3,000 residues, at least about 4,000 residues, or at least about 5,000 residues.

Embodiment 59F. A nucleic acid encoding the peptide of any one of Embodiments 1F to 56F, or the protein of Embodiment 57 or 58F.

Embodiment 60F. A solid synthesis device conjugated to the peptide of any one of Embodiments 1F to 56F, the protein of Embodiment 57F or 58F, or the nucleic acid of Embodiment 59F.

Embodiment 61F. A pharmaceutical composition comprising a peptide of any one of Embodiments 1F to 56F, or the protein of Embodiment 57F or 58F, or the nucleic acid of Embodiment 59F, and a pharmaceutically acceptable excipient or carrier.

Embodiment 62F. The pharmaceutical composition of Embodiment 61F, wherein the pharmaceutical composition further comprises an additional therapeutic, diagnostic, and/or imaging moiety.

Embodiment 63F. The pharmaceutical composition of Embodiment 61F or 62F, wherein the pharmaceutical composition is conjugated to or co-formulated with an additional therapeutic agent or therapeutic moiety.

Embodiment 64F. The pharmaceutical composition of any one of Embodiments 61F or 62F, wherein the pharmaceutical composition is administered orally, parenterally, by inhalation spray, topically, rectally, nasally, buccally, sub-lingually, vaginally or via an implanted reservoir.

Embodiment 65F. The pharmaceutical composition of any one of Embodiments 61F or 62F, wherein the pharmaceutical composition is administered via intravenous injection, intramuscular injection, subcutaneous injection, or depot injection.

Embodiment 66F. A method of selectively increasing the activity of MC4R over MC1R comprising administering a peptide of any one of Embodiments 1F to 56F or the pharmaceutical composition of Embodiments 61F to 65F to a subject in need thereof.

Embodiment 67F. The method of Embodiment 66F, wherein the method prevents, reduces, or eliminates hyperpigmentation in the subject when compared to an untreated or pre-treatment subject, or subject treated with a control.

Embodiment 68F. The method of Embodiment 67F, wherein the hyperpigmentation is reduced by at least about 10%, or at least about 20%, or at least about 30%, or at least about 40%, or at least about 50%, or at least about 60%, or at least about 70%, or at least about 75%, or at least about 80%, or at least about 85%, or at least about 90%, or at least about 95%, or at least about 100% when compared to an untreated or pre-treatment subject, or subject treated with a control.

Embodiment 69F. The method of Embodiment 67F, wherein the hyperpigmentation is reduced by at least about 1 fold, or at least about 2 fold, or at least about 3 fold, or at least about 4 fold, or at least about 5 fold or at least about 6 fold or at least about 7 fold, or at least about 8 fold, or at least about 9 fold, or at least about 10 fold, or at least about 50 fold, or at least about 100 fold, or at least about 500 fold, or at least about 1000 fold when compared to an untreated or pre-treatment subject, or subject treated with a control.

Embodiment 70F. A method of treating a disease or disorder associated with MC4R function comprising administering a peptide of any one of Embodiments 1F to 56F or the pharmaceutical composition of Embodiments 61F to 65F to a subject in need thereof.

Embodiment 71F. The method of Embodiment 70F, wherein the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency, and Prader-Willi syndrome.

Embodiment 72F. The method of Embodiment 70F or 71F, wherein the method further comprises co-administration of an additional therapeutic agent.

Embodiment 73F. The method of any one of Embodiments 70F to 72F, wherein the additional therapeutic agent comprises incretin, an incretin analogue, or a modulator of an incretin receptor.

Embodiment 74F. The method of Embodiment 73F, wherein the modulator is an agonist.

Embodiment 75F. The method of Embodiment 74F, wherein the agonist is an agonist of GLP-1, GIP, and/or glucagon receptor.

Embodiment 76F. The method of Embodiment 74F, wherein the agonist is a GLP-1 analogue.

Embodiment 77F. The method of Embodiment 76F, wherein the GLP-1 analogue comprises a non-canonical amino acid.

Embodiment 78F. The method of Embodiment 76F, wherein the GLP-1 analogue comprises tirzepatide, tirzepatide analogue, liraglutide, retatrutide, exenatide, lixisenatide, semaglutide or semaglutide derivative.

Embodiment 79F. The method of Embodiment 73F, wherein the subject is receiving and/or previously received treatment comprising incretin, an incretin analogue, or a modulator of an incretin receptor.

Embodiment 80F. The method of Embodiment 73F, wherein the subject has not or is not receiving treatment comprising incretin, an incretin analogue, or a modulator of an incretin receptor.

Embodiment 81F. A method of treating obesity or abnormal or excessive fat accumulation comprising administering a peptide of any one of Embodiments 1F to 56F or the pharmaceutical composition of Embodiments 61F to 65F to a subject in need thereof.

Embodiment 82F. A method of treating a disease or disorder associated with MC4R function comprising administering a peptide of any one of Embodiments 1F to 56F or the pharmaceutical composition of Embodiments 61F to 65F to a subject receiving and/or previously received incretin.

Embodiment 83F. A method of treating a disease or disorder associated with MC4R function comprising administering a peptide of any one of Embodiments 1F to 56F or the pharmaceutical composition of Embodiments 61F to 65F to a subject not receiving incretin.

Embodiment 84F. The method of any one of Embodiments 65F to 83F, wherein the peptide or pharmaceutical composition is administered from a prefilled dosage form device, such as syringe or autoinjector.

### EXAMPLES

### Example 1: Peptides Synthesis

This Example provides a description of the materials and methods for synthesizing the presently disclosed compounds. Non limiting examples include the synthesis of molecule 1158, as provided below herein, based on the followings steps:

(7R,12R,15S,18S,21R,24S)-7-((R)-2-acetamido-4-guanidinobutanamido)-24-(3-amino-3-oxopropyl)-15-((6-fluoro-1H-indol-3-yl)methyl)-21-(4-fluorobenzyl)-18-(3-guanidinopropyl)-11,11-dimethyl-6,14,17,20,23,26-hexaoxo-2-oxa-9,10-dithia-5,13,16,19,22,25-hexaazaspiro[3.22]hexacosane-12-carboxamide [Ac-DNar-Cys-Aib(O-cyclic)-Gln-DPhe(4-F)-Arg-Trp(6-F)-Pen-NH2(Cys&Pen Bridge]

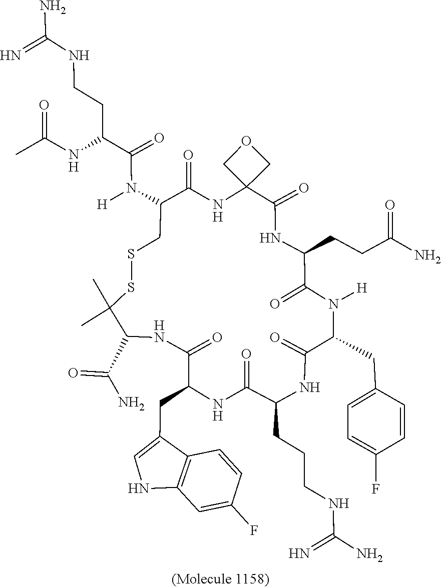

### Step 1. Preparation of Peptidyl Resin by Solid Phase Peptide Synthesis Using an Fmoc Protection Strategy

### Resin Preparation and Coupling

Sieber Amide Resin was swelled using N,N-dimethylformamide over 1 h. The Fmoc-amino acid and coupling reagent Hexafluorophosphate Benzotriazole Tetramethyl Uronium (HBTU) and N-methylmorpholine (NMM) were added to the resin. The reaction was carried out under a nitrogen atmosphere at room temperature. After the reaction was completed, the suspension was filtered, and the resin was washed 3 times with DMF.

### Deprotection

20% Piperidine in DMF was added into the reaction vessel to remove the Fmoc group. The suspension was kept at room temperature for 0.5h while a stream of nitrogen was bubbled through it. The suspension was filtered, and the resin was washed with DMF (5×). The full protected peptidyl resin was then cleaved using TFA for 0.5h, filtered and the filtrate was precipitated using cold diethyl ether. The mixture was centrifuged and washed with ether (3×), then dried under vacuum to obtain the crude peptide.

### Step 2. Macrocyclization and Purification

### Oxidization

The crude peptide was dissolved in 20% ACN/H2O, then an appropriate amount of acetic acid was added to adjust the solution to be acidic. A dropwise iodine/methanol was subsequently added and the reaction was stopped after few minutes by the addition of ascorbic acid.

### Purification

After oxidization, the solution was loaded onto the C18 column and eluted with a gradient from 28% to 58% Mobile Phase B (0.1% TFA in (80% ACN+20% H2O)) over 60 minutes to obtain the desired fractions with a purity greater than 75%. These fractions were used for next step purification. The desired fractions were loaded onto the C18 column and eluted with a gradient from 10% to 70% Mobile Phase B (0.1% formic acid in (80% ACN+20% H2O)) over 60 minutes to obtain 96% purity final fractions. These fractions were then pooled and trifluoroacetic acid (TFA) was added for lyophilization of the desired product (7R,12R,15S,18S,21R,24S)-7-((R)-2-acetamido-4-guanidinobutanamido)-24-(3-amino-3-oxopropyl)-15-((6-fluoro-1H-indol-3-yl)methyl)-21-(4-fluorobenzyl)-18-(3-guanidinopropyl)-11,11-dimethyl-6,14,17,20,23,26-hexaoxo-2-oxa-9,10-dithia-5,13,16,19,22,25-hexaazaspiro[3.22]hexacosane-12-carboxamide as a white solid.

LCMS (x2-2H)=593.8.

### Example 2: Designing MC4R Agonist Peptides

This Example describes selective melanocortin 4 receptor (MC4R) agonist peptides, and corresponding methods of making same, e.g., without limitation, for the treating and/or prevention of eating disorders such as overeating, metabolic disorders typified by positive energy imbalance, emotional or mental disorders, and dietary or syndromic obesity. Peptides described herein are MC4R agonistic peptides that display superior selectivity towards MC4R as compared with the other melanocortin receptors (such as MC1R). Further, peptides described herein display varying activity on G-protein coupled pathways stemming from the MC4R, namely Gs-coupled (a.k.a. cAMP), Gq-coupled, and B-arrestin dependent signaling pathways. Without being bound a particular theory, the peptides exhibit superior in vitro selectivity and potency, in vivo effectiveness, pharmacokinetic attributes, and/or stability when compared to other melanocortin receptor binding peptides.

This Example discloses peptides having key amino acid components that generated selectivity between MC4R and other melanocortin receptors while retaining potency on MC4R in vitro and retaining efficacy in DIO murine models of weight loss. The compounds herein distinguish themselves, inter alia, from other compounds utilized clinically for weight loss through the MC4R pathway.

### Materials and Methods

### Peptide Design

Compounds with a core structural component of X₁ through X₈ were provided, with extensions of various lengths emanating from either end of the core peptides. The peptides were capped with N-terminal acetyl and C-terminal amide groups. Some of the peptides were cyclized into macrocycles using either lactam or disulfide bridges. In addition to variations of the X₁ through X₈ core structure, some peptides include one or more of: amino-acid extensions at the N terminus, the C terminus, or both the N and C termini as well as lipidation of the peptides, optionally after extension from either (or both) end(s).

Initial computational model predictions of the binding of peptides against both MC1R and MC4R receptors and human intervention at multiple points of the design cycles were made to inform peptide designs. Following functional screening with MC1R and MC4R, the difference between these two values was used to determine the rank order for the selectivity of the peptides for increased MC4R binding and minimal MC1R binding. These molecules were then evaluated based on their suitability for synthesis and additional developability criteria.

Peptides, including comparator molecules were tested by assays listed below. Selectivity between receptors was assessed in part using cAMP assays, which test the activity of peptides through the Gs pathway.

### Peptide Screening

Peptides and comparator molecules were tested by assays listed below. Selectivity between receptors was assessed in part using cAMP assays, which test the activity of peptides through the Gs pathway.

Table 4 discloses a list of in vitro assays utilized in this Example.

TABLE 4
List of in vitro assays utilized in this Example.

| Pathway | | | | |
|---|---|---|---|---|
| Assayed | Receptor | Species | Cell Line Utilized | Signal Detection Method |
| Gs (cAMP) | MC1R | human | Monoclonal HEK293 | HitHunter cAMP XS+ assay (Enzyme |
| | | | overexpressing cell (cAMP | Fragment Complementation (EFC) with |
| | | | Hunter cell line) | B-galactosidase (B-Gal)) |
| Gs (cAMP) | MC2R | human | HEK293T transduced with | Green Downward cADDis biosensor |
| | | | BacMam virus | |
| Gs (cAMP) | MC3R | human | Monoclonal HEK293 | HitHunter cAMP XS+ assay (Enzyme |
| | | | overexpressing cell (cAMP | Fragment Complementation (EFC) with |
| | | | Hunter cell line) | B-galactosidase (B-Gal)) |
| Gs (cAMP) | MC4R | human | Monoclonal HEK293 | HitHunter cAMP XS+ assay (Enzyme |
| | | | overexpressing cell (cAMP | Fragment Complementation (EFC) with |
| | | | Hunter cell line) | B-galactosidase (B-Gal)) |
| Gs (cAMP) | MC5R | human | Monoclonal HEK293 | HitHunter cAMP XS+ assay (Enzyme |
| | | | overexpressing cell (cAMP | Fragment Complementation (EFC) with |
| | | | Hunter cell line) | B-galactosidase (B-Gal)) |
| B-arrestin | MC4R | human | Monoclonal HEK293 | HitHunter B-arrestin assay (Enzyme |
| | | | overexpressing cell | Fragment Complementation (EFC) with |
| | | | (PathHunter cell line) | B-galactosidase (B-Gal)) |

The cAMP Assay (Human Receptors)

The in vitro functional activity of peptides was determined via DiscoverX Hit Hunter® cAMP assays, adhering to manufacturer-specified protocols. Assays involved the use of HEK293 cells, each of which were engineered to express MC1R or MC4R receptors.

In accordance with standard procedures, cAMP Hunter cell lines, extracted from freezer stocks, were cultured in 20 μL volumes in white-walled, 384-well microplates. These cells were incubated at 37° C. for an appropriate duration before commencing testing procedures. The identification of cAMP modulation was determined using the DiscoverX HitHunter assay.

For gauging the response from agonists, cells were incubated with a specific peptide, after which the media was aspirated and replaced with a solution of 10 μL of HBSS/10 mM Hepes, known as Assay Buffer. A subsequent dilution of sample stocks yielded a 4× sample in the assay buffer, which was then added to the cells in equal quantities, followed by a 30-minute incubation at 37° C. The ensuing concentration of DMSO, the vehicle, was maintained at 1%. Melanotan-II functioned as positive control, with related dose-response curves of the peptides being generated from threefold serial dilutions from a top dose concentration of 1 mM (for MC1R and MC4R) completed in duplicate on a single plate. After compound incubation, cells were incubated with 5 μL of cAMP XS+Ab reagent and 20 μL of the cAMP XS+ED/CL lysis cocktail for one hour. This was followed by incubation with 20 μL of the cAMP XS+EA reagent for three hours at room temperature. Chemiluminescent signal was collected using a PerkinElmer instrument.

Compound activity was evaluated using a Chemical and Biological Information System software (CBIS) data analysis suite. The results described were calculated on an average of the two points per concentration generated by the assay described above. Curve fitting provided EC50 values. For Gs agonist mode assay, Emax was normalized as a percentage activity of the Melanotan II control after subtraction of vehicle, which was calculated using the formula: % Activity=100%×(mean RLU of test sample−mean RLU of vehicle control)/(mean RLU of Melanotan II control−mean RLU of vehicle control).

### The B-Arrestin Assay (Human Receptors)

Beta-arrestin recruitment activity was evaluated in vitro for peptides by employing the DiscoverX PathHunter® β-Arrestin assay, adhering to the provided manufacturer's protocol. In these assays, HEK293 cells, which display stable expression of MC4R, were utilized.

In accordance with standard procedures, PathHunter cell lines were expanded from freezer stocks. Cells were cultivated at 37° C. in a total volume of 20 μL within white-walled, 384-well microplates prior to testing. For gauging the response from agonists, cells were incubated with a specific peptide at various concentrations. Melanotan-II was utilized as positive control.

Intermediate dilution of sample stocks at each concentration was used to generate 5× sample concentrations in assay buffer. The cells were then treated with 5 μL of the resulting 5× sample to reach a 1× concentration of a peptide at each dilution step, per well, and incubated at 37° C. for 120 minutes, while maintaining the vehicle concentration at 1%. 10-point dose-response curves were generated by performing threefold serial dilutions of peptides from a peak dose concentration of 1 mM, and peptides were tested in duplicate per plate. Signal was detected by addition of 15 μL (50% v/v) of PathHunter Detection reagent cocktail, followed by a 2-hour incubation at room temperature. Chemiluminescence from each well on the microplates was measured using a PerkinElmer Envision™ instrument.

Compound activity was analyzed using CBIS data analysis suite (ChemInnovation, CA). For agonist mode assays, percentage activity was measured as per the formula: % Activity=100%×(mean RLU of test sample−mean RLU of vehicle control)/(mean MAX control ligand−mean RLU of vehicle control). These results for each peptide were fit with a 4 parameter curve, and calculated EC50, calculated Emax, and observed Max efficacy values were recorded.

### Selectivity Calculations

Selectivity between any two receptors was calculated by a division of the EC50 values between the two receptors, for each individual peptide.

3-Day Murine Acute Feeding and Weight Loss Assay with Daily Dosing of MC4R Agonist

3-day efficacy studies were conducted using DIO mice to evaluate the effect of MC4R agonistic peptides on murine weight loss. Mice were purchased from the Jackson Laboratory, which been fed a 60% high-fat diet in an 18-20-week diet-induced obesity (DIO) mouse model. These mice were then acclimated to their new environment in a vivarium for two weeks, which also served as time to re-establish baseline weight of the rodents in preparation for future experiments. Each mouse was housed separately to facilitate precise measurement of food intake. Although single housing can potentially induce stress, the impact was minimized as the rodents were already singly housed upon arrival. A day prior to the initiation of the experiment (Day −1), the subjects were weighed, and randomization was conducted. Mice were randomized into cohorts based on their weight and age, ensuring that all specimens fall within a two-week age range of each other. On Day 0, both the mice and their food supplies were weighed, and the first daily bolus injection of 5 mg/kg of test peptide was administered subcutaneously between the scapulae. The rodents were then monitored cageside for 30 minutes. This procedure was replicated on Day 1 and Day 2. The experiment concluded on Day 3 with the final weighing of the mice and food. Weight loss data were shown as the mean±SD of the percent weight loss since Day −1. All mice utilized in studies were C57/BL6 DIO male mice, aged 18-20 weeks, with N=5 mice/group.

### Results

Comparator molecules tested include those currently in clinic, those clinical programs were terminated or FDA approved. A baseline activity of these molecules across the assays described above is included in Table 5, below.

TABLE 5
Peptides (and small molecules) that serve as comparator compounds,
and their receptor potency and efficacy values for human receptor
assays. Selectivity and bias for these compounds is also displayed.

| | | | | | Bias: MC4R |
|---|---|---|---|---|---|
| | | | | Selectivity: | B-arrestin v |
| | | | | MC4R v MC1R | MC4R cAMP |
| | | | | (Larger | (Larger |
| | | | | numbers | numbers |
| | | | | indicate | indicate bias |
| Compound | MC1R | MC4R | MC4R Barr | selectivity | towards |
| Name | EC50 (nM) | EC50 (nM) | EC50 (nM) | towards MC4R) | B-arrestin) |
| LY2112688 | 0.96 | 0.36 | 0.45 | 2.69 | 0.79 |
| Setmelanotide | 0.94 | 0.89 | 0.76 | 1.06 | 1.17 |
| Ring Peptide | 28.52 | 32.58 | 9.07 | 0.88 | 3.59 |
| Compound from | | | | | |
| KR2002-0038400 | | | | | |
| NDP-aMSH | 0.18 | 2.27 | 0.80 | 0.08 | 2.85 |
| Melanotan-II | 0.13 | 1.60 | 0.57 | 0.08 | 2.80 |
| a-MSH | 2.39 | 33.72 | 85.96 | 0.07 | 0.39 |
| Bremelanotide | 0.17 | 2.44 | 1.19 | 0.07 | 2.05 |
| RM-718 | 295.73 | 1.27 | 0.92 | 233.34 | 1.38 |

### Selectivity for MC4R vs MC1R

Table 6 (A-B) lists the calculated MC4R vs MC1R selectivity of peptides tested. Setmelanotide has a selectivity of 1.06, and Melanotan II has a selectivity of 0.08. When comparing the selectivity of MC4R v MC1R, larger values indicate selectivity towards MC4R. When comparing the bias of MC4R B-arrestin v MC4R cAMP, larger values indicate bias towards B-arrestin. Table 6 (B) also includes the base peak and molecular ion peak identified using mass spectrometry (second column from left).

Table 6 (A and B). The in vitro assay activity and calculated selectivity and calculated bias of the foregoing exemplary molecules that are selective for MC4R vs MC1R in cAMP functional screening and may also be biased for B-arrestin over cAMP signal.

TABLE 6 (A)

| | | Bias: MC4R |
|---|---|---|
| Molecule | Selectivity: | B-arrestin v |
| Name | MC4R v MC1R | MC4R cAMP |
| 1119 | *** | ++ |
| 1094 | *** | ++ |
| 1106 | *** | ++ |
| 1122 | *** | ++ |
| 1107 | *** | ++ |
| 1093 | *** | ++ |
| 1092 | *** | ++ |
| 1124 | *** | ++ |
| 1015 | *** | + |
| 1035 | *** | + |
| 1091 | *** | ++ |
| 1096 | *** | ++ |
| 1043 | *** | + |
| 1012 | *** | +++ |
| 1049 | *** | + |
| 1041 | *** | + |
| 1099 | *** | +++ |
| 1030 | *** | +++ |
| 1121 | *** | + |
| 1042 | *** | + |
| 1024 | *** | + |
| 1064 | *** | + |
| 1037 | *** | + |
| 1019 | *** | + |
| 1085 | *** | + |
| 1016 | *** | + |
| 1158 | *** | |

TABLE 6 (B)

| | | | | Bias: MC4R |
|---|---|---|---|---|
| Molecule | Base | Molecular | Selectivity: | B-arrestin v |
| Name | Peak (m/z) | Ion | MC4R v MC1R | MC4R cAMP |
| 1122 | 393.6 | x3-3H | *** | ++ |
| 1124 | 403.3 | x3-3H | *** | ++ |
| 1126 | 397.3 | x3-3H | *** | + |
| 25 | 576.3 | x2-2H | *** | + |
| 26 | 388.3 | x3-3H | *** | + |
| 27 | 402.6 | x3-3H | *** | + |
| 28 | 593.9 | x2-2H | *** | + |
| 29 | 400 | x3-3H | *** | + |
| 30 | 414.2 | x3-3H | *** | ++ |
| 31 | 610.9 | x2-2H | *** | ++ |
| 32 | 596.8 | x2-2H | *** | ++ |
| 33 | 611.4 | x2-2H | *** | ++ |
| 34 | 618.3 | x2-2H | *** | ++ |
| 35 | 409.6 | x3-3H | *** | + |
| 36 | 414.9 | x3-3H | *** | + |
| 37 | 603.8 | x2-2H | *** | ++ |
| 38 | 611.8 | x2-2H | *** | ++ |
| 39 | 603.8 | x2-2H | *** | + |
| 1158 | 593.8 | x2-2H | *** | + |
| 41 | 399.9 | x3-3H | *** | + |
| 42 | 414.3 | x3-3H | *** | ++ |
| 45 | 418.9 | x3-3H | *** | ++ |
| 46 | 561.9 | x2-2H | *** | ++ |
| 47 | 384.6 | x3-3H | *** | ++ |
| 48 | 393 | x3-3H | *** | +++ |
| 49 | 397.7 | x3-3H | *** | +++ |
| 50 | 585.9 | x2-2H | *** | ++ |
| 51 | 568.8 | x2-2H | *** | ++ |
| 52 | 389.3 | x3-3H | *** | ++ |
| 53 | 398.6 | x3-3H | *** | ++ |
| 55 | 405 | x3-3H | *** | ++ |
| 56 | 596.85 | x2-2H | *** | + |
| 57 | 393.6 | x3-3H | *** | + |
| 58 | 579.8 | x2-2H | *** | + |
| 59 | 402.3 | x3-3H | *** | ++ |
| 61 | 411.6 | x3-3H | *** | ++ |
| 63 | 397.6 | x3-3H | *** | + |
| 65 | 407 | x3-3H | *** | + |
| 67 | 392.9 | x3-3H | *** | ++ |
| 69 | 602.9 | x2-2H | *** | ++ |
| 70 | 592.8 | x2-2H | *** | ++ |
| 71 | 388.2 | x3-3H | *** | ++ |
| 72 | 393 | x3-3H | *** | ++ |
| 73 | 402.3 | x3-3H | *** | ++ |
| 74 | 397.6 | x3-3H | *** | ++ |
| 75 | 402.3 | x3-3H | *** | + |
| 76 | 411.6 | x3-3H | *** | ++ |
| 77 | 397.6 | x3-3H | *** | + |
| 78 | 585.8 | x2-2H | *** | + |
| 79 | 406.9 | x3-3H | *** | + |
| 80 | 599.8 | x2-2H | *** | + |
| 81 | 614.4 | x2-2H | *** | + |
| 82 | 621.4 | x2-2H | *** | ++ |
| 83 | 566.3 | x2-2H | *** | ++ |
| 84 | 381.6 | x3-3H | *** | ++ |
| 85 | 583.3 | x2-2H | *** | ++ |
| 86 | 580.3 | x2-2H | *** | + |
| 87 | 390.9 | x3-3H | *** | ++ |
| 88 | 597.35 | x2-2H | *** | + |
| 89 | 559.3 | x2-2H | *** | ++ |
| 90 | 376.9 | x3-3H | *** | + |
| 91 | 576.4 | x2-2H | *** | ++ |
| 92 | 573.3 | x2-2H | *** | ++ |
| 93 | 386.3 | x3-3H | *** | + |
| 94 | 590.3 | x2-2H | *** | ++ |
| 95 | 394.3 | x3-3H | *** | ++ |
| 96 | 567.3 | x2-2H | *** | ++ |
| 97 | 560.3 | x2-2H | *** | ++ |
| 99 | 388.3 | x3-3H | *** | ++ |
| 100 | 397.6 | x3-3H | *** | ++ |
| 101 | 585.8 | x2-2H | *** | ++ |
| 102 | 394.3 | x3-3H | *** | ++ |
| 103 | 567.3 | x2-2H | *** | ++ |
| 104 | 560.3 | x2-2H | *** | ++ |
| 106 | 388.3 | x3-3H | *** | ++ |
| 107 | 397.6 | x3-3H | *** | ++ |
| 108 | 585.8 | x2-2H | *** | ++ |
| 109 | 403.6 | x3-3H | *** | ++ |
| 110 | 578.9 | x2-2H | *** | +++ |
| 125 | 686.2 | x3-3H | *** | ++ |
| 131 | 690.9 | x3-3H | *** | ++ |
| 137 | 608.3 | x2-2H | *** | + |
| 140 | 1023 | x2-2H | *** | + |
| 141 | 1016 | x2-2H | *** | + |
| 142 | 1030 | x2-2H | *** | + |
| 144 | 998 | x2-2H | *** | + |
| 148 | 681 | x3-3H | *** | ++ |
| 149 | 1014 | x2-2H | *** | + |
| 150 | 1007 | x2-2H | *** | + |
| 151 | 1021 | x2-2H | *** | ++ |
| 152 | 584.8 | x2-2H | *** | + |
| 153 | 584.8 | x2-2H | *** | + |
| 154 | 409.3 | x3-3H | *** | + |
| 155 | 415.3 | x3-3H | *** | + |
| 156 | 409.3 | x3-3H | *** | + |
| 157 | 613.9 | x2-2H | *** | + |
| 158 | 599.8 | x2-2H | *** | + |
| 159 | 402.9 | x3-3H | *** | + |
| 160 | 593.9 | x2-2H | *** | ++ |
| 161 | 605 | x2-2H | *** | +++ |
| 162 | 590.9 | x2-2H | *** | ++ |
| 163 | 397 | x3-3H | *** | ++ |
| 164 | 584.9 | x2-2H | *** | ++ |
| 166 | 1030 | x2-2H | *** | + |
| 168 | 607.8 | x2-2H | *** | ++ |
| 169 | 593.8 | x2-2H | *** | + |
| 170 | 396.3 | x3-3H | *** | + |
| 203 | | | *** | + |
| 1012 | 574.8 | x2-2H | *** | +++ |
| 1016 | 402 | x3-3H | *** | + |
| 1019 | 392.6 | x3-3H | *** | + |
| 1020 | 397.3 | x3-3H | *** | + |
| 1024 | 406.6 | x3-3H | *** | + |
| 1030 | 424.6 | x3-3H | *** | +++ |
| 1035 | 398.7 | x3-3H | *** | + |
| 1037 | 379.3 | x3-3H | *** | + |
| 1039 | 383.3 | x3-3H | *** | + |
| 1041 | 403.2 | x3-3H | *** | + |
| 1042 | 394 | x3-3H | *** | + |
| 1043 | 397.3 | x3-3H | *** | + |
| 1044 | 397.3 | x3-3H | *** | + |
| 1049 | 397.3 | x3-3H | *** | + |
| 1129 | 562.5 | x4-4H | *** | + |
| 1128 | 754 | x3-3H | *** | + |
| 1064 | 392.6 | x3-3H | *** | + |
| 1130 | 721.4 | x3-3H | *** | ++ |
| 1085 | 403.2 | x3-3H | *** | + |
| 1091 | 592.7 | x2-2H | *** | ++ |
| 1092 | 570.7 | x2-2H | *** | ++ |
| 1093 | 579.7 | x2-2H | *** | ++ |
| 1094 | 390.6 | x3-3H | *** | ++ |
| 1096 | 571.7 | x2-2H | *** | ++ |
| 1099 | 572.8 | x2-2H | *** | +++ |
| 1106 | 594.2 | x2-2H | *** | ++ |
| 1107 | 601.3 | x2-2H | *** | ++ |
| 1111 | 401.3 | x3-3H | *** | + |
| 1119 | 404.9 | x3-3H | *** | ++ |
| 1121 | 397.6 | x3-3H | *** | + |

*denotes MC4R vs MC1R selectivity having a range of 0.01 to <1.00,
**denotes MC4R vs MC1R selectivity having a range of ≥1.00 to ≤7.40,
***denotes MC4R vs MC1R selectivity of about >7.40.
+denotes MC4R B-arrestin v MC4R cAMP bias having a range of >0.00 to ≤2.00,
++denotes MC4R B-arrestin v MC4R cAMP bias having a range of 2.00 to ≤10.00,
+++denotes MC4R B-arrestin v MC4R cAMP bias having a value of >10.00.

Table 7 shown below lists families of molecules that have the X₃ position (e.g., Aib(O-cyclic) in common. The X₃ residue chosen for investigation was based on X₃ and X₄ combinatorial pairings that elicited the greatest selectivity between MC4R and MC1R. Without being bound to a particular theory, throughout this Table, the peptide sequences illustrated how selectivity between the MC4R and MC1R receptors increased when a specific X₃ and X₄ pairing was identified. The X₁, X₅, and X₇ positions contributed to selectivity, as was seen in Table 7 through the contribution of the X₅ position to improvements in selectivity (Table 7, Molecule 1092, Molecule 1093, and Molecule 1158). However, the substitution interplay was most evident in analogues where certain X₃ and X₄ pairings lead to retention of high MC4R functional potency, with significant decrement of MC1R potency (to generate selectivity).

Without being bound to a particular theory, the family of peptides that contained Aib(O-cyclic) demonstrated that certain pairings produced improved selectivity, such as the Gln at X₄. This data also illustrated how the identity of the X₅ position contributes to selectivity. When comparing the selectivity of MC4R v MC1R, larger values indicate selectivity towards MC4R. When comparing the bias of MC4R B-arrestin v MC4R cAMP, larger values indicate bias towards B-arrestin.

TABLE 7
Compounds with Aib(O-cyclic) at X₃. All peptides are N-acetylated,
have a disulfide linkage and contain a C-terminal amide. When comparing the selectivity
of MC4R v MC1R, larger values indicate selectivity towards MC4R. When comparing the bias
of MC4R B-arrestin v MC4R cAMP, larger values indicate bias towards B-arrestin.
Table 7: Compounds with Aib(O-cyclic) at X₃

| Mole- | | | | | | | | | |
|---|---|---|---|---|---|---|---|---|---|
| cule | | | | | | | | | |
| name | X₋₄ | X₋₃ | X₋₂ | X₋₁ | X₁ | X₂ | X₃ | X₄ | X₅ |
| 1150 | Lys* | D-Arg | Gly | D-Arg | D-Nar | Cys | Aib(O- | [symbol] | D-Phe(4-F) |
| | | | | | | | cyclic) | | |
| 1142 | | Lys* | Gly | D-Arg | D-Nar | Cys | Aib(O- | [symbol] | D-Phe(4-F) |
| | | | | | | | cyclic) | | |
| 1144 | | Lys* | PEG1 | PEG1 | D-Nar | Cys | Aib(O- | [symbol] | D-Phe(4-F) |
| | | | | | | | cyclic) | | |
| 1151 | Lys* | D-Arg | PEG1 | D-Arg | Beta- | Cys | Aib(O- | [symbol] | D-Phe(4-F) |
| | | | | | homoArg | | cyclic) | | |
| 1152 | Lys* | D-Arg | Gly | D-Arg | D-Nar | Cys | Aib(O- | 3Pal | D-Phe(4-F) |
| | | | | | | | cyclic) | | |
| 1153 | Lys* | D-Arg | Gly | D-Arg | D-Nar | Cys | Aib(O- | Orn | D-Phe(4-F) |
| | | | | | | | cyclic) | | |
| 1122 | | | | | D-Nar | Cys | Aib(O- | 3Pal | D-Phe(4-F) |
| | | | | | | | cyclic) | | |
| 1123 | | | | | D-Nar | Cys | Aib(O- | Orn | D-Phe(4-F) |
| | | | | | | | cyclic) | | |
| 25 | | | | | D-Nar | Glu | Aib(O- | [symbol] | D-Phe(4-F) |
| | | | | | | | cyclic) | | |
| 28 | | | | | Beta- | Cys | Aib(O- | [symbol] | D-Phe(4-F) |
| | | | | | homoArg | | cyclic) | | |
| 1158 | | | | | D-Nar | Cys | Aib(O- | [symbol] | D-Phe(4-F) |
| | | | | | | | cyclic) | | |
| 43 | | | | | D-Nar | hCys | Aib(O- | [symbol] | D-Phe(4-F) |
| | | | | | | | cyclic) | | |
| [symbol] | | | | | D-Nar | Cys | Aib(O- | [symbol] | D-Phe |
| | | | | | | | cyclic) | | |
| 51 | | | | | Arg | Cys | Aib(O- | [symbol] | D-Phe |
| | | | | | | | cyclic) | | |
| [symbol] | | | | | D-Nar | Cys | Aib(O- | Thr | D-Phe(4-F) |
| | | | | | | | cyclic) | | |
| [symbol] | | | | | D-Nar | Cys | Aib(O- | Thr | D-Phe(4-F) |
| | | | | | | | cyclic) | | |
| 89 | | | | | D-Nar | Cys | Aib(O- | Ser | D-Phe(4-F) |
| | | | | | | | cyclic) | | |
| 92 | | | | | D-Nar | Cys | Aib(O- | Ser | D-Phe(4-F) |
| | | | | | | | cyclic) | | |
| 111 | Lys* | Gly | Gly | Gly | D-Nar | Cys | Aib(O- | [symbol] | D-Phe(4-F) |
| | | | | | | | cyclic) | | |
| 112 | | Lys* | Gly | Gly | D-Nar | Cys | Aib(O- | [symbol] | D-Phe(4-F) |
| | | | | | | | cyclic) | | |
| 113 | | | Lys* | Gly | D-Nar | Cys | Aib(O- | [symbol] | D-Phe(4-F) |
| | | | | | | | cyclic) | | |
| 114 | | | | Lys* | D-Nar | Cys | Aib(O- | [symbol] | D-Phe(4-F) |
| | | | | | | | cyclic) | | |
| 115 | | | | | D-Nar | Cys | Aib(O- | [symbol] | D-Phe(4-F) |
| | | | | | | | cyclic) | | |
| 116 | | | | | D-Nar | Cys | Aib(O- | [symbol] | D-Phe(4-F) |
| | | | | | | | cyclic) | | |
| 117 | | | | | D-Nar | Cys | Aib(O- | [symbol] | D-Phe(4-F) |
| | | | | | | | cyclic) | | |
| 118 | | | | | D-Nar | Cys | Aib(O- | [symbol] | D-Phe(4-F) |
| | | | | | | | cyclic) | | |
| 119 | | | | | D-Nar | Cys | Aib(O- | [symbol] | D-Phe(4-F) |
| | | | | | | | cyclic) | | |
| 120 | | | | | D-Nar | Cys | Aib(O- | [symbol] | D-Phe(4-F) |
| | | | | | | | cyclic) | | |
| 121 | | | | | D-Nar | Cys | Aib(O- | [symbol] | D-Phe(4-F) |
| | | | | | | | cyclic) | | |
| 122 | | | | | D-Nar | Cys | Aib(O- | [symbol] | D-Phe(4-F) |
| | | | | | | | cyclic) | | |
| 137 | | | | | Beta- | Cys | Aib(O- | [symbol] | D-Phe(4-F) |
| | | | | | homoArg | | cyclic) | | |
| 139 | | | Lys* | Gly | D-Nar | Cys | Aib(O- | [symbol] | D-Phe |
| | | | | | | | cyclic) | | |
| 140 | | | | Lys* | Arg | Cys | Aib(O- | [symbol] | D-Phe(4-F) |
| | | | | | | | cyclic) | | |
| 141 | | | | Lys* | D-Nar | Cys | Aib(O- | [symbol] | D-Phe(4-F) |
| | | | | | | | cyclic) | | |
| 142 | | | | Lys* | Beta- | Cys | Aib(O- | [symbol] | D-Phe(4-F) |
| | | | | | homoArg | | cyclic) | | |
| 143 | | | | Lys* | Arg | Cys | Aib(O- | [symbol] | D-Phe |
| | | | | | | | cyclic) | | |
| 144 | | | | Lys* | D-Nar | Cys | Aib(O- | [symbol] | D-Phe |
| | | | | | | | cyclic) | | |
| 145 | | | | Lys* | Beta- | Cys | Aib(O- | [symbol] | D-Phe |
| | | | | | homoArg | | cyclic) | | |
| 146 | | | | Lys* | Arg | Cys | Aib(O- | [symbol] | D-Phe |
| | | | | | | | cyclic) | | |
| 147 | | | | Lys* | D-Nar | Cys | Aib(O- | [symbol] | D-Phe |
| | | | | | | | cyclic) | | |
| 148 | | | | Lys* | Beta- | Cys | Aib(O- | [symbol] | D-Phe |
| | | | | | homoArg | | cyclic) | | |
| 149 | | | | Lys* | Arg | Cys | Aib(O- | [symbol] | D-Phe(4-F) |
| | | | | | | | cyclic) | | |
| 150 | | | | Lys* | D-Nar | Cys | Aib(O- | [symbol] | D-Phe(4-F) |
| | | | | | | | cyclic) | | |
| 151 | | | | Lys* | Beta- | Cys | Aib(O- | [symbol] | D-Phe(4-F) |
| | | | | | homoArg | | cyclic) | | |
| 152 | | | | | D-Nar | Cys | Aib(O- | [symbol] | D-Phe |
| | | | | | | | cyclic) | | |
| 153 | | | | | D-Nar | Cys | Aib(O- | [symbol] | D-Phe(4-F) |
| | | | | | | | cyclic) | | |
| [symbol] | | | | Lys* | D-Nar | Pen | Aib(O- | [symbol] | D-Phe(4-F) |
| | | | | | | | cyclic) | | |
| 167 | | | | Lys* | D-Nar | Pen | Aib(O- | [symbol] | D-Phe(4-F) |
| | | | | | | | cyclic) | | |
| 168 | | | | | D-Nar | Pen | Aib(O- | [symbol] | D-Phe(4-F) |
| | | | | | | | cyclic) | | |
| 169 | | | | | D-Nar | Pen | Aib(O- | [symbol] | D-Phe(4-F) |
| | | | | | | | cyclic) | | |
| 170 | | | | | D-Nar | Cys | Aib(O- | Lys | D-Phe(4-F) |
| | | | | | | | cyclic) | | |
| 186 | | | | | D-Nar | Cys | Aib(O- | [symbol] | [symbol] |
| | | | | | | | cyclic) | | |
| 187 | | | | | D-Nar | Cys | Aib(O- | [symbol] | [symbol] |
| | | | | | | | cyclic) | | |
| 188 | | | | | D-Nar | Cys | Aib(O- | [symbol] | [symbol] |
| | | | | | | | cyclic) | | |
| 189 | | | | | D-Nar | Cys | Aib(O- | [symbol] | [symbol] |
| | | | | | | | cyclic) | | |
| 190 | | | | | D-Nar | Cys | Aib(O- | [symbol] | [symbol] |
| | | | | | | | cyclic) | | |
| 191 | | | | | D-Nar | Cys | Aib(O- | [symbol] | [symbol] |
| | | | | | | | cyclic) | | |
| 192 | | | | | D-Nar | Cys | Aib(O- | [symbol] | [symbol] |
| | | | | | | | cyclic) | | |
| 193 | | | | | D-Nar | Cys | Aib(O- | [symbol] | 5,5,5- |
| | | | | | | | cyclic) | | Trifluoro- |
| | | | | | | | | | D- [symbol] |
| 194 | | | | | D-Nar | Cys | Aib(O- | [symbol] | 3-(Trifluoro- |
| | | | | | | | cyclic) | | methyl)- |
| | | | | | | | | | D-alanine |
| 195 | | | | | D-Nar | Cys | Aib(O- | [symbol] | 3-Cyano- |
| | | | | | | | cyclic) | | D-alanine |
| 196 | | | | | D-Nar | Cys | Aib(O- | [symbol] | 3- |
| | | | | | | | cyclic) | | Cyclopropyl- |
| | | | | | | | | | D-alanine |
| 197 | | | | | D-Nar | Cys | Aib(O- | [symbol] | (R)-2-Amino- |
| | | | | | | | cyclic) | | 4-cyclo- |
| | | | | | | | | | propyl- |
| | | | | | | | | | butanoic |
| | | | | | | | | | acid |
| 198 | | | | | D-Nar | Cys | Aib(O- | [symbol] | [symbol] -Amino- |
| | | | | | | | cyclic) | | 2-pyridine- |
| | | | | | | | | | [symbol] acid |
| 199 | | | | | D-Nar | Cys | Aib(O- | [symbol] | [symbol] -Amino- |
| | | | | | | | cyclic) | | 3-pyridine |
| | | | | | | | | | [symbol] acid |
| 200 | | | | | D-Nar | Cys | Aib(O- | [symbol] | [symbol] -Amino- |
| | | | | | | | cyclic) | | 4-pyridine |
| | | | | | | | | | [symbol] acid |
| 1058 | | | | | D-Nar | Cys | Aib(O- | His | D-Phe |
| | | | | | | | cyclic) | | |
| 1092 | | | | | D-Nar | Cys | Aib(O- | [symbol] | D-Phe |
| | | | | | | | cyclic) | | |
| 1093 | | | | | D-Nar | Cys | Aib(O- | [symbol] | D-Phe(4-F) |
| | | | | | | | cyclic) | | |
| 1102 | | | | | D-Nar | Cys | Aib(O- | hGln | D-Phe |
| | | | | | | | cyclic) | | |
| 1103 | | | | | D-Nar | Cys | Aib(O- | Cit | D-Phe |
| | | | | | | | cyclic) | | |
| 1106 | | | | | D-Nar | Cys | Aib(O- | Cit | D-Phe(4-F) |
| | | | | | | | cyclic) | | |
| 1107 | | | | | D-Nar | Cys | Aib(O- | [symbol] | D-Phe(4-F) |
| | | | | | | | cyclic) | | |

| | | | | | | | | | Bias: |
|---|---|---|---|---|---|---|---|---|---|
| | | | | | | | | Selec- | MC4R B- |
| Mole- | | | | | | | | tivity: | arrestin |
| cule | | | | | | | | MC4R v | v MC4R |
| name | X₆ | X₇ | X₈ | X₉ | X₁₀ | X₁₁ | X₁₂ | MC1R | cAMP |
| 1150 | Arg | Trp(6-F) | Cys | | | | | * | + |
| 1142 | Arg | Trp(6-F) | Cys | | | | | ** | + |
| 1144 | Arg | Trp(6-F) | Cys | | | | | ** | ++ |
| 1151 | Arg | Trp(6-F) | Cys | | | | | * | ++ |
| 1152 | Arg | Trp(6-F) | Cys | | | | | * | ++ |
| 1153 | Arg | Trp(6-F) | Cys | | | | | * | ++ |
| 1122 | Arg | Trp(6-F) | Cys | | | | | *** | ++ |
| 1123 | Arg | Trp(6-F) | Cys | | | | | ** | + |
| 25 | Arg | Trp(6-F) | [symbol] | | | | | *** | + |
| 28 | Arg | Trp(6-F) | Cys | | | | | *** | + |
| 1158 | Arg | Trp(6-F) | Pen | | | | | *** | + |
| 43 | Arg | Trp(6-F) | Pen | | | | | ** | ++ |
| [symbol] | Arg | Trp | Cys | | | | | *** | ++ |
| 51 | Arg | Trp | Cys | | | | | *** | ++ |
| [symbol] | Arg | Trp(6-F) | Cys | | | | | *** | ++ |
| [symbol] | Arg | Trp(6-F) | Pen | | | | | *** | + |
| 89 | Arg | Trp(6-F) | Cys | | | | | *** | ++ |
| 92 | Arg | Trp(6-F) | Pen | | | | | *** | ++ |
| 111 | Arg | Trp(6-F) | Cys | | | | | * | ++ |
| 112 | Arg | Trp(6-F) | Cys | | | | | ** | ++ |
| 113 | Arg | Trp(6-F) | Cys | | | | | ** | ++ |
| 114 | Arg | Trp(6-F) | Cys | | | | | ** | ++ |
| 115 | Arg | Trp(6-F) | Cys | Gly | Gly | Gly | Lys* | ** | ++ |
| 116 | Arg | Trp(6-F) | Cys | Gly | Gly | Lys* | | * | ++ |
| 117 | Arg | Trp(6-F) | Cys | Gly | Lys* | | | ** | ++ |
| 118 | Arg | Trp(6-F) | Cys | Lys* | | | | * | ++ |
| 119 | Arg | Trp(6-F) | Cys | PEG1 | PEG1 | Lys* | | ** | ++ |
| 120 | Arg | Trp(6-F) | Cys | D-Arg | Gly | Lys* | | ** | ++ |
| 121 | Arg | Trp(6-F) | Cys | Pro | Phe | Lys* | | ** | ++ |
| 122 | Arg | Trp(6-F) | Cys | Lys | Pro | Val | Lys* | * | ++ |
| 137 | Arg | Trp(6-F) | Cys | | | | | *** | + |
| 139 | Arg | Trp(6-F) | Cys | | | | | ** | ++ |
| 140 | Arg | Trp(6-F) | Pen | | | | | *** | + |
| 141 | Arg | Trp(6-F) | Pen | | | | | *** | + |
| 142 | Arg | Trp(6-F) | Pen | | | | | *** | + |
| 143 | Arg | Trp | Pen | | | | | ** | ++ |
| 144 | Arg | Trp | Pen | | | | | *** | + |
| 145 | Arg | Trp | Pen | | | | | ** | ++ |
| 146 | Arg | Trp(6-F) | Pen | | | | | ** | + |
| 147 | Arg | Trp(6-F) | Pen | | | | | ** | + |
| 148 | Arg | Trp(6-F) | Pen | | | | | *** | ++ |
| 149 | Arg | Trp | Pen | | | | | *** | + |
| 150 | Arg | Trp | Pen | | | | | *** | + |
| 151 | Arg | Trp | Pen | | | | | *** | ++ |
| 152 | Arg | Trp(6-F) | Pen | | | | | *** | + |
| 153 | Arg | Trp | Pen | | | | | *** | + |
| [symbol] | Arg | Trp(6-F) | Pen | | | | | *** | + |
| 167 | Arg | Trp(6-F) | Cys | | | | | * | +++ |
| 168 | Arg | Trp(6-F) | Pen | | | | | *** | ++ |
| 169 | Arg | Trp(6-F) | Cys | | | | | *** | + |
| 170 | Arg | Trp(6-F) | Pen | | | | | *** | + |
| 186 | Arg | Trp(6-F) | Pen | | | | | N/A | N/A |
| 187 | Arg | Trp(6-F) | Pen | | | | | N/A | N/A |
| 188 | Arg | Trp(6-F) | Pen | | | | | N/A | N/A |
| 189 | Arg | Trp(6-F) | Pen | | | | | N/A | N/A |
| 190 | Arg | Trp(6-F) | Pen | | | | | N/A | N/A |
| 191 | Arg | Trp(6-F) | Pen | | | | | N/A | N/A |
| 192 | Arg | Trp(6-F) | Pen | | | | | N/A | N/A |
| 193 | Arg | Trp(6-F) | Pen | | | | | N/A | N/A |
| 194 | Arg | Trp(6-F) | Pen | | | | | N/A | N/A |
| 195 | Arg | Trp(6-F) | Pen | | | | | N/A | N/A |
| 196 | Arg | Trp(6-F) | Pen | | | | | N/A | N/A |
| 197 | Arg | Trp(6-F) | Pen | | | | | N/A | N/A |
| 198 | Arg | Trp(6-F) | Pen | | | | | N/A | N/A |
| 199 | Arg | Trp(6-F) | Pen | | | | | N/A | N/A |
| 200 | Arg | Trp(6-F) | Pen | | | | | N/A | N/A |
| 1058 | Arg | Trp(6-F) | Cys | | | | | ** | + |
| 1092 | Arg | Trp(6-F) | Cys | | | | | *** | ++ |
| 1093 | Arg | Trp(6-F) | Cys | | | | | *** | ++ |
| 1102 | Arg | Trp(6-F) | Cys | | | | | ** | +++ |
| 1103 | Arg | Trp(6-F) | Cys | | | | | ** | +++ |
| 1106 | Arg | Trp(6-F) | Cys | | | | | *** | ++ |
| 1107 | Arg | Trp(6-F) | Cys | | | | | *** | ++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00. [symbol]
indicates data missing or illegible when filed

Tables 12-17 below show families of molecules that have the X₃ position in common. The X₃ residues chosen for investigation were based on X₃ and X₄ combinatorial pairings identified in Table 9 that elicited the greatest selectivity between MC4R and MC1R. Without being bound to a particular theory, throughout these Tables, the peptide sequences illustrated how selectivity between the MC4R and MC1R receptors increased when a specific X₃ and X₄ pairing was identified. The X₁, X₅, and X₇ positions contributed to selectivity, as was seen in Table 15 through the contribution of the X₅ position to improvements in selectivity (Table 15, Molecule 1092, Molecule 1093 and Molecule 1158, and in Table 14, Molecule 1101 and Molecule 1100). However, the substitution interplay was most evident in analogues where certain X₃ and X₄ pairings lead to retention of high MC4R functional potency, with significant decrement of MC1R potency (to generate selectivity). When comparing the selectivity of MC4R v MC1R, larger values indicate selectivity towards MC4R. When comparing the bias of MC4R B-arrestin v MC4R cAMP, larger values indicate bias towards B-arrestin.

TABLE 12
Compounds with Phg at X₃. All peptides are N-acetylated,
have a disulfide linkage and contain a C-terminal amide.

| | | | | | | | | | | Bias: |
|---|---|---|---|---|---|---|---|---|---|---|
| | | | | | | | | | Selec- | MC4R B- |
| Mole- | | | | | | | | | tivity: | arrestin |
| cule | | | | | | | | | MC4R v | v MC4R |
| Name | X₁ | X₂ | X₃ | X₄ | X₅ | X₆ | X₇ | X₈ | MC1R | cAMP |
| 1119 | D-Nar | Cys | Phg | 3-Pal | D-Phe(4-F) | Arg | Trp(6-F) | Cys | *** | ++ |
| 1111 | D-Nar | Cys | Phg | His | D-Phe(4-F) | Arg | Trp(6-F) | Cys | ** | + |
| 1112 | D-Nar | Cys | Phg | His | D-Phe(4-F) | Arg | Trp(5-Me) | Cys | ** | + |
| 1113 | D-Nar | Cys | Phg | His | D-Phe(4-F) | Arg | Trp(6-Me) | Cys | ** | + |
| 1034 | Beta- | Cys | Phg | His | D-Phe | Arg | Trp | Cys | ** | + |
| | homoArg | | | | | | | | | |
| 1110 | D-Nar | Cys | Phg | His | D-Phe | Arg | Trp(6-F) | Cys | * | ++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Another example of the non-predictable pairing of X₃ and X₄ necessary to generate selectivity was the difference between the peptide Molecule 1094 and related peptides that vary in the X₄ position, namely Molecule 1036, or Molecule 1084, or Molecule 1100. Without being bound to a particular theory, the structures showed how the correct pairing of the X₃ and X₄ position was important in generating a transition from no selectivity to −60× selectivity between MC4R and MC1R. When comparing the selectivity of MC4R v MC1R, larger values indicate selectivity towards MC4R. When comparing the bias of MC4R B-arrestin v MC4R cAMP, larger values indicate bias towards B-arrestin.

TABLE 13
Compounds with D-aMeOrn at X₃. All peptides are N-acetylated,
have a disulfide linkage and contain a C-terminal amide.

| | | | | | | | | | | Bias: |
|---|---|---|---|---|---|---|---|---|---|---|
| | | | | | | | | | Selec- | MC4R B- |
| Mole- | | | | | | | | | tivity: | arrestin |
| cule | | | | | | | | | MC4R v | v MC4R |
| Name | X₁ | X₂ | X₃ | X₄ | X₅ | X₆ | X₇ | X₈ | MC1R | cAMP |
| 1094 | D-Nar | Cys | D-aMeOrn | Gln | D-Phe | Arg | Trp(6-F) | Cys | *** | ++ |
| 1124 | D-Nar | Cys | D-aMeOrn | 3-Pal | D-Phe(4-F) | Arg | Trp(6-F) | Cys | *** | ++ |
| 1036 | D-Nar | Cys | D-aMeOrn | His | D-Phe | Arg | Trp(6-F) | Cys | ** | + |
| 1101 | D-Nar | Cys | D-aMeOrn | hGln | D-Phe(4-F) | Arg | Trp(6-F) | Cys | ** | +++ |
| 1125 | D-Nar | Cys | D-aMeOrn | Orn | D-Phe(4-F) | Arg | Trp(6-F) | Cys | ** | + |
| 1100 | D-Nar | Cys | D-aMeOrn | hGln | D-Phe | Arg | Trp(6-F) | Cys | ** | +++ |
| 1055 | D-Arg | Cys | D-aMeOrn | His | D-Phe | Arg | Trp(6-Me) | Cys | ** | + |
| 1109 | D-Nar | Cys | D-aMeOrn | 4-Pal | D-Phe | Arg | Trp(6-F) | Cys | ** | +++ |
| 1010 | Arg | Cys | D-aMeOrn | His | D-Phe | Arg | Trp | Cys | ** | + |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Without being bound to a particular theory, the family of peptides that contained Aib(O-cyclic) demonstrated that certain pairings produced improved selectivity, such as the Gln at X₄. This data also illustrated how the identity of the X₅ position contributes to selectivity.

TABLE 14
Compounds with Aib(O-cyclic) at X₃. All peptides are N-acetylated, have a disulfide
linkage and contain a C-terminal amide. When comparing the selectivity of MC4R
v MC1R, larger values indicate selectivity towards MC4R. When comparing the bias
of MC4R B-arrestin v MC4R cAMP, larger values indicate bias towards B-arrestin.

| | | | | | | | | | | Bias: |
|---|---|---|---|---|---|---|---|---|---|---|
| | | | | | | | | | Selec- | MC4R B- |
| Mole- | | | | | | | | | tivity: | arrestin |
| cule | | | | | | | | | MC4R v | v MC4R |
| Name | X₁ | X₂ | X₃ | X₄ | X₅ | X₆ | X₇ | X₈ | MC1R | cAMP |
| 1106 | D-Nar | Cys | Aib(O- | Cit | D-Phe(4-F) | Arg | Trp(6-F) | Cys | *** | ++ |
| | | | cyclic) | | | | | | | |
| 1122 | D-Nar | Cys | Aib(O- | 3-Pal | D-Phe(4-F) | Arg | Trp(6-F) | Cys | *** | ++ |
| | | | cyclic) | | | | | | | |
| 1107 | D-Nar | Cys | Aib(O- | hCit | D-Phe(4-F) | Arg | Trp(6-F) | Cys | *** | ++ |
| | | | cyclic) | | | | | | | |
| 1093 | D-Nar | Cys | Aib(O- | Gln | D-Phe(4-F) | Arg | Trp(6-F) | Cys | *** | ++ |
| | | | cyclic) | | | | | | | |
| 1092 | D-Nar | Cys | Aib(O- | Gln | D-Phe | Arg | Trp(6-F) | Cys | *** | ++ |
| | | | cyclic) | | | | | | | |
| 1103 | D-Nar | Cys | Aib(O- | Cit | D-Phe | Arg | Trp(6-F) | Cys | ** | +++ |
| | | | cyclic) | | | | | | | |
| 1102 | D-Nar | Cys | Aib(O- | hGln | D-Phe | Arg | Trp(6-F) | Cys | ** | +++ |
| | | | cyclic) | | | | | | | |
| 1058 | D-Nar | Cys | Aib(O- | His | D-Phe | Arg | Trp(6-F) | Cys | ** | + |
| | | | cyclic) | | | | | | | |
| 1123 | D-Nar | Cys | Aib(O- | Orn | D-Phe(4-F) | Arg | Trp(6-F) | Cys | ** | + |
| | | | cyclic) | | | | | | | |
| 1158 | D-Nar | Cys | Aib(O- | Gln | D-Phe(4-F) | Arg | Trp(6-F) | Pen | *** | |
| | | | cyclic) | | | | | | | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 15
Table 15. Compounds with L-aMeGlu at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.

| | | | | | | | | | | | Bias: |
|---|---|---|---|---|---|---|---|---|---|---|---|
| | | | | | | | | | | Selec- | MC4R B- |
| Mole- | | | | | | | | | | tivity: | arrestin v |
| cule | | | | | | | | | | MC4R v | MC4R |
| Name | X₋₁ | X₁ | X₂ | X₃ | X₄ | X₅ | X₆ | X₇ | X₈ | MC1R | cAMP |
| 1020 | | D-Nar | Cys | L-aMeGlu | His | D-Phe | Arg | Trp(6-Me) | Cys | *** | + |
| 1035 | | D-Nar | Cys | L-aMeGlu | His | D-Phe | Arg | Trp(6-F) | Cys | *** | + |
| 1043 | | D-Nar | Cys | L-aMeGlu | His | D-Phe | Arg | Trp(5-Me) | Cys | *** | + |
| 1041 | | D-Nar | Cys | L-aMeGlu | His | D-Phe(4-F) | Arg | Trp(6-Me) | Cys | *** | + |
| 1030 | | Beta- | Cys | L-aMeGlu | His | D-Phe(3-CF3) | Arg | TRP | Cys | *** | +++ |
| | | homoArg | | | | | | | | | |
| 1024 | | Beta- | Cys | L-aMeGlu | His | D-Phe | Arg | Trp(6-Me) | Cys | *** | + |
| | | homoArg | | | | | | | | | |
| 1019 | | D-Nar | Cys | L-aMeGlu | His | D-Phe | Arg | Trp | Cys | *** | + |
| 1085 | | D-Nar | Cys | L-aMeGlu | His | D-Phe(3-F) | Arg | Trp(6-Me) | Cys | *** | + |
| 1016 | | D-Arg | Cys | L-aMeGlu | His | D-Phe | Arg | Trp(6-Me) | Cys | *** | + |
| 1083 | | D-Nar | Cys | L-aMeGlu | His | D-Phe(2,3- | Arg | Trp(6-F) | Cys | ** | + |
| | | | | | | diF) | | | | | |
| 1057 | | Arg | Cys | L-aMeGlu | His | D-Phe(3-F) | Arg | Trp(6-F) | Cys | ** | + |
| 1040 | | D-Nar | Cys | L-aMeGlu | His | D-Phe(3-F) | Arg | Trp(6-F) | Cys | ** | + |
| 1046 | | D-Nar | Cys | L-aMeGlu | His | D-Phe(3,4,5- | Arg | Trp(6-Me) | Cys | ** | ++ |
| | | | | | | triF) | | | | | |
| 1088 | | D-Nar | Cys | L-aMeGlu | His | D-Phe(2,4,5- | Arg | Trp(6-F) | Cys | ** | + |
| | | | | | | triF) | | | | | |
| 1038 | | D-Nar | Cys | L-aMeGlu | His | D-Phe(4-F) | Arg | Trp(6-F) | Cys | ** | + |
| 1079 | | D-Nar | Cys | L-aMeGlu | His | D-Phe | Arg | Trp(5-Cl) | Cys | ** | + |
| 1003 | | Arg | Cys | L-aMeGlu | His | D-Phe | Arg | Trp | Cys | ** | + |
| 1090 | | D-Nar | Cys | L-aMeGlu | Gln | D-Phe | Arg | Trp(6-F) | Cys | ** | ++ |
| 1050 | | D-Nar | Cys | L-aMeGlu | His | D-Phe(3-CF3) | Arg | Trp(6-Me) | Cys | ** | +++ |
| 1007 | | D-Arg | Cys | L-aMeGlu | His | D-Phe | Arg | Trp | Cys | ** | + |
| 1022 | | Beta- | Cys | L-aMeGlu | His | D-Phe | Arg | Trp | Cys | ** | + |
| | | homoArg | | | | | | | | | |
| 1080 | | D-Nar | Cys | L-aMeGlu | His | D-Phe | Arg | Trp(6-Br) | Cys | ** | + |
| 1067 | | D-Nar | Asp | L-aMeGlu | His | D-Phe | Arg | Trp(6-Me) | Dap | ** | + |
| 1104 | | D-Nar | Cys | L-aMeGlu | Cit | D-Phe | Arg | Trp(6-F) | Cys | ** | ++ |
| 1084 | | D-Nar | Cys | L-aMeGlu | His | D-Phe(3-Cl) | Arg | Trp(6-F) | Cys | ** | ++ |
| 1115 | | Nar | Cys | L-aMeGlu | His | D-Phe(4-F) | Arg | Trp(6-F) | Cys | ** | + |
| 1028 | | Beta- | Cys | L-aMeGlu | His | D-Phe | Arg | Trp(6-F) | Cys | ** | ++ |
| | | homoArg | | | | | | | | | |
| 1087 | | D-Nar | Cys | L-aMeGlu | His | D-Phe(2,4- | Arg | Trp(6-F) | Cys | ** | + |
| | | | | | | diF) | | | | | |
| 1054 | | L-hArg | Cys | L-aMeGlu | His | D-Phe(4-F) | Arg | Trp(6-F) | Cys | ** | + |
| 1029 | | L-hArg | Cys | L-aMeGlu | His | D-Phe | Arg | Trp(6-Me) | Cys | ** | ++ |
| 1066 | | D-Nar | Glu | L-aMeGlu | His | D-Phe | Arg | Trp(6-Me) | Dap | ** | ++ |
| 1078 | | D-Nar | Cys | L-aMeGlu | His | D-Phe | Arg | Trp(7-F) | Cys | ** | + |
| 1025 | Gly | Beta- | Cys | L-aMeGlu | His | D-Phe | Arg | Trp | Cys | ** | ++ |
| | | homoArg | | | | | | | | | |
| 1031 | | Beta- | Cys | L-aMeGlu | His | D-Phe(3-Cl) | Arg | TRP | Cys | ** | ++ |
| | | homoArg | | | | | | | | | |
| 1053 | | Arg | Cys | L-aMeGlu | His | D-Phe(4-F) | Arg | Trp(6-F) | Cys | ** | + |
| 1060 | | D-Arg | Cys | L-aMeGlu | His | D-Phe(4-F) | Arg | Trp(6-Me) | Cys | | + |
| 1077 | | D-Nar | Cys | L-aMeGlu | His | D-Phe | Arg | Trp(5-F) | Cys | ** | + |
| 1114 | | Nar | Cys | L-aMeGlu | His | D-Phe | Arg | Trp(6-F) | Cys | ** | + |
| 1018 | Gly | D-Arg | Cys | L-aMeGlu | His | D-Phe | Arg | Trp | Cys | ** | + |
| 1056 | | Arg | Cys | L-aMeGlu | His | D-Phe(4-F) | Arg | Trp(6-Me) | Cys | ** | + |
| 1047 | | D-Nar | Cys | L-aMeGlu | His | D-Phe(3,4,5- | Arg | Trp(6-F) | Cys | ** | ++ |
| | | | | | | triF) | | | | | |
| 1081 | | D-Nar | Cys | L-aMeGlu | His | D-Phe(3-F) | Arg | Trp(5-F) | Cys | ** | + |
| 1075 | | D-Nar | Cys | L-aMeGlu | His | D-Phe | Arg | Trp(6-CF3) | Cys | ** | + |
| 1086 | | D-Nar | Cys | L-aMeGlu | His | D-Phe(3-Me) | Arg | Trp(6-F) | Cys | ** | ++ |
| 1061 | | L-hArg | Cys | L-aMeGlu | His | D-Phe(4-F) | Arg | Trp(6-Me) | Cys | ** | + |
| 1076 | | D-Nar | Cys | L-aMeGlu | His | D-Phe | Arg | Trp(4-F) | Cys | ** | + |
| 1116 | | D-Nar | Cys | L-aMeGlu | 3-Pal | D-Phe | Arg | Trp(6-Me) | Cys | ** | +++ |
| 1048 | | D-Nar | Cys | L-aMeGlu | His | D-Phe(3-Cl) | Arg | Trp(6-Me) | Cys | ** | +++ |
| 1074 | | D-Nar | Cys | L-aMeGlu | His | D-Phe(3,4- | Arg | Trp(6-F) | Cys | ** | + |
| | | | | | | diF) | | | | | |
| 1118 | | D-Nar | Cys | L-aMeGlu | 4-Pal | D-Phe | Arg | Trp(6-Me) | Cys | ** | ++ |
| 1051 | | D-Nar | Cys | L-aMeGlu | His | D-Phe(4-Cl) | Arg | Trp(6-F) | Cys | ** | + |
| 1059 | | Beta- | Cys | L-aMeGlu | His | D-Phe(4-F) | Arg | Trp(6-Me) | Cys | ** | + |
| | | homoArg | | | | | | | | | |
| 1045 | | D-Nar | Cys | L-aMeGlu | His | D-Phe(4-F) | Arg | Trp(6-Cl) | Cys | ** | + |
| 1032 | | Beta- | Cys | L-aMeGlu | His | D-Phe | Arg | Trp(6-Cl) | Cys | ** | + |
| | | homoArg | | | | | | | | | |
| 1082 | | D-Nar | Cys | L-aMeGlu | His | D-Phe(2,4- | Arg | Trp(6-F) | Cys | ** | ++ |
| | | | | | | diCl) | | | | | |
| 1073 | | D-Nar | Cys | L-aMeGlu | His | D-Phe(2-F, | Arg | Trp(6-F) | Cys | ** | + |
| | | | | | | 4-Cl) | | | | | |
| 1021 | | D-Nar | Cys | L-aMeGlu | His | D-Phe(4-Me) | Arg | Trp | Cys | ** | ++ |
| 1026 | | Beta- | Cys | L-aMeGlu | His | D-Phe(4-Cl) | Arg | Trp(6-Me) | Cys | * | ++ |
| | | homoArg | | | | | | | | | |
| 1017 | | D-Arg | Cys | L-aMeGlu | His | D-Phe(4-Me) | Arg | Trp | Cys | * | ++ |
| 1023 | | Beta- | Cys | L-aMeGlu | His | D-Phe(4-Me) | Arg | Trp | Cys | * | ++ |
| | | homoArg | | | | | | | | | |
| 1071 | | D-Nar | Cys | L-aMeGlu | His | D-Phe(3-F, | Arg | Trp(6-F) | Cys | * | ++ |
| | | | | | | 4-Me) | | | | | |
| 1072 | | D-Nar | Cys | L-aMeGlu | His | D-Phe(4-CF3) | Arg | Trp(6-F) | Cys | * | +++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 16
Table 16. Compounds with Cyclo-Leu at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1012 | Arg | Cys | Cyclo- | Gln | D-Phe | Arg | Trp | Cys | *** | +++ |
| | | | Leu | | | | | | | |
| 1108 | D-Nar | Cys | Cyclo- | 3-Pal | D-Phe | Arg | Trp(6-F) | Cys | ** | +++ |
| | | | Leu | | | | | | | |
| 1006 | Beta- | Cys | Cyclo- | His | D-Phe | Arg | Trp | Cys | ** | + |
| | homoArg | | Leu | | | | | | | |
| 1005 | D-Arg | Cys | Cyclo- | His | D-Phe | Arg | Trp | Cys | ** | + |
| | | | Leu | | | | | | | |
| 1052 | Beta- | Cys | Cyclo- | His | D-Phe | Arg | Trp(6-F) | Cys | ** | + |
| | homoArg | | Leu | | | | | | | |
| 1008 | Beta- | Cys | Cyclo- | His | D-Phe | Arg | Trp(6-Me) | Cys | ** | + |
| | homoArg | | Leu | | | | | | | |
| 1001 | Arg | Cys | Cyclo- | His | D-Phe | Arg | Trp | Cys | ** | + |
| | | | Leu | | | | | | | |
| 1009 | D-Arg | Cys | Cyclo- | His | D-Phe | Arg | Trp(6-Me) | Cys | ** | + |
| | | | Leu | | | | | | | |
| 1027 | Beta- | Cys | Cyclo- | His | D- | Arg | TRP | Cys | * | ++ |
| | homoArg | | Leu | | Phe(4-Cl) | | | | | |
| 1015 | D-Arg | Cys | Cyclo- | His | D- | Arg | Trp(6-Me) | Cys | * | +++ |
| | | | Leu | | Phe(4-Me) | | | | | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 17
Table 17. Compounds with L-aMeAsp at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1121 | D-Nar | Glu | L- | His | D- | Arg | Trp(6-F | Dap | *** | + |
| | | | aMeAsp | | Phe(4-F) | | | | | |
| 1042 | D-Nar | Cys | L- | His | D-Phe | Arg | Trp(6-F) | Cys | *** | + |
| | | | aMeAsp | | | | | | | |
| 1064 | D-Nar | Cys | L- | His | D-Phe | Arg | Trp(6-Me) | Cys | *** | + |
| | | | aMeAsp | | | | | | | |
| 1062 | D-Nar | Cys | L- | His | D- | Arg | Trp(6-Me) | Cys | ** | + |
| | | | aMeAsp | | Phe(4-F) | | | | | |
| 1068 | D-Nar | Glu | L- | His | D-Phe | Arg | Trp(6-Me) | Dap | ** | + |
| | | | aMeAsp | | | | | | | |
| 1065 | beta- | Cys | L- | His | D-Phe | Arg | Trp(6-Me) | Cys | ** | + |
| | homoArg | | aMeAsp | | | | | | | |
| 1004 | Arg | Cys | L- | His | D-Phe | Arg | Trp | Cys | ** | + |
| | | | aMeAsp | | | | | | | |
| 1089 | D-Nar | Cys | L- | His | D- | Arg | Trp(6-F) | Cys | ** | ++ |
| | | | aMeAsp | | Phe(3-CF3) | | | | | |
| 1063 | D-Arg | Cys | L- | His | D- | Arg | Trp(6-Me) | Cys | ** | + |
| | | | aMeAsp | | Phe(4-F) | | | | | |
| 1120 | D-Nar | Glu | L- | His | D-Phe | Arg | Trp(6-F) | Dap | ** | + |
| | | | aMeAsp | | | | | | | |
| 1069 | D-Nar | Asp | L- | His | D-Phe | Arg | Trp(6-Me) | Dap | ** | ++ |
| | | | aMeAsp | | | | | | | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Without being bound to a particular theory, the data demonstrates that holding X₃ constant demonstrated that certain X₃ positions were amenable to retaining MC4R potency while losing MC1R potency. The data of Tables 8, 18-20 illustrates how the X₄ position itself was not sufficient to generate the selectivity that the combination of X₃ and X₄ positions generated, even with the X₁, X₅, and/or X₇ positions modified to improve selectivity/potency on MC4R.

TABLE 18
Peptides with 3-Pal at X₄. All peptides are N-acetylated,
have a disulfide linkage and contain a C-terminal amide.

| Molecule | | | | | | | | |
|---|---|---|---|---|---|---|---|---|
| Name | X₁ | X₂ | X₃ | X₄ | X₅ | X₆ | X₇ | X₈ |
| 1119 | D-Nar | Cys | Phg | 3-Pal | D-Phe(4-F) | Arg | Trp(6-F) | Cys |
| 1122 | D-Nar | Cys | Aib(O- | 3-Pal | D-Phe(4-F) | Arg | Trp(6-F) | Cys |
| | | | cyclic) | | | | | |
| 1124 | D-Nar | Cys | D-aMeOrn | 3-Pal | D-Phe(4-F) | Arg | Trp(6-F) | Cys |
| 1108 | D-Nar | Cys | Cyclo-Leu | 3-Pal | D-Phe | Arg | Trp(6-F) | Cys |
| 1116 | D-Nar | Cys | L-aMeGlu | 3-Pal | D-Phe | Arg | Trp(6-Me) | Cys |

TABLE 19
Table 19: Molecule 1119, Molecule 1122, and Molecule 1124 retain
high cAMP potency at MC4R, while other analogues with 3-Pal at
X₄ do not. Molecule 1108 retains B-arrestin potency at MC4R
but loses cAMP potency, and Molecule 1116 loses potency across
both cAMP and B-arrestin. When comparing the selectivity of
MC4R v MC1R, larger values indicate selectivity towards MC4R.
When comparing the bias of MC4R B-arrestin v MC4R cAMP, larger
values indicate bias towards B-arrestin.

| | | Bias: MC4R |
|---|---|---|
| Compound | Selectivity: | B-arrestin v |
| Name | MC4R v MC1R | MC4R cAMP |
| 1119 | *** | ++ |
| 1122 | *** | ++ |
| 1124 | *** | ++ |
| 1108 | ** | +++ |
| 1116 | ** | +++ |

*denotes MC4R vs MC1R selectivity having a range of 0.01 to <1.00,
**denotes MC4R vs MC1R selectivity having a range of ≥1.00 to ≤7.40,
***denotes MC4R vs MC1R selectivity of about >7.40.
+denotes MC4R B-arrestin v MC4R cAMP bias having a range of >0.00 to ≤2.00,
++denotes MC4R B-arrestin v MC4R cAMP bias having a range of 2.00 to ≤10.00,
+++denotes MC4R B-arrestin v MC4R cAMP bias having a value of >10.00.

Without being bound to a particular theory, pairing Gln at X₄ with either D-aMeOrn or Aib(O-cyclic) at X₃ outperformed the next best substitution at X₃ by >2× on MC4R vs MC1R selectivity. When comparing the selectivity of MC4R v MC1R, larger values indicate selectivity towards MC4R. When comparing the bias of MC4R B-arrestin v MC4R cAMP, larger values indicate bias towards B-arrestin.

TABLE 20
Compounds with Gln at X₄

| | | Bias: MC4R |
|---|---|---|
| Compound | Selectivity: | B-arrestin v |
| Name | MC4R v MC1R | MC4R cAMP |
| 1094 | *** | ++ |
| 1093 | *** | ++ |
| 1092 | *** | ++ |
| 1091 | *** | ++ |
| 1096 | *** | ++ |
| 1012 | *** | +++ |
| 1099 | *** | +++ |
| 1070 | ** | ++ |
| 1013 | ** | +++ |
| 1098 | ** | ++ |
| 1011 | ** | ++ |
| 1090 | ** | ++ |
| 1097 | ** | ++ |
| 1014 | ** | ++ |
| 1158 | *** | + |

*denotes MC4R vs MC1R selectivity having a range of 0.01 to <1.00,
**denotes MC4R vs MC1R selectivity having a range of ≥1.00 to ≤7.40,
***denotes MC4R vs MC1R selectivity of about >7.40.
+denotes MC4R B-arrestin v MC4R cAMP bias having a range of >0.00 to ≤2.00,
++denotes MC4R B-arrestin v MC4R cAMP bias having a range of 2.00 to ≤10.00,
+++denotes MC4R B-arrestin v MC4R cAMP bias having a value of >10.00.

Table 8 shown below lists families of molecules that have the X₄ position (e.g., Gln) in common.

The data of Table 8 illustrates how the X₄ position itself was not sufficient to generate the selectivity that the combination of X₃ and X₄ positions generated, even with the X₁, X₅, and/or X₇ positions modified to improve selectivity/potency on MC4R.

Without being bound to a particular theory, pairing Gln at X₄ with Aib(O-cyclic) at X₃ generally outperformed the next best substitution at X₃ by >2× on MC4R vs MC1R selectivity. When comparing the selectivity of MC4R v MC1R, larger values indicate selectivity towards MC4R. When comparing the bias of MC4R B-arrestin v MC4R cAMP, larger values indicate bias towards B-arrestin.

TABLE 8
Compounds with Gln at X₄

| | | | | | | | | | | | | | | | | | | Bias: MC4R |
|---|---|---|---|---|---|---|---|---|---|---|---|---|---|---|---|---|---|---|
| | | | | | | | | | | | | | | | | | Selectivity: | B-arrestin |
| Molecule | | | | | | | | | | | | | | | | | MC4R v | v MC4R |
| name | X₋₄ | X₋₃ | X₋₂ | X₋₁ | X₁ | X₂ | X₃ | X₄ | X₅ | X₆ | X₇ | X₈ | X₉ | X₁₀ | X₁₁ | X₁₂ | MC1R | cAMP |
| 1148 | Lys* | D-Arg | Gly | D-Arg | D-Nar | Cys | D-aMeOrn | Gln | D-Phe | Arg | Trp(6-F) | Cys | | | | | * | + |
| 1149 | | Lys* | Gly | D-Arg | D-Nar | Cys | D-aMeOrn | Gln | D-Phe | Arg | Trp(6-F) | Cys | | | | | * | + |
| 1137 | | Lys* | PEG1 | PEG1 | D-Nar | Cys | D-aMeOrn | Gln | D-Phe | Arg | Trp(6-F) | Cys | | | | | ** | ++ |
| 1138 | Lys* | D-Arg | PEG1 | D-Arg | Beta- | Cys | D-aMeOrn | Gln | D-Phe | Arg | Trp(6-F) | Cys | | | | | ** | ++ |
| | | | | | homoArg | | | | | | | | | | | | | |
| 1150 | Lys* | D-Arg | Gly | D-Arg | D-Nar | Cys | Aib(O- | Gln | D-Phe(4-F) | Arg | Trp(6-F) | Cys | | | | | * | + |
| | | | | | | | cyclic) | | | | | | | | | | | |
| 1142 | | Lys* | Gly | D-Arg | D-Nar | Cys | Aib(O- | Gln | D-Phe(4-F) | Arg | Trp(6-F) | Cys | | | | | ** | + |
| | | | | | | | cyclic) | | | | | | | | | | | |
| 1144 | | Lys* | PEG1 | PEG1 | D-Nar | Cys | Aib(O- | Gln | D-Phe(4-F) | Arg | Trp(6-F) | Cys | | | | | ** | ++ |
| | | | | | | | cyclic) | | | | | | | | | | | |
| 1151 | Lys* | D-Arg | PEG1 | D-Arg | Beta- | Cys | Aib(O- | Gln | D-Phe(4-F) | Arg | Trp(6-F) | Cys | | | | | * | ++ |
| | | | | | homoArg | | cyclic) | | | | | | | | | | | |
| 25 | | | | | D-Nar | Glu | Aib(O- | Gln | D-Phe(4-F) | Arg | Trp(6-F) | Dap | | | | | *** | + |
| | | | | | | | cyclic) | | | | | | | | | | | |
| 26 | | | | | D-Nar | Glu | D-aMeOrn | Gln | D-Phe | Arg | Trp(6-F) | Dap | | | | | *** | + |
| 28 | | | | | Beta- | Cys | Aib(O- | Gln | D-Phe(4-F) | Arg | Trp(6-F) | Cys | | | | | *** | + |
| | | | | | homoArg | | cyclic) | | | | | | | | | | | |
| 29 | | | | | Beta- | Cys | D-aMeOrn | Gln | D-Phe | Arg | Trp(6-F) | Cys | | | | | *** | + |
| | | | | | homoArg | | | | | | | | | | | | | |
| 31 | | | | | Beta- | Cys | Phg | Gln | D-Phe(4-F) | Arg | Trp(6-F) | Cys | | | | | *** | ++ |
| | | | | | homoArg | | | | | | | | | | | | | |
| 32 | | | | | D-Nar | Cys | Phg | Gln | D-Phe(4-F) | Arg | Trp(6-F) | Cys | | | | | *** | ++ |
| 37 | | | | | D-Nar | Cys | Phe | Gln | D-Phe(4-F) | Arg | Trp(6-F) | Cys | | | | | *** | ++ |
| 38 | | | | | D-Nar | Cys | Tyr | Gln | D-Phe(4-F) | Arg | Trp(6-F) | Cys | | | | | *** | ++ |
| 39 | | | | | D-Nar | Cys | D-Phe | Gln | D-Phe(4-F) | Arg | Trp(6-F) | Cys | | | | | *** | + |
| 1158 | | | | | D-Nar | Cys | Aib(O-cyclic) | Gln | D-Phe(4-F) | Arg | Trp(6-F) | Pen | | | | | *** | + |
| 41 | | | | | D-Nar | Cys | D-aMeOrn | Gln | D-Phe | Arg | Trp(6-F) | Pen | | | | | *** | + |
| 43 | | | | | D-Nar | hCys | Aib(O-cyclic) | Gln | D-Phe(4-F) | Arg | Trp(6-F) | Pen | | | | | ** | ++ |
| 44 | | | | | D-Nar | hCys | D-aMeOrn | Gln | D-Phe | Arg | Trp(6-F) | Pen | | | | | ** | ++ |
| 46 | | | | | D-Nar | Cys | Aib(O-cyclic) | Gln | D-Phe | Arg | Trp | Cys | | | | | *** | ++ |
| 47 | | | | | D-Nar | Cys | D-aMeOrn | Gln | D-Phe | Arg | Trp | Cys | | | | | *** | ++ |
| 50 | | | | | Arg | Cys | Phg | Gln | D-Phe | Arg | Trp | Cys | | | | | *** | ++ |
| 51 | | | | | Arg | Cys | Aib(O-cyclic) | Gln | D-Phe | Arg | Trp | Cys | | | | | *** | ++ |
| 52 | | | | | Arg | Cys | D-aMeOrn | Gln | D-Phe | Arg | Trp | Cys | | | | | *** | ++ |
| 53 | | | | | Arg | Cys | D-aMeOrn | Gln | D-Phe | Arg | Trp | Pen | | | | | *** | ++ |
| 56 | | | | | D-Nar | Cys | D-Phg | Gln | D-Phe(4-F) | Arg | Trp(6-F) | Cys | | | | | *** | + |
| 58 | | | | | D-Nar | Cys | D-Iva | Gln | D-Phe(4-F) | Arg | Trp(6-F) | Cys | | | | | *** | + |
| 60 | | | | | D-Nar | Cys | bAc5c | Gln | D-Phe(4-F) | Arg | Trp(6-F) | Cys | | | | | * | + |
| 62 | | | | | Beta- | Cys | bAc5c | Gln | D-Phe(4-F) | Arg | Trp(6-F) | Cys | | | | | ** | ++ |
| | | | | | homoArg | | | | | | | | | | | | | |
| 64 | | | | | D-Nar | Cys | bAc4c | Gln | D-Phe(4-F) | Arg | Trp(6-F) | Cys | | | | | *** | + |
| 66 | | | | | Beta- | Cys | bAc4c | Gln | D-Phe(4-F) | Arg | Trp(6-F) | Cys | | | | | ** | + |
| | | | | | homoArg | | | | | | | | | | | | | |
| 68 | | | | | D-Nar | Cys | bAc3c | Gln | D-Phe(4-F) | Arg | Trp(6-F) | Cys | | | | | ** | + |
| 70 | | | | | Beta- | Cys | bAc3c | Gln | D-Phe(4-F) | Arg | Trp(6-F) | Cys | | | | | *** | ++ |
| | | | | | homoArg | | | | | | | | | | | | | |
| 78 | | | | | D-Nar | Cys | Cyclo-Leu | Gln | D-Phe(4-F) | Arg | Trp(6-F) | Cys | | | | | *** | + |
| 80 | | | | | Beta- | Cys | Cyclo-Leu | Gln | D-Phe(4-F) | Arg | Trp(6-F) | Cys | | | | | *** | + |
| | | | | | homoArg | | | | | | | | | | | | | |
| 101 | | | | | Beta- | Cys | D-aMeSer | Gln | D-Phe | Arg | Trp(6-F) | Cys | | | | | *** | ++ |
| | | | | | homoArg | | | | | | | | | | | | | |
| 108 | | | | | Beta- | Cys | L-aMeSer | Gln | D-Phe | Arg | Trp(6-F) | Cys | | | | | *** | ++ |
| | | | | | homoArg | | | | | | | | | | | | | |
| 110 | | | | | D-Nar | Cys | D-aMeSer | Gln | D-Phe(4-F) | Arg | Trp(5-Me) | Cys | | | | | *** | +++ |
| 111 | Lys* | Gly | Gly | Gly | D-Nar | Cys | Aib(O-cyclic) | Gln | D-Phe(4-F) | Arg | Trp(6-F) | Cys | | | | | * | ++ |
| 112 | | | | | D-Nar | Cys | Aib(O-cyclic) | Gln | D-Phe(4-F) | Arg | Trp(6-F) | Cys | | | | | ** | ++ |
| 113 | | | | | D-Nar | Cys | Aib(O-cyclic) | Gln | D-Phe(4-F) | Arg | Trp(6-F) | Cys | | | | | ** | ++ |
| 114 | | | | Lys* | D-Nar | Cys | Aib(O-cyclic) | Gln | D-Phe(4-F) | Arg | Trp(6-F) | Cys | | | | | ** | ++ |
| 115 | | | | | D-Nar | Cys | Aib(O-cyclic) | Gln | D-Phe(4-F) | Arg | Trp(6-F) | Cys | Gly | Gly | Gly | Lys* | ** | ++ |
| 116 | | | | | D-Nar | Cys | Aib(O-cyclic) | Gln | D-Phe(4-F) | Arg | Trp(6-F) | Cys | Gly | Gly | Lys* | | * | ++ |
| 117 | | | | | D-Nar | Cys | Aib(O-cyclic) | Gln | D-Phe(4-F) | Arg | Trp(6-F) | Cys | Gly | Lys* | | | ** | ++ |
| 118 | | | | | D-Nar | Cys | Aib(O-cyclic) | Gln | D-Phe(4-F) | Arg | Trp(6-F) | Cys | Lys* | | | | * | ++ |
| 119 | | | | | D-Nar | Cys | Aib(O-cyclic) | Gln | D-Phe(4-F) | Arg | Trp(6-F) | Cys | PEG1 | PEG1 | Lys* | | ** | ++ |
| 120 | | | | | D-Nar | Cys | Aib(O-cyclic) | Gln | D-Phe(4-F) | Arg | Trp(6-F) | Cys | D-Arg | Gly | Lys* | | ** | ++ |
| 121 | | | | | D-Nar | Cys | Aib(O-cyclic) | Gln | D-Phe(4-F) | Arg | Trp(6-F) | Cys | Pro | Phe | Lys* | | ** | ++ |
| 122 | | | | | D-Nar | Cys | Aib(O-cyclic) | Gln | D-Phe(4-F) | Arg | Trp(6-F) | Cys | Lys | Pro | Val | Lys* | * | ++ |
| 130 | | Lys* | Gly | Gly | D-Nar | Cys | D-aMeOrn | Gln | D-Phe | Arg | Trp(6-F) | Cys | | | | | ** | ++ |
| 131 | | | Lys* | Gly | D-Nar | Cys | D-aMeOrn | Gln | D-Phe | Arg | Trp(6-F) | Cys | | | | | *** | ++ |
| 132 | | | | Lys* | D-Nar | Cys | D-aMeOrn | Gln | D-Phe | Arg | Trp(6-F) | Cys | | | | | ** | ++ |
| 133 | | | | | D-Nar | Cys | D-aMeOrn | Gln | D-Phe | Arg | Trp(6-F) | Cys | Lys* | | | | * | ++ |
| 134 | | | | | D-Nar | Cys | D-aMeOrn | Gln | D-Phe | Arg | Trp(6-F) | Cys | Gly | Lys* | | | ** | ++ |
| 135 | | | | | D-Nar | Cys | D-aMeOrn | Gln | D-Phe | Arg | Trp(6-F) | Cys | Gly | Gly | Lys* | | ** | ++ |
| 136 | | | | | D-Nar | Cys | D-aMeOrn | Gln | D-Phe | Arg | Trp(6-F) | Cys | Lys | Pro | Val | Lys* | * | ++ |
| 140 | | | | Lys* | Arg | Cys | Aib(O-cyclic) | Gln | D-Phe(4-F) | Arg | Trp(6-F) | Pen | | | | | *** | + |
| 141 | | | | Lys* | D-Nar | Cys | Aib(O-cyclic) | Gln | D-Phe(4-F) | Arg | Trp(6-F) | Pen | | | | | *** | + |
| 142 | | | | Lys* | BetahomoArg | Cys | Aib(O-cyclic) | Gln | D-Phe(4-F) | Arg | Trp(6-F) | Pen | | | | | *** | + |
| 143 | | | | Lys* | Arg | Cys | Aib(O-cyclic) | Gln | D-Phe | Arg | Trp | Pen | | | | | ** | ++ |
| 144 | | | | Lys* | D-Nar | Cys | Aib(O-cyclic) | Gln | D-Phe | Arg | Trp | Pen | | | | | *** | + |
| 145 | | | | Lys* | BetahomoArg | Cys | Aib(O-cyclic) | Gln | D-Phe | Arg | Trp | Pen | | | | | ** | ++ |
| 146 | | | | Lys* | Arg | Cys | Aib(O-cyclic) | Gln | D-Phe | Arg | Trp(6-F) | Pen | | | | | ** | + |
| 147 | | | | Lys* | D-Nar | Cys | Aib(O-cyclic) | Gln | D-Phe | Arg | Trp(6-F) | Pen | | | | | ** | + |
| 148 | | | | Lys* | BetahomoArg | Cys | Aib(O-cyclic) | Gln | D-Phe | Arg | Trp(6-F) | Pen | | | | | *** | ++ |
| 149 | | | | Lys* | Arg | Cys | Aib(O-cyclic) | Gln | D-Phe(4-F) | Arg | Trp | Pen | | | | | *** | + |
| 150 | | | | Lys* | D-Nar | Cys | Aib(O-cyclic) | Gln | D-Phe(4-F) | Arg | Trp | Pen | | | | | *** | + |
| 151 | | | | Lys* | BetahomoArg | Cys | Aib(O-cyclic) | Gln | D-Phe(4-F) | Arg | Trp | Pen | | | | | *** | ++ |
| 152 | | | | | D-Nar | Cys | Aib(O-cyclic) | Gln | D-Phe | Arg | Trp(6-F) | Pen | | | | | *** | + |
| 153 | | | | | D-Nar | Cys | Aib(O-cyclic) | Gln | D-Phe(4-F) | Arg | | Pen | | | | | *** | + |
| 154 | | | | | Beta- | Cys | D-aMeOrn | Gln | D-Phe | Arg | Trp(6-F) | Pen | | | | | *** | + |
| | | | | | homoArg | | | | | | | | | | | | | |
| 155 | | | | | Beta- | Cys | D-aMeOrn | Gln | D-Phe(4-F) | Arg | Trp(6-F) | Pen | | | | | *** | + |
| | | | | | homoArg | | | | | | | | | | | | | |
| 156 | | | | | Beta- | Cys | D-aMeOrn | Gln | D-Phe(4-F) | Arg | Trp | Pen | | | | | *** | + |
| | | | | | homoArg | | | | | | | | | | | | | |
| 157 | | | | | Beta- | Cys | Cyclo-Leu | Gln | D-Phe(4-F) | Arg | Trp(6-F) | Pen | | | | | *** | + |
| | | | | | homoArg | | | | | | | | | | | | | |
| 158 | | | | | D-Nar | Cys | Cyclo-Leu | Gln | D-Phe(4-F) | Arg | Trp(6-F) | Pen | | | | | *** | + |
| 160 | | | | | D-Nar | Cys | D-Iva | Gln | D-Phe(4-F) | Arg | Trp(6-F) | Pen | | | | | *** | ++ |
| 161 | | | | | Beta- | Cys | Cyclo-Leu | Gln | D-Phe | Arg | Trp(6-F) | Pen | | | | | *** | +++ |
| | | | | | homoArg | | | | | | | | | | | | | |
| 162 | | | | | D-Nar | Cys | Cyclo-Leu | Gln | D-Phe | Arg | Trp(6-F) | Pen | | | | | *** | ++ |
| 164 | | | | | D-Nar | Cys | D-Iva | Gln | D-Phe | Arg | Trp(6-F) | Pen | | | | | *** | ++ |
| 165 | | | | Lys* | Arg | Cys | Aib | Gln | D-Phe(4-F) | Arg | Trp(6-F) | Pen | | | | | * | +++ |
| 166 | | | | Lys* | D-Nar | Pen | Aib(O-cyclic) | Gln | D-Phe(4-F) | Arg | Trp(6-F) | Pen | | | | | *** | + |
| 167 | | | | Lys* | D-Nar | Pen | Aib(O-cyclic) | Gln | D-Phe(4-F) | Arg | Trp(6-F) | Cys | | | | | * | +++ |
| 168 | | | | | D-Nar | Pen | Aib(O-cyclic) | Gln | D-Phe(4-F) | Arg | Trp(6-F) | Pen | | | | | *** | ++ |
| 169 | | | | | D-Nar | Pen | Aib(O-cyclic) | Gln | D-Phe(4-F) | Arg | Trp(6-F) | Cys | | | | | *** | + |
| 171 | | | | | D-Nar | Cys | (3S)-3- | Gln | D-Phe(4-F) | Arg | Trp(6-F) | Pen | | | | | N/A | N/A |
| | | | | | | | Aminotetrahydro- | | | | | | | | | | | |
| | | | | | | | 3-furancaboxylic | | | | | | | | | | | |
| | | | | | | | acid | | | | | | | | | | | |
| 172 | | | | | D-Nar | Cys | (3R)-3- | Gln | D-Phe(4-F) | Arg | Trp(6-F) | Pen | | | | | N/A | N/A |
| | | | | | | | Aminotetrahydro- | | | | | | | | | | | |
| | | | | | | | 3-furancaboxylic | | | | | | | | | | | |
| | | | | | | | acid | | | | | | | | | | | |
| 173 | | | | | D-Nar | Cys | (3S)-3- | Gln | D-Phe(4-F) | Arg | Trp(6-F) | Pen | | | | | N/A | N/A |
| | | | | | | | Aminotetrahydro- | | | | | | | | | | | |
| | | | | | | | 3-thiphenecarboxylic | | | | | | | | | | | |
| | | | | | | | acid | | | | | | | | | | | |
| 174 | | | | | D-Nar | Cys | (3R)-3- | Gln | D-Phe(4-F) | Arg | Trp(6-F) | Pen | | | | | N/A | N/A |
| | | | | | | | Aminotetrahydro- | | | | | | | | | | | |
| | | | | | | | 3-thiphenecarboxylic | | | | | | | | | | | |
| | | | | | | | acid | | | | | | | | | | | |
| 175 | | | | | D-Nar | Cys | N-Boc-(3S)-3- | Gln | D-Phe(4-F) | Arg | Trp(6-F) | Pen | | | | | N/A | N/A |
| | | | | | | | amino-1,3- | | | | | | | | | | | |
| | | | | | | | pyrrolidinedicarboxylate | | | | | | | | | | | |
| 176 | | | | | D-Nar | Cys | N-Boc-(3R)-3- | Gln | D-Phe(4-F) | Arg | Trp(6-F) | Pen | | | | | N/A | N/A |
| | | | | | | | amino-1,3- | | | | | | | | | | | |
| | | | | | | | pyrrolidinedicarboxylate | | | | | | | | | | | |
| 177 | | | | | D-Nar | Cys | 3-Amino-3-thietane- | Gln | D-Phe(4-F) | Arg | Trp(6-F) | Pen | | | | | N/A | N/A |
| | | | | | | | carboxylic acid | | | | | | | | | | | |
| 178 | | | | | D-Nar | Cys | 3-Aminothletane-3- | Gln | D-Phe(4-F) | Arg | Trp(6-F) | Pen | | | | | N/A | N/A |
| | | | | | | | carboxylic acid 1,1- | | | | | | | | | | | |
| | | | | | | | dioxide | | | | | | | | | | | |
| 179 | | | | | D-Nar | Cys | N-Boc-3-amino-1,3- | Gln | D-Phe(4-F) | Arg | Trp(6-F) | Pen | | | | | N/A | N/A |
| | | | | | | | azetidinedicarboxylate | | | | | | | | | | | |
| 180 | | | | | D-Nar | Cys | 1-Amino-3,3- | Gln | D-Phe(4-F) | Arg | Trp(6-F) | Pen | | | | | N/A | N/A |
| | | | | | | | dimethylcyclobutane- | | | | | | | | | | | |
| | | | | | | | carboxylic acid | | | | | | | | | | | |
| 181 | | | | | D-Nar | Cys | 5-Aminospiro[2.3]hexane- | Gln | D-Phe(4-F) | Arg | Trp(6-F) | Pen | | | | | N/A | N/A |
| | | | | | | | 5-carboxylic acid | | | | | | | | | | | |
| 182 | | | | | D-Nar | Cys | 6-Amino-2- | Gln | D-Phe(4-F) | Arg | Trp(6-F) | Pen | | | | | N/A | N/A |
| | | | | | | | oxaspiro[3.3]heptane- | | | | | | | | | | | |
| | | | | | | | 6-carboxylic acid | | | | | | | | | | | |
| 183 | | | | | D-Nar | Cys | 2-amino-2- | Gln | D-Phe(4-F) | Arg | Trp(6-F) | Pen | | | | | N/A | N/A |
| | | | | | | | ethylbutanoic acid | | | | | | | | | | | |
| 184 | | | | | D-Nar | Cys | (1S)-1-Amino-2,3- | Gln | D-Phe(4-F) | Arg | Trp(6-F) | Pen | | | | | N/A | N/A |
| | | | | | | | dihydro-1H-indene-1- | | | | | | | | | | | |
| | | | | | | | carboxylic acid | | | | | | | | | | | |
| 185 | | | | | D-Nar | Cys | (1R)-1-Amino-2,3- | Gln | D-Phe(4-F) | Arg | Trp(6-F) | Pen | | | | | N/A | N/A |
| | | | | | | | dihydro-1H-indene-1- | | | | | | | | | | | |
| | | | | | | | carboxylic acid | | | | | | | | | | | |
| 186 | | | | | D-Nar | Cys | Aib(O-cyclic) | Gln | L-methionine | Arg | Trp(6-F) | Pen | | | | | N/A | N/A |
| | | | | | | | | | sulfoxide | | | | | | | | | |
| 187 | | | | | D-Nar | Cys | Aib(O-cyclic) | Gln | L-methionine | Arg | Trp(6-F) | Pen | | | | | N/A | N/A |
| | | | | | | | | | sulfone | | | | | | | | | |
| 188 | | | | | D-Nar | Cys | Aib(O-cyclic) | Gln | (2S)-2-Amino-4- | Arg | Trp(6-F) | Pen | | | | | N/A | N/A |
| | | | | | | | | | cyanobutanoic | | | | | | | | | |
| | | | | | | | | | acid | | | | | | | | | |
| 189 | | | | | D-Nar | Cys | Aib(O-cyclic) | Gln | 3-(acetylamino)- | Arg | Trp(6-F) | Pen | | | | | N/A | N/A |
| | | | | | | | | | L-alanine | | | | | | | | | |
| 190 | | | | | D-Nar | Cys | Aib(O-cyclic) | Gln | O-Carbamoyl-L- | Arg | Trp(6-F) | Pen | | | | | N/A | N/A |
| | | | | | | | | | serine | | | | | | | | | |
| 191 | | | | | D-Nar | Cys | Aib(O-cyclic) | Gln | 2-Hydroxy-L- | Arg | Trp(6-F) | Pen | | | | | N/A | N/A |
| | | | | | | | | | tryptophen | | | | | | | | | |
| 192 | | | | | D-Nar | Cys | Aib(O-cyclic) | Gln | 3-(Trimethylsilyl)- | Arg | Trp(6-F) | Pen | | | | | N/A | N/A |
| | | | | | | | | | D-alanine | | | | | | | | | |
| 193 | | | | | D-Nar | Cys | Aib(O-cyclic) | Gln | 5,5,5-Trifluoro-D- | Arg | Trp(6-F) | Pen | | | | | N/A | N/A |
| | | | | | | | | | norvaline | | | | | | | | | |
| 194 | | | | | D-Nar | Cys | Aib(O-cyclic) | Gln | 3-(Trifluoromethyl)- | Arg | Trp(6-F) | Pen | | | | | N/A | N/A |
| | | | | | | | | | D-alanine | | | | | | | | | |
| 195 | | | | | D-Nar | Cys | Aib(O-cyclic) | Gln | 3-Cyano-D- | Arg | Trp(6-F) | Pen | | | | | N/A | N/A |
| | | | | | | | | | alanine | | | | | | | | | |
| 196 | | | | | D-Nar | Cys | Aib(O-cyclic) | Gln | 3-Cyclopropyl-D- | Arg | Trp(6-F) | Pen | | | | | N/A | N/A |
| | | | | | | | | | alanine | | | | | | | | | |
| 197 | | | | | D-Nar | Cys | Aib(O-cyclic) | Gln | (R)-2-Amino-4- | Arg | Trp(6-F) | Pen | | | | | N/A | N/A |
| | | | | | | | | | cyclopropylbutanoic | | | | | | | | | |
| | | | | | | | | | acid | | | | | | | | | |
| 198 | | | | | D-Nar | Cys | Aib(O-cyclic) | Gln | (αR)-α-Amino-2- | Arg | Trp(6-F) | Pen | | | | | N/A | N/A |
| | | | | | | | | | pyridine- | | | | | | | | | |
| | | | | | | | | | propenoic acid | | | | | | | | | |
| 199 | | | | | D-Nar | Cys | Aib(O-cyclic) | Gln | (αR)-α-Amino-3- | Arg | Trp(6-F) | Pen | | | | | N/A | N/A |
| | | | | | | | | | pyridine- | | | | | | | | | |
| | | | | | | | | | propenoic acid | | | | | | | | | |
| 200 | | | | | D-Nar | Cys | Aib(O-cyclic) | Gln | (αR)-α-Amino-4- | Arg | Trp(6-F) | Pen | | | | | N/A | N/A |
| | | | | | | | | | pyridine- | | | | | | | | | |
| | | | | | | | | | propenoic acid | | | | | | | | | |
| 1011 | | | | | Arg | Cys | D-Ala | Gln | D-Phe | Arg | Trp | Cys | | | | | ** | ++ |
| 1012 | | | | | Arg | Cys | Cyclo-Leu | Gln | D-Phe | Arg | Trp | Cys | | | | | *** | +++ |
| 1013 | | | | | Arg | Cys | Ala(2-Me) | Gln | D-Phe | Arg | Trp | Cys | | | | | ** | +++ |
| 1014 | | | | | Beta- | Cys | D-Dab | Gln | D-Phe | Arg | Trp | Cys | | | | | ** | ++ |
| | | | | | homoArg | | | | | | | | | | | | | |
| 1070 | | | | | Arg | Cys | D-Ala | Gln | D-Phe | Arg | Trp | Cys | | | | | ** | ++ |
| 1090 | | | | | D-Nar | Cys | L-aMeGlu | Gln | D-Phe | Arg | Trp(6-F) | Cys | | | | | ** | ++ |
| 1091 | | | | | D-Nar | Cys | hGlu | Gln | D-Phe | Arg | Trp(6-F) | Cys | | | | | *** | ++ |
| 1092 | | | | | D-Nar | Cys | Aib(O-cyclic) | Gln | D-Phe | Arg | Trp(6-F) | Cys | | | | | *** | ++ |
| 1093 | | | | | D-Nar | Cys | Aib(O-cyclic) | Gln | D-Phe(4-F) | Arg | Trp(6-F) | Cys | | | | | *** | ++ |
| 1094 | | | | | D-Nar | Cys | D-aMeOrn | Gln | D-Phe | Arg | Trp(6-F) | Cys | | | | | *** | ++ |
| 1096 | | | | | D-Nar | Cys | D-aMeSer | Gln | D-Phe | Arg | Trp(6-F) | Cys | | | | | *** | ++ |
| 1097 | | | | | D-Nar | Cys | D-aMeSer | Gln | D-Phe(4-F) | Arg | Trp(6-F) | Cys | | | | | ** | ++ |
| 1898 | | | | | D-Nar | Cys | bhGlu | Gln | D-Phe | Arg | Trp(6-F) | Cys | | | | | ** | ++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

### Selectivity for MC4R or MC1R

Table 11 lists the calculated MC4R vs MC1R selectivity of some exemplary peptides tested. Setmelanotide has a selectivity of 1.06, and Melanotan II has a selectivity of 0.08. When comparing the selectivity of MC4R v MC1R, larger values indicate selectivity towards MC4R. When comparing the bias of MC4R B-arrestin v MC4R cAMP, larger values indicate bias towards B-arrestin.

TABLE 11
Table 11: The in vitro assay activity and calculated selectivity
and calculated bias of the foregoing exemplary molecules that
are selective for MC4R vs MC1R in cAMP functional screening
and may also be biased for B-arrestin over cAMP signal.

| | | Bias: MC4R |
|---|---|---|
| Molecule | Selectivity: | B-arrestin v |
| Name | MC4R v MC1R | MC4R cAMP |
| 1119 | *** | ++ |
| 1094 | *** | ++ |
| 1106 | *** | ++ |
| 1122 | *** | ++ |
| 1107 | *** | ++ |
| 1093 | *** | ++ |
| 1092 | *** | ++ |
| 1124 | *** | ++ |
| 1015 | *** | + |
| 1035 | *** | + |
| 1091 | *** | ++ |
| 1096 | *** | ++ |
| 1043 | *** | + |
| 1012 | *** | +++ |
| 1049 | *** | + |
| 1041 | *** | + |
| 1099 | *** | +++ |
| 1030 | *** | +++ |
| 1121 | *** | + |
| 1042 | *** | + |
| 1024 | *** | + |
| 1064 | *** | + |
| 1037 | *** | + |
| 1019 | *** | + |
| 1085 | *** | + |
| 1016 | *** | + |
| 1158 | *** | |

*denotes MC4R vs MC1R selectivity having a range of 0.01 to <1.00,
**denotes MC4R vs MC1R selectivity having a range of ≥1.00 to ≤7.40,
***denotes MC4R vs MC1R selectivity of about >7.40.
+denotes MC4R B-arrestin v MC4R cAMP bias having a range of >0.00 to ≤2.00,
++denotes MC4R B-arrestin v MC4R cAMP bias having a range of 2.00 to ≤10.00,
+++denotes MC4R B-arrestin v MC4R cAMP bias having a value of >10.00.

Tables 12-17 below show exemplary families of molecules that have the X₃ position in common. The X₃ residues chosen for investigation were based on X₃ and X₄ combinatorial pairings identified in Table 9 that elicited the greatest selectivity between MC4R and MC1R. Without being bound to a particular theory, throughout these Tables, the peptide sequences illustrated how selectivity between the MC4R and MC1R receptors increased when a specific X₃ and X₄ pairing was identified. The X₁, X₅, and X₇ positions contributed to selectivity, as was seen in Table 14 through the contribution of the X₅ position to improvements in selectivity (Table 14, Molecule 1092, Molecule 1093 and Molecule 1158, and in Table 13, Molecule 1101 and Molecule 1100). However, the substitution interplay was most evident in analogues where certain X₃ and X₄ pairings lead to retention of high MC4R functional potency, with significant decrement of MC1R potency (to generate selectivity). When comparing the selectivity of MC4R v MC1R, larger values indicate selectivity towards MC4R. When comparing the bias of MC4R B-arrestin v MC4R cAMP, larger values indicate bias towards B-arrestin.

TABLE 12
Compounds with Phg at X₃. All peptides are N-acetylated, have a disulfide linkage and contain a C-terminal amide.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1119 | D-Nar | Cys | Phg | 3-Pal | D-Phe(4-F) | Arg | Trp(6-F) | Cys | *** | ++ |
| 1111 | D-Nar | Cys | Phg | His | D-Phe(4-F) | Arg | Trp(6-F) | Cys | ** | + |
| 1112 | D-Nar | Cys | Phg | His | D-Phe(4-F) | Arg | Trp(5-Me) | Cys | ** | + |
| 1113 | D-Nar | Cys | Phg | His | D-Phe(4-F) | Arg | Trp(6-Me) | Cys | ** | + |
| 1034 | Beta- | Cys | Phg | His | D-Phe | Arg | Trp | Cys | ** | + |
| | homoArg | | | | | | | | | |
| 1110 | D-Nar | Cys | Phg | His | D-Phe | Arg | Trp(6-F) | Cys | * | ++ |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Another example of the non-predictable pairing of X₃ and X₄ necessary to generate selectivity was the difference between the peptide Molecule 1094 and related peptides that vary in the X₄ position, namely Molecule 1036, or Molecule 1084, or Molecule 1100. Without being bound to a particular theory, the structures showed how the pairing of the X₃ and X₄ position was important in generating a transition from no selectivity to ˜60× selectivity between MC4R and MC1R. When comparing the selectivity of MC4R v MC1R, larger values indicate selectivity towards MC4R. When comparing the bias of MC4R B-arrestin v MC4R cAMP, larger values indicate bias towards B-arrestin.

TABLE 13
Exemplary compounds with D-aMeOrn at X₃. All peptides are N-acetylated,
have a disulfide linkage and contain a C-terminal amide.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1094 | D-Nar | Cys | D- | Gln | D-Phe | Arg | Trp(6-F) | Cys | *** | ++ |
| | | | aMeOrn | | | | | | | |
| 1124 | D-Nar | Cys | D- | 3-Pal | D-Phe(4-F) | Arg | Trp(6-F) | Cys | *** | ++ |
| | | | aMeOrn | | | | | | | |
| 1036 | D-Nar | Cys | D- | His | D-Phe | Arg | Trp(6-F) | Cys | ** | + |
| | | | aMeOrn | | | | | | | |
| 1101 | D-Nar | Cys | D- | hGIn | D-Phe(4-F) | Arg | Trp(6-F) | Cys | ** | +++ |
| | | | aMeOrn | | | | | | | |
| 1125 | D-Nar | Cys | D- | Orn | D-Phe(4-F) | Arg | Trp(6-F) | Cys | ** | + |
| | | | aMeOrn | | | | | | | |
| 1100 | D-Nar | Cys | D- | hGIn | D-Phe | Arg | Trp(6-F) | Cys | ** | +++ |
| | | | aMeOrn | | | | | | | |
| 1055 | D-Arg | Cys | D- | His | D-Phe | Arg | Trp(6-Me) | Cys | ** | + |
| | | | aMeOrn | | | | | | | |
| 1109 | D-Nar | Cys | D- | 4-Pal | D-Phe | Arg | Trp(6-F) | Cys | ** | +++ |
| | | | aMeOrn | | | | | | | |
| 1010 | Arg | Cys | D- | His | D-Phe | Arg | Trp | Cys | ** | + |
| | | | aMeOrn | | | | | | | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Without being bound to a particular theory, the family of peptides that contained Aib(O-cyclic) demonstrated that certain pairings produced improved selectivity, such as the Gln at X₄. This data also illustrated how the identity of the X₅ position contributes to selectivity.

TABLE 14
Exemplary compounds with Aib(O-cyclic) at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.

| | | | | | | | | | | Bias: MC4R |
|---|---|---|---|---|---|---|---|---|---|---|
| Molecule | | | | | | | | | Selectivity: | B-arrestin v |
| Name | X | X₂ | X₃ | X₄ | X₅ | X₆ | X₇ | X₈ | MC4R v MC1R | MC4R cAMP |
| 1106 | D-Nar | Cys | Aib(O- | Cit | D-Phe(4-F) | Arg | Trp(6-F) | Cys | *** | ++ |
| | | | cyclic) | | | | | | | |
| 1122 | D-Nar | Cys | Aib(O- | 3-Pal | D-Phe(4-F) | Arg | Trp(6-F) | Cys | *** | ++ |
| | | | cyclic) | | | | | | | |
| 1107 | D-Nar | Cys | Aib(O- | hCit | D-Phe(4-F) | Arg | Trp(6-F) | Cys | *** | ++ |
| | | | cyclic) | | | | | | | |
| 1093 | D-Nar | Cys | Aib(O- | Gln | D-Phe(4-F) | Arg | Trp(6-F) | Cys | *** | ++ |
| | | | cyclic) | | | | | | | |
| 1092 | D-Nar | Cys | Aib(O- | Gln | D-Phe | Arg | Trp(6-F) | Cys | *** | ++ |
| | | | cyclic) | | | | | | | |
| 1103 | D-Nar | Cys | Aib(O- | Cit | D-Phe | Arg | Trp(6-F) | Cys | ** | +++ |
| | | | cyclic) | | | | | | | |
| 1102 | D-Nar | Cys | Aib(O- | hGln | D-Phe | Arg | Trp(6-F) | Cys | ** | +++ |
| | | | cyclic) | | | | | | | |
| 1058 | D-Nar | Cys | Aib(O- | His | D-Phe | Arg | Trp(6-F) | Cys | ** | + |
| | | | cyclic) | | | | | | | |
| 1123 | D-Nar | Cys | Aib(O- | Orn | D-Phe(4-F) | Arg | Trp(6-F) | Cys | ** | + |
| | | | cyclic) | | | | | | | |
| 1158 | D-Nar | Cys | Aib(O- | Gln | D-Phe(4-F) | Arg | Trp(6-F) | Pen | *** | |
| | | | cyclic) | | | | | | | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 15
Exemplary compounds with L-aMeGlu at X₃. All peptides are N-acetylated, have a disulfide linkage and contain
a C-terminal amide. When comparing the selectivity of MC4R v MC1R, larger values indicate selectivity towards
MC4R. When comparing the bias of MC4R B-arrestin v MC4R cAMP, larger values indicate bias towards B-arrestin.

| | | | | | | | | | | | Bias: MC4R |
|---|---|---|---|---|---|---|---|---|---|---|---|
| Molecule | | | | | | | | | | Selectivity: | B-arrestin v |
| Name | X₋₁ | X₁ | X₂ | X₃ | X₄ | X₅ | X₆ | X₇ | X₈ | MC4R v MC1R | MC4R cAMP |
| 1020 | | D-Nar | Cys | L- | His | D-Phe | Arg | Trp(6-Me) | Cys | *** | + |
| | | | | aMeGlu | | | | | | | |
| 1035 | | D-Nar | Cys | L- | His | D-Phe | Arg | Trp(6-F) | Cys | *** | + |
| | | | | aMeGlu | | | | | | | |
| 1043 | | D-Nar | Cys | L- | His | D-Phe | Arg | Trp(5-Me) | Cys | *** | ++ |
| | | | | aMeGlu | | | | | | | |
| 1041 | | D-Nar | Cys | L- | His | D-Phe(4-F) | Arg | Trp(6-Me) | Cys | *** | + |
| | | | | aMeGlu | | | | | | | |
| 1030 | | Beta- | Cys | L- | His | D-Phe(3-CF3) | Arg | TRP | Cys | *** | +++ |
| | | homoArg | | aMeGlu | | | | | | | |
| 1024 | | Beta- | Cys | L- | His | D-Phe | Arg | Trp(6-Me) | Cys | *** | + |
| | | homoArg | | aMeGlu | | | | | | | |
| 1019 | | D-Nar | Cys | L- | His | D-Phe | Arg | Trp | Cys | *** | + |
| | | | | aMeGlu | | | | | | | |
| 1085 | | D-Nar | Cys | L- | His | D-Phe(3-F) | Arg | Trp(6-Me) | Cys | *** | + |
| | | | | aMeGlu | | | | | | | |
| 1016 | | D-Arg | Cys | L- | His | D-Phe | Arg | Trp(6-Me) | Cys | *** | + |
| | | | | aMeGlu | | | | | | | |
| 1083 | | D-Nar | Cys | L- | His | D-Phe(2,3-diF) | Arg | Trp(6-F) | Cys | ** | + |
| | | | | aMeGlu | | | | | | | |
| 1057 | | Arg | Cys | L- | His | D-Phe(3-F) | Arg | Trp(6-F) | Cys | ** | + |
| | | | | aMeGlu | | | | | | | |
| 1040 | | D-Nar | Cys | L- | His | D-Phe(3-F) | Arg | Trp(6-F) | Cys | ** | + |
| | | | | aMeGlu | | | | | | | |
| 1046 | | D-Nar | Cys | L- | His | D- | Arg | Trp(6-Me) | Cys | ** | |
| | | | | aMeGlu | | Phe(3,4,5-triF) | | | | | ++ |
| 1088 | | D-Nar | Cys | L- | His | D- | Arg | Trp(6-F) | Cys | ** | + |
| | | | | aMeGlu | | Phe(2,4,5-triF) | | | | | |
| 1038 | | D-Nar | Cys | L- | His | D-Phe(4-F) | Arg | Trp(6-F) | Cys | ** | + |
| | | | | aMeGlu | | | | | | | |
| 1079 | | D-Nar | Cys | L- | His | D-Phe | Arg | Trp(5-Cl) | Cys | ** | + |
| | | | | aMeGlu | | | | | | | |
| 1003 | | Arg | Cys | L- | His | D-Phe | Arg | Trp | Cys | ** | + |
| | | | | aMeGlu | | | | | | | |
| 1090 | | D-Nar | Cys | L- | Gln | D-Phe | Arg | Trp(6-F) | Cys | ** | ++ |
| | | | | aMeGlu | | | | | | | |
| 1050 | | D-Nar | Cys | L- | His | D-Phe(3-CF3) | Arg | Trp(6-Me) | Cys | ** | +++ |
| | | | | aMeGlu | | | | | | | |
| 1007 | | D-Arg | Cys | L- | His | D-Phe | Arg | Trp | Cys | ** | + |
| | | | | aMeGlu | | | | | | | |
| 1022 | | Beta- | Cys | L- | His | D-Phe | Arg | Trp | Cys | ** | + |
| | | homoArg | | aMeGlu | | | | | | | |
| 1080 | | D-Nar | Cys | L- | His | D-Phe | Arg | Trp(6-Br) | Cys | ** | + |
| | | | | aMeGlu | | | | | | | |
| 1067 | | D-Nar | Asp | L- | His | D-Phe | Arg | Trp(6-Me) | Dap | ** | + |
| | | | | aMeGlu | | | | | | | |
| 1104 | | D-Nar | Cys | L- | Cit | D-Phe | Arg | Trp(6-F) | Cys | ** | ++ |
| | | | | aMeGlu | | | | | | | |
| 1084 | | D-Nar | Cys | L- | His | D-Phe(3-Cl) | Arg | Trp(6-F) | Cys | ** | ++ |
| | | | | aMeGlu | | | | | | | |
| 1115 | | Nar | Cys | L- | His | D-Phe(4-F) | Arg | Trp(6-F) | Cys | ** | + |
| | | | | aMeGlu | | | | | | | |
| 1028 | | Beta- | Cys | L- | His | D-Phe | Arg | Trp(6-F) | Cys | ** | ++ |
| | | homoArg | | aMeGlu | | | | | | | |
| 1087 | | D-Nar | Cys | L- | His | D-Phe(2,4-diF) | Arg | Trp(6-F) | Cys | ** | + |
| | | | | aMeGlu | | | | | | | |
| 1054 | | L-hArg | Cys | L- | His | D-Phe(4-F) | Arg | Trp(6-F) | Cys | ** | + |
| | | | | aMeGlu | | | | | | | |
| 1029 | | L-hArg | Cys | L- | His | D-Phe | Arg | Trp(6-Me) | Cys | ** | ++ |
| | | | | aMeGlu | | | | | | | |
| 1066 | | D-Nar | Glu | L- | His | D-Phe | Arg | Trp(6-Me) | Dap | ** | ++ |
| | | | | aMeGlu | | | | | | | |
| 1078 | | D-Nar | Cys | L- | His | D-Phe | Arg | Trp(7-F) | Cys | ** | + |
| | | | | aMeGlu | | | | | | | |
| 1025 | Gly | Beta- | Cys | L- | His | D-Phe | Arg | Trp | Cys | ** | ++ |
| | | homoArg | | aMeGlu | | | | | | | |
| 1031 | | Beta- | Cys | L- | His | D-Phe(3-Cl) | Arg | TRP | Cys | ** | ++ |
| | | homoArg | | aMeGlu | | | | | | | |
| 1053 | | Arg | Cys | L- | His | D-Phe(4-F) | Arg | Trp(6-F) | Cys | ** | ++ |
| | | | | aMeGlu | | | | | | | |
| 1060 | | D-Arg | Cys | L- | His | D-Phe(4-F) | Arg | Trp(6-Me) | Cys | ** | ++ |
| | | | | aMeGlu | | | | | | | |
| 1077 | | D-Nar | Cys | L- | His | D-Phe | Arg | Trp(5-F) | Cys | ** | + |
| | | | | aMeGlu | | | | | | | |
| 1114 | | Nar | Cys | L- | His | D-Phe | Arg | Trp(6-F) | Cys | ** | + |
| | | | | aMeGlu | | | | | | | |
| 1018 | Gly | D-Arg | Cys | L- | His | D-Phe | Arg | Trp | Cys | ** | + |
| | | | | aMeGlu | | | | | | | |
| 1056 | | Arg | Cys | L- | His | D-Phe(4-F) | Arg | Trp(6-Me) | Cys | ** | + |
| | | | | aMeGlu | | | | | | | |
| 1047 | | D-Nar | Cys | L- | His | D- | Arg | Trp(6-F) | Cys | ** | ++ |
| | | | | aMeGlu | | Phe(3,4,5-triF) | | | | | |
| 1081 | | D-Nar | Cys | L- | His | D-Phe(3-F) | Arg | Trp(5-F) | Cys | ** | + |
| | | | | aMeGlu | | | | | | | |
| 1075 | | D-Nar | Cys | L- | His | D-Phe | Arg | Trp(6-CF3) | Cys | ** | + |
| | | | | aMeGlu | | | | | | | |
| 1086 | | D-Nar | Cys | L- | His | D-Phe(3-Me) | Arg | Trp(6-F) | Cys | ** | ++ |
| | | | | aMeGlu | | | | | | | |
| 1061 | | L-hArg | Cys | L- | His | D-Phe(4-F) | Arg | Trp(6-Me) | Cys | ** | + |
| | | | | aMeGlu | | | | | | | |
| 1076 | | D-Nar | Cys | L- | His | D-Phe | Arg | Trp(4-F) | Cys | ** | + |
| | | | | aMeGlu | | | | | | | |
| 1116 | | D-Nar | Cys | L- | 3-Pal | D-Phe | Arg | Trp(6-Me) | Cys | ** | +++ |
| | | | | aMeGlu | | | | | | | |
| 1048 | | D-Nar | Cys | L- | His | D-Phe(3-Cl) | Arg | Trp(6-Me) | Cys | ** | +++ |
| | | | | aMeGlu | | | | | | | |
| 1074 | | D-Nar | Cys | L- | His | D- | Arg | Trp(6-F) | Cys | ** | + |
| | | | | aMeGlu | | Phe(3,4-diF) | | | | | |
| 1118 | | D-Nar | Cys | L- | 4-Pal | D-Phe | Arg | Trp(6-Me) | Cys | ** | ++ |
| | | | | aMeGlu | | | | | | | |
| 1051 | | D-Nar | Cys | L- | His | D-Phe(4-Cl) | Arg | Trp(6-F) | Cys | ** | + |
| | | | | aMeGlu | | | | | | | |
| 1059 | | Beta- | Cys | L- | His | D-Phe(4-F) | Arg | Trp(6-Me) | Cys | ** | + |
| | | homoArg | | aMeGlu | | | | | | | |
| 1045 | | D-Nar | Cys | L- | His | D-Phe(4-F) | Arg | Trp(6-Cl) | Cys | ** | + |
| | | | | aMeGlu | | | | | | | |
| 1032 | | Beta- | Cys | L- | His | D-Phe | Arg | Trp(6-Cl) | Cys | ** | + |
| | | homoArg | | aMeGlu | | | | | | | |
| 1082 | | D-Nar | Cys | L- | His | D- | Arg | Trp(6-F) | Cys | ** | ++ |
| | | | | aMeGlu | | Phe(2,4-diCl) | | | | | |
| 1073 | | D-Nar | Cys | L- | His | D- | Arg | Trp(6-F) | Cys | ** | + |
| | | | | aMeGlu | | Phe(2-F, 4-Cl) | | | | | |
| 1021 | | D-Nar | Cys | L- | His | D-Phe(4-Me) | Arg | Trp | Cys | ** | ++ |
| | | | | aMeGlu | | | | | | | |
| 1026 | | Beta- | Cys | L- | His | D-Phe(4-Cl) | Arg | Trp(6-Me) | Cys | * | ++ |
| | | homoArg | | aMeGlu | | | | | | | |
| 1017 | | D-Arg | Cys | L- | His | D-Phe(4-Me) | Arg | Trp | Cys | * | ++ |
| | | | | aMeGlu | | | | | | | |
| 1023 | | Beta- | Cys | L- | His | D-Phe(4Me) | Arg | Trp | Cys | * | ++ |
| | | homoArg | | aMeGlu | | | | | | | |
| 1071 | | D-Nar | Cys | L- | His | D- | Arg | Trp(6-F) | Cys | * | ++ |
| | | | | aMeGlu | | Phe(3-F, 4-Me) | | | | | |
| 1072 | | D-Nar | Cys | L- | His | D-Phe(4-CF3) | Arg | Trp(6-F) | Cys | * | +++ |
| | | | | aMeGlu | | | | | | | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 16
Compounds with Cyclo-Leu at X₃. All peptides are N-acetylated, have a disulfide linkage and contain a C-terminal
amide. When comparing the selectivity of MC4R v MC1R, larger values indicate selectivity towards MC4R. When
comparing the bias of MC4R B-arrestin v MC4R cAMP, larger values indicate bias towards B-arrestin.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1012 | Arg | Cys | Cyclo- | Gln | D-Phe | Arg | Trp | Cys | *** | +++ |
| | | | Leu | | | | | | | |
| 1108 | D-Nar | Cys | Cyclo- | 3-Pal | D-Phe | Arg | Trp(6-F) | Cys | ** | +++ |
| | | | Leu | | | | | | | |
| 1006 | Beta- | Cys | Cyclo- | His | D-Phe | Arg | Trp | Cys | ** | + |
| | homoArg | | Leu | | | | | | | |
| 1005 | D-Arg | Cys | Cyclo- | His | D-Phe | Arg | Trp | Cys | ** | + |
| | | | Leu | | | | | | | |
| 1052 | Beta- | Cys | Cyclo- | His | D-Phe | Arg | Trp(6-F) | Cys | ** | + |
| | homoArg | | Leu | | | | | | | |
| 1008 | Beta- | Cys | Cyclo- | His | D-Phe | Arg | Trp(6-Me) | Cys | ** | + |
| | homoArg | | Leu | | | | | | | |
| 1001 | Arg | Cys | Cyclo- | His | D-Phe | Arg | Trp | Cys | ** | + |
| | | | Leu | | | | | | | |
| 1009 | D-Arg | Cys | Cyclo- | His | D-Phe | Arg | Trp(6-Me) | Cys | ** | + |
| | | | Leu | | | | | | | |
| 1027 | Beta- | Cys | Cyclo- | His | D- | Arg | TRP | Cys | * | ++ |
| | homoArg | | Leu | | Phe(4-Cl) | | | | | |
| 1015 | D-Arg | Cys | Cyclo- | His | D- | Arg | Trp(6-Me) | Cys | * | +++ |
| | | | Leu | | Phe(4-Me) | | | | | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

TABLE 17
Compounds with L-aMeAsp at X₃. All peptides are N-acetylated, have a disulfide linkage and contain a C-terminal
amide. When comparing the selectivity of MC4R v MC1R, larger values indicate selectivity towards MC4R. When
comparing the bias of MC4R B-arrestin v MC4R cAMP, larger values indicate bias towards B-arrestin.

| | | | | | | | | | | Bias: MC4R |
|---|---|---|---|---|---|---|---|---|---|---|
| Molecule | | | | | | | | | Selectivity: | B-arrestin v |
| Name | X₁ | X₂ | X₃ | X₄ | X₅ | X₆ | X₇ | X₈ | MC4R v MC1R | MC4R cAMP |
| 1121 | D-Nar | Glu | L- | His | D-Phe(4-F) | Arg | Trp(6-F) | Dap | *** | + |
| | | | aMeAsp | | | | | | | |
| 1042 | D-Nar | Cys | L- | His | D-Phe | Arg | Trp(6-F) | Cys | *** | + |
| | | | aMeAsp | | | | | | | |
| 1064 | D-Nar | Cys | L- | His | D-Phe | Arg | Trp(6-Me) | Cys | | + |
| | | | aMeAsp | | | | | | | |
| 1062 | D-Nar | Cys | L- | His | D-Phe(4-F) | Arg | Trp(6-Me) | Cys | ** | + |
| | | | aMeAsp | | | | | | | |
| 1068 | D-Nar | Glu | L- | His | D-Phe | Arg | Trp(6-Me) | Dap | ** | + |
| | | | aMeAsp | | | | | | | |
| 1065 | beta- | Cys | L- | His | D-Phe | Arg | Trp(6-Me) | Cys | ** | + |
| | homoArg | | aMeAsp | | | | | | | |
| 1004 | Arg | Cys | L- | His | D-Phe | Arg | Trp | Cys | ** | + |
| | | | aMeAsp | | | | | | | |
| 1089 | D-Nar | Cys | L- | His | D- | Arg | Trp(6-F) | Cys | ** | ++ |
| | | | aMeAsp | | Phe(3-CF3) | | | | | |
| 1063 | D-Arg | Cys | L- | His | D- | Arg | Trp(6-Me) | Cys | ** | + |
| | | | aMeAsp | | Phe(4-F) | | | | | |
| 1120 | D-Nar | Glu | L- | His | D-Phe | Arg | Trp(6-F) | Dap | ** | + |
| | | | aMeAsp | | | | | | | |
| 1069 | D-Nar | Asp | L- | His | D-Phe | Arg | Trp(6-Me) | Dap | ** | ++ |
| | | | aMeAsp | | | | | | | |

* denotes MC4R vs MC1R selectivity having a range of 0.01 to <1.00,
** denotes MC4R vs MC1R selectivity having a range of ≥1.00 to ≤7.40,
*** denotes MC4R vs MC1R selectivity of about >7.40.
+ denotes MC4R B-arrestin v MC4R cAMP bias having a range of >0.00 to ≤2.00,
++ denotes MC4R B-arrestin v MC4R cAMP bias having a range of 2.00 to ≤10.00,
+++ denotes MC4R B-arrestin v MC4R cAMP bias having a value of >10.00.

Without being bound to particular theory, the data demonstrates that holding X₃ constant demonstrated that certain X₃ positions were amenable to retaining MC4R potency while losing MC1R potency. The data of Tables 18-21 illustrates how the X₄ position itself was not sufficient to generate the selectivity that the combination of X₃ and X₄ positions generated, even with the X₁, X₅, and/or X₇ positions modified to improve selectivity/potency on MC4R.

TABLE 18
Exemplary peptides with 3-Pal at X₄. All peptides are N-acetylated,
have a disulfide linkage and contain a C-terminal amide.

| Molecule | | | | | | | | |
|---|---|---|---|---|---|---|---|---|
| Name | X₁ | X₂ | X₃ | X₄ | X₅ | X₆ | X₇ | X₈ |
| 1119 | D-Nar | Cys | Phg | 3-Pal | D-Phe(4-F) | Arg | Trp(6-F) | Cys |
| 1122 | D-Nar | Cys | Aib(O- | 3-Pal | D-Phe(4-F) | Arg | Trp(6-F) | Cys |
| | | | cyclic) | | | | | |
| 1124 | D-Nar | Cys | D-aMeOrn | 3-Pal | D-Phe(4-F) | Arg | Trp(6-F) | Cys |
| 1108 | D-Nar | Cys | Cyclo-Leu | 3-Pal | D-Phe | Arg | Trp(6-F) | Cys |
| 1116 | D-Nar | Cys | L-aMeGlu | 3-Pal | D-Phe | Arg | Trp(6-Me) | Cys |

TABLE 19
Molecule 1119, Molecule 1122, and Molecule 1124 retain high
cAMP potency at MC4R, while other analogues with 3-Pal at X₄
do not. Molecule 1108 retains B-arrestin potency at MC4R
but loses cAMP potency, and Molecule 1116 loses potency across
both cAMP and B-arrestin. When comparing the selectivity
of MC4R v MC1R, larger values indicate selectivity towards
MC4R. When comparing the bias of MC4R B-arrestin v MC4R cAMP,
larger values indicate bias towards B-arrestin.

| | | Bias: MC4R |
|---|---|---|
| Compound | Selectivity: | B-arrestin v |
| Name | MC4R v MC1R | MC4R cAMP |
| 1119 | *** | ++ |
| 1122 | *** | ++ |
| 1124 | *** | ++ |
| 1108 | ** | +++ |
| 1116 | ** | +++ |

*denotes MC4R vs MC1R selectivity having a range of 0.01 to <1.00,
**denotes MC4R vs MC1R selectivity having a range of ≥1.00 to ≤7.40,
***denotes MC4R vs MC1R selectivity of about >7.40.
+denotes MC4R B-arrestin v MC4R cAMP bias having a range of >0.00 to ≤2.00,
++denotes MC4R B-arrestin v MC4R cAMP bias having a range of 2.00 to ≤10.00,
+++denotes MC4R B-arrestin v MC4R cAMP bias having a value of >10.00.

Without being bound to a particular theory, pairing Gln at X₄ with either D-aMeOrn or Aib(O-cyclic) at X₃ outperformed the next best substitution at X₃ by >2× on MC4R vs MC1R selectivity. When comparing the selectivity of MC4R v MC1R, larger values indicate selectivity towards MC4R. When comparing the bias of MC4R B-arrestin v MC4R cAMP, larger values indicate bias towards B-arrestin.

TABLE 20
Exemplary compounds with Gln at X₄

| | | Bias: MC4R |
|---|---|---|
| Compound | Selectivity: | B-arrestin v |
| Name | MC4R v MC1R | MC4R cAMP |
| 1094 | *** | ++ |
| 1093 | *** | ++ |
| 1092 | *** | ++ |
| 1091 | *** | ++ |
| 1096 | *** | ++ |
| 1012 | *** | +++ |
| 1099 | *** | +++ |
| 1070 | ** | ++ |
| 1013 | ** | +++ |
| 1098 | ** | ++ |
| 1011 | ** | ++ |
| 1090 | ** | ++ |
| 1097 | ** | ++ |
| 1014 | ** | ++ |
| 1158 | *** | |

*denotes MC4R vs MC1R selectivity having a range of 0.01 to <1.00,
**denotes MC4R vs MC1R selectivity having a range of ≥1.00 to ≤7.40,
***denotes MC4R vs MC1R selectivity of about >7.40.
+denotes MC4R B-arrestin v MC4R cAMP bias having a range of >0.00 to ≤2.00,
++denotes MC4R B-arrestin v MC4R cAMP bias having a range of 2.00 to ≤10.00,
+++denotes MC4R B-arrestin v MC4R cAMP bias having a value of >10.00.

While positions at X₁, X₃, X₅, and X₇ were initially chosen based on the properties of computer aided designs and functional testing of individual and combinatorial substitutions, the improvements in selectivity were unexpectedly based on specific combinations of amino acids at the X₃ and X₄ positions in combination with X₁, X₅, and/or X₇ positional changes.

In addition, it was observed that when X₈ is Pen, selectivity was unexpectedly improved compared to other X₈ groups (e.g. when compared to Cys at X₈). Peptides with X₈=Pen were found to be 2×-10× more selective for MC4R v MC1R compared to peptides with X₈=Cys.

Murine Weight Loss Data with Various Peptides

3-day efficacy study was conducted in DIO mice to evaluate the effect on murine weight of the MC4R agonistic peptides described herein. In summary, highly selective peptides demonstrated efficacy in DIO mouse models (FIG. 1).

### 3-Day Murine Acute Feeding and Weight Loss Assay, Once Daily Dosing

In a 3-day acute feeding and weight loss assay with daily subcutaneous dosing, peptides described herein with selectivity for MC4R vs MC1R generated weight loss similar to comparator molecule 1, setmelanotide (FIG. 2).

14-Day Murine Weight Loss Assay with Daily MC4R Agonist Dosing

In a 14-day assay, efficacy studies were performed using diet-induced obesity (DIO) C57BL/6 male mice (aged 18-20 weeks) to evaluate the weight loss effects of MC4R agonist peptides (e.g., molecule 1158). Mice were obtained from Gem Pharmatech, having been maintained on a 60% high-fat diet for 18-20 weeks prior to arrival. Upon delivery, mice were acclimated to the vivarium for two weeks to re-establish baseline weight and adjust to their environment. To enable accurate food intake measurements, mice were single-housed, which minimized stress as they had already been singly housed before arrival. On Day −1, mice were weighed and randomized into cohorts matched by weight and age (within a two-week age range). On Day 0, mice were weighed, and each mouse received a subcutaneous injection between the scapulae of 10 mg/kg test peptide in a formulation to support daily dosing. Post-injection, mice were observed cageside for 30 minutes. This procedure was repeated daily through Day 13. The study concluded on Day 13 with final measurements of body weight. Weight loss was reported as mean±SD percent change from Day 0 relative to a vehicle-treated group. Each treatment group included eight mice (N=8/group) (FIG. 3).

### Lipidated Variants of Peptides

Lipidated variants of Molecule 1119, Molecule 1094, and Molecule 1093 were generated. Additional variants of these 3 parent compounds with 3-Pal and Orn at X₄ for Molecule 1094 and Molecule 1093 were generated. Finally, the X₅ position for Molecule 1094 was edited to D-Phe(4-F) to modulate selectivity (Table 21).

Table 2 provided elsewhere herein, provides a detailed list of various lipidated peptides.

Tables 9 and 21, as listed below herein, provide an exemplary listing of lipidated variant peptides.

TABLE 9
Exemplary Lipidated Variant Peptides.

| Molecule | | | | | | | | | | | | | | | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | N-Term | C-term | Cyclic |
| 1150 | Lys* | D-Arg | Gly | D-Arg | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1142 | | Lys* | Gly | D-Arg | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1144 | | Lys^{a} | PEG1 | PEG1 | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1151 | Lys* | D-Arg | PEG1 | D-Arg | Beta- | Cys | Aib(O- | Gln | D- | Arg | Trp(6-F) | Cys | Ac | NH₂ | Disulfide |
| | | | | | homoArg | | cyclic) | | Phe(4-F) | | | | | | |
| 1152 | Lys* | D-Arg | Gly | D-Arg | D-Nar | Cys | Aib(O- | 3-Pal | D- | Arg | Trp(6-F) | Cys | Ac | NH₂ | Disulfide |
| | | | | | | | cyclic) | | Phe(4-F) | | | | | | |
| 1153 | Lys* | D-Arg | Gly | D-Arg | D-Nar | Cys | Aib(O- | Orn | D- | Arg | Trp(6-F) | Cys | 1153 | Lys* | D-Arg |
| | | | | | | | cyclic) | | Phe(4-F) | | | | | | |

Lys* = L-Lys(AEEAc-AEEAc-L-γ-Glu-17-carboxyheptadecanoyl)

TABLE 21
Lipidated Variant Peptides.

| Molecule | | | | | | | | | | | | | | | |
|---|---|---|---|---|---|---|---|---|---|---|---|---|---|---|---|
| Name | X₋₄ | X₋₃ | X₋₂ | X₋₁ | X₁ | X₂ | X₃ | X₄ | X₅ | X₆ | X₇ | X₈ | N-Term | C-term | Cyclic |
| 1146 | Lys* | D-Arg | Gly | D-Arg | D-Nar | Cys | Phg | 3-Pal | D- | Arg | Trp(6-F) | Cys | Ac | NH₂ | Disulfide |
| | | | | | | | | | Phe(4-F) | | | | | | |
| 1139 | | Lys* | Gly | D-Arg | D-Nar | Cys | Phg | 3-Pal | D- | Arg | Trp(6-F) | Cys | Ac | NH₂ | Disulfide |
| | | | | | | | | | Phe(4-F) | | | | | | |
| 1145 | | Lys* | PEG1 | PEG1 | D-Nar | Cys | Phg | 3-Pal | D- | Arg | Trp(6-F) | Cys | Ac | NH₂ | Disulfide |
| | | | | | | | | | Phe(4-F) | | | | | | |
| 1147 | Lys* | D-Arg | PEG1 | D-Arg | Beta- | Cys | Phg | 3-Pal | D- | Arg | Trp(6-F) | Cys | Ac | NH₂ | Disulfide |
| | | | | | homoArg | | | | Phe(4-F) | | | | | | |
| 1148 | Lys* | D-Arg | Gly | D-Arg | D-Nar | Cys | D- | Gln | D-Phe | Arg | Trp(6-F) | Cys | Ac | NH₂ | Disulfide |
| | | | | | | | aMeOrn | | | | | | | | |
| 1149 | | Lys* | Gly | D-Arg | D-Nar | Cys | D- | Gln | D-Phe | Arg | Trp(6-F) | Cys | Ac | NH₂ | Disulfide |
| | | | | | | | aMeOrn | | | | | | | | |
| 1137 | | Lys* | PEG1 | PEG1 | D-Nar | Cys | D- | Gln | D-Phe | Arg | Trp(6-F) | Cys | Ac | NH₂ | Disulfide |
| | | | | | | | aMeOrn | | | | | | | | |
| 1136 | Lys* | D-Arg | PEG1 | D-Arg | Beta- | Cys | D- | Gln | D-Phe | Arg | Trp(6-F) | Cys | Ac | NH₂ | Disulfide |
| | | | | | homoArg | | aMeOrn | | | | | | | | |
| 1150 | Lys* | D-Arg | Gly | D-Arg | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1142 | | Lys* | Gly | D-Arg | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1144 | | Lys* | PEG1 | PEG1 | D-Nar | Cys | Aib(O- | Gln | D- | Arg | Trp(6-F) | Cys | Ac | NH₂ | Disulfide |
| | | | | | | | cyclic) | | Phe(4-F) | | | | | | |
| 1151 | Lys* | D-Arg | PEG1 | D-Arg | Beta- | Cys | Aib(O- | Gln | D- | Arg | Trp(6-F) | Cys | Ac | NH₂ | Disulfide |
| | | | | | homoArg | | cyclic) | | Phe(4-F) | | | | | | |
| 1152 | Lys* | D-Arg | Gly | D-Arg | D-Nar | Cys | Aib(O- | 3- | D- | Arg | Trp(6-F) | Cys | Ac | NH₂ | Disulfide |
| | | | | | | | cyclic) | Pal | Phe(4-F) | | | | | | |
| 1153 | Lys* | D-Arg | Gly | D-Arg | D-Nar | Cys | Aib(O- | Orn | D- | Arg | Trp(6-F) | Cys | Ac | NH₂ | Disulfide |
| | | | | | | | cyclic) | | Phe(4-F) | | | | | | |
| 1154 | Lys* | D-Arg | Gly | D-Arg | D-Nar | Cys | D- | 3- | D- | Arg | Trp(6-F) | Cys | Ac | NH₂ | Disulfide |
| | | | | | | | aMeOrn | Pal | Phe(4-F) | | | | | | |
| 1155 | Lys* | D-Arg | Gly | D-Arg | D-Nar | Cys | D- | Orn | D- | Arg | Trp(6-F) | Cys | Ac | NH₂ | Disulfide |
| | | | | | | | aMeOrn | | Phe(4-F) | | | | | | |
| 1156 | Lys* | D-Arg | Gly | D-Arg | D-Nar | Cys | D- | 3-Pal | D-Phe | Arg | Trp(6-F) | Cys | Ac | NH₂ | Disulfide |
| | | | | | | | aMeOrn | | | | | | | | |
| 1157 | Lys* | D-Arg | Gly | D-Arg | D-Nar | Cys | D- | Orn | D-Phe | Arg | Trp(6-F) | Cys | Ac | NH₂ | Disulfide |
| | | | | | | | aMeOrn | | | | | | | | |

Lys* = L-Lys(AEEAc-AEEAc-L-γ-Glu-17-carboxyheptadecanoyl)

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

As used herein, all headings are simply for organization and are not intended to limit the disclosure in any manner. The content of any individual section may be equally applicable to all sections.

### INCORPORATION BY REFERENCE

All patents and publications referenced herein are hereby incorporated by reference in their entireties.

1. A peptide comprising the amino acid sequence of formula (I): 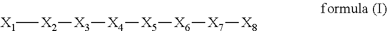
wherein in formula (I):
X₃ is 3-Aminooxetane-3-carboxylic acid (Aib(O-cyclic));
X₄ is glutamine (Gln), homocitrulline (hCit), citrulline (Cit), 3-(3-pyridyl)-L-alanine (3-Pal), L-homoglutamine (hGln), histidine (His), or L-ornithine (Orn); and
X₁, X₂, X₅, X₆, X₇, and X₈ are each independently a canonical or non-canonical amino acid.

2. The peptide of claim 1, wherein the peptide of formula (I) is a peptide of any one of formula (IIa), formula (Ib), formula (Ic), formula (Id), formula (Ie), or formula (If): 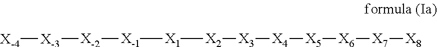
wherein in formula (IIa):
X₋₁, X₋₂, X₋₃, X₋₄, X₁, X₂, X₃, X₄, X₅, X₆, X₇, and X₈ are each independently an amino acid selected from Table 1, Table 2, Table 3, Table A1, Table A1A, Table A2, and Table A2A or a linker; 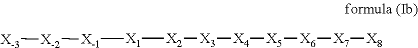
wherein in formula (Ib):
X₋₁, X₋₂, X₋₃, X₁, X₂, X₃, X₄, X₅, X₆, X₇, and X₈ are each independently an amino acid selected from Table 1, Table 2, Table 3, Table A1, Table A1A, Table A2, and Table A2A or a linker; 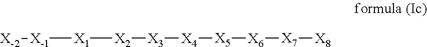
wherein in formula (Ic):
X₋₁, X₋₂, X₁, X₂, X₃, X₄, X₅, X₆, X₇, and X₈ are each independently an amino acid selected from Table 1, Table 2, Table 3, Table A1, Table A1A, Table A2, and Table A2A or a linker; 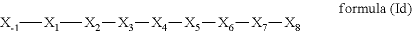
wherein in formula (Id):
X₋₁, X₁, X₂, X₃, X₄, X₅, X₆, X₇, and X₈ are each independently an amino acid selected from Table 1, Table 2, Table 3, Table A1, Table A1A, Table A2, and Table A2A or a linker; 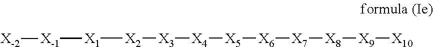
wherein in formula (Ie):
X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker; 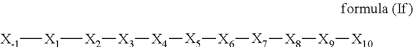
wherein in formula (If):
X₋₁, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker.

3. The peptide of any claim 1, wherein X₄ is Gln.

4. The peptide of claim 1, wherein X₅ is selected from 4-fluoro-D-phenylalanine (D-Phe(4-F)), D-phenylalanine (D-Phe), and 4-methyl-D-phenylalanine (D-Phe(4-Me)), optionally wherein X₅ is D-Phe(4-F).

5. The peptide of claim 1, wherein X₆ is arginine (Arg).

6. The peptide of claim 1, wherein X₇ is 6-fluoro-L-tryptophan (Trp(6-F)).

7. The peptide of claim 1, wherein X₈ is penicillamine (Pen) or cysteine (Cys), optionally wherein X₈ is penicillamine (Pen).

8. The peptide of claim 1, wherein X₁ is selected from D-norarginine (D-Nar) and beta-homo-L-arginine (Beta-homoArg), optionally wherein X₁ is D-norarginine (D-Nar).

9. The peptide of claim 1, wherein X₂ is Cys.

10. (canceled)

11. (canceled)

12. The peptide of claim 1, wherein the peptide is a cyclic peptide, optionally wherein the cyclic peptide comprises a disulfide bridge or a lactam bridge, and wherein the cyclic peptide is of any one of formula (II), formula (IIa), formula (IIb), formula (IIc), formula (IId), formula (IIe), or formula (IIf): 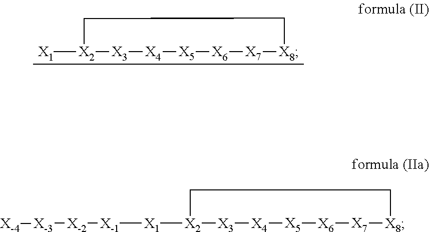
wherein in formula (IIa): X₋₁, X₋₂, X₋₃, X₋₄, X₁, X₂, X₃, X₄, X₅, X₆, X₇, and X₈ are each independently an amino acid selected from Table 1, Table 2, Table 3, Table A1, Table A1A, Table A2, and Table A2A or a linker; 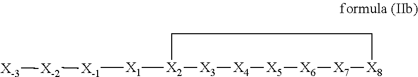
wherein in formula (IIb): X₋₁, X₋₂, X₋₃, X₁, X₂, X₃, X₄, X₅, X₆, X₇, and X₈ are each independently an amino acid selected from Table 1, Table 2, Table 3, Table A1, Table A1A, Table A2, and Table A2A or a linker; 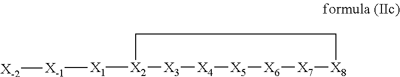
wherein in formula (IIc): X₋₁, X₋₂, X₁, X₂, X₃, X₄, X₅, X₆, X₇, and X₈ are each independently an amino acid selected from Table 1, Table 2, Table 3, Table A1, Table A1A, Table A2, and Table A2A or a linker; 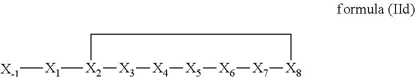
wherein in formula (IId): X₋₁, X₁, X₂, X₃, X₄, X₅, X₆, X₇, and X₈ are each independently an amino acid selected from Table 1, Table 2, Table 3, Table A1, Table A1A, Table A2, and Table A2A or a linker; 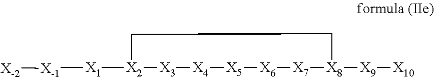
wherein in formula (IIe): X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker; 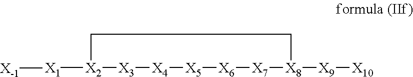
wherein in formula (IIf): X₋₁, X₋₂, X₁, X₂, X₃, X₄, X₅, X₆, X₇, X₈, X₉, and X₁₀ are each independently an amino acid selected from Table 1, Table 2, Table 3, Table A1, Table A1A, Table A2, and Table A2A or a linker.

13. The peptide of claim 1, wherein the peptide is capped with N-terminal acetyl and/or C-terminal amide groups.

14. The peptide of claim 1, wherein the peptide is selected from Table A1, Table A1A, Table A2, Table A2A, Table 1 and Table 2.

15. A peptide consisting of the amino acid sequence as set forth in formula (IV): 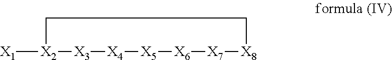
wherein in formula (IV):
X₁ is D-norarginine (D-Nar);
X₂ is cysteine (Cys);
X₃ is 3-Aminooxetane-3-carboxylic acid (Aib(O-cyclic));
X₄ is glutamine (Gln);
X₅ is 4-fluoro-D-phenylalanine (D-Phe(4-F));
X₆ is arginine (Arg);
X₇ is 6-fluoro-L-tryptophan (Trp(6-F)); and
X₈ is penicillamine (Pen), wherein 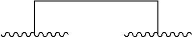 [SMILES: *CC*]
represents a disulfide bridge, and the peptide is capped with N-terminal acetyl.

16. The peptide of claim 1, wherein the peptide demonstrates one or more of (a)-(h):
(a) increased selectivity for MC4R over MC1R when administered to a subject compared to a control;
(b) increased selectivity for MC4R over MC1R when administered to a subject as measured by an in vitro, ex vivo, or in vivo assay when compared to a control;
(c) an increased ratio of MC4R intracellular signaling to MC1R intracellular signaling when administered to a subject compared to a control;
(d) increased selectivity for MC4R intracellular signaling to MC1R intracellular signaling as measured by an in vitro, ex vivo, or in vivo assay when compared to a control;
(e) enhanced melanocortin 4 receptor (MC4R) function in a subject when compared to before the peptide is administered or to a pre-treatment or non-treatment state, or a subject treated with control;
(f) decreased melanocortin 1 receptor (MC1R) function in a subject when compared to before the peptide is administered or to a pre-treatment or non-treatment state, or a subject treated with control;
(g) enhanced melanocortin 4 receptor (MC4R) function as measured by an in vitro, ex vivo, or in vivo assay when compared to a control;
(h) decreased melanocortin 1 receptor (MC1R) function as measured by an in vitro, ex vivo, or in vivo assay when compared to a control.

17. A protein comprising the peptide of claim 1.

18. A nucleic acid encoding the peptide of claim 1.

19. A solid synthesis device conjugated to the peptide of claim 1.

20. A pharmaceutical composition comprising a peptide of claim 1, and a pharmaceutically acceptable excipient or carrier.

21. A method of selectively increasing the activity of MC4R over MC1R comprising administering a peptide of claim 1 to a subject in need thereof, optionally wherein the method prevents, reduces, or eliminates hyperpigmentation in the subject when compared to an untreated or pre-treatment subject, or subject treated with a control.

22. A method of treating a disease or disorder associated with MC4R function comprising administering a peptide of claim 1 to a subject in need thereof, optionally wherein the disease or disorder is selected from genetic obesity, obesity, abnormal or excessive fat accumulation, hypothalamic obesity, surgically-acquired obesity, a metabolic disorder, an eating disorder, hypothyroidism, Cushing syndrome, obesity due to proopiomelanocortin (POMC) variance or deficiency, obesity due to proprotein convertase subtilisin/kexin type 1 (PCSK1) variance or deficiency, obesity due to leptin receptor (LEPR) variant or deficiency, Bardet-Biedl syndrome (BBS), MC4R allele variant or deficiency, and Prader-Willi syndrome.
